(19) United States
(12) Patent Application Publication
(10) Pub. No.: US 2015/0283227 A1
(43) Pub. Date: Oct. 8, 2015

(54) SYNTHETIC ENV PROTEINS

(71) Applicants: DUKE UNIVERSITY, Durham, NC (US); SLOAN-KETTERING INSTITUTE FOR CANCER RESEARCH, New York, NY (US); DANAFARBER CANCER INSTITUTE, Boston, MA (US)

(72) Inventors: Barton F. Haynes, Durham, NC (US); Hua-Xin Liao, Durham, NC (US); S. Munir Alam, Durham, NC (US); Samuel Danishefsky, New York, NY (US); Baptiste Aussedat, New York, NY (US); Peter K. Park, New York, NY (US); Yusuf Vohra, New York, NY (US); Joseph Sodroski, Boston, MA (US)

(21) Appl. No.: 14/438,591

(22) PCT Filed: Oct. 28, 2013
(86) PCT No.: PCT/US13/67063

§ 371 Date: Apr. 24, 2015

Related U.S. Application Data
(60) Provisional application No. 61/719,304, filed on Oct. 26, 2012.
(60) Provisional application No. 61/862,442, filed on Aug. 5, 2013.
(60) Provisional application No. 61/888,956, filed on Oct. 9, 2013.

(51) Int. Cl. A61K 39/21; C07K 16/10; C12N 7/00; C07K 14/005

(52) U.S. Cl. CPC A61K 39/21; C07K 14/005; C07K 16/1063; C12N 7/00; C07K 2317/76; C12N 2740/16134; C12N 2740/16122

(57) ABSTRACT

The present invention relates, in general, to human immunodeficiency virus-1 (HIV-1), and, in particular to a vaccine for HIV-1 and to methods of making and using same. The present invention provides synthetic glycosylated HIV-1 peptides, method for their preparation and use.

This application claims the benefit of priority from U.S. Ser. No. 61/719,304 filed Oct. 26, 2012, U.S. Ser. No. 61/862,442 filed Aug. 5, 2013, and U.S. Ser. No. 61/888,956 filed Oct. 9, 2013, the entire content of each application is herein incorporated by reference in its entirety.

This invention was made with government support under Grant Nos. A1 0678501 and A1100645 awarded by the National Institutes of Health. The government has certain rights in the invention.

### TECHNICAL FIELD

The present invention relates, in general, to human immunodeficiency virus-1 (HIV-1), and in particular, to a vaccine for HIV-1 and to methods of making and using same.

### BACKGROUND

New targets for HIV vaccine development have recently been discovered that focus on regions in the HIV envelope within the V1V2 region (McLellan et al, Nature 480:336 (2011)) and at the base of the V3 loop (Bonsignori et al, J. Virology 85:9998 (2011)). The C beta strand of V1V2 and the glycans at N160 and N156 are the targets of the V2V3 conformational broad neutralizing antibodies (BnAbs) PG9, PG16 and CH01-04 (McLellan et al, Nature 480:336 (2011), Bonsignori et al, J. Virology 85:9998 (2011)), and the N332 glycan is critical for binding of the new BnAbs, PGT 121, 125, 127, 128, 130 (Walker et al, Nature 477: 466 (2011)). While a minority of chronically infected HIV-1 persons can make antibodies to these peptide-glycan sites (i.e., N160, N156 and N332), to date, no envelope immunogen has been able to induce these types of antibodies.

The present invention relates, at least in part, to a synthetic peptide that is homogeneous and has preferred binding to the broad neutralizing antibodies PG9 and CH01 and binds weakly to the non-tier 2 neutralizing antibody, CH58, and minimally to its reverted unmutated ancestor antibody (RUA). The invention includes peptide glycans, such as the V1/V2 Man₃GlcNac₂ and the V1/V2 Man₅GlcNac₂ peptide glycans, which preferentially can induce PG9- and CH01-like BnAbs when used as an immunogen.

### SUMMARY OF THE INVENTION

The present invention relates, in general, to human immunodeficiency virus-1 (HIV-1), and in particular, to a vaccine for HIV-1 and to methods of making and using same.

In certain aspects, the invention provides a synthetic peptide comprising, consisting essentially of, consisting of sequence ITDEVRNCSFNMTTELRDKKQKVHALFYKLDIVPI (SEQ ID NO: 1), wherein the peptide is glycosylated at position Asn156 and Asn160 (amino acids are underlined). In certain embodiments, the invention provides a peptide consisting essentially of sequence ITDEVRNCSFNMTTELRDKKQKVHALFYKLDIVPI (SEQ ID NO: 1), wherein the peptide is glycosylated at position N156 and N160. In certain embodiments, the inventive peptide is not recombinantly made or naturally occurring. In certain embodiments, the peptide is glycosylated with polysaccharide comprising oligomannose. In certain embodiments, the oligomannose is trimannose or pentamannose. In certain embodiments, the oligomannose is pentamannose. In certain embodiments, the peptide has Man₅GlcNAc₂ glycans at position N156 and N160. In certain embodiments, the peptide has Man₃GlcNAc₂ glycans at position N156 and N160.

In certain aspects, the invention provides a synthetic glycopeptide of Formula Man₃GlcNAc₂ V1V2 “Compound 2/Peptide2” or of Formula Man₅GlcNAc₂ V1V2 “Compound 1/Peptide 1”. In certain embodiments, the synthetic glycopeptide is Man₃GlcNAc₂ V1V2. In certain embodiments, synthetic glycopeptide is Man₅GlcNAc₂ V1V2.

In certain aspects, the invention provides a peptide dimer comprising, consisting essentially of, or consisting of the synthetic glycopeptide of Man₅GlcNAc₂ V1V2 (Peptide 1). In certain embodiment, the dimer is disulfide-linked. In certain embodiment, the dimer is linked via oxidized Cys157. In certain aspects, the invention provides a peptide dimer comprising, consisting essentially of, or consisting of the synthetic glycopeptide of Man₃GlcNAc₂ V1V2 (Peptide 2). In certain embodiments, the dimer is disulfide-linked. In certain embodiments, the dimer is linked via oxidized Cys157. Skilled artisan would appreciate and readily determine various conditions that could produce disulfide-linked dimers (e.g., see URL: currentprotocols.com/WileyCDA/CPUnit/refId-ps1806.html). In certain embodiments, DMSO in aqueous buffer as described herein was the only one that also provided the dimers in the desired conformation.

In certain embodiment, the invention provides a composition comprising any one of the inventive peptides, wherein the composition comprises purified homogenously glycosylated peptides. In certain embodiments, about 70%, 75%, 80%, 85%, 90%, 95%, 99%, or 99.9% of the peptides in the composition are homogenously glycosylated peptides. In certain embodiments, 70%, 75%, 80%, 85%, 90%, 95%, 99%, or 99.9% of the peptides in the composition are homogenously glycosylated peptides. In certain embodiments, 70%-75%, 75.1%-80%, 80.1%-85%, 85.1%-90%, 90.1%-95%, 95.1%-99%, 96%-99%, 97%-99%, 98%-99% or 99.9% of the peptides in the composition are homogenously glycosylated peptides. In certain embodiment, the glycosylation pattern is homogenous on all peptides of SEQ ID NO: 1 in the composition. In certain embodiment, the glycosylation pattern is substantially identical on all peptides of SEQ ID NO: 1 in the composition.

In certain embodiments of the composition, the peptide comprises an oxidized Cys157. In certain embodiments of the composition, the peptide is a dimer. In certain embodiments, the dimer is disulfide-linked. In certain embodiments, the dimer is linked via oxidized Cys157. In certain embodiments, the compositions and peptides of the invention are immunogenic. In certain embodiments, the composition comprises an adjuvant.

In certain aspects, the invention provides a method of inducing an antibody or antibodies against HIV-1 in a subject, the method comprising administering to the subject composition comprising an inventive peptide or a dimer thereof, in an amount sufficient to induce the anti-HIV-1 antibody/antibodies. In certain embodiments, the composition comprises Man5GlcNAc₂ V1V2 as a dimer and an adjuvant. In certain embodiments, the dimer is disulfide-linked. In certain embodiments, the dimer is linked via oxidized Cys157.

In certain embodiments, the composition is administered as a prime, boost, or both. In certain embodiments, the antibody induced by the immunogenic compositions and methods of the invention binds an epitope comprised within Peptide 1, the dimer of Peptide 1, within Peptide 2, the dimer of Peptide 2, or the peptide of SEQ ID NO: 1.

In certain aspects the invention provides an isolated or recombinant antibody which binds an epitope comprised within Peptide 1, the dimer of Peptide 1, within Peptide 2, the dimer of Peptide 2, or the peptide of SEQ ID NO: 1. In certain embodiments, the antibody does not bind to the non-glycosylated peptide of SEQ ID NO: 1 (Aglycone V1V2 peptide of SEQ ID NO: 1). In certain embodiments, the antibody binds substantially less to the non-glycosylated peptide of SEQ ID NO: 1 (Aglycone V1V2 peptide of SEQ ID NO: 1). In certain embodiments, the binding to the monomer and dimer could be with different affinities. In certain embodiments, the antibody is monoclonal.

In certain aspects, the invention provides a method for synthesizing Peptide 1, comprising ligating glycopeptide N-terminal fragment 22 and glycopeptide C-terminal fragment 24 in NCL buffer and neutral TCEP solution (Scheme 5 step (e)). Provided herein are also methods to synthesize Peptide 2.

In certain aspects, the invention provides a method for synthesizing glycopeptide N-terminal fragment 22 (ITDEVRN is SEQ ID NO: 2), comprising joining the carboxylic acid side chain at position 156 of the thioester peptide ITDEVRD (fragment 21 Scheme 5; ITDEVRD SEQ ID NO: 3) to Man₅GlcNAc₂ (heptasaccharide 18) in the presence of PyAOP, DIEA, DMSO, optionally lyophilizing the mixture, and precipitating the glycopeptide by a treatment with 85:5:5:2 TFAphenol/water/triisopropylsilane (Scheme 5). In certain aspects, the invention provides a method for synthesizing glycopeptide C-terminal fragment 24 (CSFNMTTELRDKKQKVHALFYKLDIVPI is SEQ ID NO: 4), comprising joining the side chain at position 160 of the peptide of fragment 23 (CSFDMTTELRDKKQKVHALFYKLDIVPI is SEQ ID NO: 5) (Scheme 5) to Man₅GlcNAc₂ (heptasaccharide 18) in the presence of PyAOP, DIEA, DMSO, quenching the reaction in TFA, optionally lyophilizing the mixture, and precipitating the glycopeptide by a treatment with 90:5:3:2 TFA/thioanisole/ethanedithiol/anisole (Scheme 5 step (c, d)). Provided herein are methods to synthesize the N- and C-terminal fragments of Peptide 2.

Objects and advantages of the present invention will be clear from the description that follows.

### BRIEF DESCRIPTION OF THE DRAWINGS

FIG. 1. New RV144 V2 human mAbs—CH58 and CH59.

FIG. 2. New RV144 V2 human mAbs—CH58, CH59, HG107 and HG120.

FIG. 3. PG9, CH01 broad neutralizing HIV-1 antibodies bind to the same Env regions as CH58, CH59 RV144 Abs.

FIG. 4. V1/V2 Aglycone.

FIG. 5. V1/V2 GlcNAc₂.

FIG. 6. V1/V2 Man₃GlcNAc₂.

FIG. 7. V1/V2 Man₅GlcNAc₂.

FIG. 8. Peptide glycan designs for N331 or N332 in red (see dot above “N”) depending on the HIV isolate.

FIGS. 9A-9D. Selective binding of V1/V2 broadly neutralizing mAbs to synthetic V1/V2 glycopeptides. FIG. 9A) Only the V1/V2 mAb CH58 bound to the glycan-deficient (aglycone) peptide. V1/V2 bNAbs (PGG9, CH01) bound weakly to V1/V2 GlcNAc₂ peptide (FIG. 9B). Both BnAbs PG9 and CH01 bound avidly to the glycopeptides, V1/V2 Man₃GlcNAc₂ and V1/V2 Man₅GlcNAc₂ (FIGS. 9C, 9D)

FIGS. 10A-10D, FIG. 10A) CH58 binds more avidly to A244 V1v2 tags protein when compared to the binding of bNAbs PG9 or CH01. FIGS. 10B and 10C) BNabs PG9 and CH01 bind selectively to the glycopeptides V1/V2 Man₃GlcNAc₂ and V1/V2 Man₅GlcNAc₂. FIG. 10D) MAb CH58 binds avidly to A244 V1v2 tags protein and weakly with fast dissociation rates to V1V2 glycopeptides.

FIGS. 11A-11D. Binding of V1/V2 unmutated ancestor (UA) antibodies to synthetic V1/V2 aglycone and glycopeptides. FIG. 11A) V1V2 aglycone peptide. FIG. 11B) V1/V2 GlcNAc₂. FIG. 11C) V1/V2 Man₃GlcNAc₂. FIG. 11D) V1/V2 Man₅GlcNAc₂.

FIGS. 12A-12D. Binding of a panel of V2 and V1/V2 mAbs to aglycone (FIG. 12A), V1/V2 GlcNAc₂ (FIG. 12B), V1/V2 Man₃GlcNAc₂ (FIG. 12C) and V1/V2 Man₅GlcNAc₂ (FIG. 12D).

FIGS. 13A-13C. Binding of UAs of conformational V1 V2 (PG (FIG. 13A), CH01 (FIG. 13B)) and V2 (697D (FIG. 13C) to V1/V2 Man₅GlcNAc₂.

FIGS. 14A and 14B. Glycopeptide target structures. (FIG. 14A) Chemical structure of Man₅GlcNAc₂-Asn. (FIG. 14B) Glycopeptide fragments derived from the V1/V2 region gp120 bearing two N-linked Man₅GlcNAc₂ (1) or Man₃GlcNAc₂ (2) oligosaccharides at N156 and N160 (V1/V2 sequence derived from AE.CM244 strain, displayed with HXB2 numbering). The N- and C-termini are modified with acetyl and carboxamide moieties, respectively, to increase stability to exopeptidases and avoid the formation of non-natural charges at the ends of the peptides.

FIG. 15. Synthetic strategy to access Man₅GlcNAc₂ heptasaccharide 4.

FIG. 16. Synthesis of tetrasaccharide core 11.

FIG. 17. Synthesis of Man₃GlcNAc₂ pentasaccharide 15.

FIG. 18. Synthesis of branched trimannoside 7.

FIG. 19. Synthesis of Man₅GlcNAc₂ heptasaccharide 3.

FIG. 20. Synthesis of glycopeptide 1 bearing two Man₅GlcNAc₂ units.

FIG. 21. Synthesis of glycopeptide 2 bearing two Man₃GlcNAc₂ units.

FIG. 22. Plan for generating modified glycopeptides suitable for thiol-based bioconjugation chemistry using a C-terminus cysteine.

FIG. 23. Alternative plan for generating thiol-modified glycopeptides using a modified glutamate side chain at the C-terminus.

FIG. 24. Plan for conjugating glycopeptides to carrier proteins using thiol-maleimide coupling chemistry

FIG. 25. Plan for conjugating glycopeptides to carrier proteins using thiol-ene coupling chemistry.

FIG. 26. A—ESI-MS of compound S-12. ESI calculated for C₆₄H₁₀₄N₁₀O₁₈S₂ [M+H]⁺ m/z: 1366.7. found: 1366.6; [M+2H]²⁺ m/z: 683.85. found: 683.67; [4M+3H]³⁺ m/z: 1821.93. found: 1821.81. B—UV trace from UPLC analysis of purified compound S-12; gradient: 50% to 95% acetonitrile/water over 6 min at a flow rate of 0.3 mL/min, BEH C4 column.

FIG. 27. A—ESI-MS of compound S-13. ESI calculated for C₅₄H₉₁N₁₃O₂₄S [M+H]⁺ m/z: 1339.44. found: 1339.30; [M+2H]²⁺ m/z: 670.02. found: 670.22. B—UV trace from UPLC analysis of purified compound S-13; gradient: 10% to 60% acetonitrile/water over 6 min at a flow rate of 0.3 mL/min, BEH C18 column.

FIG. 28: A—ESI-MS of compound S-14. ESI calculated for C₇₂H₁₂₁N₁₃O₃₉S [M+2H]²⁺ m/z: 913.43. found: 913.13; [2M+3H]³⁺ m/z: 1217.57. found: 1217.32; [3M+4H]⁴⁺ m/z: 1369.64. found: 1369.45. B—UV trace from UPLC analysis of purified compound S-14; gradient: 10% to 60% acetonitrile/water over 6 min at a flow rate of 0.3 mL/min, BEH C18 column.

FIG. 29: A—UV trace from UPLC analysis of the crude mixture obtained after one-flask aspartylation/deprotection. The star (*) indicates a side product of identical mass, presumably due to epimerization of the thioester; gradient: 10% to 60% acetonitrile/water over 6 min at a flow rate of 0.3 mL/min, BEH C18 column. B—ESI-MS of compound S-15. ESI calculated for C₈₄H₁₄₁N₁₃O₄₉S [M+2H]²⁺ m/z: 1075.57. found: 1075.31; [2M+3H]³⁺ m/z: 1433.76. found: 1433.60; [3M+4H]⁴⁺ m/z: 1612.85. found: 1612.67; [4M+5H]⁵⁺ m/z: 1720.31. found: 1720.47; [5M+6H]⁶⁺ m/z: 1791.91. found: 1791.95. C—UV trace from UPLC analysis of purified compound S-15; gradient: 10% to 60% acetonitrile/water over 6 min at a flow rate of 0.3 mL/min, BEH C18 column.

FIG. 30: A—ESI-MS of compound S-16. ESI calculated for C₅₄₇H₈₅₈N₁₀₄O₁₄₆S₈ [M+3H]³⁺ m/z: 1259.8. found: 1260.1; [M+4H]⁴⁺ m/z: 1679.4. found: 1679.8. B—UV trace from UPLC analysis of compound S-16; The star (*) indicates product S-16, a-c correspond to capped truncation products with the presumed structures shown below; gradient: 85% to 99% acetonitrile/water over 6 min at a flow rate of 0.3 mL/min, BEH C4 column,

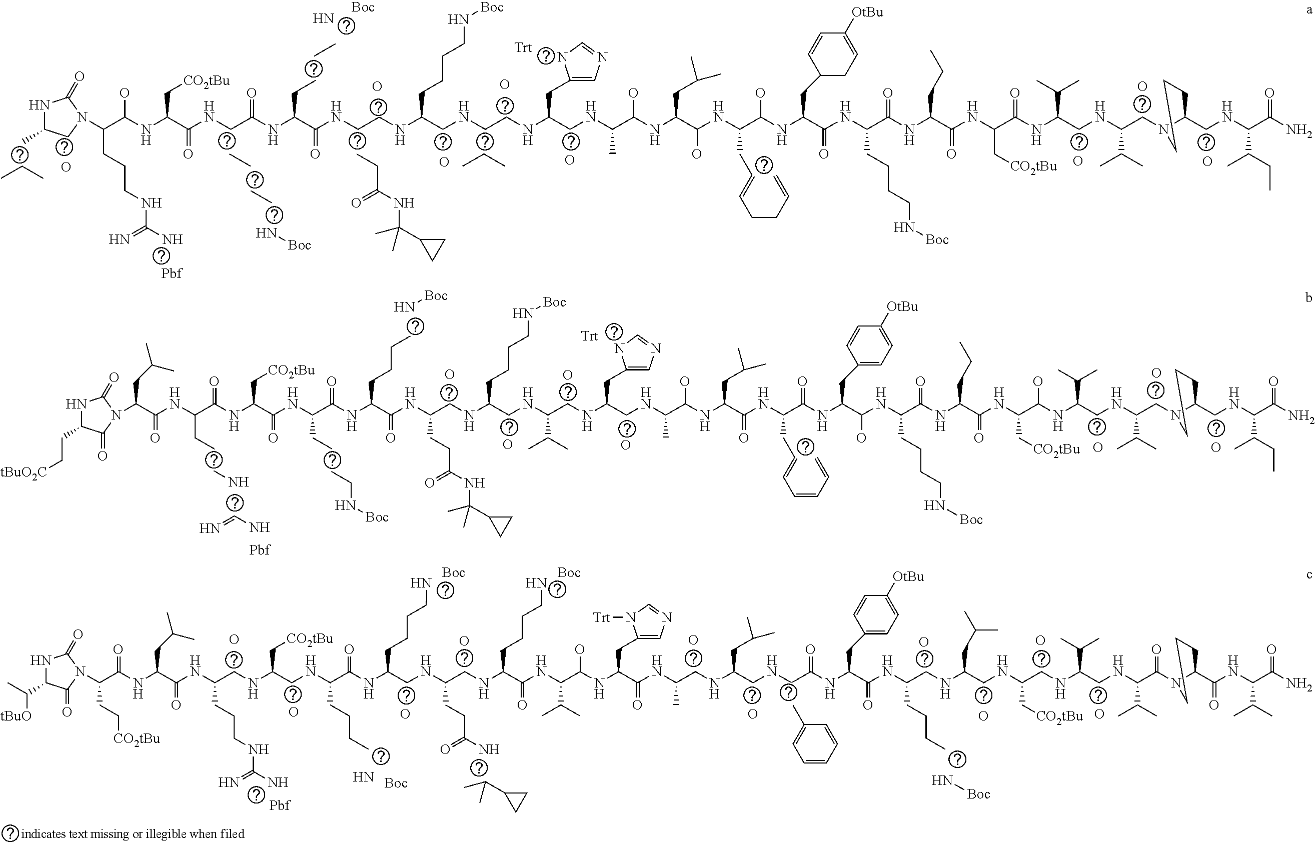

FIG. 31: A—ESI-MS of compound S-17. ESI calculated for C₁₆₈H₂₇₂N₄₂O₅₀S₂ [M+4H]⁴⁺ m/z: 937.1. found: 937.1; [M+3H]³⁺ m/z: 1249.1. found: 1248.9. B—UV trace from UPLC analysis of purified compound S-17; gradient: 10% to 60% acetonitrile/water over 6 min at a flow rate of 0.3 mL/min. BEH C4 column.

FIG. 32: A—ESI-MS of compound S-18. ESI calculated for C₁₈₆H₃₀₂N₄₂O₆₅S₂ [M+5H]⁵⁺ m/z: 847.15. found: 846.9; [M+4H]⁴⁺ m/z: 1058.69. found: 1058.58; [M+3H]³⁺ m/z: 1411.25. found: 1411.02; [2M+5H]⁵⁺ m/z: 1693.3. found: 1692.86. B—UV trace from UPLC analysis of purified compound S-18; gradient: 10% to 60% acetonitrile/water over 6 min at a flow rate of 0.3 mL/min, BEH C4 column.

FIG. 33: A—ESI-MS of compound 24. ESI calculated for C₁₉₈H₃₂₂N₄₂O₇₅S₂ [M+4H]⁴⁺ m/z: 1139.76. found: 1139.60; [M+3H]³⁺ m/z: 1519.34. found: 1519.04; [2M+5H]⁵⁺ m/z: 1823.01. found: 1822.56. B—UV trace from UPLC analysis of purified compound 24; gradient: 10% to 60% acetonitrile/water over 6 min at a flow rate of 0.3 mL/min, BEH C4 column.

FIG. 34: A—ESI-MS of compound S-19. ESI calculated for C₁₈₈H₃₀₅N₅₁O₅₄S₂ [M+5H]⁵⁺ m/z: 842.6. found: 842.3; [M+4H]⁴⁺ m/z: 1053.0. found: 1052.8; [M+3H]³⁺ m/z: 1403.6. found: 1403.4; [2M+5H]⁵⁺ m/z: 1684.1. found: 1684.2. B—UV trace from UPLC analysis of purified compound S-19; gradient: 10% to 60% acetonitrile/water over 6 min at a flow rate of 0.3 mL/min, BEH C4 column.

FIG. 35: A—ESI-MS of compound 3. ESI calculated for C₂₂₀H₃₅₇N₅₅O₇₄S₂ [M+5H]⁵⁺ m/z: 1005.1. found: 1006.0; [M+4H]⁴⁺ m/z: 1256.2. found: 1256.6; [M+3H]³⁺ m/z: 1674.5. found: 1675.3. B—UV trace from UPLC analysis of purified compound 3; gradient: 10% to 60% acetonitrile/water over 6 min at a flow rate of 0.3 mL/min, BEH C4 column.

FIG. 36: A—ESI-MS of compound 2. ESI calculated for C₂₅₆H₄₁₇N₅₅O₁₀₄S₂ [M+5H]⁵⁺ m/z: 1198.8. found: 1199.6; [M+4H]⁴⁺ m/z: 1499.4. found: 1499.2; [M+5H]⁵⁺ m/z: 1998.8. found: 1998.8. B—UV trace from UPLC analysis of purified compound 2; gradient: 10% to 60% acetonitrile/water over 6 min at a flow rate of 0.3 mL/min, BEH C4 column.

FIG. 37: A—ESI-MS of compound 1. ESI calculated for C₂₈₀H₄₅₇N₅₅O₁₂₄S₂ [M+6H]⁶⁺ m/z: 1108.0. found: 1107.8; [M+5H]⁵⁺ m/z: 1329.4. found: 1329.3; [M+4H]⁴⁺ m/z: 1661.5. found: 1661.3; [M+3H]³⁺ m/z: 2215.0. found: 2214.7. B—UV trace from UPLC analysis of purified compound 1; gradient: 10% to 60% acetonitrile/water over 6 min at a flow rate of 0.3 mL/min, BEH C4 column. C—UV trace from UPLC analysis of the native chemical ligation performed to access 1 (*); peak a corresponds to the cyclized product shown above, peak b corresponds to 22, and peak c corresponds to the transthioesterification product of 22 with MPAA; gradient: 20% to 55% acetonitrile/water over 6 min at a flow rate of 0.3 mL/min, BEH C8 column.

FIG. 38: Design of gp120 V1V2 domain broadly neutralizing epitope mimics. (A) Crystal structure of a scaffolded V1V2 domain from the CAP45 strain of HIV-1 (red ribbons) in complex with PG9 Fab (gray surface) (PDB ID 3U4E with scaffold hidden). The glycans at N160 and N156 are depicted with colored spheres representing atoms of the mannose (green) and N-acetylglucosamine (blue) residues. Disulfide bonds are shown as yellow sticks. Dashed arrows indicate where the disordered region of the V2 loop would be connected. Figure was created using PyMOL. (B) Schematic of the Greek key topology of the V1V2 domain. Strands are represented as arrows and disulfide bonds as yellow bars. (C) Chemical structure of Man₅GlcNAc₂-Asn. (D) Structures of candidate BnAb antigens, derived from residues 148-184 of the A244 strain gp120 (HXB2 numbering), encompassing the B and C β-strands (approximate location shown with red arrows) of the V1V2 domain, and bearing two N-linked Man₅GlcNAc₂, Man₃GlcNAc₂, or GlcNAc₂ oligosaccharides.

FIG. 39: Binding of mAb PG9 to gp120 V1V2 glycopeptides. SPR sensorgrams showing binding of mAb PG9 to V1V2 glycopeptides derivatized with Man₅GlcNAc₂ (A) and Man₃GlcNAc₂ (B). V1V2 Man₅GlcNAc₂ binding curves are shown for glycopeptide concentrations at 5, 10, 20, 30 and 40 μg/mL and V1V2 Man₃GlcNAc₂ at 1, 2, 5, 10 and 20 μg/mL. Control SPR sensograms showing minimal to no binding of mAb PG9 to V1V2 GlcNAc₂ (C), V1V2 aglycone (D), Man₅GlcNAc₂ glycan alone (E), and Man₃GlcNAc₂ glycan alone (F). V1V2 GlcNAc₂ and aglycone peptides were injected at 200 μg/mL (C, D) and Man₅GlcNAc₂ and Man₃GlcNAc₂ glycans at 25 μg/mL (E, F) over PG9 captured on anti-human IgG (Fc-specific) surfaces. SPR data were derived following subtraction of non-specific signal on a control anti-RSV mAb (Synagis, red curve in C-F).

FIG. 40: Selected NMR Spectra.

FIGS. 41A-41C. V1V2 glycopeptides form disulfide linked dimers. (FIG. 41A) SDS-PAGE analysis of V1V2 glycopeptides showing dimer under non-reducing and monomers under reducing conditions. Data are representative of at least three independent experiments. (FIG. 41B) Size exclusion chromatography of oxidized Man3 (FIG. 41B) and Man5 (FIG. 41C)-derivatized glycopeptides showing a single dimeric peak. Molecular sizes of protein standards are marked. The Vₑ (10.87 mL) of Man3 C157A mutant, which does not form disulfide-linked dimer, is marked with an arrow and asterisk.

FIGS. 42A-42D. Selective binding of V1V2 BnAbs to mannose derivatized V1V2 glycopeptides but not to aglycone or GlcNAc₂ V1V2 peptides. SPR curves showing preferential binding of PG9 and CH01 BnAbs to Man5—(FIG. 42A) and Man3—(FIG. 42B) GlcNAc₂ V1V2 glycopeptides but not to GlcNAc₂ (FIG. 42C) and aglycone (FIG. 42D) peptides. By contrast, V2 mAbs CH58 and CH59 bound to both aglycone (FIG. 42C) and GlcNAc₂ (FIG. 42D) V1V2 peptides. Each V1V2 peptide was oxidized by solubilization in DMSO and injected over the indicated MAb at 50 ug/mL. Data shown is after reference subtraction of non-specific signal measured over the control mAb (Synagis). Binding data are representative of at least three experiments for Man5 and Man3 V1V2 peptides and two experiments for GlcNAc₂ and aglycone V1V2.

FIGS. 43A-43D. Circular dichroism (CD) analyses of the secondary structure of the synthetic V1V2 peptides. V1V2 peptides derivatized with oligomannose units, Man5 (FIG. 43A) or Man3 (FIG. 43B)-GlcNAc₂ V1V2 or only the proximal GlcNAc₂-V1V2 (FIG. 43C) peptides show predominantly ordered secondary structure with β-strand and helical conformation. In (FIG. 43D), Man3 and Man5 V1V2 glycopeptides were oxidized by iodine treatment and CD analysis performed as above. CD spectra of each of the V1V2 peptides were taken at least two times. V1V2 peptides were solubilized in DMSO and allowed to fully dimerize in 20% DMSO-phosphate buffer for about 20 h. The CD spectra deconvolution analysis (K2D3) of Man 5 glycopeptide gave an estimated 23% β-strand, Man3 V1V2 glycopeptide gave 33%β-strand and 17% for GlcNAc₂ V1V2.

FIGS. 44A and 44B. Circular dichroism (CD) secondary structure and antigenicity of C157A mutant Man3 V1V2 glycopeptide. FIG. 44A) CD spectrum of C157A Man3-GlcNAc₂ mutant showing glycopeptide in random coil conformation and lack of the signature β-sheet features. FIG. 44B) CH58 mAb but not the V1V2 BnAbs (PG9, CH01) bound to the C157A mutant V1V2 Man3 GlcNAc2 peptide (injected at 50 μg/mL). A second experiment in which Man3 C157A glycopeptide was initially solubilized in 20% DMSO (as described in Example 6), gave similar binding to CH58 mAb but not to either PG9 or CH01 V1V2 BnAbs.

FIGS. 45A-45F. Surface plasmon resonance (SPR) measurements of PG9 and CH01 BnAb binding to dimerized V1V2 glycopeptides. V1V2 BnAbs PG9 (FIGS. 45A and 45B) and CH01 (FIGS. 45C and 45D) binding to varying concentrations of Man5 GlcNAc₂ (FIGS. 45A and 45C) and Man3 GlcNAc₂ V1V2 (FIGS. 45B and 45D). CH58 mAb binding to Man5 GlcNAc2 (FIG. 45E) and Man3 GlcNAc2 (FIG. 45F). V1V2 glycopeptides were injected at concentrations ranging from 1 to 10 μg/mL for PG9 and CH01, and from 1-50 μg/mL for CH58 mAb; and data are representative of at least three measurements for PG9 and CH01 binding to either Man5 or Man3 V1V2 glycopeptides. V1V2 peptides were solubilized in 20% DMSO overnight to allow complete dimer formation.

FIGS. 46A-46F. Binding of BnAb UCAs and CH58 UCA to synthetic V1V2 glycopeptides. Man5 GlcNAc₂ V1V2 glycopeptide was at concentrations ranging from 2 to 25 μg/mL binding to PG9 UCA (FIG. 46A) or CH01 UCA (FIG. 46B). Man3 GlcNAc₂ V1V2 at concentrations ranging from 1-8 μg/mL binding to PG9 UCA (FIG. 46C) or CH01 UCA (FIG. 46D). Man5 (FIG. 46E) and Man3 (FIG. 46F) glycopeptides were injected at concentrations ranging from 1-10 μg/mL over CH58 UCA captured on anti-IgG immobilized surface as above. Both peptides were solubilized in 20% DMSO overnight to allow complete dimer formation as described in Example 6.

FIG. 47. Schematic of V1V2 peptides (Aussedat et al., 2013, J Am Chem Soc, Epub ahead of print).

FIGS. 48A-48D. Spontaneously oxidized (air oxidation, FIGS. 48A and 48B) or iodine oxidized V1V2 glycopeptides (FIG. 48C and FIG. 48D) show binding to V2 mAb CH58 but weak or no binding to PG9 and CH01 bNAbs. Binding of glycopeptide Man5 (FIGS. 48A and 48C) or Man3 (FIGS. 48B and 48D) V1V2 at 50 ug/mL are shown. Binding curves of the BnAbs are color coded for CH01 in blue, and PG9 in red, while V2 Mab CH58 is shown in green.

FIGS. 49A-49D. Solubilization of V1V2 peptide in DMSO promotes adoption of an ordered secondary structure.

FIG. 50. SDS-PAGE analysis under non-reducing (NR) or reducing (R) condition shows relative proportions of disulfide-linked dimers in each of the indicated V1V2 glycopeptides. Both aglycone and GlcNAc2 V1V2 peptides solubilized in DMSO show the presence of monomers and dimers.

### DETAILED DESCRIPTION OF THE INVENTION

The RV144 ALVAC-HIV/AIDSVAX gp120 B/E vaccine trial in Thailand was partially successful and showed an estimated vaccine efficacy of 31.2% (Rerks-Ngarm et al, New Eng J. Med. 361:2209 (2009)). In a recent study of the correlates of infection risk in the trial, it was demonstrated that one correlate of reduced risk of infection was antibodies to the V1V2 gp120 region (Haynes et al, NEJM in re-review (2012)).

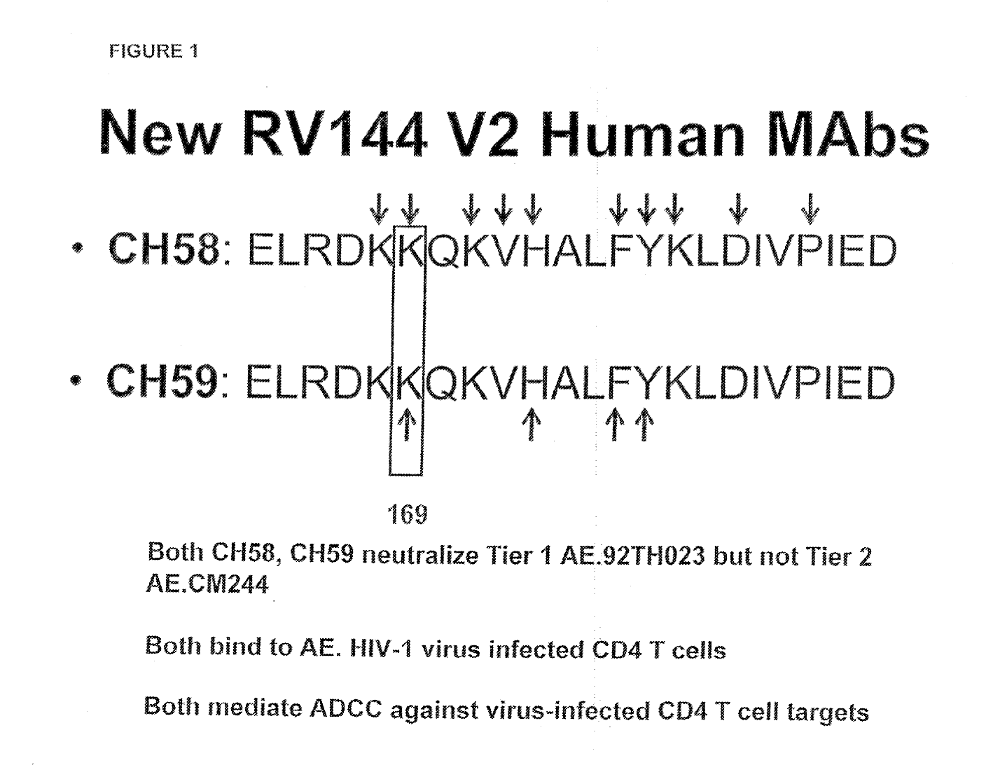

Analysis of the breakthrough HIV-1 infections in the RV144 trials demonstrated immune pressure at K169 in the C beta strand of V1V2 (Rolland et al, Nature 490:417-420, doi:10.1038/nature11519 (2012)). Two V2 gp120 antibodies isolated from RV144 trial subjects (mAbs CH58 and CH59) bind to this precise site of V1/V2 (see FIGS. 1-3), raising the hypothesis that this type of easily induced V2 antibody, if induced in high amounts, may be able to be protective (Haynes et al, NEJM in re-review (2012), Haynes et al, submitted (2012)). Thus, design of peptide-glycan conjugates that can optimally induce CD4 T cell and antibodies to the C beta strand N156, N160 gp120-glycan site is expected to be a key pathway for induction of better potentially protective antibodies than were induced in RV144. Moreover, a prerequisite for induction of BnAb activity appears to be induction of not only protein antibody reactivity but antibodies that bind directly to glycans. Thus, a major target of design of these constructs is to determine if they can induce antibodies to the N156 and N160 glycans. The first step in this work is to determine if the mature PG9 and CH01 V1/V2 antibodies can bind to synthetic peptide-glycan conjugates, and if so, then use the peptide-glycan as an immunogen.

One reason that BnAbs are not induced is that antibody responses to conserved BnAb Env epitopes are subdominant, i.e., are not made in sufficient amounts to be present in plasma after immunization. However, after long periods of time, 10-20% of subjects can indeed make BnAbs of varying specificities. One reason that subdominant BnAbs are not robustly induced is that the induction of the BnAb is controlled by host tolerance mechanisms (Verkoczy et al, PNAS (USA) 107:181-6 (2010); Verkoczy et al, J. Immunol. 187:3785-97 (2011); Verkoczy et al, Current Opin. Immunol. 23:383-90 (2011)). A second reason that subdominant BnAbs may not be robustly induced is that the immunogen may be sufficiently heterogeneous such that only a minority of the immunogen is in the correct conformation, or there may be dominant non-neutralizing epitopes on the immunogen that divert the immune response or fill the limited germinal center space with dominant non-neutralizing antibodies such that subdominant BnAb clonal lineage cannot compete. Such a scenario regarding diversion of the B cell response by dominant epitopes has been suggested for antibody responses to HIV-1 Env targeting the V3 loop region (Nara and Garrity, Vaccine 16:1780-88 (1998)).

One component of the solution to induction of BnAbs that target both peptide and glycan portions of HIV-1 Env is to design peptide-glycan immunogens that retain the epitope of the BnAb. A second key to induction of BnAbs is to design peptide-glycan immunogens that are optimally presented by the immune system but that do not include dominant epitopes. Avci et al have recently elucidated the mechanism for glycoconjugate vaccine activation of the adaptive immune system for induction of optimal anti-glycan CD4 T helper and glycan antibody responses (Avci et al, Nature Med. 17:1602 (2011)). Finally, it would be key to construct synthetic peptide immunogens that are completely homogeneous so as to maximally stimulate B cell responses to only the epitope desired.

There are two major peptide-glycan epitopes of BnAbs on gp120 Env, one at N332 and the other at N156/N160 (McLellan et al, Nature 480: 336 (2011), Moore et al, Nature Medicine published online Oct. 21, 2012 doi: 10.1038/nm.2985). Peptide-glycan immunogens reflective of the N156. N160 gp120 BnAb epitopes have been constructed and their ability to bind to BnAbs PG9 and CH01 determined (McLellan et al, Nature 480:336 (2011), Bonsignori et al, J. Virology 85:9998 (2011)). These peptide glycans will be used for immunization testing in non-human primates for the ability to induce HIV-1 envelope-directed antibody responses against the V1/V2 N156/N160 peptide-glycan epitope that neutralize HIV quasispecies.

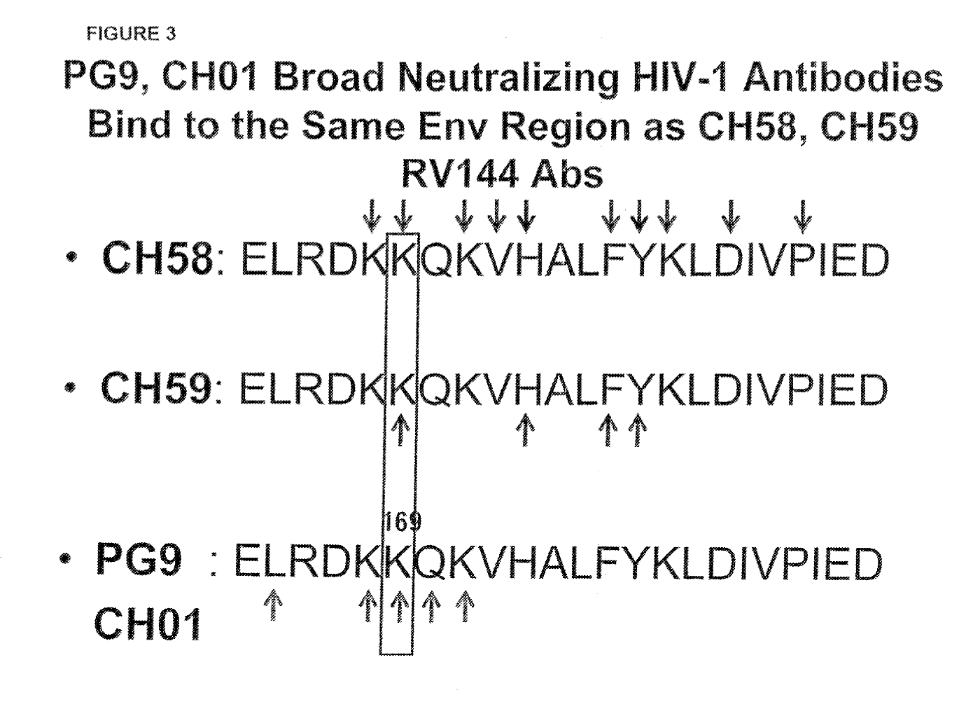

V2 antibodies induced by the RV144 ALVAC/AIDSVAX vaccine (human mAbs CH58, CH59) (U.S. Provisional Application No. 61/580,475, filed Dec. 27, 2011 and U.S. Provisional Application No. 61/613,222, filed Mar. 20, 2012) are relatively easy to induce and bind to V2 peptide at the amino acid footprints in FIGS. 1-3, that include amino acid K at 169. These antibodies do not bind glycans. Importantly, the K169 is also in the peptide footprint of the PG9 and CH01 BnAbs and K169 is critical for their binding (FIG. 3) (Doria-Rose et al, J. Virol. 86: 8319-23 (2012)).

Similarly, the N332 gp120 site has been reported to be a target of the initial (easy to induce) antibody neutralizing antibody response made soon after HIV infection (Haynes et al, submitted, 2012, U.S. Provisional Application No. 61/580,475, filed Dec. 27, 2011 and U.S. Provisional Application No. 61/613,222, filed Mar. 20, 2012). It is also a component of the epitope of some glycan-targeted BnAbs. Unlike the PGT anti-glycan antibodies (Pejchal et al, Science 334:1097 (2011)), the easy to induce neutralizing N332 response arises early after infection, and is not broadly neutralizing. Rather these “autologous” neutralizing antibodies generally only neutralize the viral strain that induced these antibodies in the host. Nonetheless, their dependence on N332 glycan is a clue that these antibodies can indeed be made if presented in the correct structure. Moreover, Moore et al, (Nature Medicine published online Oct. 21, 2012 doi: 10.1038/nm.2985) have recently shown that escape of the autologous virus from aa332 can induce an asparagine to occur there followed by induction of BnAb activity focused at N332. Shown in the Examples that follow is the successful synthesis of the PG9/CH01 V1/V2 epitope with binding both to the mature PG9/CH01 and to the unmutated common ancestors (UCAs) of these antibodies. The latter binding is critical for their use as immunogens since the UCAs are representative of the naïve B cell receptors of these lineages (Haynes et al, Nature Biotechnology 30:423-433 (2012)).

BnAbs to the N332 and N156/N160 peptide-glycan gp120 epitopes are more difficult to induce (McLellan et al, Nature 480:336 (2011), Bonsignori et al, J. Virology 85:9998 (2011)) and have not been induced by vaccination. Importantly, components of these sites can be targets of dominant, less broadly neutralizing HIV-1 antibodies (like CH58 and CH59) that are more easily made and in some cases induced by vaccines (Tang, H et al. J. Virology 85: 9286 (2011), Haynes et al, submitted 2012, U.S. Provisional Application No. 61/580,475, filed Dec. 27, 2011 and U.S. Provisional Application No. 61/613,222, filed Mar. 20, 2012).

The present invention relates, at least in part, to a synthetic peptide that is homogeneous in content, antigenicity and glycosylation forms, and that has preferred binding to the broad neutralizing antibodies PG9 and CH01 and minimally binds the non-tier 2 neutralizing antibody CH58 or its RUA. The invention includes peptide glycans, such as the V1/V2 Man₃GlcNac₂ and the V1/V2 Man₅GlcNac₂ peptide glycans, that preferentially induce PG9- and CH01-like BnAbs when administered to a subject (e.g., a human subject) as an immunogenic composition. The invention also includes immunogenic compositions comprising such immunogens.

The immunogens of the invention can be formulated as DNAs (Santra et al, Nature Med. 16:324-8 (2010)) and as inserts in vectors including rAdenovirus (Barouch et al, Nature Med. 16:319-23 (2010)), recombinant mycobacteria (i.e., BCG or M. smegmatis) (Yu et al, Clinical Vaccine Immunol. 14:886-093 (2007; ibid 13: 1204-11 (2006)), and recombinant vaccinia type of vectors (Santra, Nature Med. 16: 324-8 (2010)). The immunogens of the invention can also be administered as a protein boost in combination with a variety of vectored Env primes (i.e., HIV-1 Envs expressed in non-HIV viral or bacterial vectors) (Barefoot et al. Vaccine 26:6108-18 (2008)), or as protein alone (Liao et al, Virology 353:268-82 (2006)). The protein can be administered with an adjuvant such as MF59, AS01B, polyI, polyC or alum and administered, for example, subcutaneously or intramuscularly. Alternatively, the protein or vectored immunogen can be administered mucosally such as via intranasal immunization or by other mucosal route (Torrieri D L et al Mol. Ther. Oct. 19 2010, E put ahead of print).

Immunogens of the invention are suitable for use in generating an immune response in a patient (e.g., a human patient) to HIV-1. The mode of administration of the HIV-1 protein/polypeptide/peptide, or encoding sequence, can vary with the immunogen, the patient and the effect sought, similarly, the dose administered. As noted above, typically, the administration route will be intramuscular or subcutaneous injection (intravenous and intraperitoneal can also be used). Additionally, the formulations can be administered via the intranasal route, or intrarectally or vaginally as a suppository-like vehicle. Optimum dosing regimens can be readily determined by one skilled in the art. The immunogens are preferred for use prophylactically, however, their administration to infected individuals may reduce viral load.

In addition to the above-described immunogens designed for induction of BnAbs, the invention also includes isolated monoclonal antibodies resulting from that induction, and fragments thereof (e.g., scFv, Fv, Fab′, Fab and F(ab′)₂ fragments), and the use thereof in methods of treating or preventing HIV-1 in a subject (e.g., a human subject). The invention further includes compositions comprising such antibodies fragments thereof, and a carrier. Suitable dose ranges can depend on the antibody and on the nature of the formulation and route of administration. Optimum doses can be determined by one skilled in the art without undue experimentation. Doses of antibodies in the range of 10 ng to 20 μg/ml can be suitable (both administered and induced).

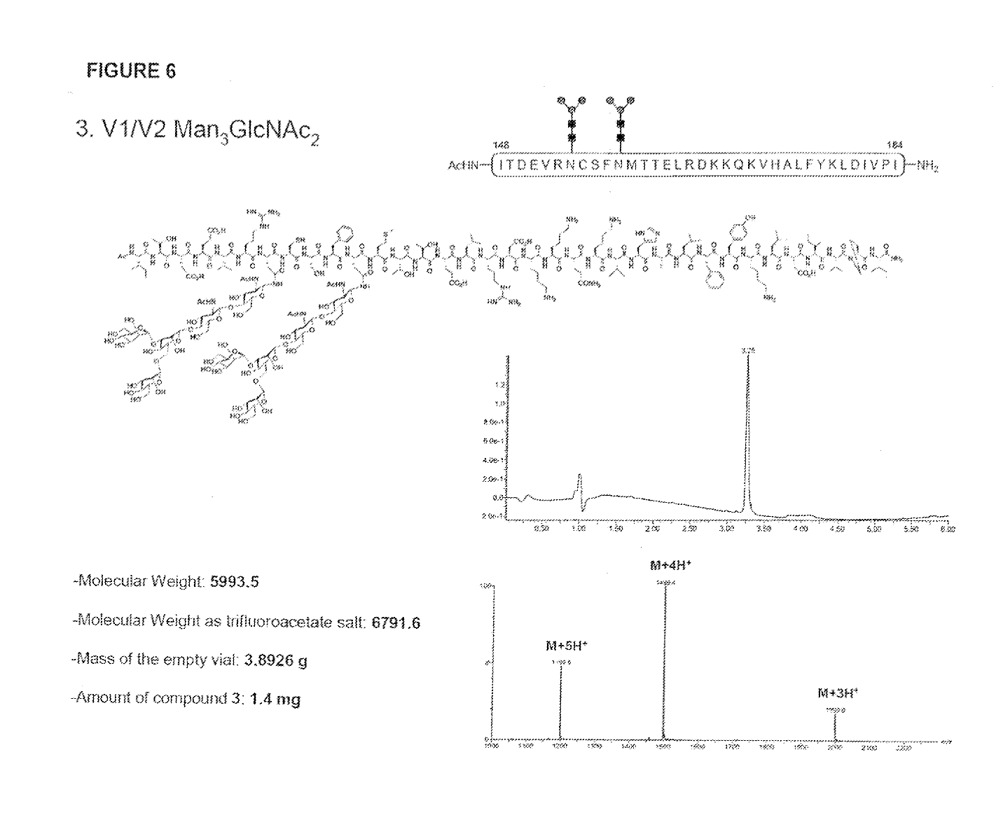

The structures of the peptide-glycans that have been produced on the N160, N156 are shown throughout the application, inter alia in FIGS. 6, 7 and 47, and the glycan structures that will be produced on the N332 region will be from sequences and glycans to which PGT antibodies bind (Pejchal et al, Science 334:1097 (2011)).

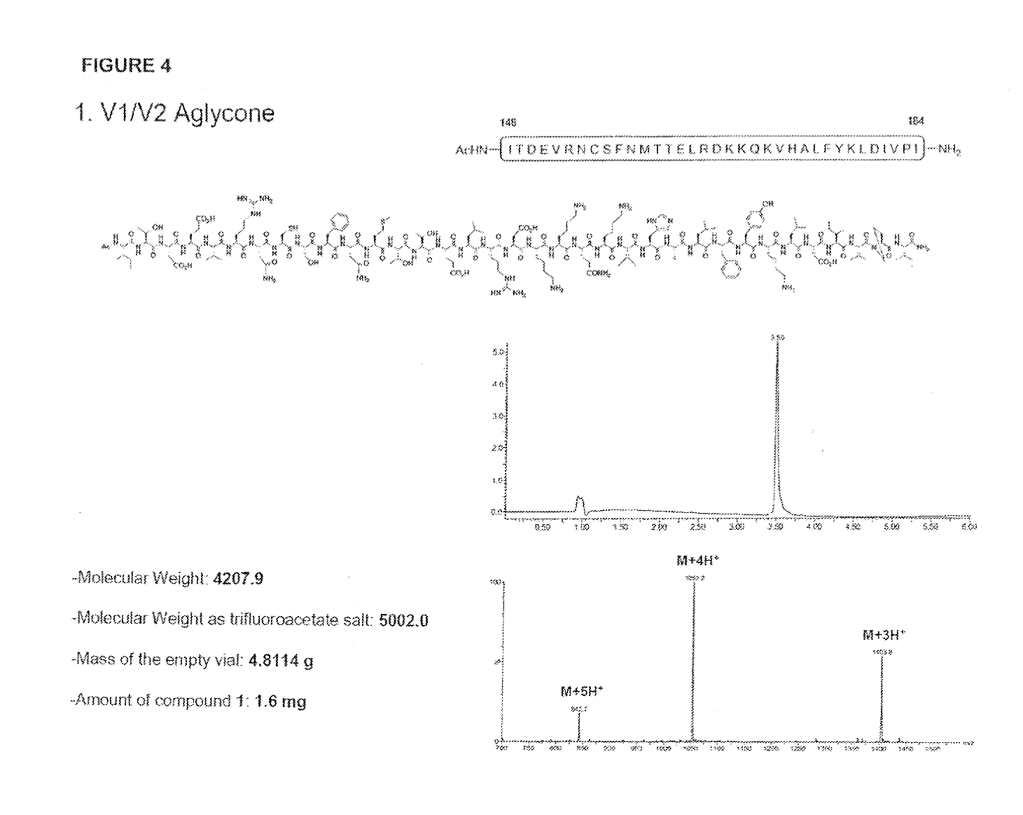

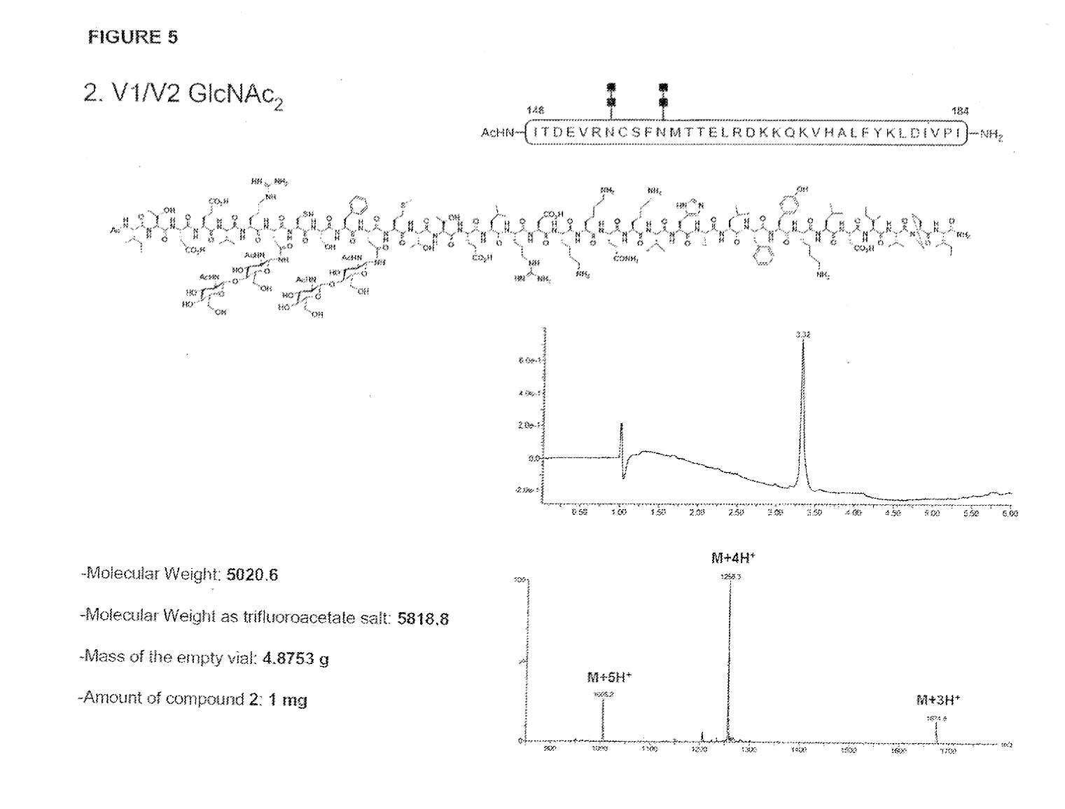

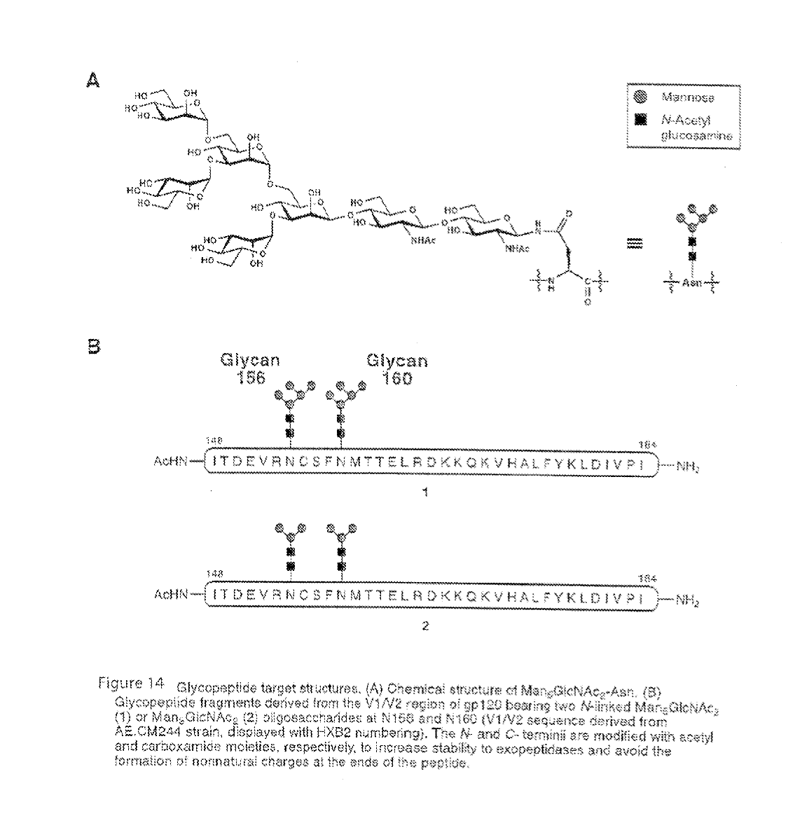

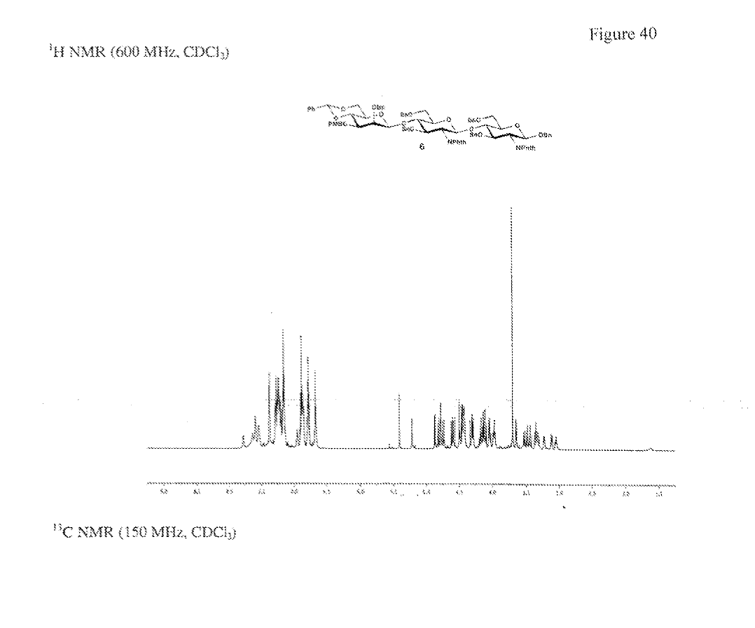

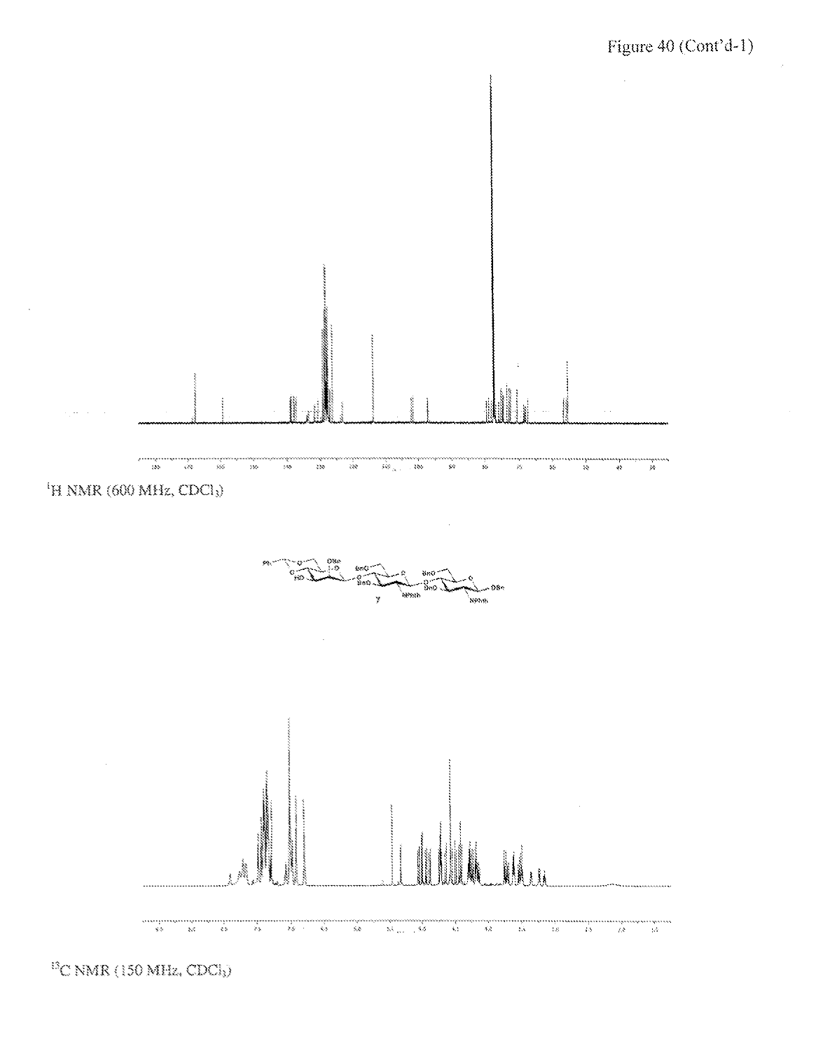

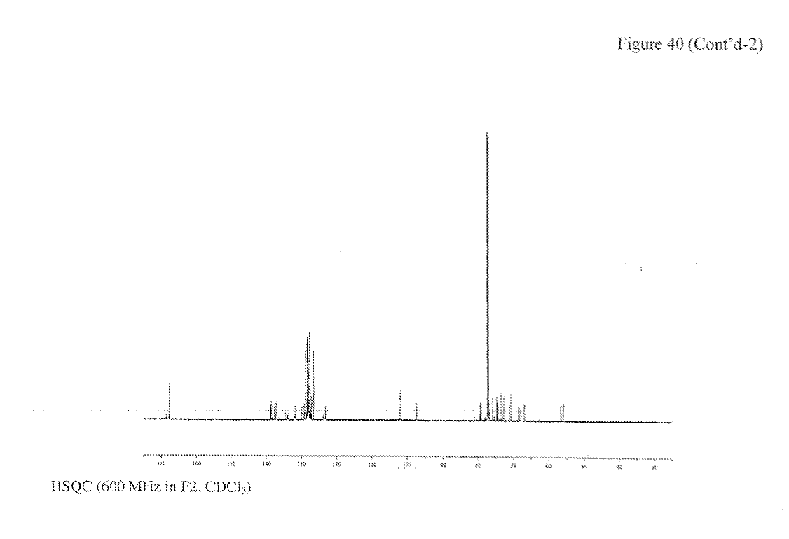

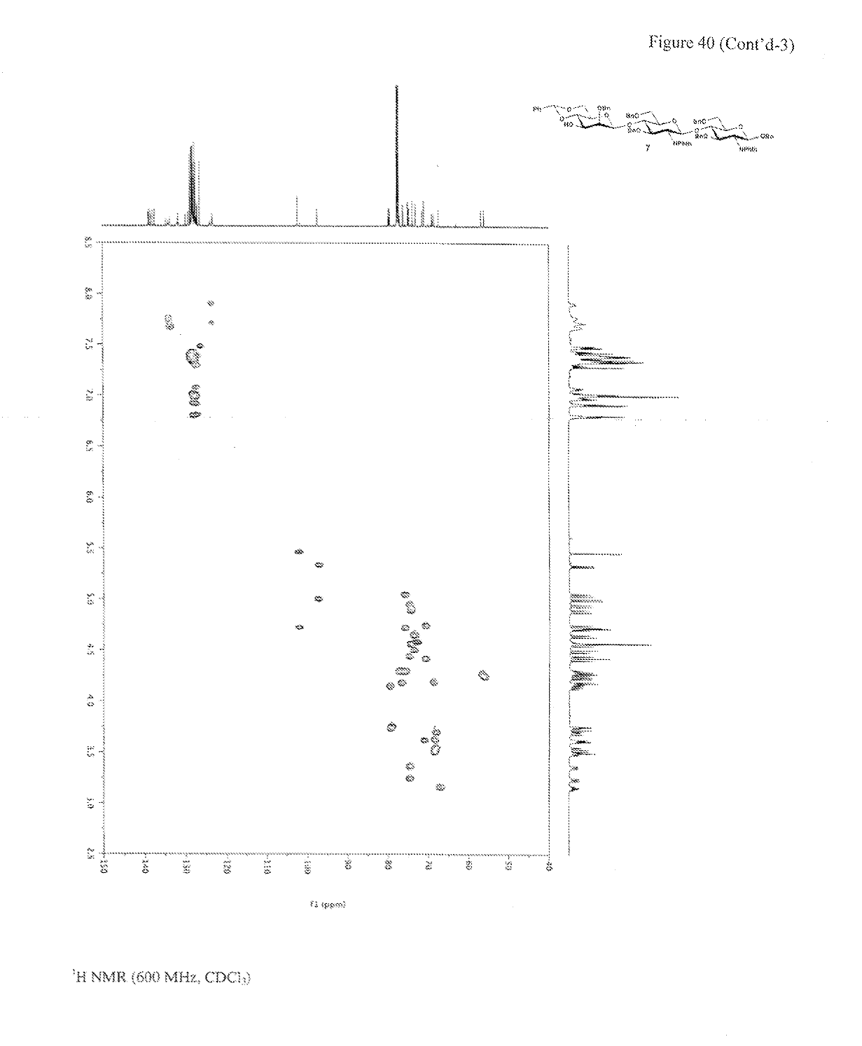

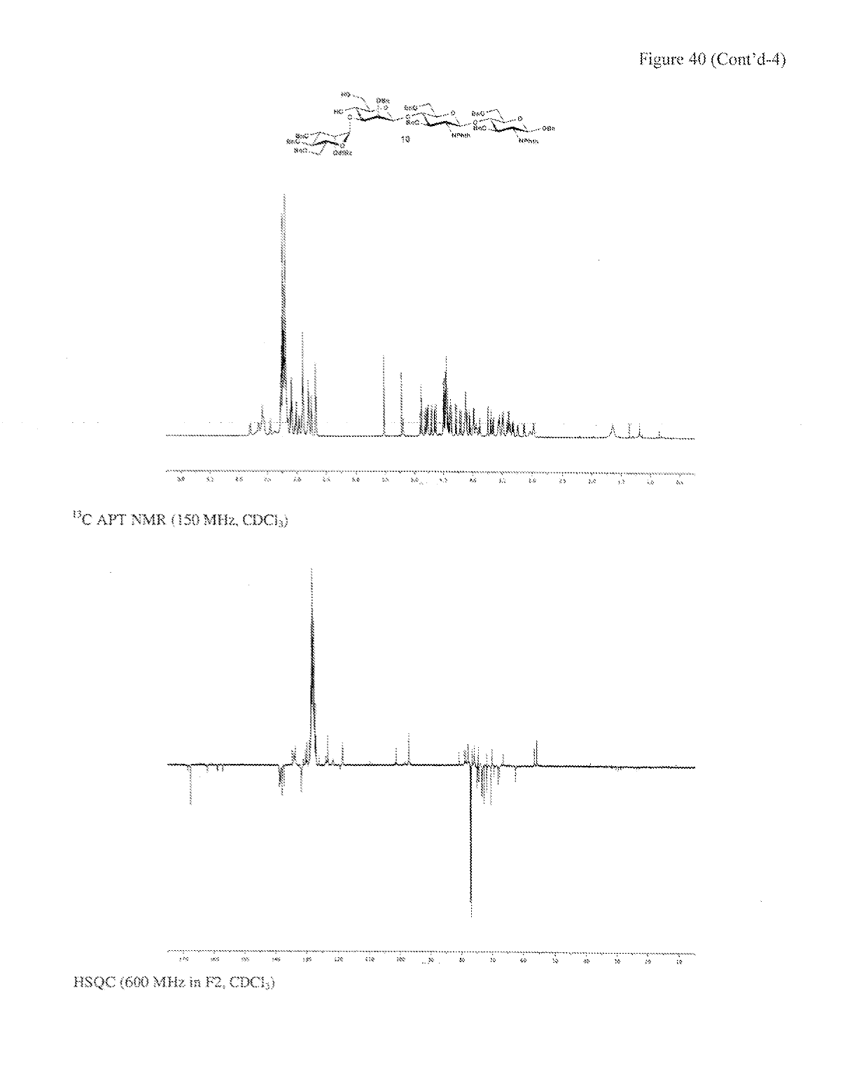

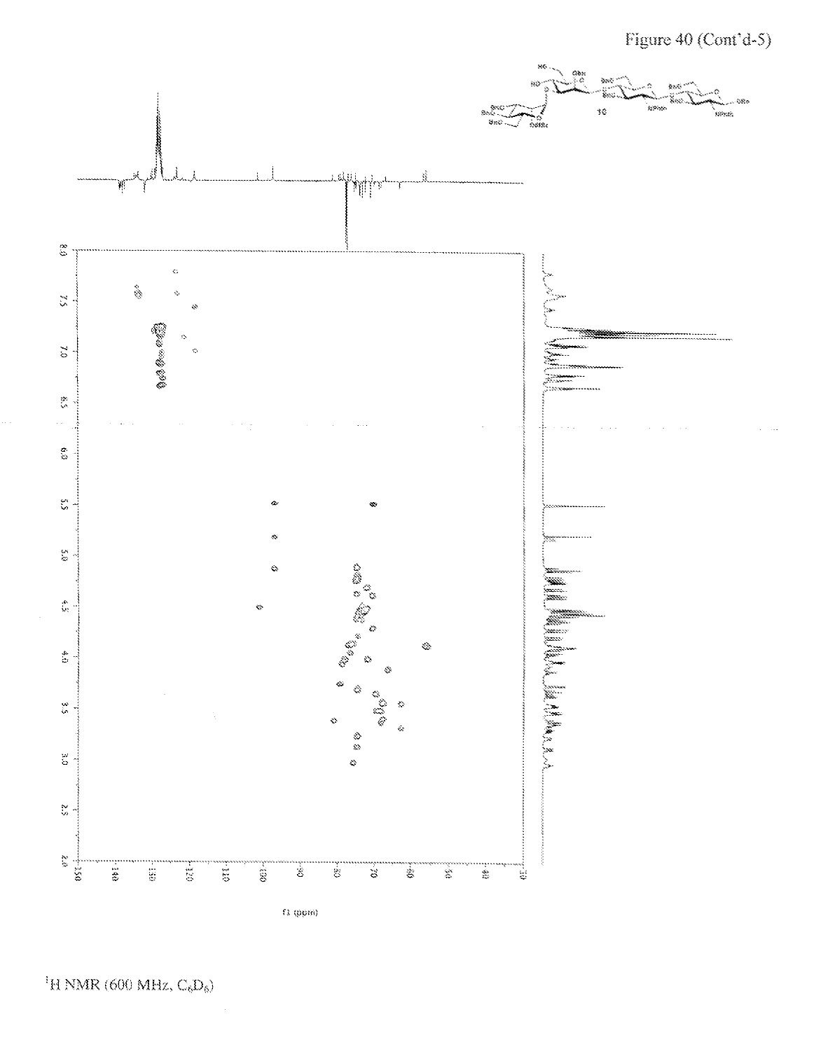

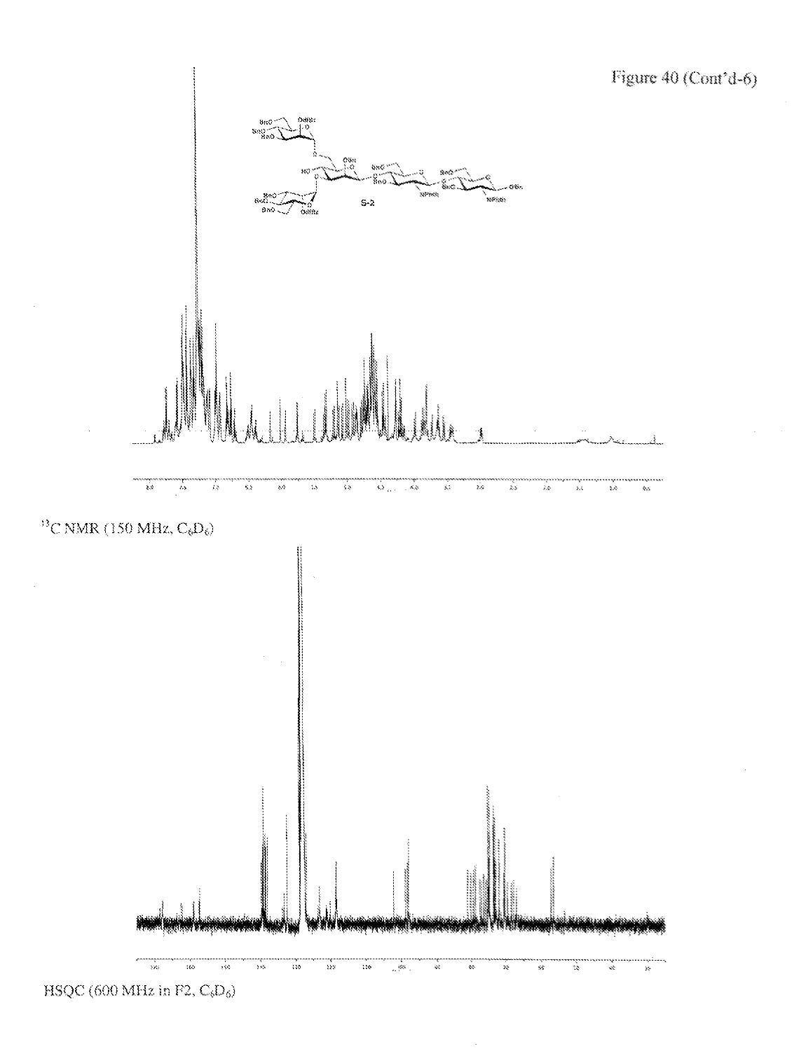

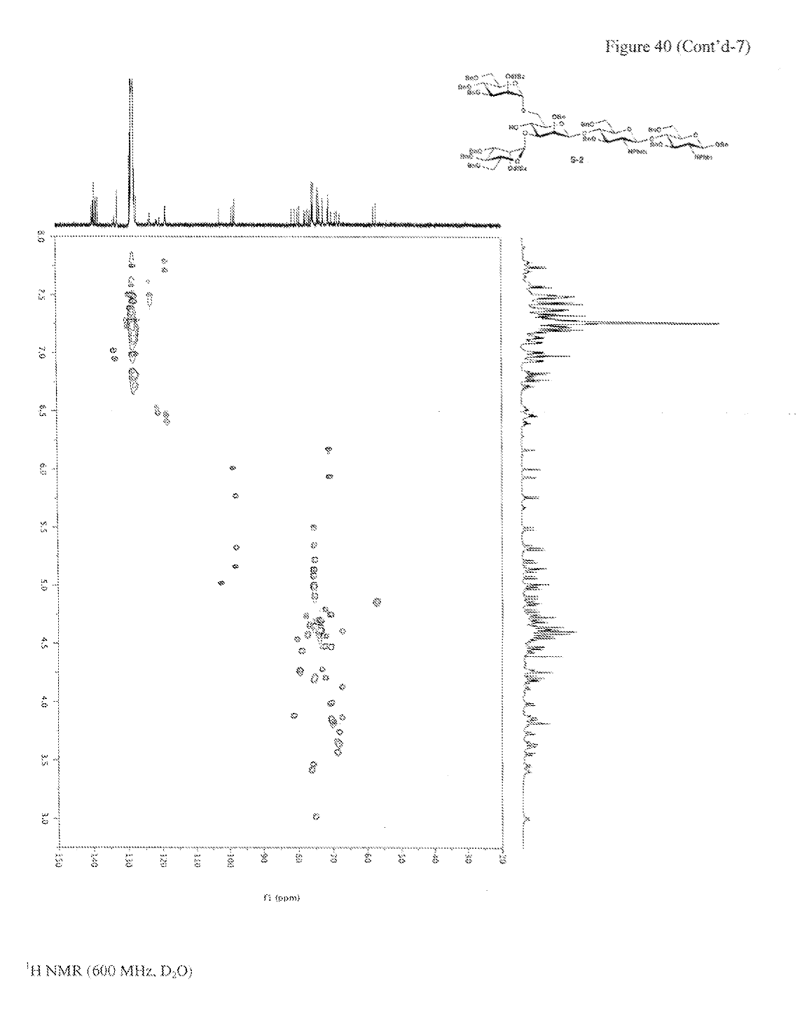

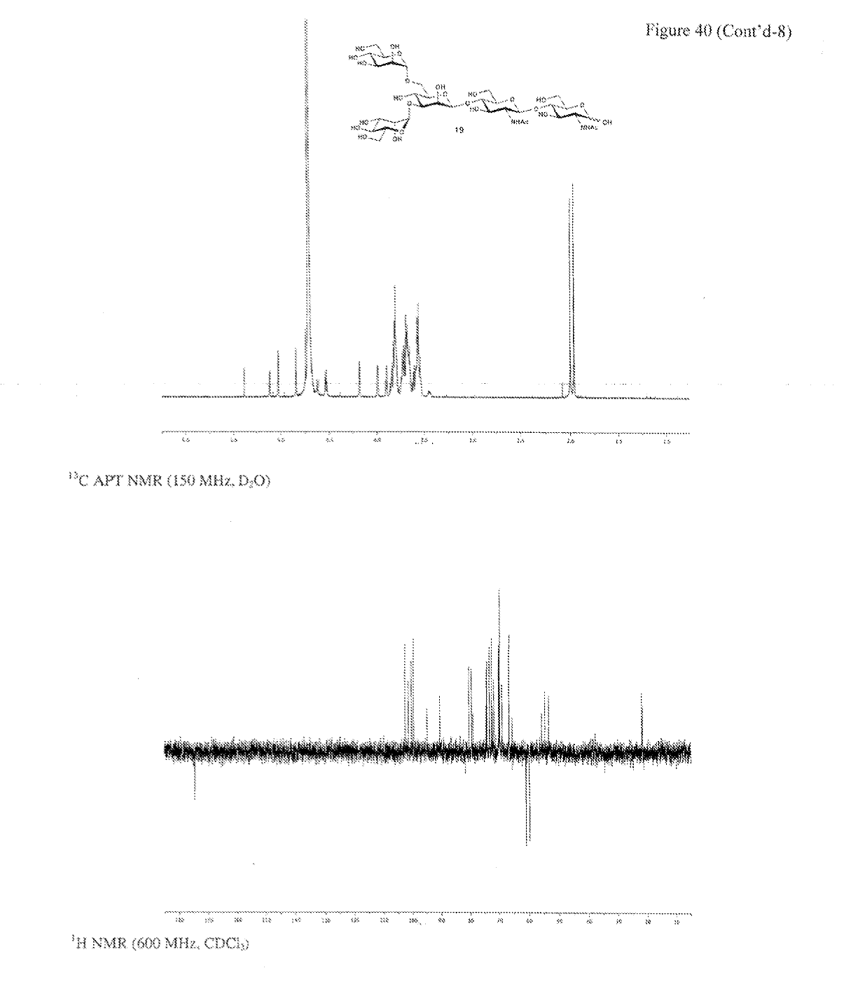

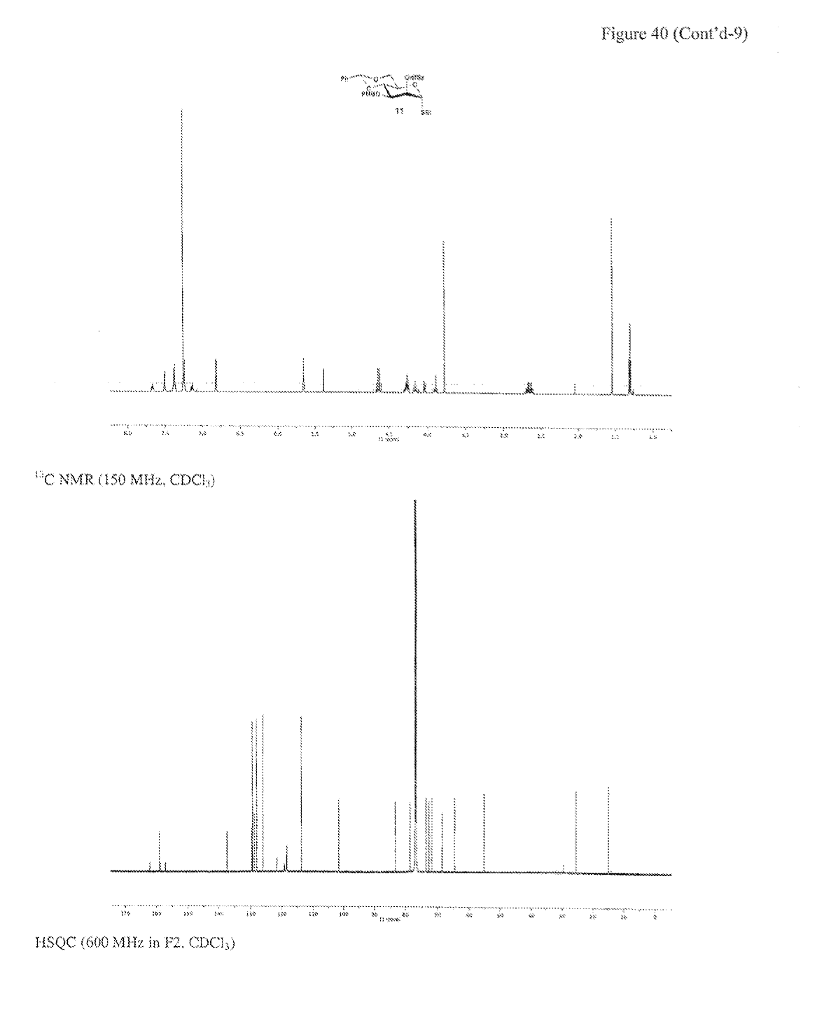

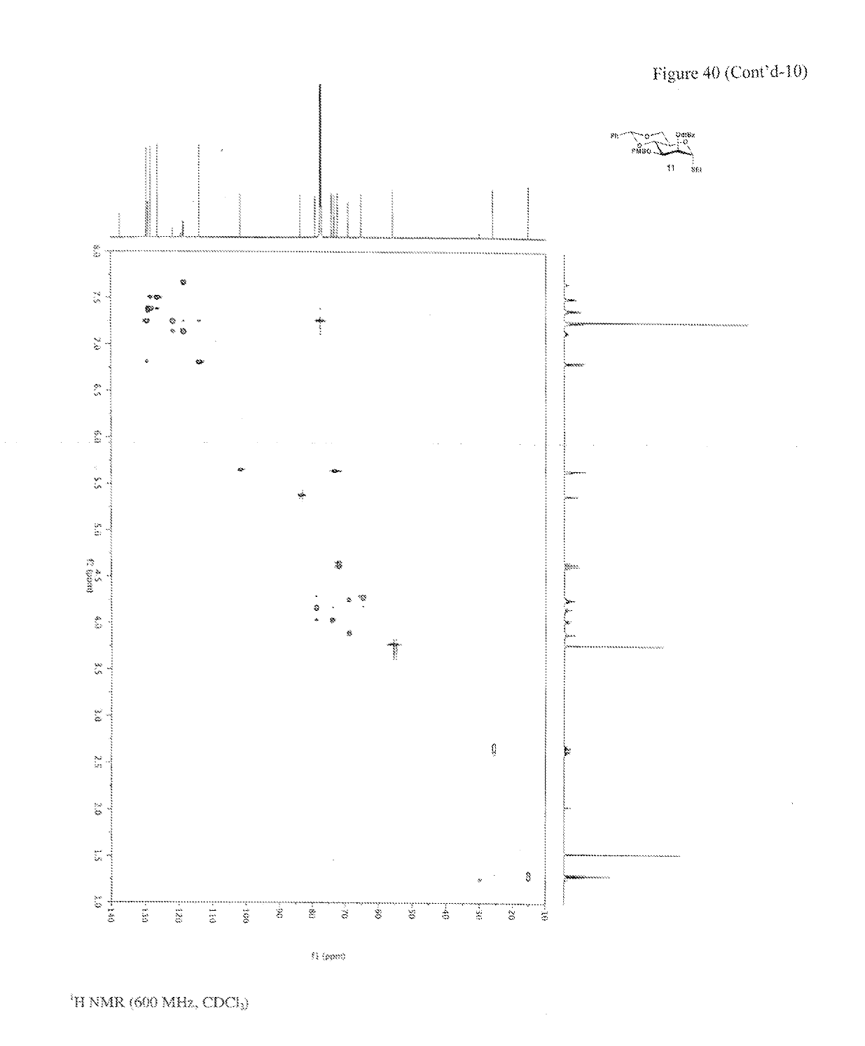

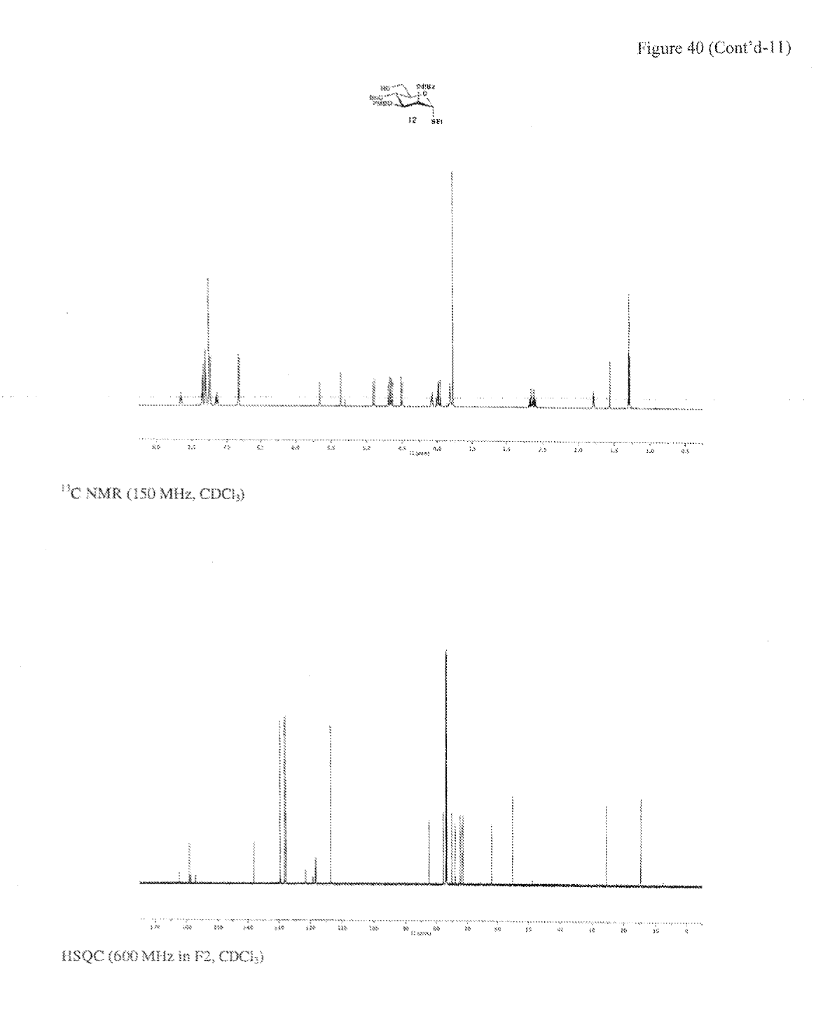

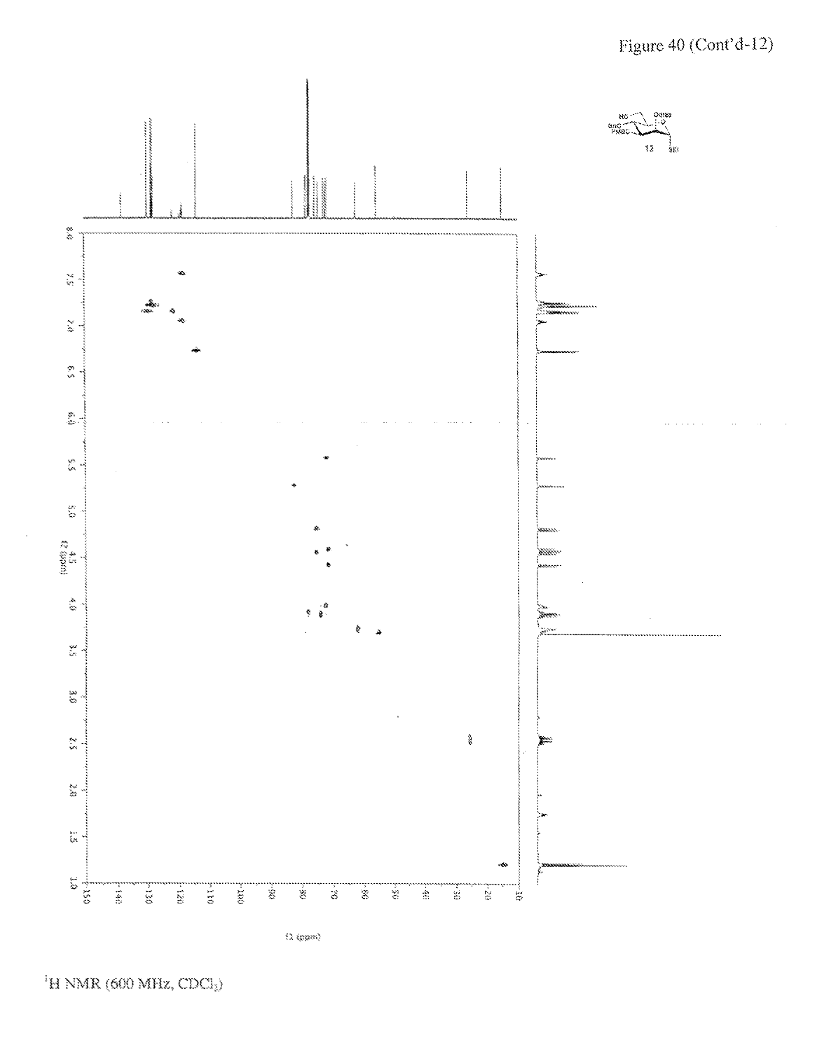

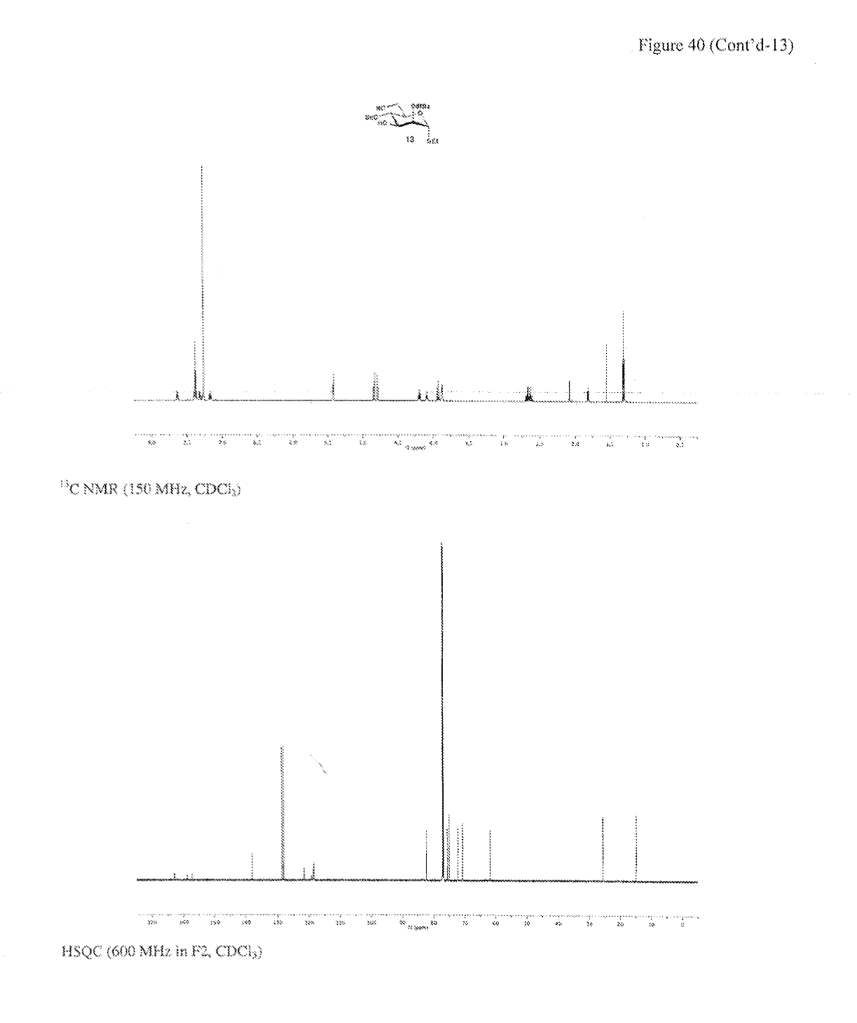

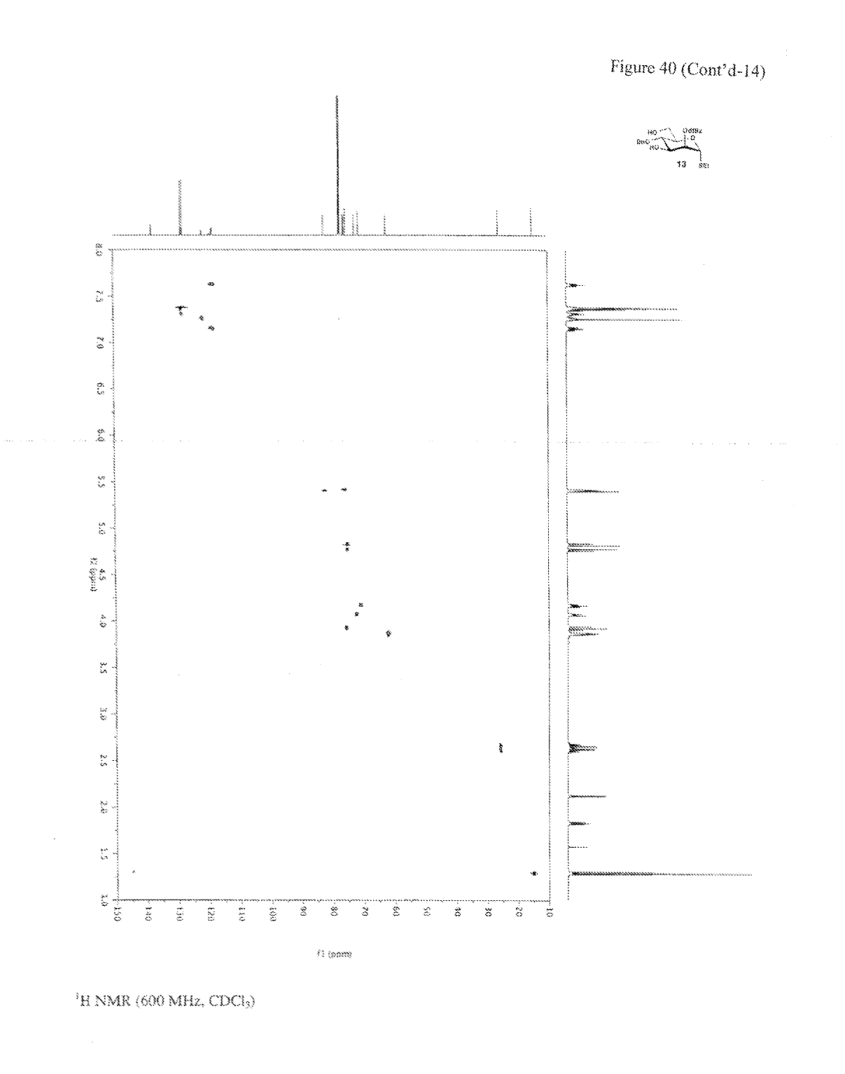

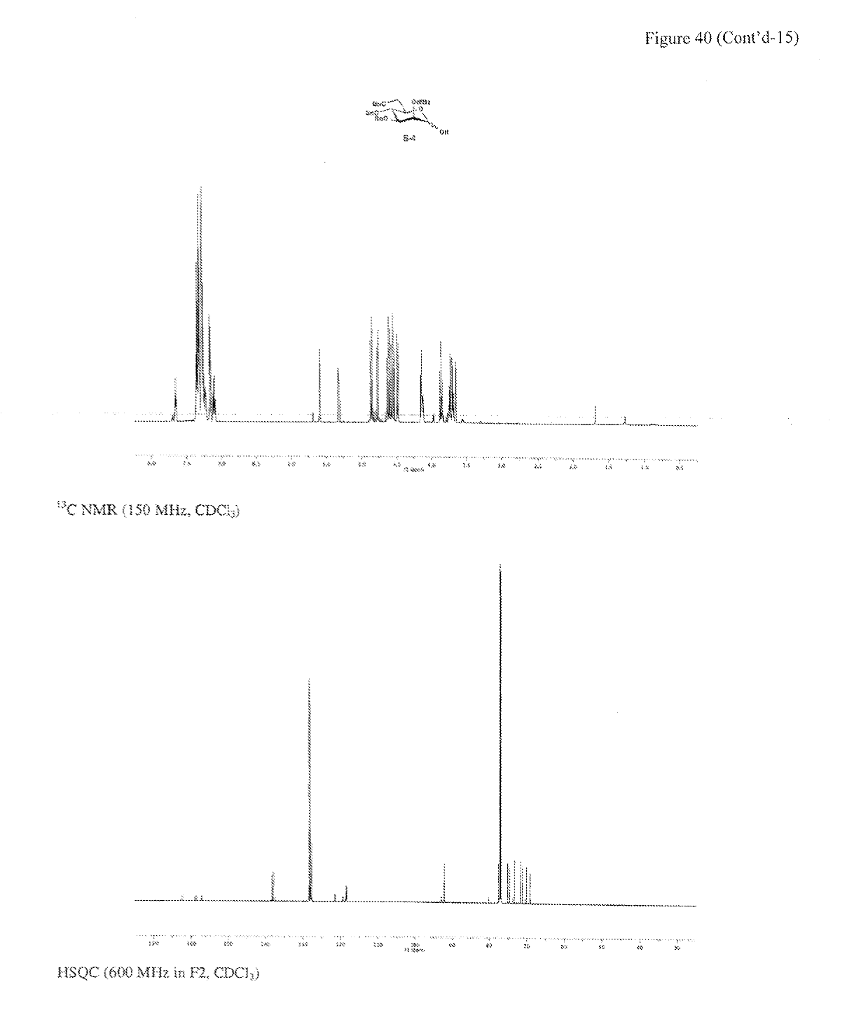

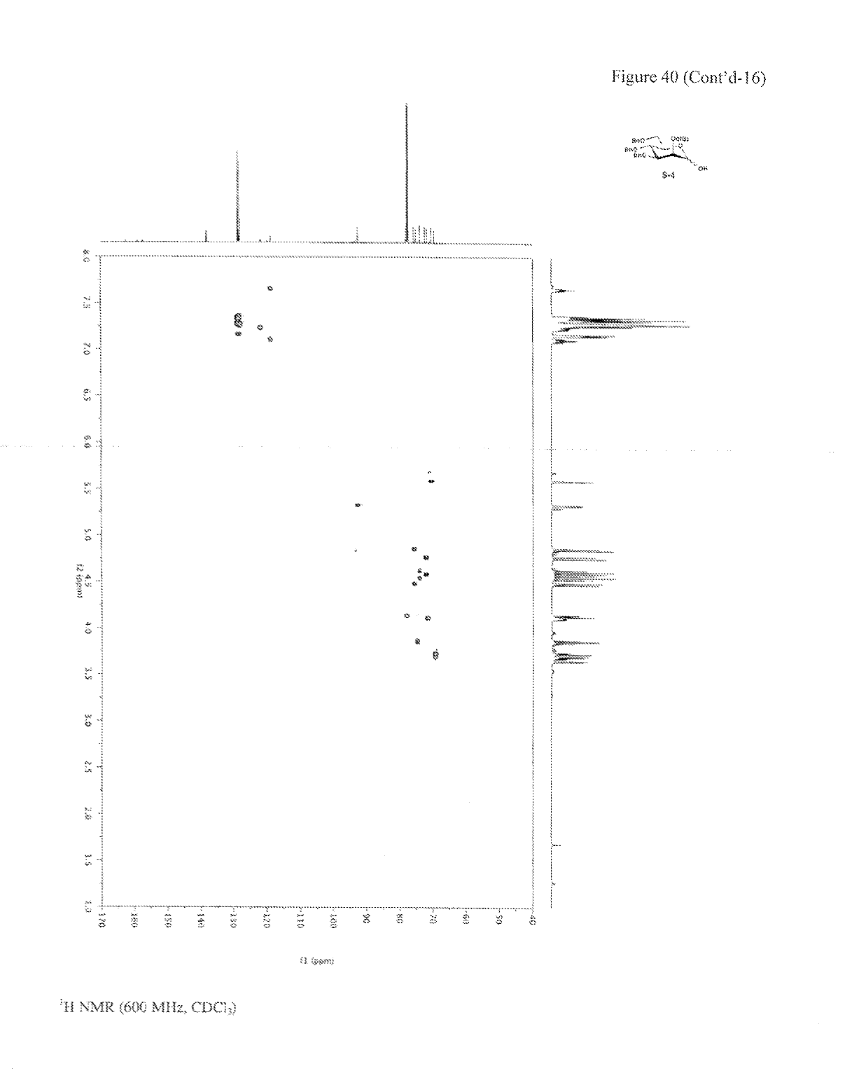

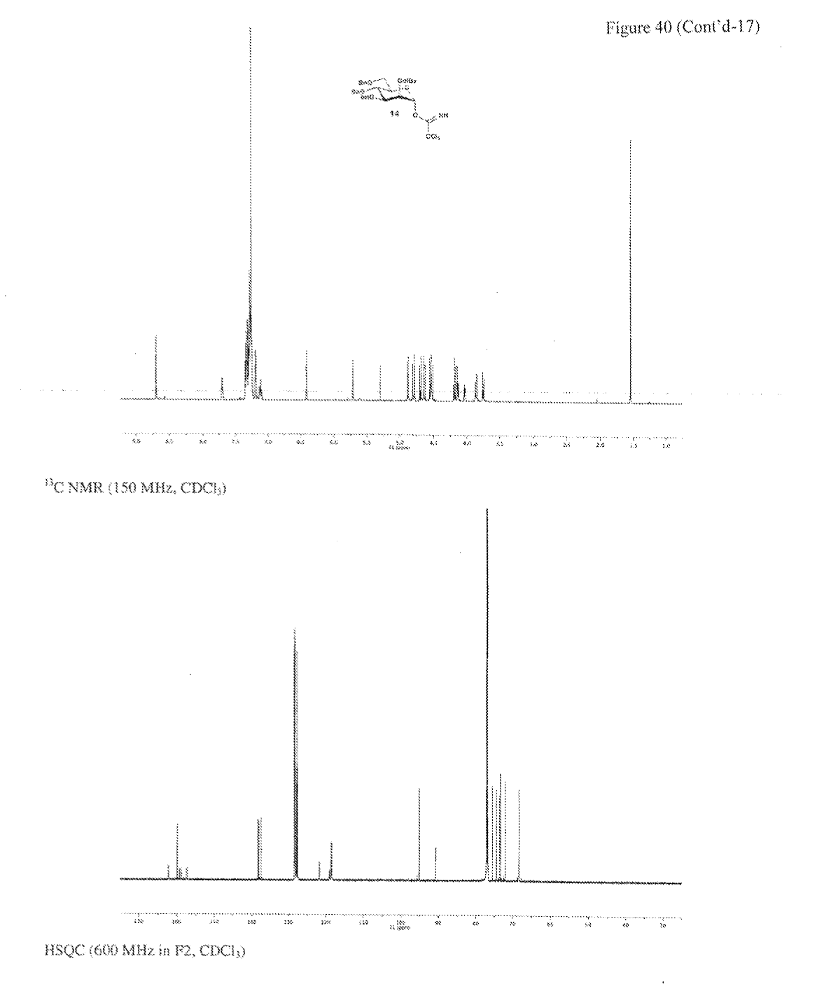

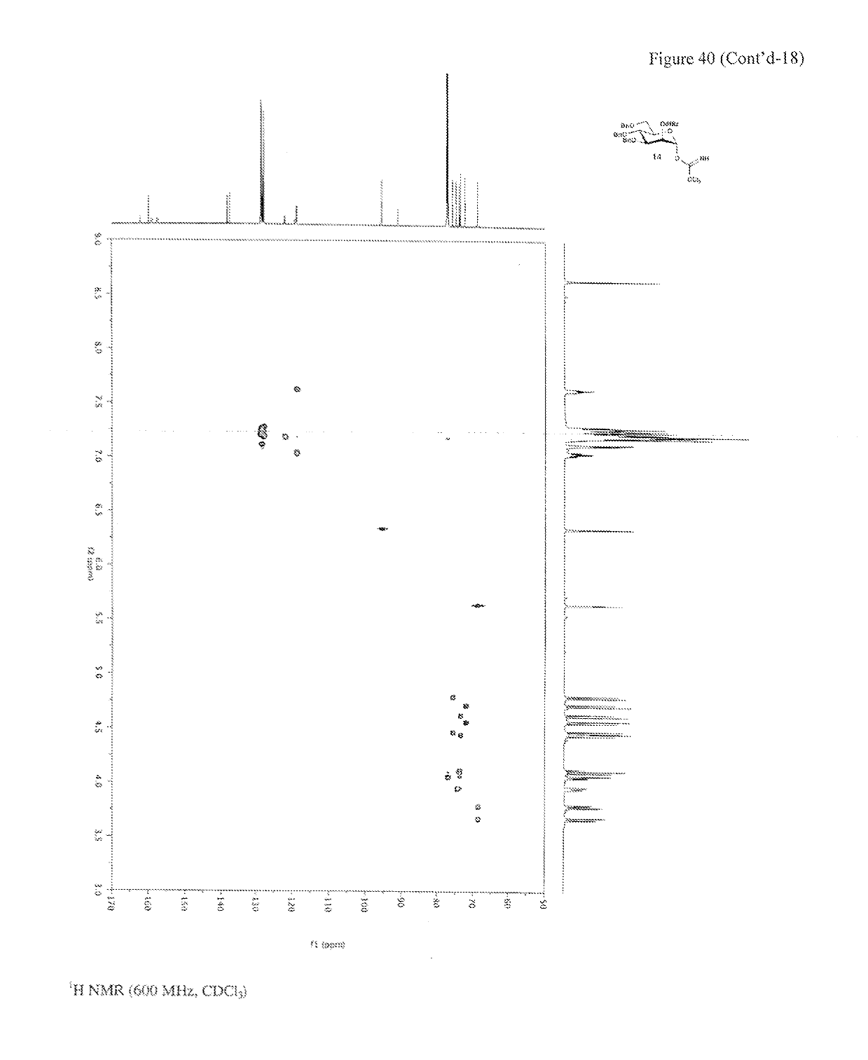

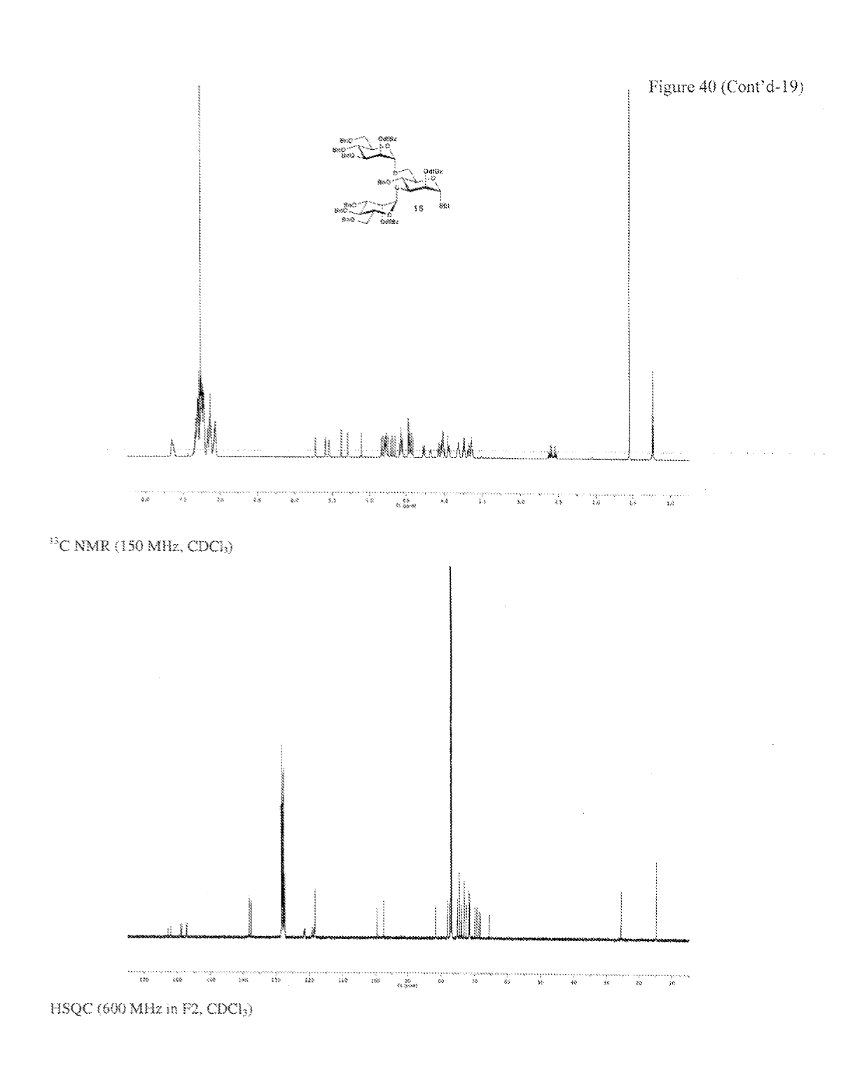

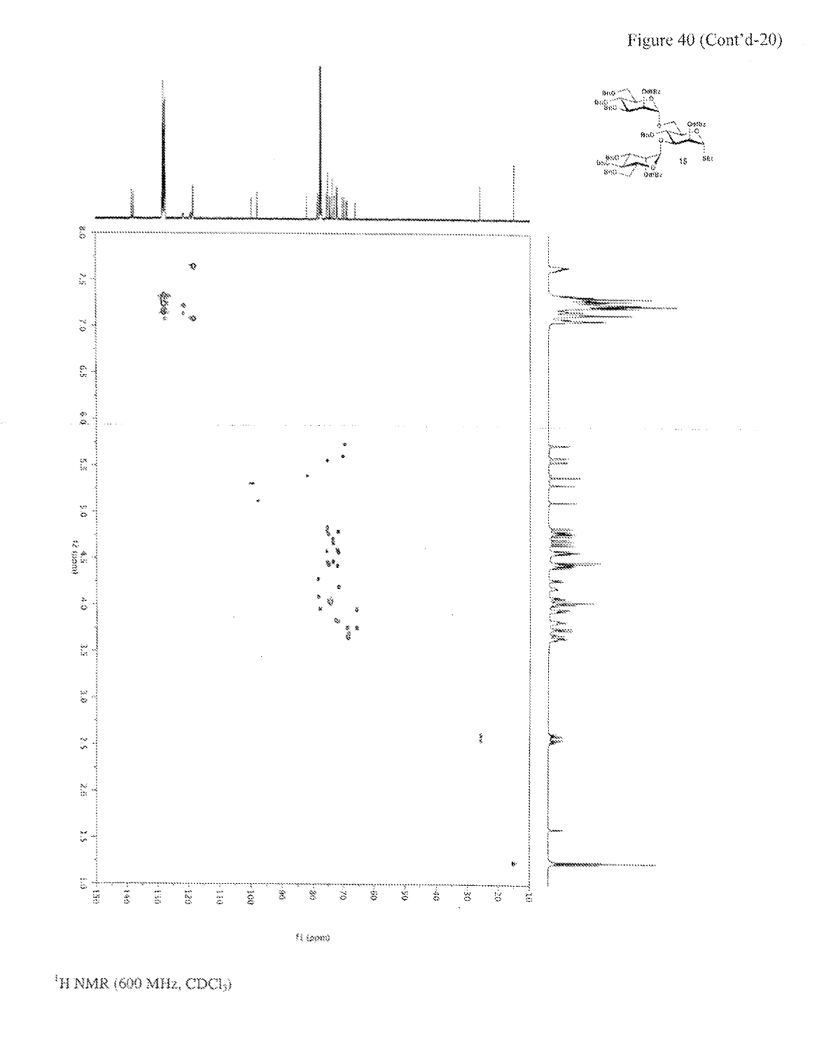

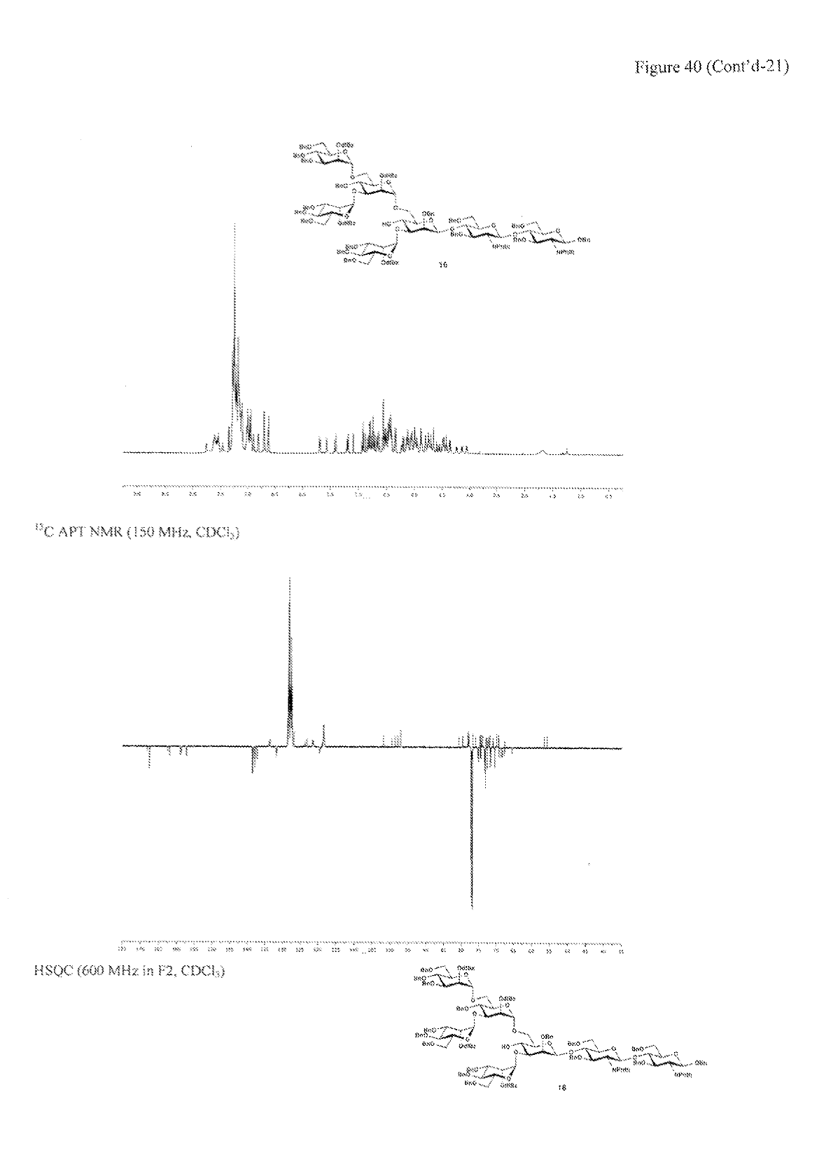

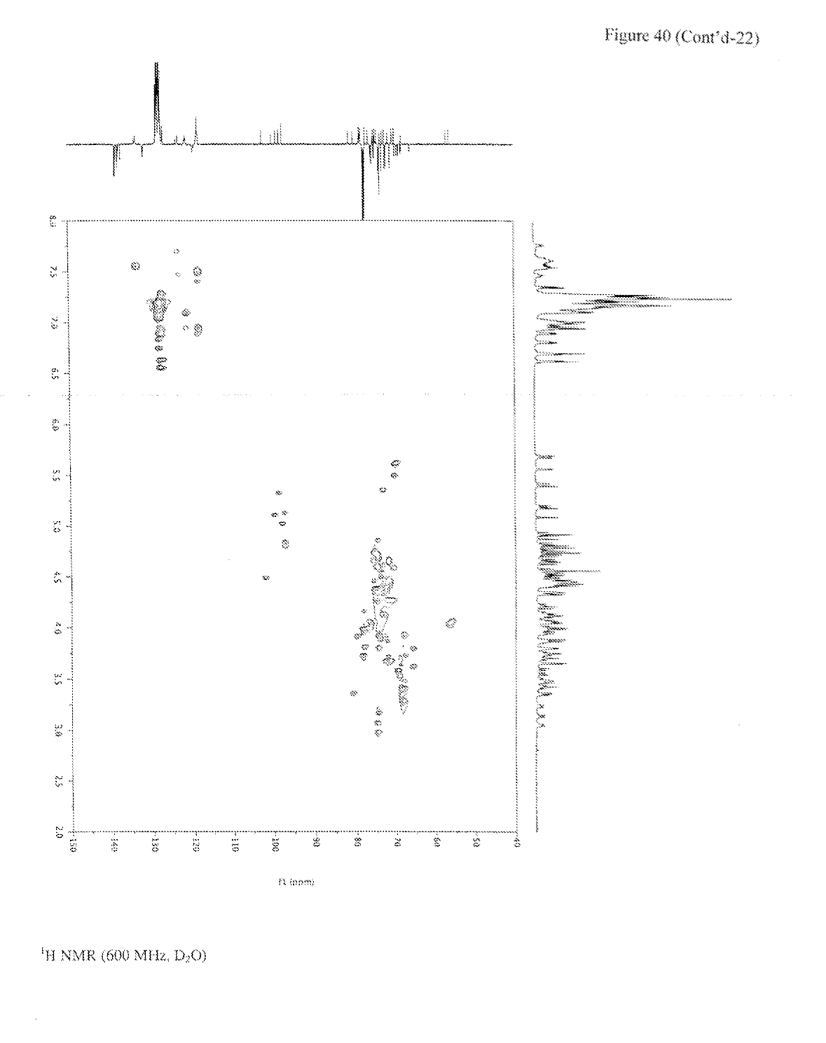

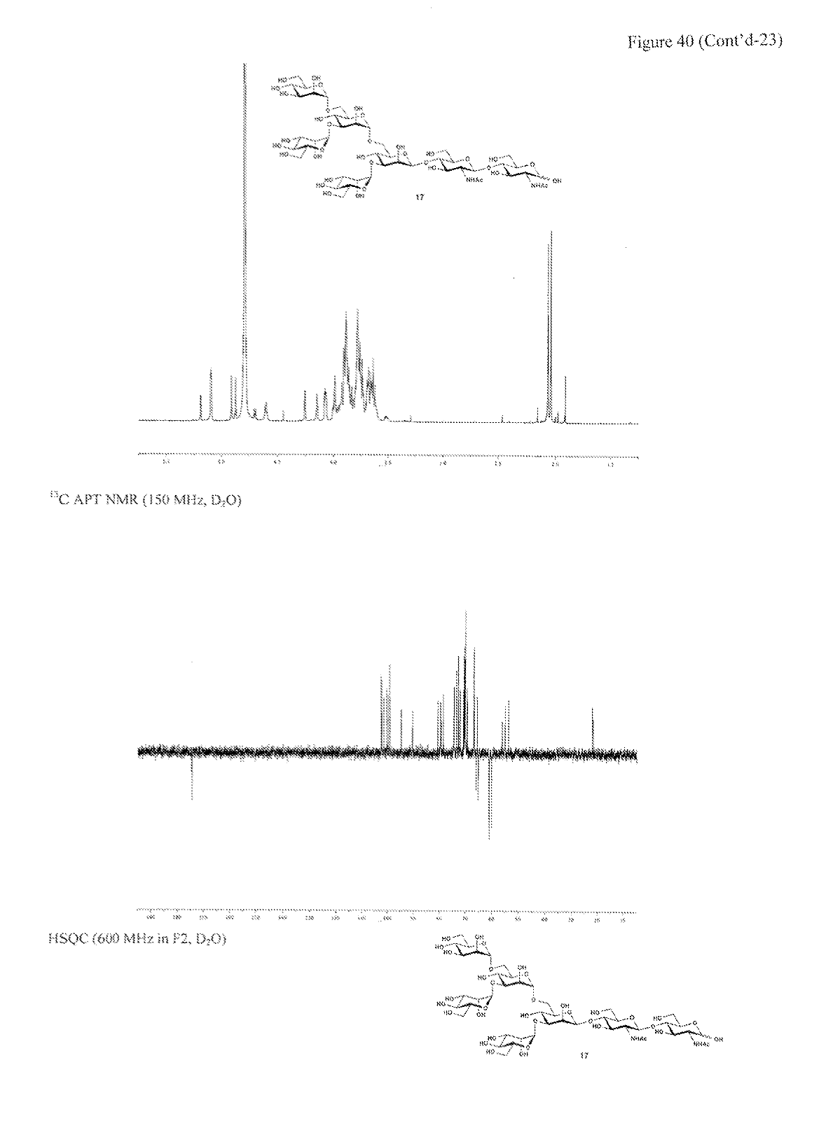

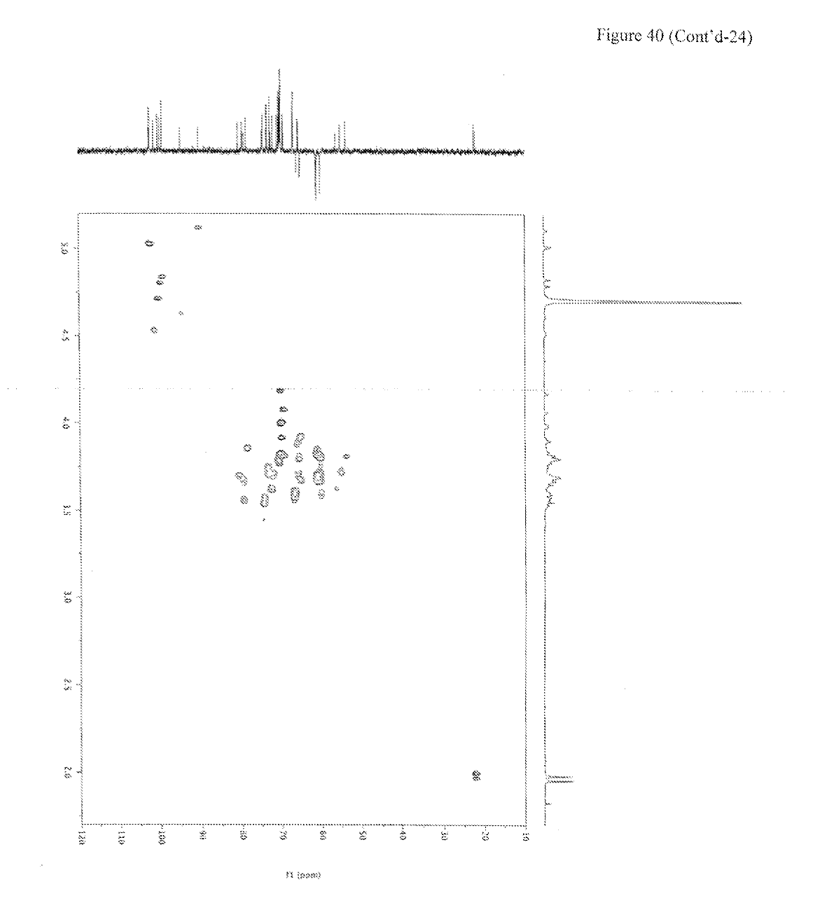

The methods used to make the peptide glycan immunogens in FIGS. 4-7 are partially described in: Wang et al, Angew. Chem. Int. Ed. 51: Epub ahead of print DOI: 1002/anie.201206090, 2012; ibid Wang et al, doi: 10.1002/anie.201205038, 2012; J. Amer. Chem. Soc 133: 1597-602, 2011, and then iterated in detail in the FIGS. 14-24, 26-50 and Examples 2, 3 5, and 6 below. Details of the synthesis of gp120 V1/V2 region glycopeptides details of conjugating glycopeptides to carrier proteins are provided in Example 2-Example 5.

Certain aspects of the invention can be described in greater detail in the non-limiting Examples that follows. (See also Prov. Appln. Nos. 61/719,304 filed Oct. 26, 2012 and 61/862,442 filed Aug. 5, 2013, the entire contents of which are incorporated herein by reference.)

### Example 1

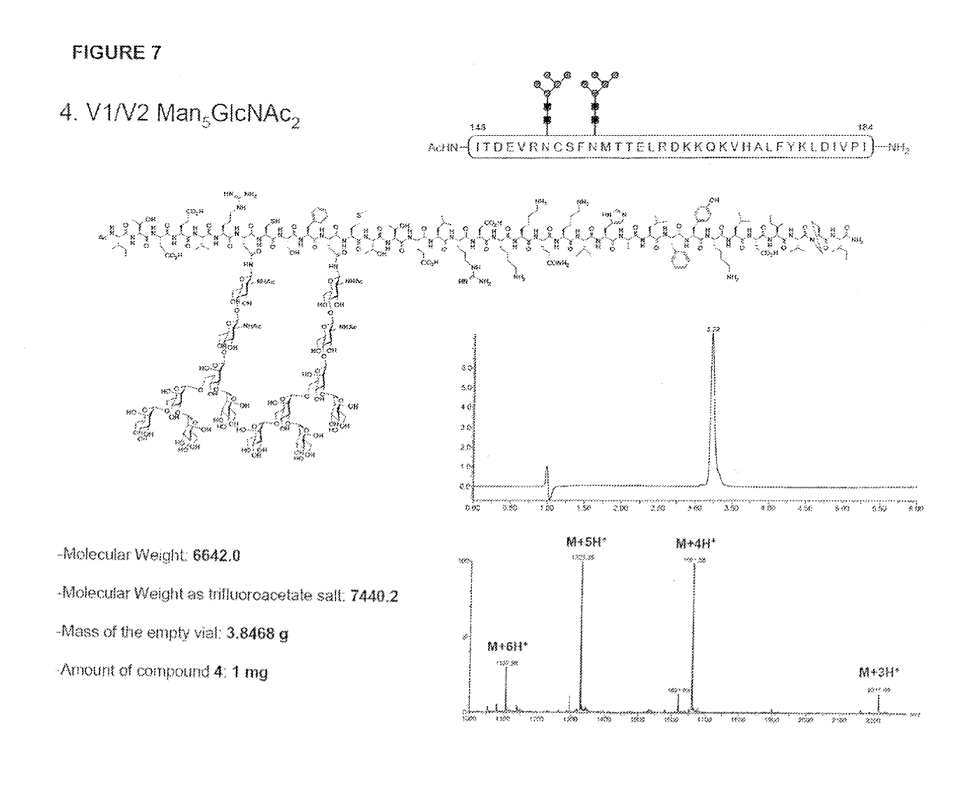

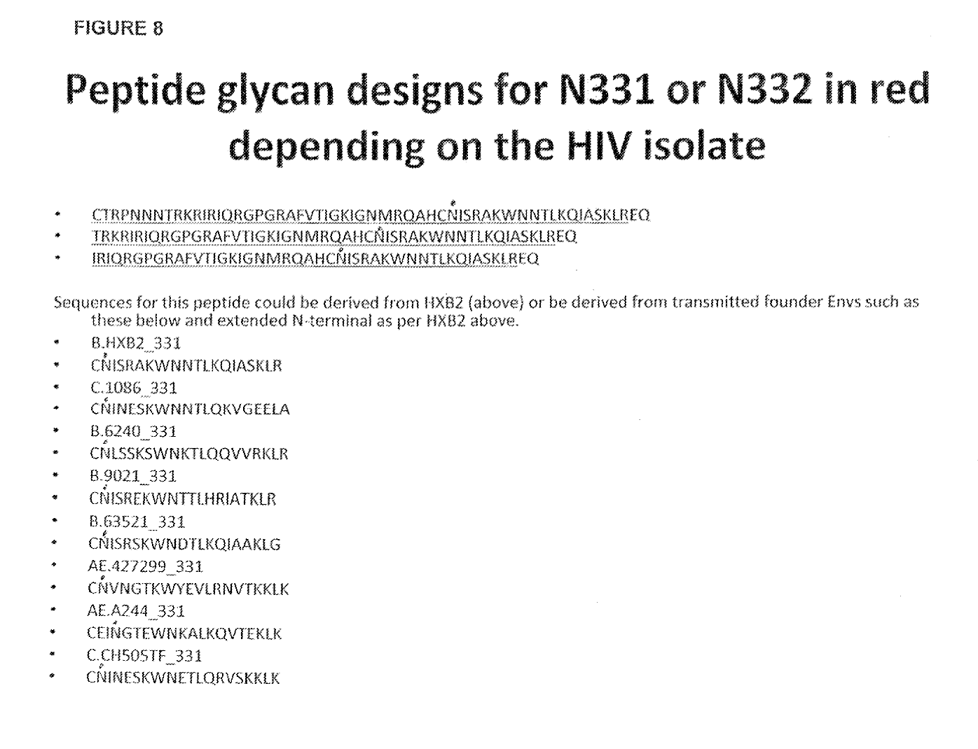

Models of the V1/V2 peptides and their glycans are shown in FIGS. 4-7. FIGS. 4-7 show the sequence of the V1/V2 peptide ITDEVRNCSFNMTTELRDKKQKVHALFYKLDIVPI with N156 and N160 glycans present in FIGS. 6 and 7. This peptide sequence is from AE.CM244 HIV strain, and was so chosen because the PG9, PG16 and CH01-04 antibodies bind well to this sequence in the C beta strand of V1/V2 in this virus. The peptide for N332 targeting would have the base and right-hand side (C-terminal portion) of the V3 loop with N332 (FIG. 8). The glycans to be synthesized at N332 (or N331 as need be) would be man8 or man9 glycans as shown in Pejchal et al (Science 334:1097 (2011)).

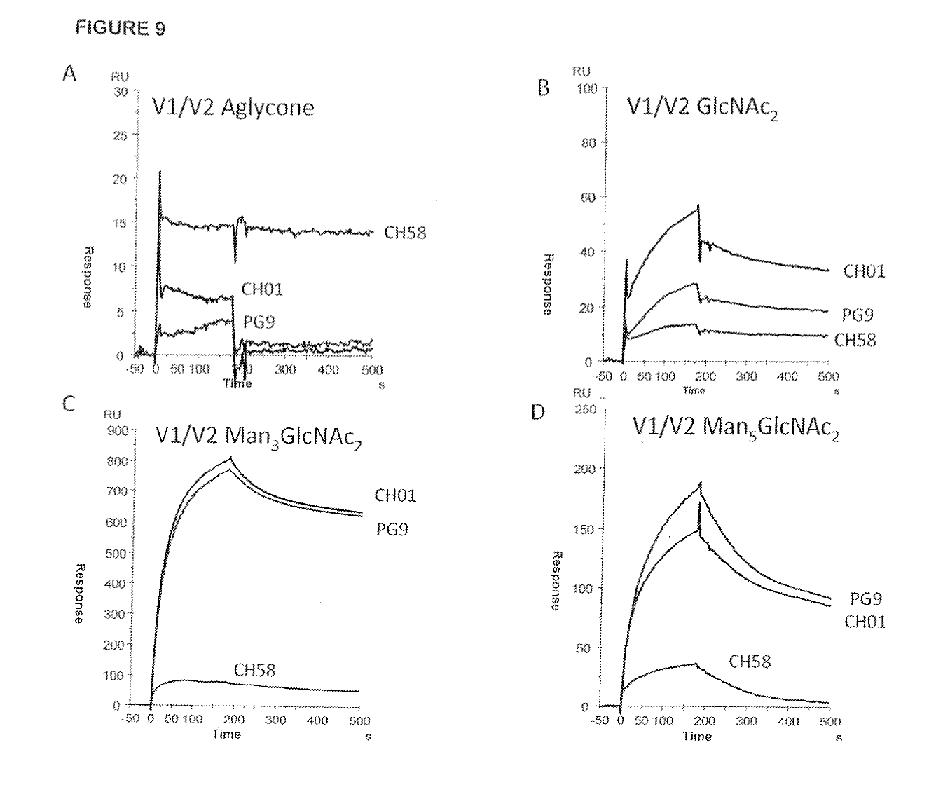

FIG. 9 shows the selective binding of V1/V2 broadly neutralizing mAbs to synthetic V1/V2 glycopeptides. Only the V1/V2 mAb CH58 bound to the glycan-deficient (aglycone) peptide (FIG. 9A). V1/V2 bNAbs (PGG9, CH01) bound weakly to V1/V2 GlcNAc₂ peptide (FIG. 9B). In contrast, both bNAbs PG9 and CH01 bound avidly to the glycopeptides, V1/V2 Man₃GlcNAc₂ and V1/V2 Man₅GlcNAc₂ (FIGS. 9C and 9D). CH58 bound weakly to both glycopeptides. Each of the mAbs was captured on a human Fc specific IgG directly immobilized on a BIAcore CM5 sensor chip. Each of the V1/V2 peptides (3 min at 50 uL/min) was injected over the mAb captured surface and SPR binding was monitored on a BIAcore 3000 instrument. Non-specific binding of peptides was subtracted following measurement of signal on a surface with a control ant-RSV mab Synagis.

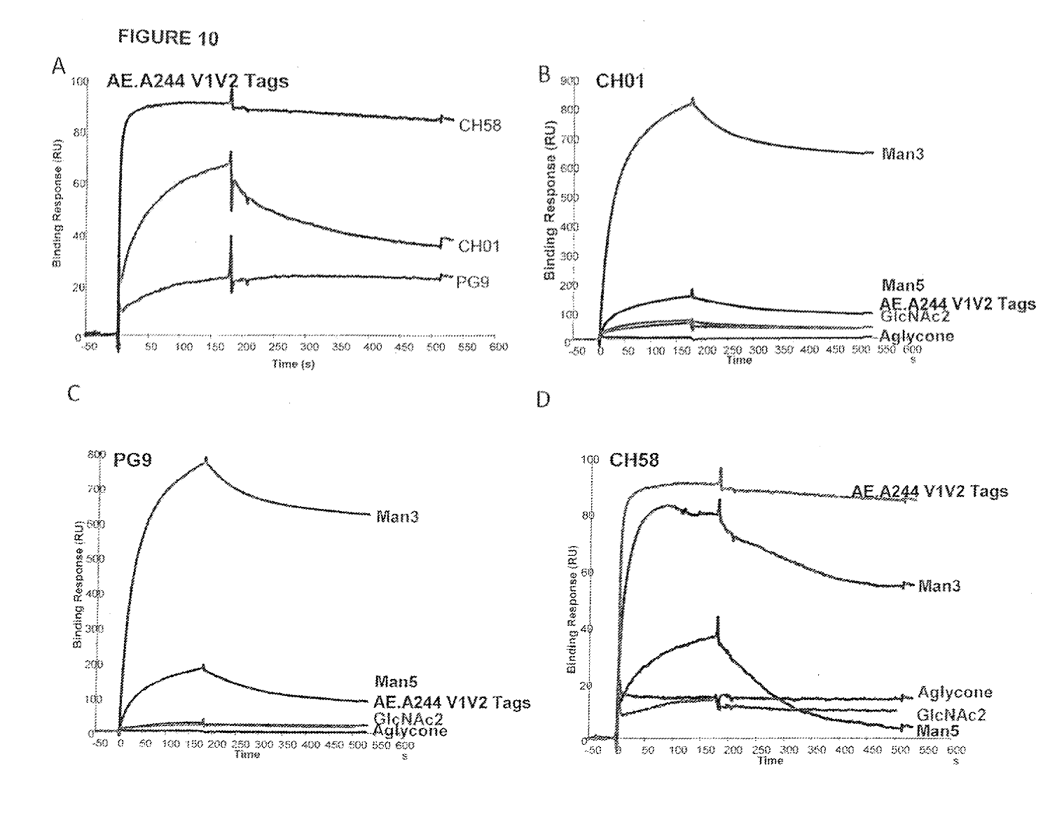

As shown in FIG. 10A, CH58 binds more avidly to A244 V1v2 tags protein when compared to the binding of bNAbs PG9 or CH01. BNabs PG9 and CH01 bind selectively to the glycopeptides V1/V2 Man₃GlcNAc₂ and V1/V2 Man₅GlcNAc₂ (FIGS. 10B and 10C). MAb CH58 binds avidly to A244 V1v2 tags protein and weakly with fast dissociation rates to V1V2 glycopeptides (FIG. 10D). SPR binding assay was performed as described in FIG. 9.

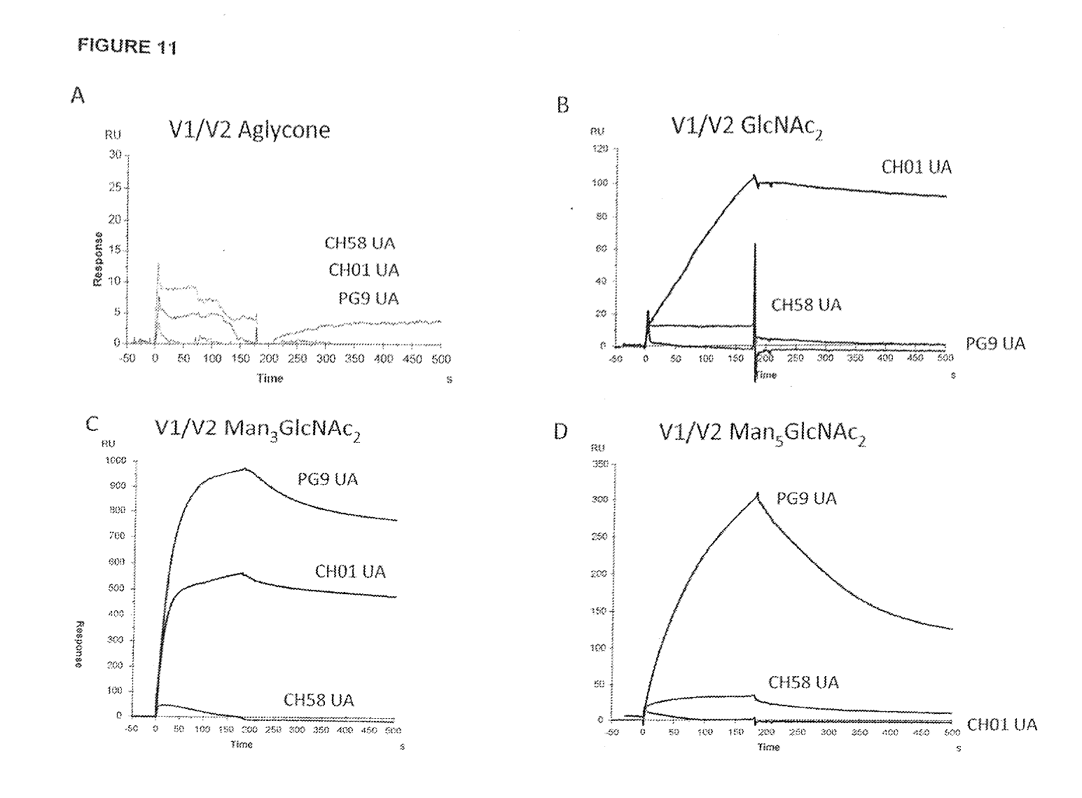

FIG. 11 shows binding of V1/V2 unmutated ancestor (UA) antibodies to synthetic V1/V2 aglycone and glycopeptides. UAs of both bNAbs PG9 and CH01 bind only to glycopeptides. UAs of CH58, PG9 or CH02 show no binding to the V1V2 aglycone peptide (FIG. 11A), V1/V2 GlcNAc₂ bound to CH01 UA with slow association indicating weak affinity interaction, but showed no binding to CH58 UA or PG9 UA (FIG. 11B). UAs of both PG9 and CH01 but not of CH58 binds to V1/V2 Man₃GlcNAc₂ (FIG. 11C). UA of PG9 binds avidly to V1/V2 Man₅GlcNAc₂, while CH58 UA binds weakly (FIG. 11D).

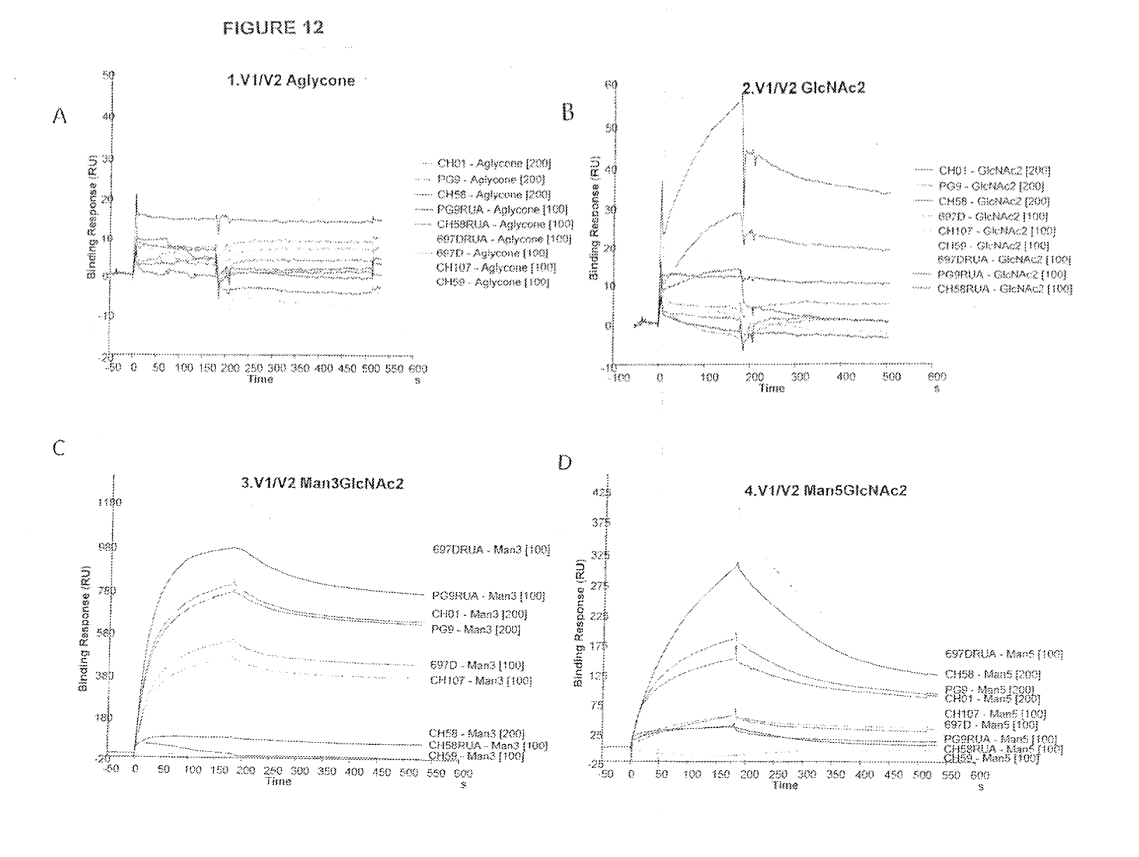

FIG. 12 shows binding of a panel of V2 and V1/V2 mAbs to aglycone (FIG. 12A), V1/V2 GlcNAc₂ (FIG. 12B), V1/V2 Man₃GlcNAc₂ (FIG. 12C) and V1/V2 Man₅GlcNAc₂ (FIG. 12D). Both conformational V2 mAbs (697D) and V1V2 mAbs (PG9, CH01) and their UAs bind to the glycopeptides but not to the aglycone peptide.

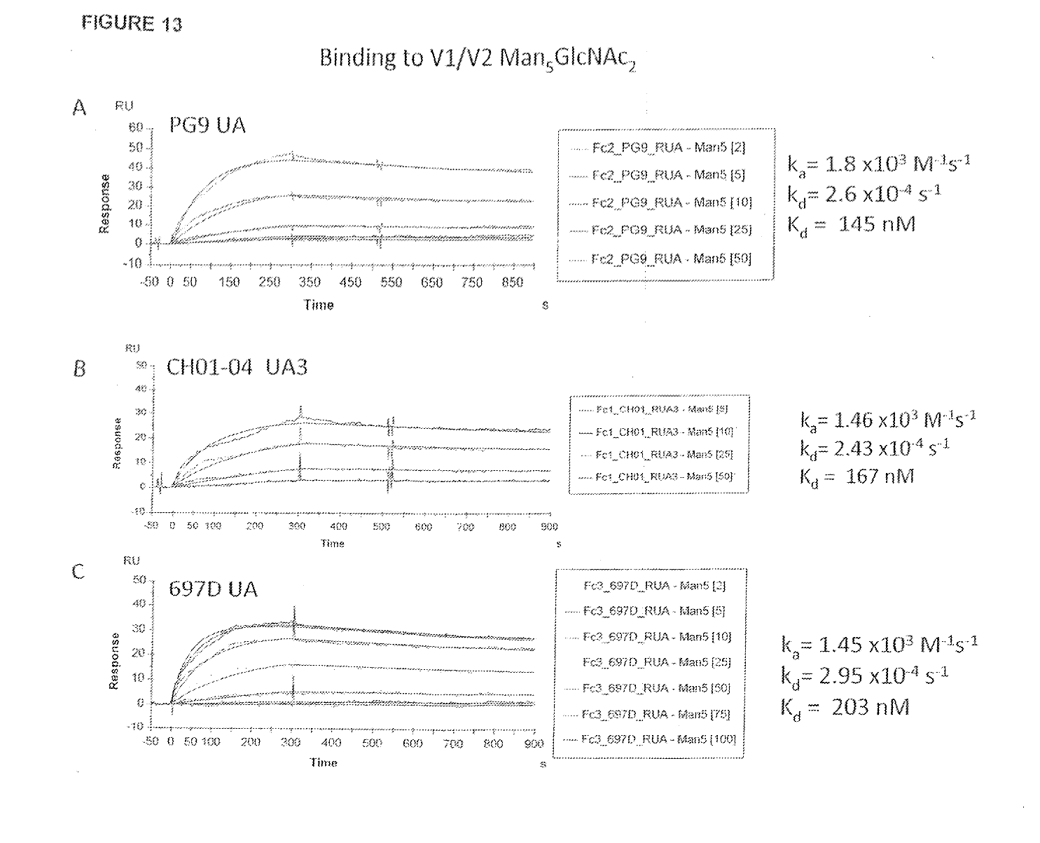

Binding of UAs of conformational V1 V2 (PG, CH01) and V2 (697D) to V1/V2 Man₅GlcNAc₂ is shown in FIG. 13. The binding Kd (disassociation constant) of the UAs ranges from about 0.15 to 0.2 μM. Varying concentrations of the V1V2 glycopeptide ranging from 2 to 100 μg/mL was injected over each of the listed mAbs and binding Kd ws calculated by global curve fitting analysis to 1:1 Langmuir model

### Example 2

A synthetic route has been developed to access gp120-based glycopeptide fragments that encompass the important elements of the V1/V2 binding surface known to interface with the BnAb PG9 (McLellan et al, Nature 480:336-343 (2011)), exemplified by compounds 1 and 2 (FIG. 14). The overall strategy relies on the paradigm of convergent N-linked glycopeptide assembly (Cohen-Anisfeld et al, J. Am. Chem. Soc. 115:10531-10537 (1993), Miller et al, Angew. Chem. Int. Ed. 42:431-434 (2003)), wherein the requisite carbohydrate and peptide domains are synthesized independently, and joined using the aspartylation conditions previously described (Cohen-Anisfeld et al, J. Am. Chem. Soc. 115:10531-10537 (1993), Miller et al, Angew. Chem. Int. Ed. 42:431-434 (2003), Wang et al, Angew. Chem. Int. Ed. [Online early access]. DOI: 10.1002/anie.201205038. Published online: Sep. 25, 2012, Ullmann et al, Angew. Chem. Int. Ed. [Online early access]. DOI: 10.1002/anie.201204272. Published online: Sep. 3, 2012).

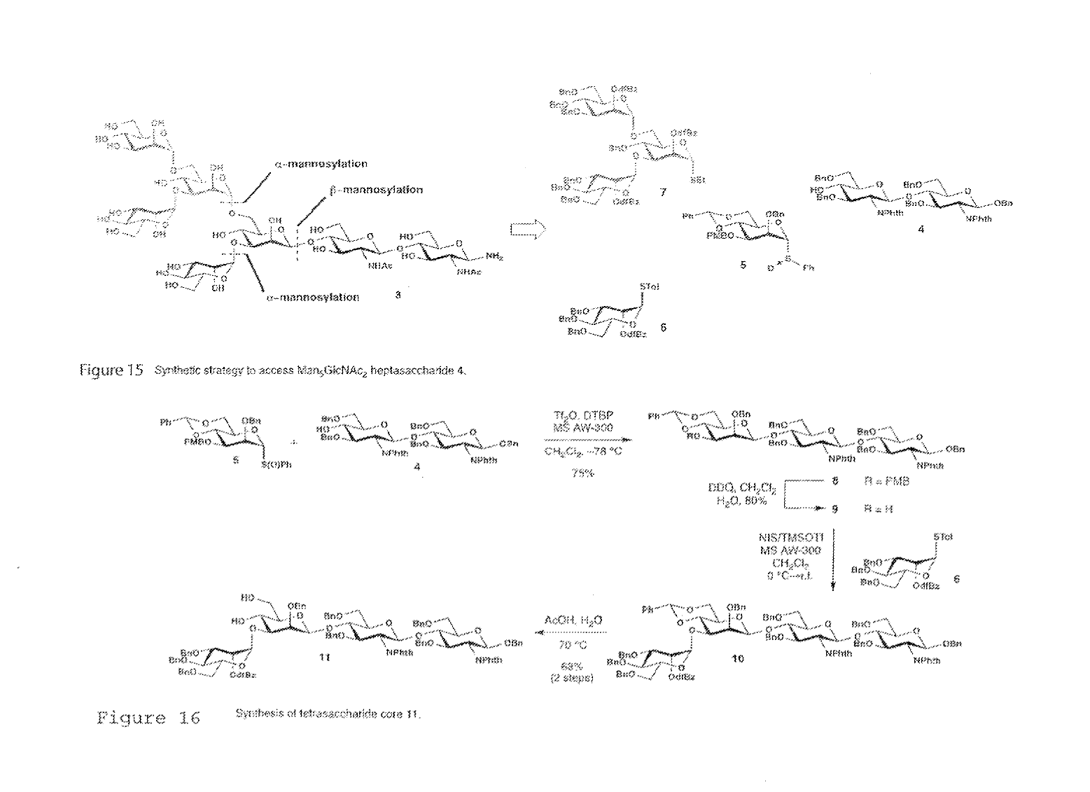

The plan for accessing the Man₅GlcNAc₂ glycan 3 is outlined in FIG. 15. It is envisioned that the key β-mannosyl linkage would be constructed by coupling disaccharide acceptor 4 (Ogawa et al, Carbohydr. Res. 228:157-170 (1983)) with mannosyl donor 5 (Crich et al, J. Am. Chem. Soc. 123:5826-5828 (2001)) using the method of Crich et al (J. Am. Chem. Soc. 126:15081-15086 (2004)). The remaining mono- and tri-mannosyl units would be introduced sequentially using donors 6 and 7, respectively.

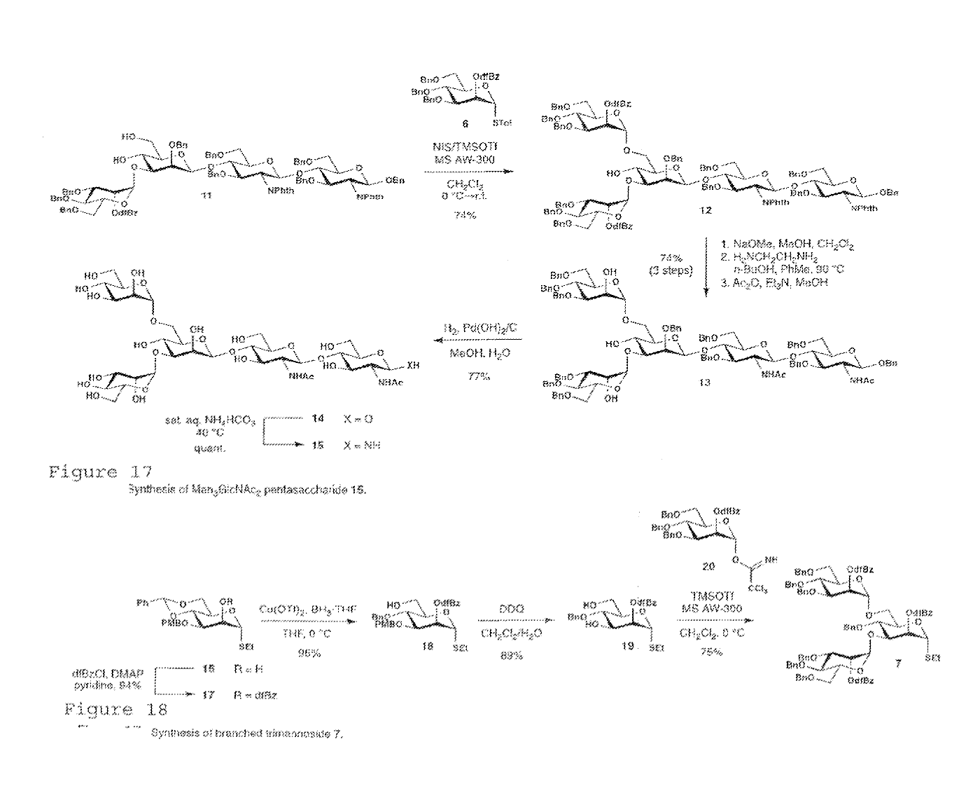

The planned β-mannosylation of disaccharide acceptor 4 using donor 5 proceeded in 75% yield under Crich's conditions, furnishing trisaccharide 8 (FIG. 16). The PMB group was removed in 80% yield, then coupling of the resulting acceptor 9 with thioglycoside donor 6 was accomplished under NIS/TMSOTf activation conditions, yielding tetrasaccharide 10. Cleavage of the benzylidene acetal with aqueous acetic acid afforded diol 11 in 63% overall yield from 9.

Acceptor 11 is a common intermediate en route to the synthesis of the pentasaccharide Man₃GlcNAc₂ and the heptasaccharide Man₅GlcNAc₂, depending on the choice of donor used to glycosylate the C-6 hydroxyl group. This moiety was selectively coupled with mannosyl donor 6 to provide the fully protected Man₃GlcNAc₂ unit 12 in 74% yield (FIG. 17). A three-step sequence involving ester saponification, phthalimide cleavage, and N-acetylation furnished partially deprotected pentasaccharide 13 in 74% overall yield. Global debenzylation proceeded smoothly via hydrogenolysis to give fully deprotected pentasaccharide 14 as a mixture of anomeric alcohols in 77% yield. This compound was quantitatively converted to the anomeric amine 15 under Kochetkov amination conditions (Likhosherstov et al, Carbohydr. Res. 146, C1-C5 (1986), Nagorny et al, J. Am. Chem. Soc. 131:5792-5799 (2009)).

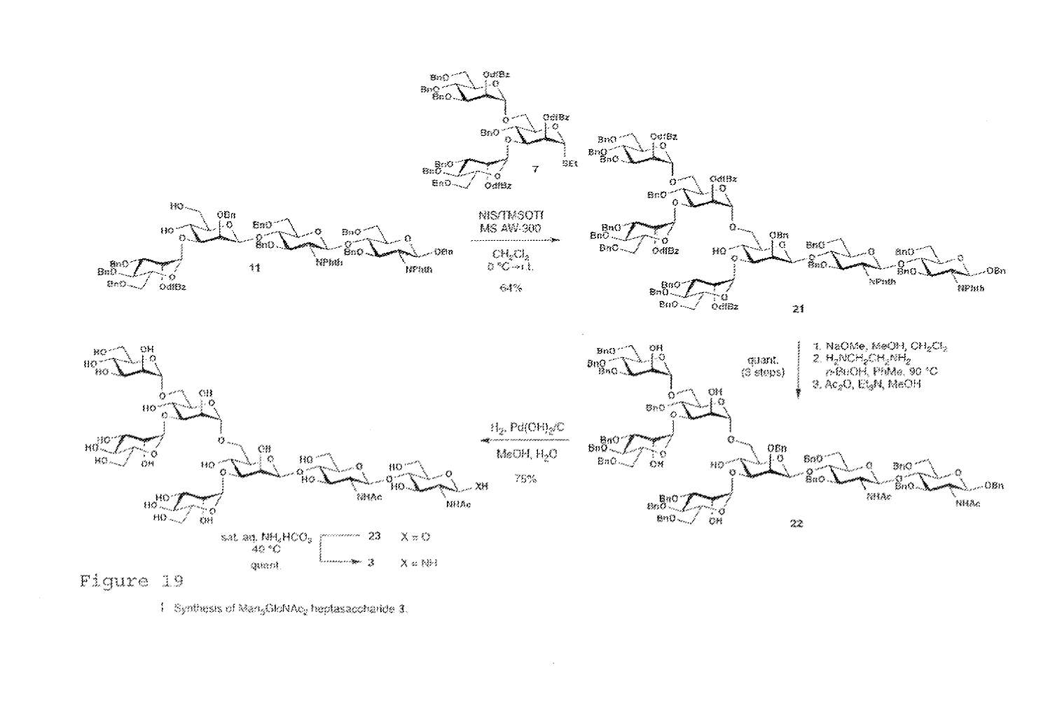

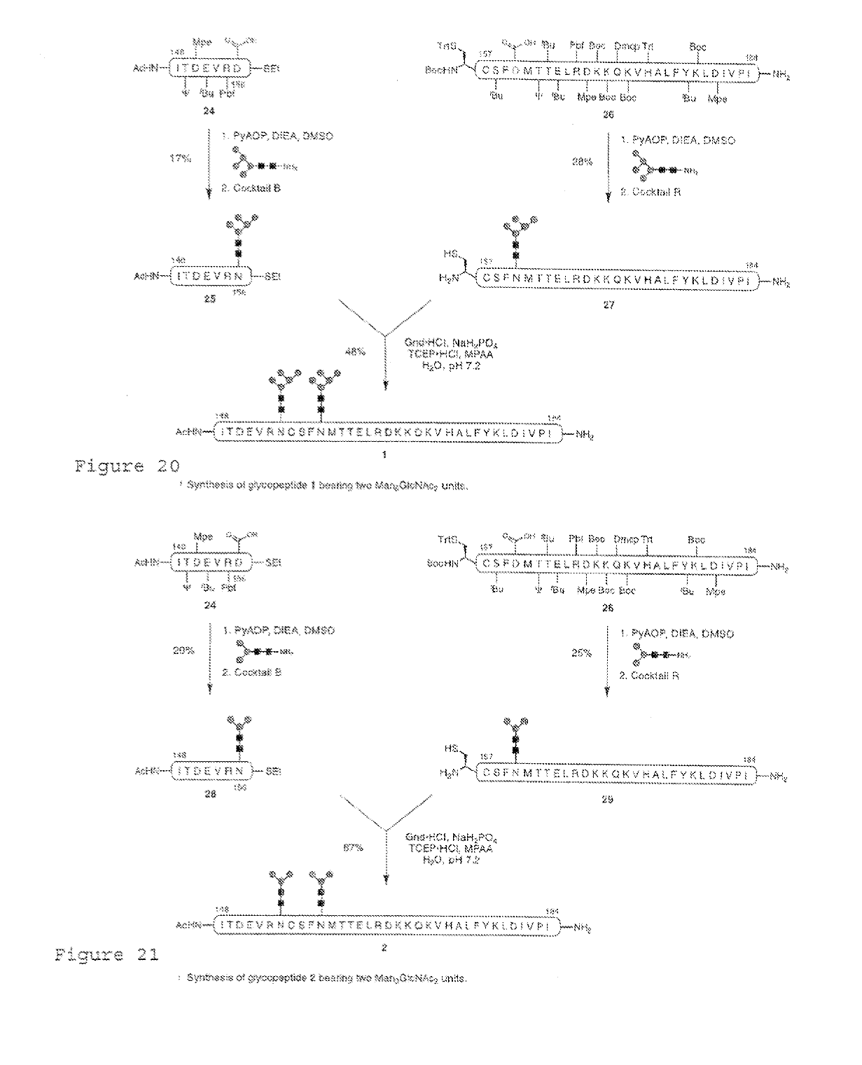

Synthesis of the required trimannosyl donor 7 for making the heptasaccharide was accomplished by straightforward elaboration of known mannosyl building block 16 (Cherif et al, J. of Carbohydr. Chem. 21:123 (2002)) (FIG. 18). Installation of the 2,5-difluorobenzoyl ester gave 17 in 94% yield. Reductive ring opening was accomplished selectively with borane-THF complex in the presence of copper triflate in 96% yield (Shie et al, Angew. Chem. Int. Ed. 44:1665-1668 (2005)). Cleavage of the PMB group afforded 3,6-diol 19, which underwent double mannosylation with imidate donor 20 in 75% yield to furnish branched trimannoside 7. The stage was now set for the key coupling between tetrasaccharide acceptor 11 and trisaccharide donor 7 (FIG. 19). In the event, 7 was activated with NIS/TMSOTf and joined with 11 to provide the fully elaborated protected heptasaccharide 21 in 64% yield. Subjection of this material to the 4-step global deprotection protocol resulted in a 75% overall yield of fully deprotected heptasaccharide 23 as a mixture of anomers. The anomeric amine 3 was subsequently generated by application of the Kotchetkov conditions.

The second phase of the synthetic effort dealt with the assembly of the peptide domains of the targeted glycopeptide constructs, and their coupling to oligosaccharides 3 and 15 (FIGS. 20 and 21). Each doubly glycosylated polypeptide was generated by joining two individually glycosylated fragments via native chemical ligation (NCL) (Dawson et al, Science 266:776-779 (1994)). Peptide thioester 24 was obtained by Fmoc SPPS and post-resin C-terminal functionalization procedures (Kuroda et al, Int. J. Pept. Prot. Res. 40:294-299 (1992)) in the context of other glycopeptides (Nagorny et al, J. Am. Chem. Soc. 131:5792-5799 (2009), Chen et al, Tetrahedron Lett. 47:8013-8016 (2006)). The free carboxylic acid side chain at position 156 was joined to the Man₅GlcNAc₂ glycosyl amine 3 under Lansbury conditions, then standard TFA-based deprotection provided glycopeptide thioester 25 in 17% yield after purification by reversed phase HPLC (FIG. 20). A similar sequence was used to convert protected peptide fragment 26 to glycopeptide 27 (28% yield). The two glycopeptide fragments were successfully joined by NCL in 48% yield to afford the fully elaborated glycopeptide 1 bearing Man₅GlcNAc₂ units at N156 and N160. FIG. 21 outlines the synthesis of glycopeptide 2 bearing two Man₃GlcNAc₂ units, which was prepared in analogous fashion.

### Example 3

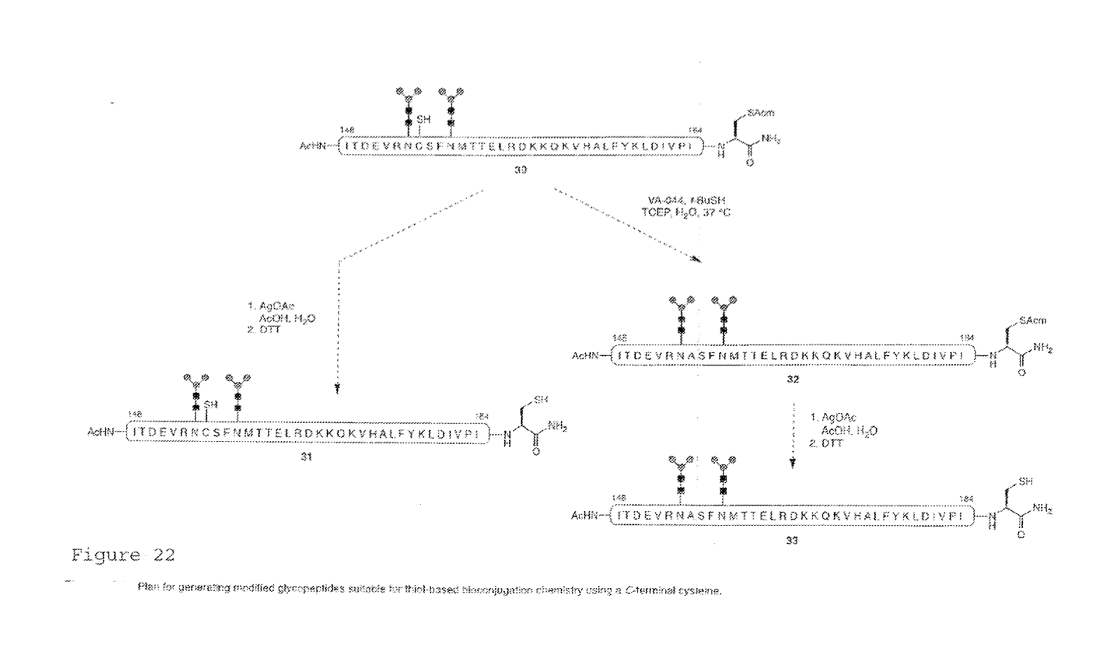

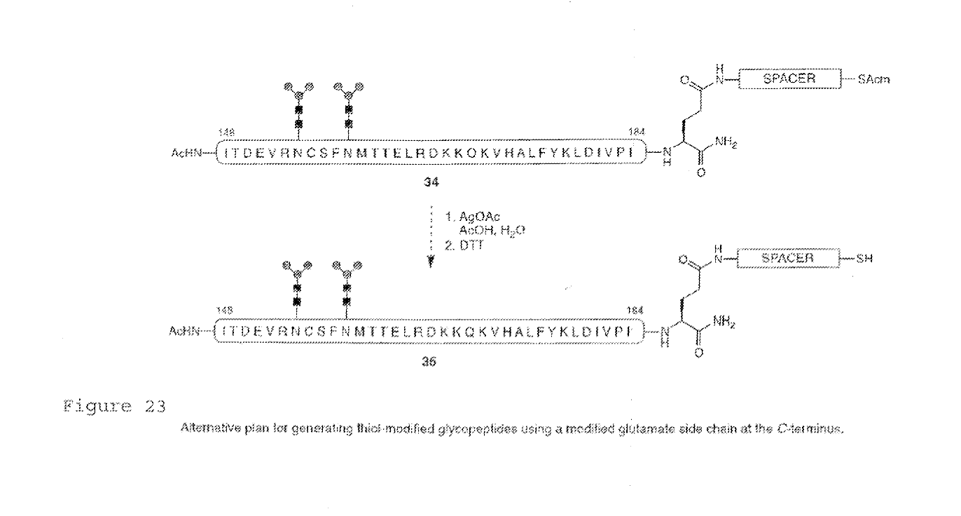

The glycopeptides can be conjugated to carrier proteins using a suitably exposed thiol function. To incorporate this chemical handle, the current synthetic route can be modified by introducing cysteine (with the sidechain protected by an Acm group) at the C-terminus during Fmoc SPPS of fragment 26. Carrying this modified peptide through the synthesis would afford a glycopeptide like 30 in the case of the Man₃GlcNAc₂-based glycopeptide (FIG. 22). Silver-promoted cleavage of the Acm group (Bang et a, J. Am. Chem. Soc. 126:1377-1383 (2004)) would furnish the free thiol 31, ready for conjugation. A potentially complicating factor is that 31 also possesses an internal thiol (at C157), which could, in principle, also react during the conjugation. However, due to its placement within the sequence between the two large glycan moieties, it is anticipated that chemistry at the internal thiol will be kinetically disfavored. Nevertheless, there is also the option of removing the sidechain of C157 using the mild protocol for metal-free dethylation (Wan et al, Angew. Chem. Int. Ed. 46:9248-9252 (2007)). Subsequent Acm removal would give glycopeptide 33, where C157 has been mutated to alanine. FIG. 23 depicts an alternate thiol functionalization scheme that would involve incorporating glutamate at the C-terminus, where the sidechain has been modified with a thiol-based linker.

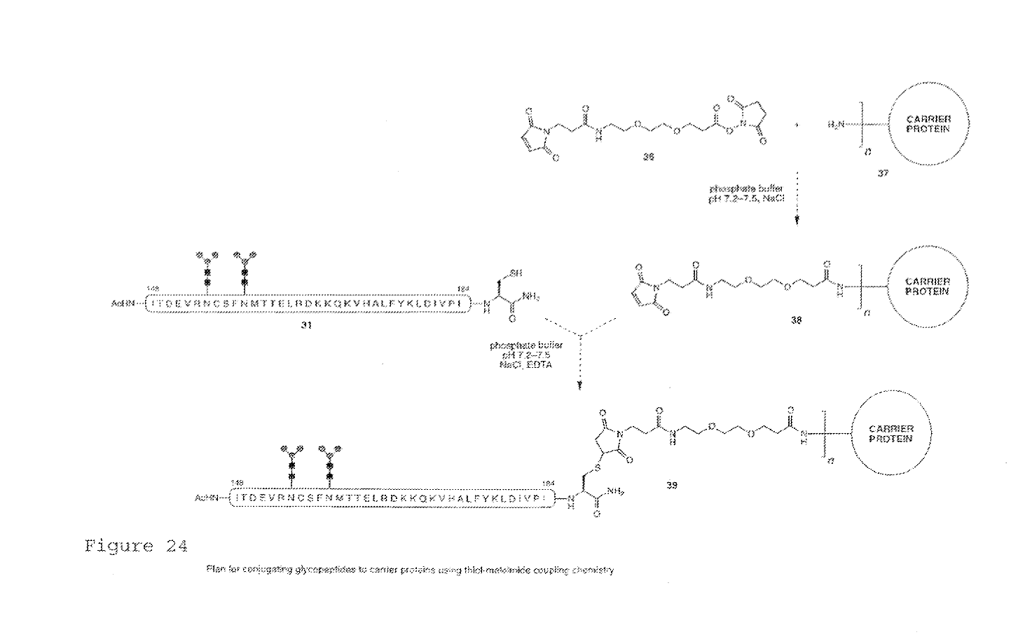

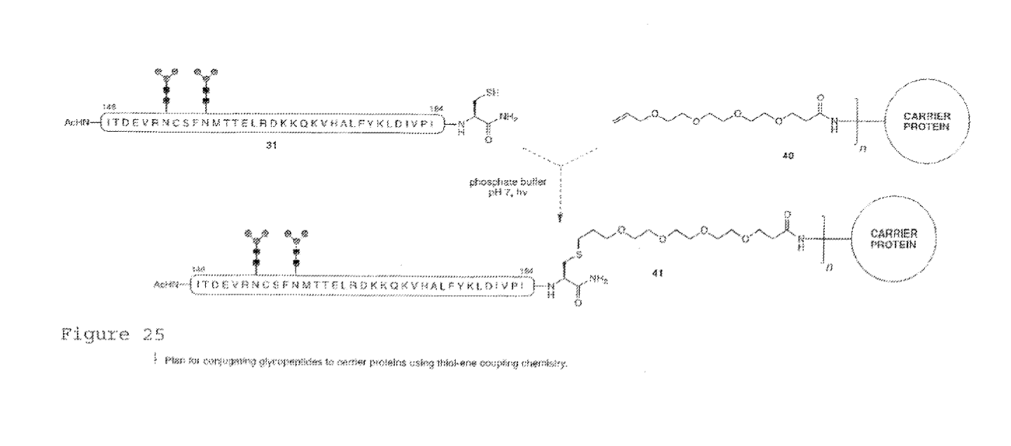

FIG. 24 outlines how thiol-bearing glycopeptides such as 31, 33, and 35 can be coupled to carrier proteins such as CRM197 (a non-toxic variant of diphtheria toxin), KLH (keyhole limpet hemocyanin), or TT (tetanus toxoid) using thiol-maleimide bioconjugation (Hermanson, G. T. In Bioconjugate Techniques (Second Edition); Academic Press: New York, pp. 743-782 (2008)). The carrier protein 37 is first functionalized using a heterobifunctional linker such as 36 (commercially available from Pierce), then the maleimide-decorated carrier 38 is combined with the glycopeptide (31 in FIG. 24) yielding vaccine constructs where multiple glycopeptides are conjugated to the carrier, as exemplified by 39.

Kunz and co-workers have shown that the thiol-ene coupling can also be applied in bioconjugation contexts (Wittrock et al, Angew. Chem. Int. Ed. 46:5226-5230 (2007)). This chemistry presents an attractive alternative to the maleimide-based procedure, as shown in FIG. 25. Suitable olefin-modified carriers 40 can be obtained using Kunz's linker strategy. Conjugation is subsequently achieved under photochemical conditions.

### Example 4

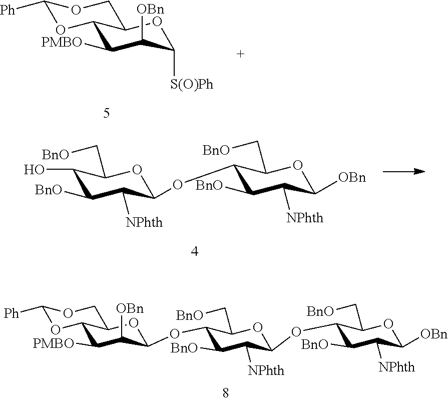

### Benzyl 2-O-benzyl-3-O-p-methoxybenzyl-4,6-O—(R)-benzylidene-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside (8)

Freshly activated AW-300 MS (2 g) were added to a solution of mannose sulfoxide 5 (1.0 g, 1.70 mmol) in anh. CH₂Cl₂ (20 ml). After 1 h at rt, the mixture was cooled to −78° C., and di-tert-butyl pyridine (0.9 ml, 4 mmol) and Tf₂O (0.3 ml, 1.8 mmol) were added. The mixture was allowed to warm up to −50° C. over 30 min, cooled to −78° C. and a solution of acceptor 4 (1.2 g, 1.14 mmol) in CH₂Cl₂ (10 ml) was added dropwise. The mixture was stirred at −78° C. for 5 h, filtered through a pad of Celite, washed with sat. NaHCO₃ solution, water, brine, dried over MgSO₄ and concentrated. Purification by chromatography on SiO₂ (Hexanes:CH₂Cl₂:EtOAc, 4:4:1) afforded 8 (1.3 g, 75%) as amorphous white solid in single diastereomeric form.

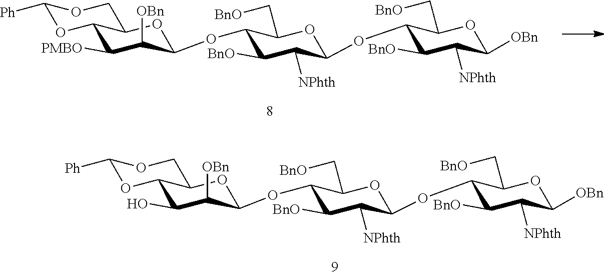

### Benzyl 2-O-benzyl-4,6-O—(R)-benzylidene-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside (9)

Trisaccharide 8 (1.3 g, 0.86 mmol) was dissolved in CH₂Cl₂ (20 ml), followed by addition of H₂O (20 ml), and the mixture treated with DDQ (1 g, 4.4 mmol). The mixture was stirred vigorously at rt, in the dark for 4 h. The reaction was quenched with a buffer solution (0.7% Ascorbic acid+1.3% citric acid+1.9% NaOH in H₂O, w/v) (5 ml), diluted with CH₂Cl₂ (20 ml), washed with water, brine, dried over MgSO₄ and concentrated. Purification by chromatography on SiO₂ (Hexanes:CH₂Cl₂:EtOAc, 4:4:1) afforded 9 (0.95 g, 80%) as amorphous white solid.

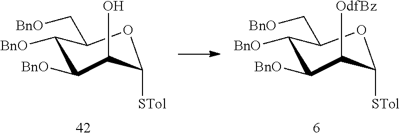

### p-Tolyl 3,4,6-tri-O-benzyl-(2,5-difluorobenzoyl)-1-thio-α-D-mannopyranoside

Thioglycoside 42 (Chayajarus et al, Org. Lett. 6:3797-3800 (2004)) (10.0 g, 18.0 mmol) and 4-dimethylaminopyridine (0.22 g, 1.8 mmol) were dissolved in pyridine (50 mL), and then 2,5-difluorobenzoyl chloride (6.7 mL, 54.0 mmol) was added. The mixture was stirred at room temperature overnight and then diluted with CH₂Cl₂ (300 mL). The mixture was washed with saturated aqueous NaHCO₃ (150 mL), water (150 mL) and 1 N HCl (150 mL), and then dried over Na₂SO₄, filtered and concentrated. Purification by silica gel chromatography (9:1 to 85:15 hexanes/ethyl acetate) afforded difluorobenzoyl ester 6 (11.0 g, 88% yield) as a clear oil.

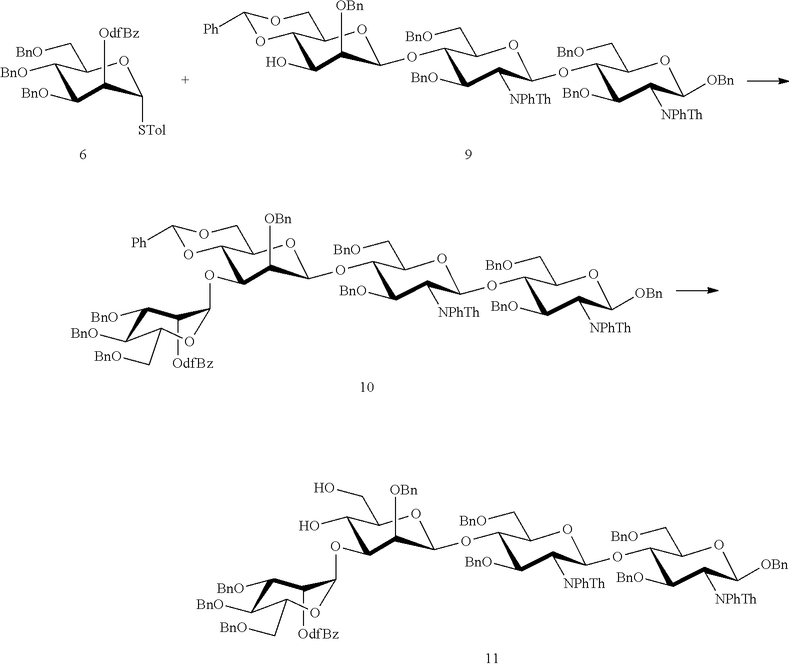

### Benzyl[3,4,6-tri-O-benzyl-2-O-(2,5-difluorobenzoyl)-α-D-mannopyranosyl-(1→3)]-2-O-benzyl-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside (11)

A mixture of trisaccharide acceptor 9 (330 mg, 0.24 mmol) and mannose thioglycoside donor 6 (215 mg, 0.31 mmol) was dissolved in anh. CH₂Cl₂ (20 ml). Freshly activated AW-300 MS (0.5 g) was added and stirred at rt for 1 h. The mixture was cooled to 0° C., NIS (75 mg, 0.33 mmol) and TMSOTf (10 μl, 0.05 mmol) were added sequentially, and the mixture was allowed to warm up to rt over 5 h. The mixture was filtered through a pad of Celite and the organic layer was washed with sat. Na₂S₂O₃, sat. NaHCO₃ solution, water, brine, dried over MgSO₄ and concentrated.

The crude material was dissolved in acetic acid (10 ml). H₂O (1.5 ml) was added dropwise with stirring and the reaction mixture was heated at 70° C. for 3 h. The mixture was co-evaporated with toluene and the crude mass was purified by chromatography on SiO₂ (Hexanes: EtOAc, 1:1) to give 11 (280 mg, 63%) as amorphous white solid.

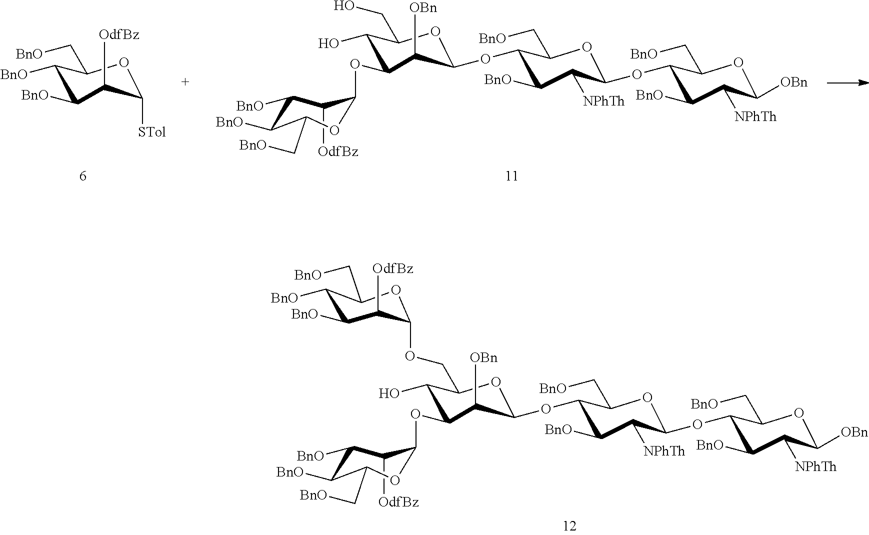

### Benzyl[3,4,6-tri-O-benzyl-2-O-(2,5-difluorobenzoyl)-α-D-mannopyranosyl-(1→3)]-[3,4,6-tri-O-benzyl-2-O-(2,5-difluorobenzoyl)-α-D-mannopyranosyl-(1→6)]-2-O-benzyl-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside (12)

A mixture of tetrasaccharide acceptor 11 (245 mg, 0.13 mmol) and mannose thioglycoside donor 6 (91 mg, 0.13 mmol) was dissolved in anh. CH₂Cl₂ (20 ml). Freshly activated AW-300 MS (0.5 g) was added and stirred at rt for 1 h. The mixture was cooled to 0° C., NIS (75 mg, 0.33 mmol) and TMSOTf (10 μl, 0.05 mmol) were added sequentially, and the mixture was allowed to warm up to rt over 5 h. The mixture was filtered through a pad of Celite and the organic layer was washed with sat. Na₂S₂O₃, sat. NaHCO₃ solution, water, brine, dried over MgSO₄ and concentrated. The residue was purified by chromatography on SiO₂ (Hexanes: EtOAc, 2:1) to give the pentasaccharide 12 (236 mg, 74%) as amorphous white solid.

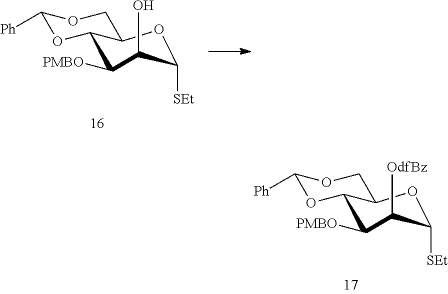

### Ethyl 4,6-O-benzylidene-2-O-(2,5-difluorobenzoyl)-3-O-p-methoxybenzyl-1-thio-α-D-mannopyranoside (17)

To a solution of alcohol 16 (Cherif et al, J. of Carbohydr. Chem. 21:123 (2002)) (768 mg, 1.77 mmol) and 4-dimethylaminopyridine (43.4 mg, 0.355 mmol) in pyridine (5.0 mL) was added 2,5-difluorobenzoyl chloride (0.44 mL, 3.55 mmol) via syringe pump over 10 min. The reaction mixture was stirred at room temperature; gradual formation of a white precipitate was observed over time. An additional portion of 2,5-difluorobenzoyl chloride (0.11 mL, 0.887 mmol) was added at 19.5 h. After a total reaction time of 44 h, MeOH (0.80 mL) was added. The resulting mixture was stirred for 1 h, then diluted with CH₂Cl₂ (80 mL) and washed with water (120 mL) The aqueous phase was back-extracted with CH₂Cl₂ (60 mL), then the combined organic layers were dried (MgSO₄), filtered, and concentrated. Purification by flash chromatography (10% EtOAc/hexanes) afforded an oily white solid that was taken up in EtOAc (80 mL) and washed with saturated aqueous NaHCO₃ (2×20 mL) (to remove residual 2.5-difluorobenzoic acid). The combined aqueous phases were back-extracted with EtOAc (40 mL). The organic layers were then combined, washed with water (20 mL) and brine (20 mL), dried (MgSO₄), filtered, and concentrated to provide difluorobenzoyl ester 17 as a yellowish foam in 94% yield (960 mg, 1.68 mmol).

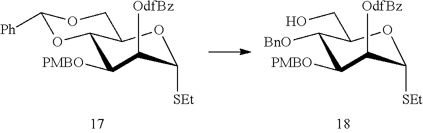

### Ethyl 4-O-benzyl-2-O-(2,5-difluorobenzoyl)-3-O-p-methoxybenzyl-1-thio-α-D-mannopyranoside (18)

To a cooled (0° C.) round-bottom flask containing benzylidene acetal 17 (960 mg, 1.68 mmol) was added borane-THF complex (1.0 M in THF, 8.4 mL, 8.40 mmol). The resulting clear, colorless solution was stirred for 10 min at 0° C. Copper(II) trifluoromethanesulfonate (60.7 mg, 0.168 mmol) was then added in one portion, giving a light brown suspension that was maintained at 0° C. for 25.5 h with vigorous stirring. The reaction was carefully quenched while cold by successive addition of triethylamine (0.24 mL) and MeOH (3.0 mL) (CAUTION: H₂ evolution!). Volatiles were removed on a rotary evaporator, and the residue was co-evaporated with MeOH a few times, resulting in a cloudy, dark brown oil. Purification by flash chromatography (20% EtOAc/hexanes) afforded alcohol 18 as a clear, very pale yellow oil in 96% yield (929 mg, 1.62 mmol).

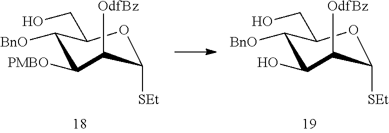

### Ethyl 4-O-benzyl-2-O-(2,5-difluorobenzoyl)-1-thio-α-D-mannopyranoside (19)

To a solution of PMB ether 18 in CH₂Cl₂ (7.2 mL) was added water (0.40 mL) and 2,3-dichloro-5,6-dicyano-1,4-benzoquinone (263 mg, 1.16 mmol), resulting in a dark greenish-black color that became reddish-orange over time. After stirring for 4 h at room temperature, the reaction was quenched with a solution of ascorbic acid/citric acid/NaOH (0.7%/1.3%/0.9% in water, 50 mL) and diluted with EtOAc (100 mL). The layers were separated, and the aqueous phase was extracted with EtOAc (2×100 mL). The combined organic layers were filtered through a pad of Celite, and the filtrate was washed with saturated aqueous NaHCO₃ (100 mL), brine (100 mL), dried (Na₂SO₄), filtered, and concentrated. Purification by flash chromatography (20% EtOAc/hexanes) afforded diol 19 as a clear, colorless oil in 89% yield (312 mg, 0.685 mmol).

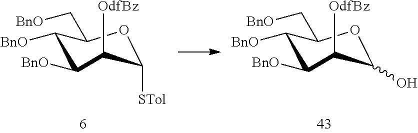

### 3,4,6-Tri-O-benzyl-2-O-(2,5-difluorobenzoyl)-D-mannopyranoside (43)

To a cooled (0° C.) solution of thioglycoside 6 (1.88 g, 2.70 mmol) in acetone/water (9:1, 40 mL) was added N-bromosuccinimide (1.44 g, 8.09 mmol). The resulting clear, orange solution was stirred at 0° C., with additional portions of N-bromosuccinimide (480 mg, 2.70 mmol) added at 1 h and 4 h. After a total reaction time of 6 h, the reaction mixture was concentrated until turbidity was evident. The residue was then taken up in EtOAc (500 mL), washed with saturated aqueous NaHCO₃ (3×120 mL), water (3×120 mL), dried (Na₂SO₄), filtered, and concentrated. Purification by flash chromatography (25% EtOAc/hexanes) afforded anomeric alcohol 43 as a clear, colorless oil in 86% yield (1.37 g, 2.33 mmol, α:β mixture).

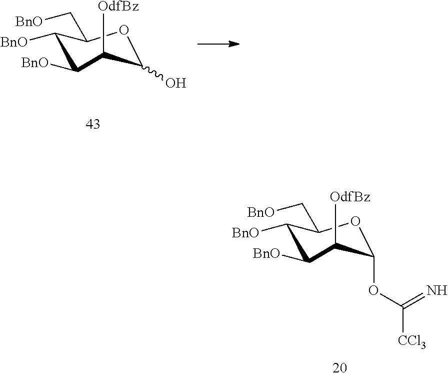

### 3,4,6-Tri-O-benzyl-2-O-(2,5-difluorobenzoyl)-α-D-mannopyranosyl trichloroacetimidate (20)

To a cooled (0° C.) solution of anomeric alcohol 43 (1.35 g, 2.29 mmol) and trichloroacetonitrile (2.3 mL, 22.9 mmol) in CH₂Cl₂ (9.0 mL) was added and 1,8-diazabicyclo[5.4.0]undec-7-ene (40 μL, 0.267 mmol) dropwise via syringe. The resulting clear, yellow solution was stirred at 0° C. for 4 h. The reaction mixture was loaded directly on a short silica gel column and purified by flash chromatography (20% EtOAc/hexanes) to afford trichloroacetimidate 20 as a clear, yellow oil in 96% yield (1.61 g, 2.19 mmol).

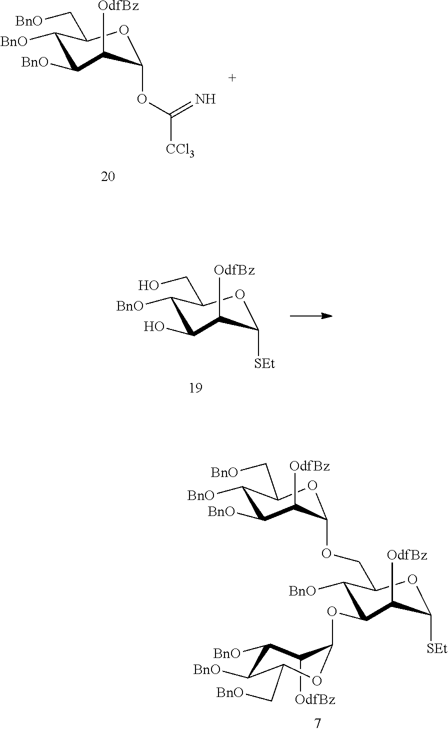

### Ethyl 3,4,6-tri-O-benzyl-2-O-(2,5-difluorobenzoyl)-α-D-mannopyranosyl-(1→3)-[3,4,6-tri-O-benzyl-2-O-(2,5-difluorobenzoyl)-α-D-mannopyranosyl-(1→6)]-4-O-benzyl-2-O-(2,5-difluorobenzoyl)-1-thio-α-D-mannopyranoside (7)

Diol acceptor 19 (294 mg, 0.647 mmol) and trichloroacetimidate donor 20 (1.17 g, 1.60 mmol) were azeotroped three times with benzene then dried for 2 h in vacuo. The residue was dissolved in CH₂Cl₂ (6.5 mL), and the clear, yellow solution was stirred in the presence of acid-washed molecular sieves (AW-300, 1.6 mm pellets, 900 mg) for 15 min at room temperature. The mixture was cooled to 0° C., then trimethylsilyl trifluoromethanesulfonate (5% in CH₂Cl₂, 0.24 mL, 66.4 μmol) was added dropwise via syringe. After stirring for 2 h at 0° C., the reaction medium was neutralized with a few drops of triethylamine, then filtered and concentrated. Purification by flash chromatography (0-1% EtOAc/CH₂Cl₂) afforded trisaccharide 7 as a white foam in 75% yield (771 mg, 0.482 mmol).

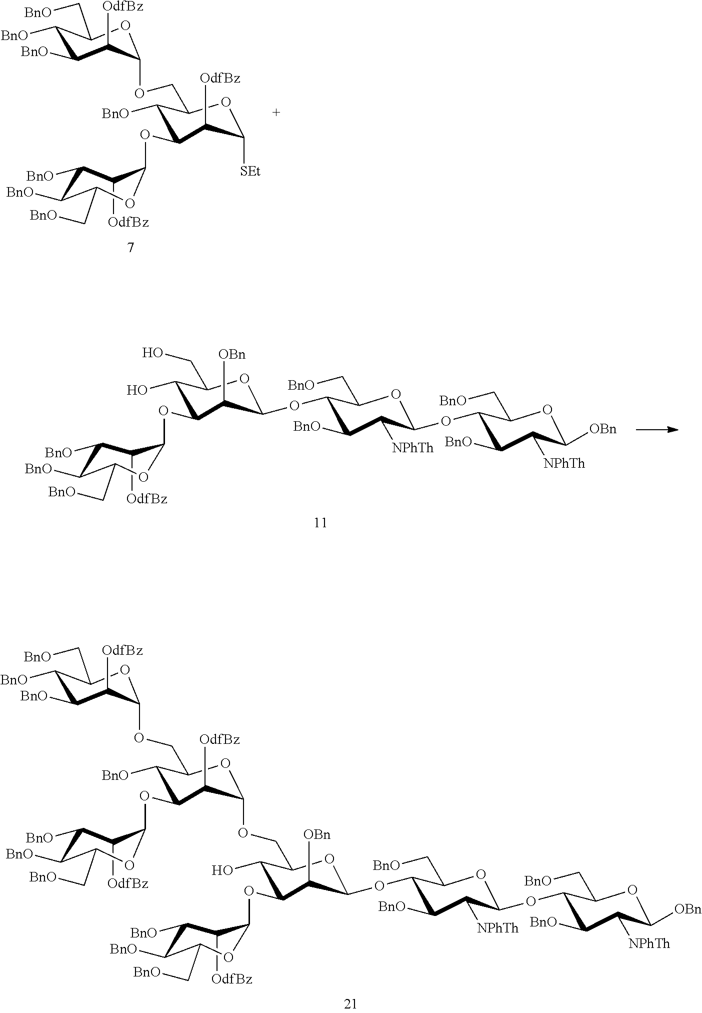

### Benzyl[3,4,6-tri-O-benzyl-2-O-(2,5-difluorobenzoyl)-α-D-mannopyranosyl-(1→3)]-[[3,4,6-tri-O-benzyl-2-O-(2,5-difluorobenzoyl)-α-D-mannopyranosyl-(1→3)]-[3,4,6-tri-O-benzyl-2-O-(2,5-difluorobenzoyl)-α-D-mannopyranosyl-(1→6)]]-4-O-benzyl-2-O-(2,5-difluorobenzoyl)-α-D-mannopyranosyl-(1→6)]-2-O-benzyl-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside (21)

A mixture of tetrasaccharide acceptor 11 (275 mg, 0.15 mmol) and trimannose thioglycoside donor 7 (255 mg, 0.16 mmol) was dissolved in anh. CH₂Cl₂ (20 ml). Freshly activated AW-300 MS (0.5 g) was added and stirred at rt for 1 h. The mixture was cooled to 0° C., NIS (50 mg, 0.22 mmol) and TMSOTf (6 μl, 0.03 mmol) were added sequentially, and the mixture was allowed to warm up to rt over 4 h. The mixture was filtered through a pad of Celite and the organic layer was washed with sat. Na₂S₂O₃, sat. NaHCO₃ solution, water, brine, dried over MgSO₄ and concentrated. The residue was purified by chromatography on SiO₂ (Hexanes: EtOAc, 2:1) to give the heptasaccharide 21 (310 mg, 64%, 75% based on recovered acceptor) as amorphous white solid.

### General Procedure for Global Deprotection

To a solution of oligosaccharide in CH₂Cl₂/MeOH:1/9, was added 1M NaOMe in MeOH to bring the pH of the mixture to 10. The mixture was stirred at rt for 12 h, quenched with dowex 50 W X8 resin, and evaporated to dryness. The residue was dissolved in toluene (4 ml), n-butanol (8 ml), ethylene diamine (2.4 ml), and heated at 90° C. for 24 h. The mixture was co-evaporated with toluene.

The residue was dissolved in MeOH (10 ml). Acetic anhydride (0.64 ml) and triethyl amine (1.0 ml) were added to the mixture and stirred at rt for 2 h. The reaction was monitored by LCMS at each stage. The residue was purified by chromatography on SiO₂ (Hexanes:CH₂Cl₂:Acetone, 1:1:1) to give the partially deprotected oligosaccharide as amorphous white solid.

The purified material was dissolved in MeOH (10 ml) at rt. H₂O (1.0 ml) was added dropwise, followed by addition of Pd(OH)₂/C under Argon atmosphere. Argon was replaced by Hydrogen and the mixture stirred at rt for 12 h under 1 atm pressure. The mixture was filtered by PTFE GL 0.45 μm cartridge, evaporated, and purified using C18 SepPak column. The product elutes in neat H₂O.

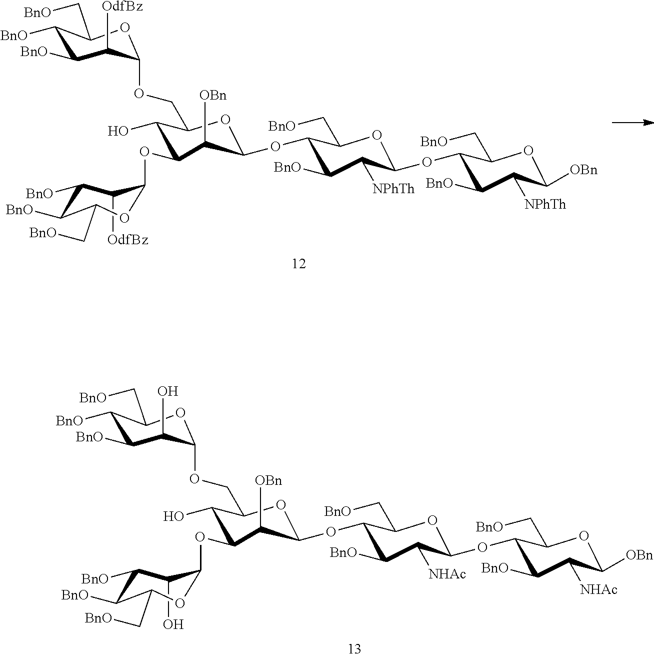

### Benzyl[3,4,6-tri-O-benzyl-α-D-mannopyranosyl-(1→3)]-[3,4,6-tri-O-benzyl-α-D-mannopyranosyl-(1→6)]-2-O-benzyl-β-D-mannopyranosyl-(1→4)-3,6-di-O-benzyl-2-deoxy-2-N-acetyl-β-D-glucopyranosyl-(1→4)-3,6-di-O-benzyl-2-deoxy-2-N-acetyl-β-D-glucopyranoside (13)

74% yield over three steps.

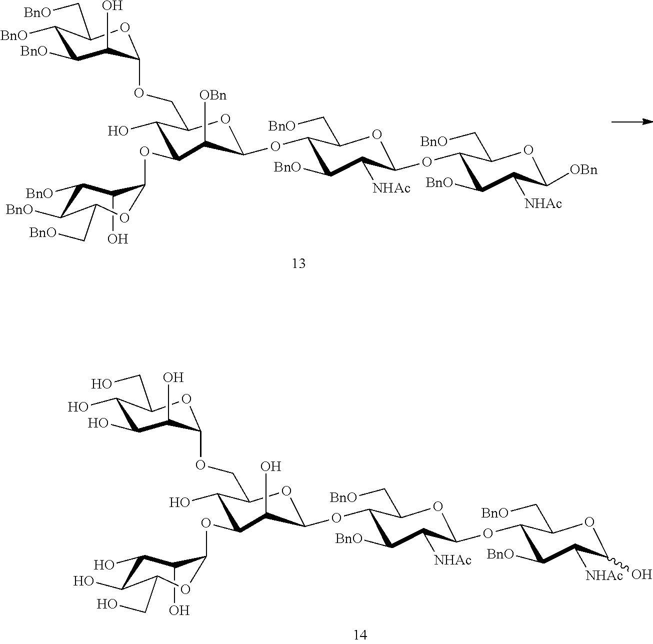

### [α-D-mannopyranosyl-(1→3)]-[α-D-mannopyranosyl-(1→6)]-β-D-mannopyranosyl-(1→4)-2-deoxy-2-N-acetyl-β-D-glucopyranosyl-(1→4)-2-deoxy-2-N-acetyl-D-glucopyranoside (14)

77% yield.

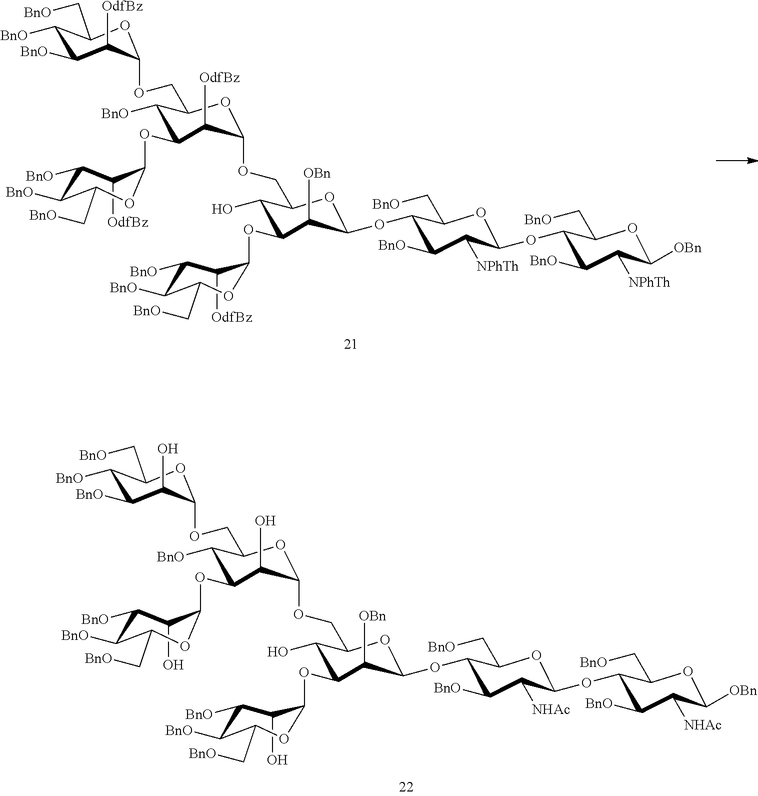

### Benzyl[3,4,6-tri-O-benzyl-α-D-mannopyranosyl-(1→3)]-[[3,4,6-tri-O-benzyl-α-D-mannopyranosyl-(1→3)]-[3,4,6-tri-O-benzyl-α-D-mannopyranosyl-(1→6)]]-4-O-benzyl-α-D-mannopyranosyl-(1→6)]-2-O-benzyl-β-D-mannopyranosyl-(1→4)-3,6-di-O-benzyl-2-deoxy-2-N-acetyl-β-D-glucopyranosyl-(1→4)-3,6-di-O-benzyl-2-deoxy-2-N-acetyl-β-D-glucopyranoside (22)

Quantitative yield over three steps.

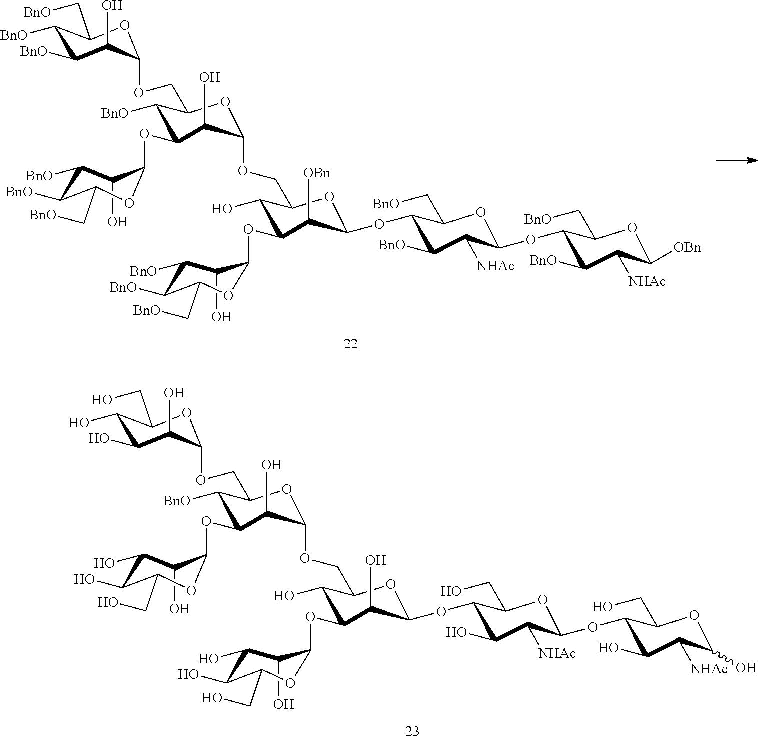

### [α-D-mannopyranosyl-(1→3)]-[[α-D-mannopyranosyl-(1→3)]-[α-D-mannopyranosyl-(1→6)]]-α-D-mannopyranosyl-(1→6)]-β-D-mannopyranosyl-(1→4)-2-deoxy-2-N-acetyl-D-glucopyranosyl-(1→4)-2-deoxy-2-N-acetyl-D-glucopyranoside (23)

75% yield.

### General Procedure for Glycan Anomeric Amine Installation (Kochetkov Reaction)

Glycan was dissolved in water (5 mL) and added to (NH₄)HCO₃ (6 g). The resultant slurry was warmed to 40° C. and stirred very slowly at this temperature for three days. After three days, the clear supernatant was filtered through a plug of cotton. The remaining material was rinsed with the same amount of cold water (2×5 mL), filtered, pooled with the clear supernatant, immediately frozen and lyophilized. The remaining material was finally dissolved in water (5 mL), filtered through a plug of cotton, frozen and lyophilized. The lyophilization was deemed complete until the mass of the product remained constant. This provided quantitatively the glycosyl amine as a white solid.

### Solid-Phase Peptide Synthesis by Fmoc-Strategy

Automated peptide synthesis was performed on an Applied Biosystems Pioneer continuous S3 flow peptide synthesizer. Peptides were synthesized under standard automated Fmoc protocols. The deblock mixture was a mixture of 100:2:2 of DMF/piperidine/DBU. The following Fmoc amino acids from NovaBiochem were employed: Fmoc-Ala-OH, Fmoc-Arg(Pbf)-OH, Fmoc-Asp(OMpe)-OH, Boc-Cys(Trt)-OH, Fmoc-Gln(Dmcp)-OH, Fmoc-Fmoc-Glu(OtBu)-OH, Fmoc-His(Trt)-OH, Fmoc-Ile-OH, Fmoc-Leu-OH, Fmoc-Lys(Boc)-OH, Fmoc-Phe-OH, Fmoc-Pro-OH, Fmoc-Ser(tBu)-OH, Fmoc-Thr(tBu)-OH, Fmoc-Tyr(tBu)-OH, Fmoc-Val-OH. The following didpeptide from NovaBiochem were used: Fmoc-Ile-Thr(Ψ^{Me,Me}Pro)-OH (NovaBiochem), Fmoc-Met-Thr(Ψ^{Me,Me}Pro)-OH (Synthesized in the laboratory by the procedure of Mutter (Waif et al, J. Am. Chem. Soc. 118:9218-9227 (1996)).

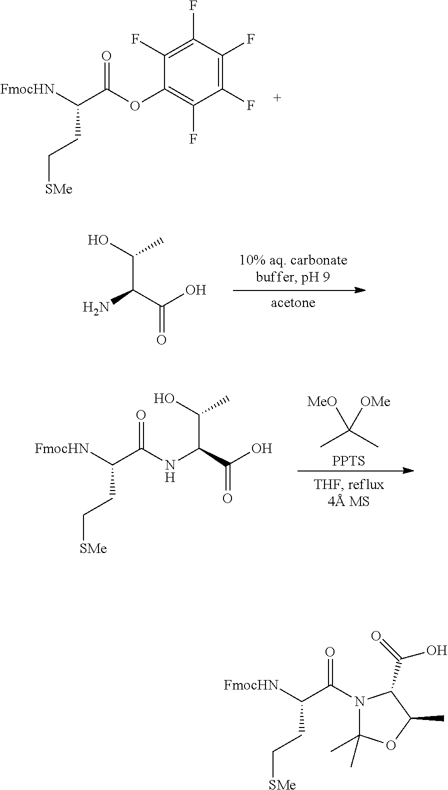

Fmoc-Met-Thr(Ψ^{Me,Me}pro)-OH.

L-threonine (1.03 g, 8.7 mmol) was dissolved in a minimal volume of aqueous sodium carbonate (10% w/v) at pH 9 (9 mL), and the solution was added to a suspension of Fmoc-Met-OPfp (1.55 g, 2.9 mmol) in acetone (23 mL). After vigorous stirring for 3 h, the reaction mixture was cooled to 0° C. and acidified with 1 N HCl to pH ˜1. The solution was then concentrated in vacuo to less than half of the initial volume and ethyl acetate (100 mL) and water (60 mL) were added. The layers were separated and the aqueous layer was extracted with ethyl acetate (2×60 mL). The combined organic extracts were washed with water (30 mL) and brine (2×30 mL), dried over MgSO₄, filtered and evaporated to dryness. The residue was crystallized from ethyl acetate/hexane to give Fmoc-Met-Thr-OH as a white solid.

The dipeptide (2.88 mmol) was then suspended in dry THF (55 mL), and pyridyl toluene-4-sulfonate (145 mg, 0.58 mmol) and 2,2-dimethoxypropane (1.8 mL, 14.4 mmol) were added. The suspension was then heated to reflux overnight under Ar, the condensate being bypassed over molecular sieves (4 Å). After cooling, triethylamine was added (120 μL, 0.86 mmol) and the mixture evaporated to dryness. The residue was taken up in ethyl acetate (100 mL), and washed with water (2×50 mL). The aqueous layer was extracted with ethyl acetate (2×60 mL) and the combined organics were dried over MgSO₄, filtered and concentrated. The residue was purified by flash chromatography over silica gel (20:1 to 10:1 CH₂Cl₂/MeOH) to give the desired pseudoproline dipeptide Fmoc-Met-Thr(Ψ^{Me,Me}pro)-OH (1.3 g, 88% yield) as a white solid.

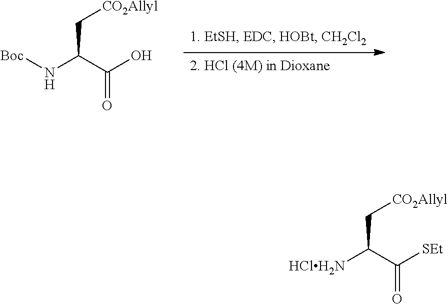

H-Asp(OAll)-SEt.HCl.

Boc-Asp(OAll)-OH (2.73 g, 10 mmol) was solubilized in dichloromethane (50 mL). To this solution EDC (1.77 mL, 10 mmol), HOBt (4.05 g, 30 mmol) and thioethanol (3.6 mL, 50 mmol) were added. The mixture was stirred for 3 h30, concentrated in vacuo and purified by flash chromatography (silica gel, 10% to 15% ethyl acetate/hexane) to afford after concentration and lyophilization Boc-Asp(OAll)-SEt (1.107 g, 3.5 mmol, 35% yield) as a white solid. Boc-Asp(OAll)-SEt (454 mg, 1.4 mmol) was directly solubilized in a solution of HCl in dioxane (4 M, 24 mL). After 1 h30 at room temperature, the solution was concentrated in vacuo, resuspended in water and lyophilized twice to afford H-Asp(OAll)-SEt.HCl as white solid (373 mg, 1.4 mmol, quantitative yield).

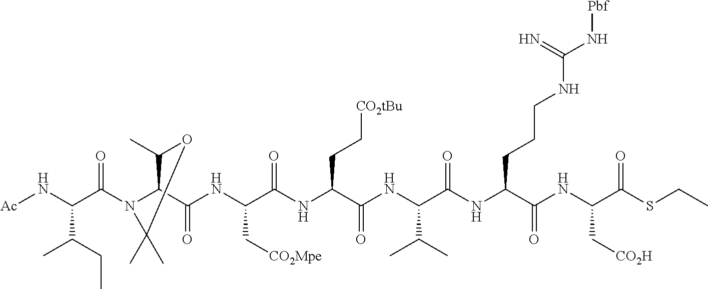

Peptide Thioester 24.

Upon completion of automated synthesis and acetylation on 0.1 mmol of Fmoc-Arg(Pbf)-NovaSynTGT resin, the peptide resin was washed into a peptide synthesis vessel with MeOH. After drying the resin was subjected to a cleavage cocktail (1:1:3 of acetic acid/trifluoroethanol/methylene chloride) for 4 times 30 min. The resulting cleavage solution were pooled and concentrated. The oily residue was resuspended in minimum amount of trifluoroethanol and precipitated with water. The resulting mixture was immediately lyophilized to afford the peptide as white solid (110 mg, 92% yield).

To a solution of this peptide (84 mg, 69.7 μmol) in chloroform (5.4 mL) was added EDC (30.2 μL, 170.8 μmol) and HOOBt (26.9 mg, 165 μmol) and finally H-Asp(Oall)-SEt.HCl (50 mg, 198 The mixture was stirred for 1 h30 min at room temperature. After concentration, the oily residue was resuspended in minimum amount of trifluoroethanol and precipitated with water containing 0.05% trifluoroacetic acid. The resulting mixture was immediately lyophilized. The peptide was solubilized in chloroform (3.2 mL), palladium tetrakis (48 mg, 42 μmol) was added, followed by phenylsilane (39 μL, 315 μmol). The reaction was stirred in the dark for 20 min and then quenched by precipitation with ice-cold diethyl ether (20 mL) The precipitate was resuspended in water/acetonitrile (1:1, 0.05% trifluoroacetic acid) and purified on sephadex LH-20 equilibrated with water/acetonitrile (1:1, 0.05% trifluoroacetic acid). The peptide containing fractions were pooled and immediately lyophilized (80 mg, 84% yield).

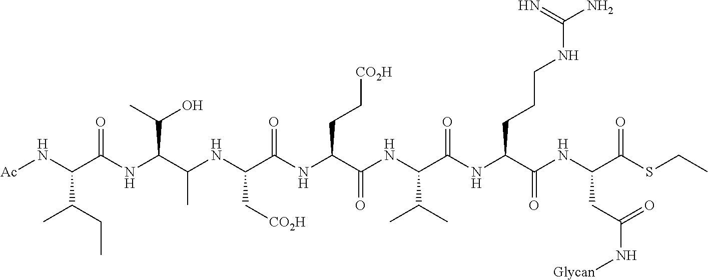

Glycopeptides 25 and 28 (“Fragment 1”).

Resulting peptide (1.6 eq.) and glycan amine (1 eq.) were combined and solubilized in anhydrous DMSO (27 mM). To this mixture, a freshly prepared solution of PyAOP in anhydrous DMSO (0.5 mg/μL) was added (4.4 eq.), followed by DIEA (3.7 eq.). The solution turned into a deep, golden-yellow color and this was stirred for 30 min. The reaction mixture was then frozen and lyophilized.

Fragment 1 glycopeptide was subjected to cocktail B (1 mL/10 mg of peptide) consisting of trifluoroacetic acid (88% by volume), water (5% by volume), phenol (5% by weight), and iPr3SiH (2% by volume). The peptide was precipitated and triturated in ice-cold diethyl ether (3×15 mL) to give a white precipitate, which was centrifuged. The precipitate was solubilized in water/acetonitrile (1:1, 0.05% trifluoroacetic acid) and lyophilized. The resulting solid was desalted by size-exclusion chromatography on biogel (Bio-Rad P4 fine) equilibrated with water/acetonitrile (4:1, 0.05% trifluoroacetic acid) and purified to homogeneity by RP-HPLC. (Man₃GlcNAc₂ 20% yield; Man₅GlcNAc₂ 17% yield)

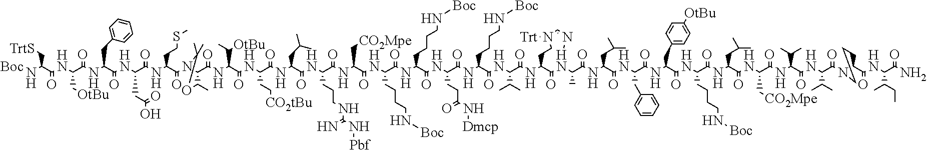

Peptide 26.

Upon completion of automated synthesis on 0.05 mmol of NovaSynTG Sieber resin, the peptide resin was washed into a peptide synthesis vessel with methanol. After drying the resin was pre-swelled in dichlormethane/dimethylformamide (1/1). A solution of palladium tetrakis in dichlormethane/dimethylformamide (1:1) (2.5 mL of 2 mg/mL) was added on the resin followed by phenylsilane (50 μL). The reaction was stirred in the dark for 20 min stirred with argon bubbling, repeated 2 times. The resin was then washed with dichloromethane/dimethylformamide (1/1), dimethylformamide, dichloromethane, methanol. After drying the resin was subjected to a cleavage cocktail (1:99 of trifluoroacetic acid/methylene chloride) for 5 times 5 min (2 mL each). The resin was then subjected to a cleavage cocktail (2:98 of trifluoroacetic acid/methylene chloride) for 5 times 5 min (2 mL each). The resulting cleavage solution were pooled into cold diethyl ether and concentrated. The oily residue was resuspended in minimum amount of trifluoroethanol and precipitated with water. The resulting mixture was immediately lyophilized to afford the peptide as white solid (145 mg, 57% yield).

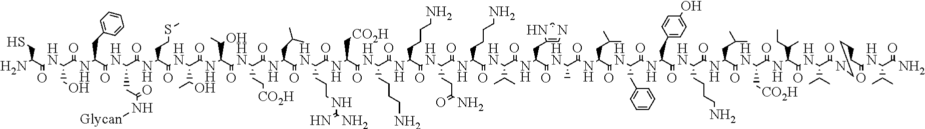

Glycopeptides 27 and 29 (“Fragment 2”).

Resulting peptide (1 eq.) and glycan amine (1.3 eq.) were combined and solubilized in anhydrous DMSO (25 mM). To this mixture, a freshly prepared solution of PyAOP in anhydrous DMSO (0.5 mg/μL) was added (2.9 eq.), followed by DIEA (2.5 eq.). The solution turned into a deep, golden-yellow color and this was stirred for 45 min. The glycopeptide was then precipitated with ice-cold water (0.05% trifluoroacetic acid, 1.5 mL), centrifuged, the precipitate was resuspended in water/acetonitrile (1:1, 0.05% trifluoroacetic acid, 1.5 mL) and lyophilized.

Fragment 2 glycopeptide was subjected to cocktail R (1 mL/10 mg of peptide) consisting of trifluoroacetic acid (90% by volume), anisole (2% by volume), thioanisole (5% by volume), 1,2-ethanedithiol (3% by volume). The peptide was precipitated and triturated in ice-cold diethyl ether (3×15 mL) to give a white precipitate, which was centrifuged. The precipitate was solubilized in water/acetonitrile (1:1, 0.05% trifluoroacetic acid) and lyophilized. The resulting solid was desalted by size-exclusion chromatography on biogel (Bio-Rad P6 fine) equilibrated with water/acetonitrile (4:1, 0.05% trifluoroacetic acid) and purified to homogeneity by RP-HPLC. (Man₃GlcNAc₂ 25% yield; Man₅GlcNAc₂ 28% yield)

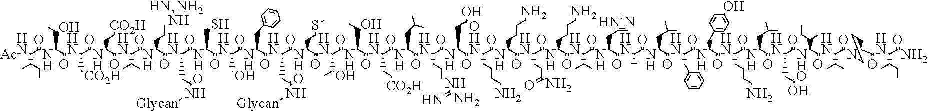

Glycopeptides 1 and 2.

The buffer required for native chemical ligation (NCL) was freshly prepared prior to the reaction. Na₂HPO₄ (56.6 mg, 0.4 mmol) was solubilized in water (1 mL), guanidine.HCl (1.146 g, 12 mmol), and TCEP.HCl (10.8 mg, 0.04 mmol) were then added, solubilized, the volume adjusted to 2 mL and the pH was brought to 7 with a solution of NaOH (5 M, 20 μL). After 15 min degassing with argon, 4-mercaptophenylacetic acid (MPAA) (67 mg, 0.4 mmol) was added and the pH was brought to 7.2 with a solution of NaOH (5 M, 120 μL). After 15 min degassing the solution was ready for use.

Freshly purified glycopeptides fragment 1 (3 eq.) and 2 (1 eq.) were combined and solubilized into NCL buffer (7.7 mM). To this mixture was added neutral TCEP solution (0.5 M, 10% by volume of the reaction mixture). After 2 h, another portion of neutral TCEP solution (0.5 M, 10% by volume of the reaction mixture) was added and the reaction stirred for total 4 h. The resulting suspension was desalted by size-exclusion chromatography on biogel (Bio-Rad P6 fine) equilibrated with water/acetonitrile (4:1, 0.05% trifluoroacetic acid) and purified by RP-HPLC. (Man₃GlcNAc₂ 67% yield; Man₅GlcNAc₂ 48% yield)

### Example 5

Few important structures in nature are more heavily glycosylated than is the envelope spike (Env) of human immunodeficiency virus type 1 (HIV-1).ⁱ A multitude of designed constructs that might simulate the unique architecture of Env have been considered and pursued in the context of potential HIV-1-directed vaccines. Yet, until recently, the only template for immunological recognition of this dense “glycan shield” has been the broadly neutralizing antibody (BnAb) 2G12.ⁱⁱ Following its discovery, many laboratories,ⁱⁱⁱ including our own,^{iv} were able to generate mimics of the oligomannose cluster that constitutes its epitope, in the hope of eliciting 2G12-like antibodies. Unfortunately, these efforts were not successful. While many factors have been cited to explain the general difficulties surrounding BnAb induction,^{v} the case of 2G12 is likely complicated further by the unusual domain-exchanged arrangement of its heavy chains, which is thought to be responsible for its unique mode of glycan recognition.^{iid}

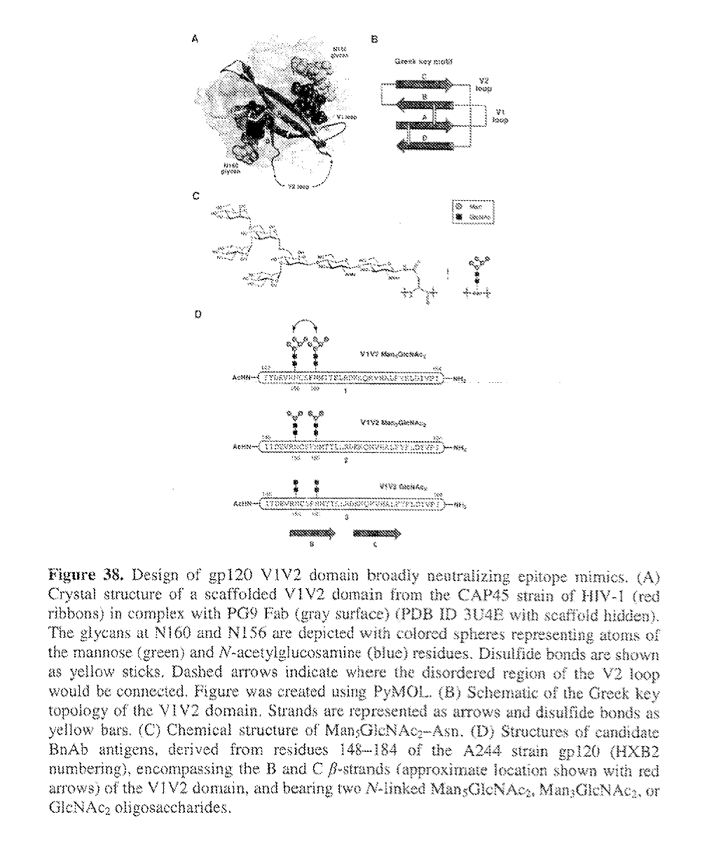

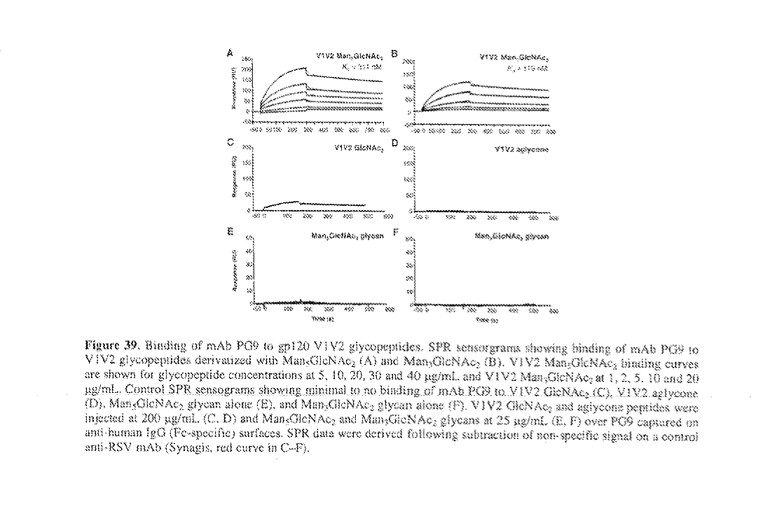

In 2009, two new and potent BnAbs, PG9 and PG16, were isolated from an HIV-1-infected donor from sub-Saharan Africa.^{vi} These monoclonal antibodies (mAbs) were found to neutralize 70-80% of circulating HIV-1 isolates. Initial epitope mapping suggested that PG9 and PG16 were targeting a new glycan-dependent Env epitope, entirely distinct from that of 2G12. A sensitivity to quarternary structure was also noted, as these BnAbs exhibited a preference for binding fully assembled trimeric viral spike over monomeric Env. Subsequently, a co-crystal structure of PG9 with gp120 variable regions 1 and 2 (V1V2) grafted onto a mini-protein scaffold revealed that the antibody engages high mannose glycans at Asn¹⁶⁰ and Asn¹⁵⁶ and an adjacent β-strand (FIG. 38A).^{vii} In contrast to 2G12, which apparently does not interact with the gp120 peptide backbone, PG9 binds an epitope that contains both carbohydrate and peptide components, while possessing a normal heavy chain arrangement.

In light of our continuing involvement in the synthesis of glycoproteins and complex glycopolypeptide motifs,^{viii} we regarded these new structural observations with particular interest. It seemed not unlikely that successful design of vaccines based on the PG9 epitope would depend crucially on close simulation of the detailed surface glycopeptide architecture of Env. Given the importance of the glycan domains in forming this conserved epitope, it seemed that access to Env constructs that are well-defined and homogeneous with respect to glycosylation state would greatly facilitate immunogen development efforts. Past work has largely relied on recombinant Env preparations, supplied as mixtures of glycoforms.^{ix} This serious heterogeneity complicates efforts to draw precise correlations between glycan composition and immunoactivity. Absent a detailed understanding of the structural biology of the problem, informed vaccine design is, naturally, much more difficult. We were stirred by the prospect that de novo chemical synthesis could provide the complex, yet homogeneous probe substrates needed for rationally based advances in this urgent endeavor.

More specifically, we hypothesized that fully synthetic, homogeneous gp120 V1V2 polypeptide domains, bearing defined glycosyl patterns, might be able to function as minimal mimics of the PG9 epitope. If such uniform, synthetically-derived constructs were able to simulate the conformation of the pertinent native envelope glycoproteins, they would provide a logical starting point for immunogen design. Moreover, a minimal construct could, in theory, present the desired BnAb epitope without interference from other potentially more immunogenic Env determinants.^{v}

Herein, we describe the chemical synthesis of gp120 V1V2 glycopeptides as single glycoforms^{x} that were found to bind the BnAb PG9 with surprisingly high affinities. During the course of this work, we had to deal with and overcome the fundamental synthetic challenge arising from the close spacing of large glycans along the peptide backbone. In engaging this challenge, we would be pressing against the limits of the prior art we had developed in the realm of glycopeptide ligations.^{xi}

General Information.

All non-aqueous reactions were carried out under an atmosphere of argon or nitrogen in flame- or oven-dried glassware with magnetic stirring unless otherwise indicated. Benzene, dichloromethane, diethyl ether, tetrahydrofuran, and toluene were purified by passage through an activated alumina column. Dichloromethane for glycosylation reactions was distilled from calcium hydride. All other commercially obtained reagents were used as received, except where specified otherwise. Flash chromatography was performed on Silicycle SiliaFlash P60 silica gel (60 Å pore size, 230-400 mesh). Analytical thin layer chromatography was performed on Silicycle SiliaPlate glass-backed plates coated with silica gel (250 μm thickness, 60 Å pore size, F-254 indicator) and visualized by exposure to ultraviolet light and/or staining with aqueous ceric ammonium molybdate solution or 5% sulfuric acid in methanol. ¹H NMR spectra were recorded on a Bruker AVANCE DRX-500 (500 MHz) or DRX-600 (600 MHz) spectrometer at 24° C., unless otherwise stated. Chemical shifts are reported in parts per million from CDCl₃, C₆D₆, D₂O, or DMSO-d₆ internal standard (7.26, 7.15, 4.79, and 2.50 ppm, respectively). Data are reported as follows: (s=singlet, d=doublet, t=triplet, q=quartet, quint=quintet, m=multiplet, dd=doublet of doublets, ddd=doublet of doublet of doublets, br=broad; coupling constant(s) in Hz; integration). Proton-decoupled ¹³C NMR spectra were recorded on a Bruker AVANCE DRX-500 (125 MHz) or DRX-600 (150 MHz) spectrometer at 24° C., unless otherwise stated. Chemical shifts are reported in ppm from CDCl₃, C₆D₆, or DMSO-d₆ internal standard (77.0, 128.0, 39.52 ppm, respectively). Peaks that are split due to coupling to ¹⁹F are reported as individual resonances. Attenuated total reflectance Fourier transform infrared (ATR-FTIR) spectra were recorded on a JASCO FT/IR-6100 spectrometer. Optical rotations were recorded on a JASCO P-2000 digital polarimeter. Low resolution electrospray ionization (ESI) mass spectra were obtained on a JEOL JMS-DX303 HF mass spectrometer or Waters Micromass ZQ mass spectrometer in the NMR Analytical Core Facility at MSKCC.

### Experimental Procedures: Carbohydrates.

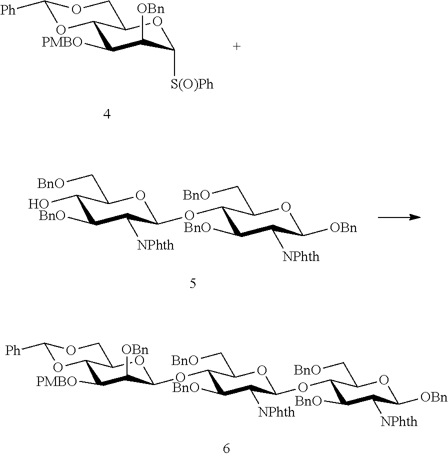

### Benzyl 2-O-benzyl-3-O-p-methoxybenzyl-4,6-O—(R)-benzylidene-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside (6)

Freshly activated AW-300 MS (8 g) were added to a solution of mannose sulfoxide 4 (Crich, D.; Li, H.; Yao, Q.; Wink, D. J.; Sommer, R. D.; Rheingold, A. L. J. Am. Chem. Soc. 2001, 123, 5826-5828.) (3.97 g, 6.78 mmol) in anhydrous CH₂Cl₂ (50 mL). After 1 h at r.t., the mixture was cooled to −78° C., and di-tert-butyl pyridine (3.5 mL, 15.8 mmol) and Tf₂O (1.2 mL, 7.23 mmol) were added. The mixture was allowed to warm up to −50° C. over 30 min, cooled to −78° C. and a solution of acceptor 5 (Walczak, M. A.; Danishefsky, S. J. J. Am. Chem. Soc. 2012, 134, 16430-16433.) (4.75 g, 4.52 mmol) in CH₂Cl₂ (50 mL) (cooled separately at −78° C.) was added dropwise via cannula. The mixture was stirred at −78° C. for 8 h, filtered through a pad of Celite, washed with saturated aqueous NaHCO₃, water, brine, dried over MgSO₄ and concentrated. Purification by flash chromatography (hexanes:CH₂Cl₂:EtOAc, 4:4:1) afforded 6 (5.87 g, 86%) as an amorphous white solid in single diastereomeric form.

¹H NMR (600 MHz, CDCl₃) δ 7.87-6.58 (m, 47H), 5.41 (s, 1H), 5.20 (d, J=8.3 Hz, 1H), 4.87 (d, J=8, 4 Hz, 1H), 4.84-4.70 (m, 4H), 4.61 (d, J=12.3 Hz, 1H), 4.58 (d, J=11.9 Hz, 1H), 4.52-4.36 (m, 6H), 4.36-4.24 (m, 3H), 4.22-4.08 (m, 4H), 4.07-4.01 (m, 2H), 4.01-3.94 (m, 2H), 3.69 (s, 3H), 3.65 (d, J=3.1 Hz, 1H), 3.53 (dd, J=11.3, 2.0 Hz, 1H), 3.49 (dd, J=11.2, 1.6 Hz, 1H), 3.44 (t, J=10.4 Hz, 1H), 3.39-3.27 (m, 3H), 3.23 (ddd, J=9.9, 3.8, 1.7 Hz, 1H), 3.11 (dt, J=10.1, 2.6 Hz, 1H), 3.05 (td, J=9.6, 4.8 Hz, 1H).

¹³C NMR (150 MHz, CDCl₃) δ 167.63, 167.56, 159.20, 138.91, 138.72, 138.67, 138.55, 137.89, 137.70, 137.22, 134.01, 133.81, 133.47, 131.72, 130.62, 129.17, 129.13, 128.81, 128.57, 128.55, 128.38, 128.34, 128.31, 128.29, 128.25, 128.23, 128.20, 128.17, 128.15, 128.11, 128.08, 127.98, 127.81, 127.80, 127.79, 127.75, 127.69, 127.65, 127.60, 127.59, 127.57, 127.56, 127.51, 127.49, 127.47, 127.44, 127.41, 127.36, 127.13, 126.92, 126.85, 126.20, 126.11, 123.67, 123.12, 113.77, 113.73, 101.90, 101.33, 97.17, 97.07, 79.37, 78.68, 78.04, 77.31, 77.24, 77.09, 76.88, 76.57, 75.78, 75.13, 74.71, 74.67, 74.55, 74.34, 73.31, 72.73, 72.32, 70.54, 68.57, 68.24, 67.97, 67.38, 56.61, 55.78, 55.31, 55.30.

IR (ATR-FTIR, thin film) 3030, 2867, 1712, 1386, 1046 cm⁻¹.

[α]²²_{D} (c 1.0, CH₂Cl₂)−32.8.

LRMS (ESI+) m/z calc'd for [M+Na]⁺ (C₉₁H₈₆N₂NaO₁₉) requires 1533.6. found 1533.9.

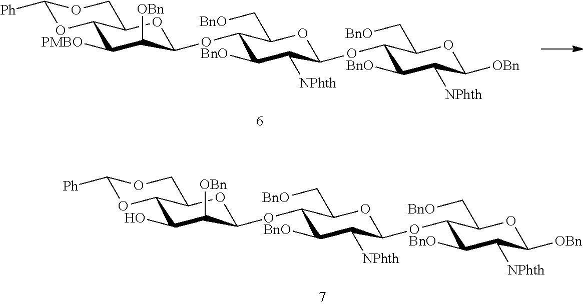

### Benzyl 2-O-benzyl-4,6-O—(R)-benzylidene-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside (7)

Trisaccharide 6 (5.35 g, 3.54 mmol) was dissolved in CH₂Cl₂ (100 mL), followed by addition of H₂O (100 mL), and the mixture treated with DDQ (2.68 g, 11.8 mmol). The mixture was stirred vigorously at r.t., in the dark for 2 h. The reaction was quenched with a buffer solution (0.7% ascorbic acid+1.3% citric acid+1.9% NaOH in H₂O, w/v) (20 mL), diluted with CH₂Cl₂ (200 mL), washed with water (2×), brine, dried over MgSO₄ and concentrated. Purification by flash chromatography (hexanes:CH₂Cl₂:EtOAc, 4:4:1) afforded 7 (4.1 g, 83%) as an amorphous white solid.

¹H NMR (600 MHz, CDCl₃) δ 7.98-6.74 (m, 43H), 5.46 (s, 1H), 5.33 (d, J=7.9 Hz, 1H), 5.05 (d, J=11.6 Hz, 1H), 5.00 (d, J=8.4 Hz, 1H), 4.94 (d, J=12.2 Hz, 1H), 4.89 (d, J=12.8 Hz, 1H), 4.78-4.69 (m, 3H), 4.65 (d, J=12.0 Hz, 1H), 4.61-4.53 (m, 3H), 4.50 (d, J=12.0 Hz, 1H), 4.43 (t, J=12.8 Hz, 2H), 4.34-4.21 (m, 4H), 4.21-4.11 (m, 3H), 3.76 (d, J=3.7 Hz, 1H), 3.73 (t, J=9.5 Hz, 1H), 3.69 (dd, J=11.3, 2.0 Hz, 1H), 3.65-3.59 (m, 2H), 3.57-3.45 (m, 3H), 3.36 (ddd, J=9.8, 3.7, 1.5 Hz, 1H), 3.24 (dt, J=9.9, 2.4 Hz, 1H), 3.16 (td, J=9.6, 4.9 Hz, 1H).

¹³C NMR (150 MHz, CDCl₃) δ 167.64, 138.81, 138.67, 138.55, 138.22, 137.75, 137.30, 137.20, 134.47, 133.48, 131.70, 129.76, 129.07, 129.02, 128.62, 128.60, 128.55, 128.42, 128.39, 128.32, 128.29, 128.24, 128.22, 128.16, 128.10, 128.08, 128.06, 128.02, 127.99, 127.98, 127.97, 127.95, 127.90, 127.84, 127.82, 127.80, 127.78, 127.57, 127.56, 127.48, 127.46, 127.44, 127.35, 127.33, 127.32, 127.18, 127.14, 126.98, 126.86, 126.84, 126.38, 126.30, 123.12, 101.95, 97.17, 97.03, 79.42, 79.20, 79.01, 77.29, 77.14, 77.08, 76.86, 76.58, 75.83, 75.74, 74.68, 74.66, 74.54, 74.31, 73.45, 73.43, 72.69, 70.96, 70.55, 68.50, 68.25, 67.82, 66.88, 56.55, 55.78.

IR (ATR-FTIR, thin film) 3477, 3030, 2871, 1775, 1712, 1386, 1075 cm⁻¹.

[α]²²D (c 1.0, CH₂Cl₂) −35.7.

LRMS (ESI+) m/z calc'd for [M+Na]⁺ (C₈₃H₇₈N₂NaO₁₈) requires 1413.5. found 1413.9.

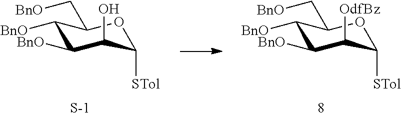

### p-Tolyl 3,4,6-tri-O-benzyl-(2,5-difluorobenzoyl)-1-thio-α-D-mannopyranoside

Thioglycoside S-1 (Chayajarus, K.; Chambers, D. J.; Chughtai, M. J.; Fairbanks, A. J. Org. Lett. 2004, 6, 3797-3800.) (10.0 g, 18.0 mmol) and 4-dimethylaminopyridine (0.22 g, 1.8 mmol) were dissolved in pyridine (50 mL), and then 2,5-difluorobenzoyl chloride (6.7 mL, 54.0 mmol) was added. The mixture was stirred at room temperature overnight and then diluted with CH₂Cl₂ (300 mL). The mixture was washed with saturated aqueous NaHCO₃ (150 mL), water (150 mL) and 1 N HCl (150 mL), and then dried over Na₂SO₄, filtered and concentrated. Purification by flash chromatography (9:1 to 85:15 hexanes/ethyl acetate) afforded difluorobenzoyl ester 8 (11.0 g, 88% yield) as a clear oil.

¹H NMR (600 MHz, CDCl₃) δ 7.72-7.65 (m, 1H), 7.42-7.20 (m, 18H), 7.13-7.05 (m, 3H), 5.85 (dd, J=2.9, 1.7 Hz, 1H), 5.59 (d, J=1.7 Hz, 1H), 4.90 (d, J=10.8 Hz, 1H), 4.81 (d, J=11.4 Hz, 1H), 4.69 (d, J=12.0 Hz, 1H), 4.64 (d, J=11.4 Hz, 1H), 4.54 (d, J=10.7 Hz, 1H), 4.50 (d, J=12.0 Hz, 1H), 4.41 (ddd, J=9.6, 4.4, 1.9 Hz, 1H), 4.14-4.04 (m, 2H), 3.89 (dd, J=10.8, 4.5 Hz, 1H), 3.79 (dd, J=10.7, 2.0 Hz, 1H), 2.32 (s, 3H).

¹³C NMR (150 MHz, CDCl₃) δ 162.21, 162.20, 162.19, 162.17, 159.14, 159.12, 158.8, 158.7, 157.43, 157.41, 157.13, 157.12, 138.3, 138.2, 138.0, 137.5, 132.4, 129.8, 129.7, 128.4, 128.3, 128.2, 128.1, 128.0, 127.8, 127.7, 127.6, 127.4, 121.64, 121.58, 121.5, 121.4, 118.52, 118.47, 118.45, 118.36, 118.30, 118.28, 86.3, 78.4, 75.3, 74.6, 73.3, 72.5, 71.9, 71.5, 68.9, 21.1.

LRMS (ESI+) m/z calc'd for [M+Na]⁺ (C₄₁H₃₈F₂O₆SNa) requires 719.2. found 719.3.

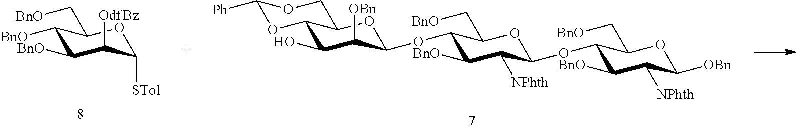

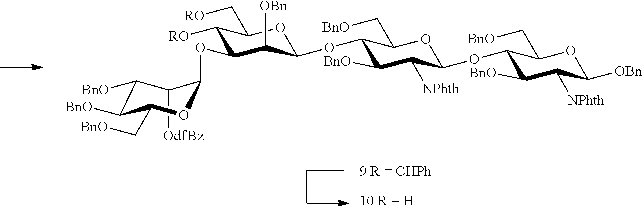

### Benzyl[3,4,6-tri-O-benzyl-2-O-(2,5-difluorobenzoyl)-α-D-mannopyranosyl-(1→3)]-2-O-benzyl-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside (10)

A mixture of trisaccharide acceptor 7 (4.0 g, 2.87 mmol) and mannose thioglycoside donor 8 (2.6 g, 3.74 mmol) was dissolved in anhydrous CH₂Cl₂ (100 mL). Freshly activated AW-300 MS (6 g) was added and stirred at r.t. for 1 h. The mixture was cooled to 0° C., NIS (0.9 g, 4.0 mmol) and TMSOTf (100 μl, 0.57 mmol) were added sequentially, and the mixture was allowed to warm up to r.t. over 5 h. The mixture was filtered through a pad of Celite and the organic layer was washed with sat aqueous Na₂S₂O₃, saturated aqueous NaHCO₃, water, brine, dried over MgSO₄ and concentrated.

The crude tetrasaccharide 9 was dissolved in acetic acid (30 mL). H₂O (4.5 mL) was added dropwise with stirring and the reaction mixture was heated at 70° C. for 3 h. The mixture was co-evaporated with toluene and the crude mass was purified by flash chromatography (hexanes:EtOAc, 1:1) to give 10 (3.39 g, 63% over two steps) as an amorphous white solid. Measurement of ¹J_{CH} coupling constants ((a) Bock, K.; Lundt, I.; Pedersen, C. Tetrahedron Lett. 1973, 14, 1037-1040. (b) Bock, K.; Pedersen, C. J. Chem. Soc., Perkin Trans. 2 1974, 293-297.) confirmed the anomeric configuration at each inter-residue glycosidic bond (data listed below).

¹H NMR (600 MHz, CDCl₃) δ 7.88-6.57 (m, 56H), 5.52 (d, J=3.0 Hz, 2H), 5.21-5.14 (m, 1H), 4.93-4.85 (m, 2H), 4.84-4.74 (m, 3H), 4.69 (d, J=11.2 Hz, 1H), 4.62 (m, J=12.0, 9.3 Hz, 2H), 4.52-4.40 (m, 8H), 4.42-4.34 (m, 2H), 4.29 (d, J=12.3 Hz, 1H), 4.22 (d, J=12.2 Hz, 1H), 4.17-4.08 (m, 4H), 4.05 (dd, J=10.7, 8.4 Hz, 1H), 3.99 (m, 2H), 3.96-3.91 (m, 1H), 3.88 (t, J=9.6 Hz, 1H), 3.74 (d, J=3.1 Hz, 1H), 3.69 (t, J=9.6 Hz, 1H), 3.65 (dd, J=10.2, 2.0 Hz, 1H), 3.55 (m, 2H), 3.51-3.45 (m, 2H), 3.43-3.34 (m, 3H), 3.31 (dd, J=11.7, 5.7 Hz, 1H), 3.23 (ddd, J=9.8, 3.9, 1.6 Hz. 1H), 3.13 (dt, J=10.1, 2.5 Hz, 1H), 2.97 (ddd, J=9.2, 5.8, 3.3 Hz, 1H).

¹³C NMR (150 MHz, CDCl₃) δ 168.52, 167.65, 162.18, 159.08, 158.75, 157.38, 157.13, 138.63, 138.56, 138.50, 138.36, 138.22, 137.79, 137.64, 137.19, 134.49, 134.07, 133.85, 133.47, 133.42, 131.69, 131.43, 130.91, 130.13, 129.77, 129.58, 129.29, 129.02, 128.82, 128.71, 128.61, 128.55, 128.51, 128.39, 128.31, 128.28, 128.22, 128.13, 128.08, 128.01, 127.96, 127.90, 127.86, 127.82, 127.79, 127.72, 127.66, 127.57, 127.48, 127.38, 127.35, 127.29, 127.20, 127.09, 127.04, 127.02, 126.92, 126.85, 125.96, 123.71, 123.12, 121.61, 121.55, 121.45, 121.39, 118.52, 118.46, 118.36, 118.29, 109.63, 101.12 (¹J_{CH}=160.3 Hz, β-Man), 97.20 (¹J_{CH}=165.3 Hz, β-GlcN), 97.14, 97.09 (¹J_{CH}=174.3 Hz, α-Man), 80.85, 79.12, 78.71, 77.88, 76.61, 76.55, 75.89, 75.86, 75.81, 75.73, 75.02, 74.75, 74.70, 74.54, 74.50, 74.49, 74.46, 73.56, 73.33, 72.74, 71.89, 71.80, 70.54, 70.14, 69.49, 68.21, 67.63, 66.39, 62.57, 56.59, 56.51, 55.77.

IR (ATR-FTIR, thin film) 3472, 2925, 1775, 1712, 1387, 1073, 698 cm⁻¹.

[α]²⁴_{D} (c 1.0, CH₂Cl₂) −15.8.

LRMS (ESI+) m/z calc'd for [M+Na]⁺ (C₁₁₀H₁₀₄F₂N₂NaO₂₄) requires 1897.7. found 1897.6.

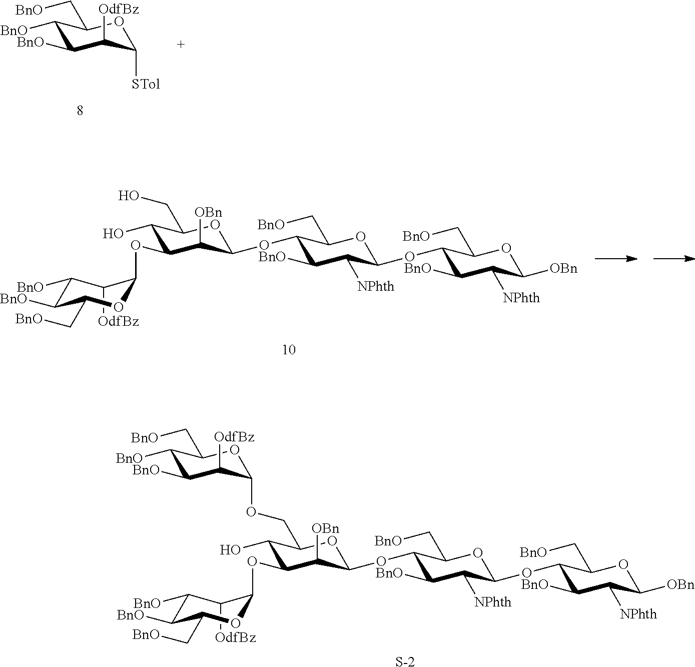

### Benzyl[3,4,6-tri-O-benzyl-2-O-(2,5-difluorobenzoyl)-α-D-mannopyranosyl-(1→3)]-[3,4,6-tri-O-benzyl-2-O-(2,5-difluorobenzoyl)-α-D-mannopyranosyl-(1→6)]-2-O-benzyl-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside (S-2)

A mixture of tetrasaccharide acceptor 10 (1.05 g, 0.56 mmol) and mannose thioglycoside donor 8 (390 mg, 0.56 mmol) was dissolved in anhydrous CH₂Cl₂ (100 mL). Freshly activated AW-300 MS (2.0 g) was added and stirred at r.t. for 1 h. The mixture was cooled to −40° C., NIS (189 mg, 0.84 mmol) and TMSOTf (20 μl, 0.11 mmol) were added sequentially, and the mixture was allowed to warm up to 0° C. over 5 h. The mixture was filtered through a pad of Celite and the organic layer was washed with saturated aqueous Na₂S₂O₃, saturated aqueous NaHCO₃, water, brine, dried over MgSO₄ and concentrated. The residue was purified by flash chromatography (hexanes:EtOAc, 2:1) to give the pentasaccharide S-2 (1.29 g, 94%) as an amorphous white solid.

The regioselectivity of glycosylation was confirmed by a range of 2D-NMR experiments. The HMBC spectrum of pentasaccharide S-2 showed a cross peak between H-1 of the newly installed α-Man (5.16 ppm) and C-6 of the central, branched β-Man (67.2 ppm) confirming that the glycosylation had occurred at the primary alcohol at the C-6 position. This assignment was also supported by the change in chemical shift of the C-6 carbon from 62.6 ppm to 67.0 ppm while C-4 remained relatively unchanged from 66.4 (in the case of diol) to 66.7 ppm (after the glycosylation). Further evidence was obtained from the NOESY spectrum, which revealed cross peaks between the H-1 of α-Man (5.16 ppm) and H-6a and H-6b of β-Man (4.12 and 3.86 ppm respectively).

¹H NMR (600 MHz, C₆D₆) δ 7.99-7.07 (m, 62H), 7.06-6.90 (m, 5H), 6.88-6.75 (m, 3H), 6.75-6.66 (m, 1H), 6.58-6.36 (m, 3H), 6.22-6.15 (m, 1H), 6.01 (d, J=1.9 Hz, 1H), 5.94 (t, J=2.4 Hz, 1H), 5.76 (d, J=8.3 Hz, 1H), 5.49 (d, J=12.8 Hz, 1H), 5.34 (d, J=11.8 Hz, 1H), 5.31 (d, J=8.5 Hz, 1H), 5.20 (d, J=13.0 Hz, 1H), 5.16-5.10 (m, 2H), 5.08 (d, J=11.3 Hz, 1H), 5.04-4.95 (m, 3H), 4.90 (d, J=12.8 Hz, 1H), 4.88-4.82 (m, 2H), 4.81-4.38 (m, 22H), 4.31-4.24 (m, 3H), 4.25-4.16 (m, 3H), 4.13 (dd, J=11.0, 3.9 Hz, 1H), 3.98 (dd, J=10.6, 1.8 Hz, 1H), 3.91-3.78 (m, 5H), 3.77-3.69 (m, 1H), 3.68-3.59 (m, 2H), 3.55 (dd, J=10.9, 1.7 Hz, 1H), 3.44 (dt, J=10.2, 2.5 Hz, 1H), 3.41 (dt, J=9.4, 3.3 Hz, 1H), 2.97 (ddd, J=10.1, 3.4, 1.6 Hz, 1H).

¹³C NMR (150 MHz, C₆D₆) δ 167.67, 162.50, 159.19, 158.98, 157.49, 157.38, 157.37, 157.29, 139.78, 139.50, 139.41, 139.33, 139.15, 139.13, 138.81, 138.75, 138.66, 138.50, 138.09, 133.14, 132.36, 129.05, 128.99, 128.95, 128.85, 128.75, 128.72, 128.67, 128.65, 128.62, 128.58, 128.56, 128.55, 128.51, 128.47, 128.44, 128.42, 128.37, 128.35, 128.31, 128.26, 128.25, 128.18, 128.13, 128.09, 128.02, 127.97, 127.95, 127.94, 127.91, 127.86, 127.73, 127.70, 127.68, 127.65, 127.58, 127.55, 127.53, 127.50, 127.49, 127.45, 127.30, 127.28, 127.03, 127.02, 123.03, 120.16, 118.62, 118.54, 118.50, 118.46, 118.41, 118.37, 118.33, 118.25, 118.19, 102.26, 98.69, 98.11, 97.76, 81.01, 80.13, 79.41, 79.28, 78.79, 77.51, 77.10, 76.47, 75.99, 75.58, 75.26, 75.21, 75.10, 75.03, 74.93, 74.85, 74.82, 73.69, 73.50, 73.40, 73.10, 72.87, 72.84, 72.02, 71.91, 70.61, 70.51, 70.33, 70.31, 69.58, 68.51, 67.88, 67.10, 67.01, 57.32, 56.55.

IR (ATR-FTIR, thin film) 2926, 1776, 1714, 1495, 1387, 1077, 698 cm⁻¹.

[α]²⁴_{D} (c 1.0, CH₂Cl₂) −13.4.

LRMS (ESI+) m/z calc'd for [M+Na]⁺ (C₁₄₄H₁₃₄F₄N₂NaO₃₀) requires 2469.9. found 2470.0.

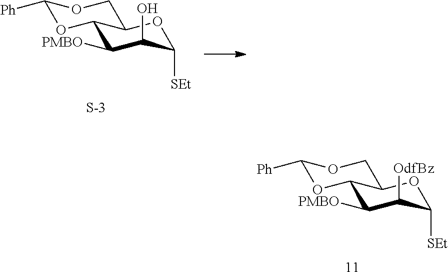

### Ethyl 4,6-O-benzylidene-2-O-(2,5-difluorobenzoyl)-3-O-p-methoxybenzyl-1-thio-α-D-mannopyranoside (11)

To a solution of alcohol S-3 (Cherif, S.; Clavel, J.-M.; Monneret, C. J. Carbohydr. Chem. 2002, 21, 123-130.) (768 mg, 1.77 mmol) and 4-dimethylaminopyridine (43.4 mg, 0.355 mmol) in pyridine (5.0 mL) was added 2,5-difluorobenzoyl chloride (0.44 mL, 3.55 mmol) via syringe pump over 10 min. The reaction mixture was stirred at room temperature; gradual formation of a white precipitate was observed over time. An additional portion of 2,5-difluorobenzoyl chloride (0.11 mL, 0.887 mmol) was added at 19.5 h. After a total reaction time of 44 h, MeOH (0.80 mL) was added. The resulting mixture was stirred for 1 h, then diluted with CH₂Cl₂ (80 mL) and washed with water (120 mL). The aqueous phase was back-extracted with CH₂Cl₂ (60 mL), then the combined organic layers were dried (MgSO₄), filtered, and concentrated. Purification by flash chromatography (10% EtOAc/hexanes) afforded an oily white solid that was taken up in EtOAc (80 mL) and washed with saturated aqueous NaHCO₃ (2×20 mL) (to remove residual 2,5-difluorobenzoic acid). The combined aqueous phases were back-extracted with EtOAc (40 mL). The organic layers were combined, washed with water (20 mL) and brine (20 mL), dried (MgSO₄), filtered, and concentrated to provide difluorobenzoyl ester 11 as a yellowish foam in 94% yield (960 mg, 1.68 mmol).

¹H NMR (600 MHz, CDCl₃) δ 7.67 (ddd, J=8.5, 5.4, 3.2 Hz, 1H), 7.53-7.48 (m, 2H), 7.41-7.34 (m, 3H), 7.26-7.22 (m, 3H), 7.14 (apparent td, J=9.4, 4.1 Hz, 1H), 6.81 (d, 2H), 5.65 (s, 2H), 5.65 (dd, J=3.3, 1.5 Hz, 1H), 5.38 (d, J=1.4 Hz, 1H), 4.66 (d, J=11.8 Hz, 1H), 4.62 (d, J=11.7 Hz, 1H), 4.31-4.23 (m, 2H), 4.19-4.14 (m, 1H), 4.04 (dd, J=9.8, 3.3 Hz, 1H), 3.92-3.86 (m, 1H), 3.78 (s, 3H), 2.72-2.59 (m, 2H), 1.30 (t, J=7.4 Hz, 3H).

¹³C NMR (150 MHz, CDCl₃) δ 162.30, 162.28, 162.27, 162.26, 159.24, 159.07, 159.05, 158.79, 158.77, 157.36, 157.35, 157.17, 157.15, 137.38, 129.69, 129.44, 128.94, 128.16, 126.13, 121.74, 121.68, 121.58, 121.52, 119.31, 119.26, 119.23, 119.18, 118.60, 118.55, 118.47, 118.43, 118.38, 118.30, 113.74, 101.64, 83.29, 78.78, 73.70, 72.85, 71.92, 68.61, 64.65, 55.23, 25.66, 14.94.

IR (ATR-FTIR, thin film) 3419, 3067, 3033, 2953, 2928, 2871, 1721, 1612, 1595, 1587, 1514, 1496, 1454, 1428, 1371, 1308, 1270, 1244, 1186, 1098, 1081, 1063, 1029, 969, 945, 908, 890, 826 cm⁻¹.

[α]²²_{D} (c 0.15, CH₂Cl₂) +19.2.

LRMS (ESI+) m/z calc'd for [M+Na]⁺ (C₃₀H₃₀F₂NaO₇S) requires 595.2. found 595.1.

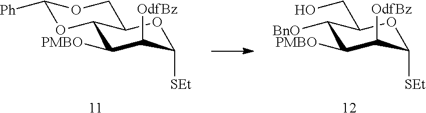

### Ethyl 4-O-benzyl-2-O-(2,5-difluorobenzoyl)-3-O-p-methoxybenzyl-1-thio-α-D-mannopyranoside (12)

To a cooled (0° C.) round-bottom flask containing benzylidene acetal 11 (960 mg, 1.68 mmol) was added borane-THF complex (1.0 M in THF, 8.4 mL, 8.40 mmol). The resulting clear, colorless solution was stirred for 10 min at 0° C. Copper(II) trifluoromethanesulfonate (60.7 mg, 0.168 mmol) was then added in one portion, giving a light brown suspension that was maintained at 0° C. for 25.5 h with vigorous stirring. The reaction was carefully quenched while cold by successive addition of triethylamine (0.24 mL) and MeOH (3.0 mL) (CAUTION: H₂ evolution!). Volatiles were removed on a rotary evaporator, and the residue was co-evaporated with MeOH a few times, resulting in a cloudy, dark brown oil. Purification by flash chromatography (20% EtOAc/hexanes) afforded alcohol 12 as a clear, very pale yellow oil in 96% yield (929 mg, 1.62 mmol). The regioselectivity of the ring opening was assigned on the basis of the multiplicity of the OH proton, and the observation of a ¹H-¹H correlation between the OH and C-6 protons in the 2D COSY.

¹H NMR (600 MHz, CDCl₃) δ 7.65 (ddd, J=8.5, 5.4, 3.3 Hz, 1H), 7.37-7.32 (m, 2H), 7.32-7.28 (m, 3H), 7.29-7.21 (m, 1H), 7.24 (d, J=8.6 Hz, 2H), 7.14 (apparent td, J=9.4, 4.2 Hz, 1H), 6.82 (d, J=8.6 Hz, 2H), 5.66 (apparent t, J=2.3 Hz, 1H), 5.36 (d, J=1.7 Hz, 1H), 4.90 (d, J=10.9 Hz, 1H), 4.68 (d, J=11.1 Hz, 1H), 4.64 (d, J=10.9 Hz, 1H), 4.51 (d, J=11.1 Hz, 1H), 4.07 (dt, J=9.3, 3.3 Hz, 1H), 4.02-3.94 (m, 2H), 3.85-3.79 (m, 2H), 3.78 (s, 3H), 2.70-2.58 (m, 2H), 1.78 (dd, J=7.6, 5.5 Hz, 1H), 1.29 (t, J=7.4 Hz, 3H).

¹³C NMR (150 MHz, CDCl₃) δ 162.39, 162.38, 162.37, 162.35, 159.29, 159.03, 159.01, 158.80, 158.79, 157.32, 157.31, 157.19, 157.17, 138.17, 129.76, 129.64, 128.38, 128.05, 127.77, 121.68, 121.62, 121.52, 121.46, 119.38, 119.33, 119.30, 119.25, 118.63, 118.58, 118.46, 118.44, 118.41, 118.28, 113.77, 82.25, 77.98, 75.18, 74.03, 72.40, 71.79, 71.40, 62.04, 55.18, 25.61, 14.85.

IR (ATR-FTIR, thin film) 3500, 3073, 3032, 2962, 2930, 2874, 2837, 1721, 1612, 1587, 1514, 1496, 1454, 1427, 1368, 1345, 1308, 1268, 1247, 1185, 1094, 1075, 1031, 968, 942, 892, 825 cm⁻¹.

[α]²²_{D} (c 1.0, CH₂Cl₂) +34.3.

LRMS (ESI+) m/z calc'd for [M+Na]⁺ (C₃₀H₃₂F₂NaO₇S) requires 597.2. found 597.2.

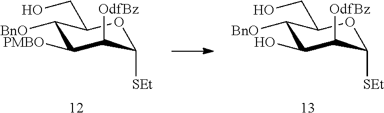

### Ethyl 4-O-benzyl-2-O-(2,5-difluorobenzoyl)-1-thio-α-D-mannopyranoside (13)

To a solution of PMB ether 12 in CH₂Cl₂ (7.2 mL) was added water (0.40 mL) and 2,3-dichloro-5,6-dicyano-1,4-benzoquinone (263 mg, 1.16 mmol), resulting in a dark greenish-black color that became reddish-orange over time. After stirring for 4 h at room temperature, the reaction was quenched with a solution of ascorbic acid/citric acid/NaOH (0.7%/1.3%/0.9% in water, 50 mL) and diluted with EtOAc (100 mL). The layers were separated, and the aqueous phase was extracted with EtOAc (2×100 mL). The combined organic layers were filtered through a pad of Celite, and the filtrate was washed with saturated aqueous NaHCO₃ (100 mL), brine (100 mL), dried (Na₂SO₄), filtered, and concentrated. Purification by flash chromatography (20% EtOAc/hexanes) afforded diol 13 as a clear, colorless oil in 89% yield (312 mg, 0.685 mmol).

¹H NMR (600 MHz, CDCl₃) δ 7.63 (ddd, J=8.5, 5.4, 3.2 Hz, 1H), 7.41-7.34 (m, 4H), 7.34-7.29 (m, 1H), 7.29-7.24 (m, 1H), 7.16 (apparent td, J=9.4, 4.1 Hz, 1H), 5.42 (dd, J=3.3, 1.6 Hz, 1H), 5.41 (d, J=1.6 Hz, 1H), 4.83 (d, J=11.3 Hz, 1H), 4.78 (d, J=11.3 Hz, 1H), 4.17 (ddd, J 9.1, 5.7, 3.2 Hz, 1H), 4.08 (dt, J=9.7, 3.1 Hz, 1H), 3.93 (t, J=9.5 Hz, 1H), 3.91-3.83 (m, 2H), 2.72-2.57 (m, 2H), 2.08 (d, J=5.7 Hz, 1H), 1.81 (dd, J=7.7, 5.4 Hz, 1H), 1.30 (t, J=7.4 Hz, 3H).

¹³C NMR (150 MHz, CDCl₃) δ 162.90, 162.89, 162.87, 162.86, 158.96, 158.95, 158.92, 158.90, 157.30, 157.28, 157.27, 157.25, 137.96, 128.65, 128.17, 128.14, 121.89, 121.83, 121.73, 121.67, 119.28, 119.23, 119.20, 119.15, 118.66, 118.61, 118.57, 118.56, 118.49, 118.44, 118.40, 118.39, 82.07, 75.70, 75.53, 75.03, 72.20, 70.84, 61.90, 25.72, 14.89.

IR (ATR-FTIR, thin film) 3454, 3126, 3076, 3032, 2967, 2929, 2879, 1722, 1627, 1595, 1496, 1454, 1429, 1310, 1270, 1242, 1187, 1092, 1074, 965, 943, 891, 827 cm⁻¹.

[α]²²_{D} (c 1.1, CH₂Cl₂) +63.9.

LRMS (ESI+) m/z calc'd for [M+Na]⁺ (C₂₂H₂₄F₂NaO₆S) requires 477.1. found 477.1.

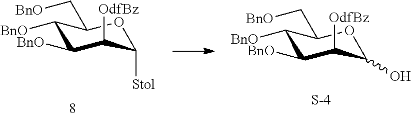

### 3,4,6-Tri-O-benzyl-2-O-(2,5-difluorobenzoyl)-D-mannopyranoside (S-4)

(The procedure for conversion to the hemiacetal is based on the following report: Motawia, M. S.; Marcussen, J.; Møller, B. L. J. Carbohydr. Chem. 1995, 14, 1279-1294.). To a cooled (0° C.) solution of thioglycoside 8 (1.88 g, 2.70 mmol) in acetone/water (9:1, 40 mL) was added N-bromosuccinimide (1.44 g, 8.09 mmol). The resulting clear, orange solution was stirred at 0° C., with additional portions of N-bromosuccinimide (480 mg, 2.70 mmol) added at 1 h and 4 h. After a total reaction time of 6 h, the reaction mixture was concentrated until turbidity was evident. The residue was then taken up in EtOAc (500 mL), washed with saturated aqueous NaHCO₃ (3×120 mL), water (3×120 mL), dried (Na₂SO₄), filtered, and concentrated. Purification by flash chromatography (25% EtOAc/hexanes) afforded anomeric alcohol S-4 as a clear, colorless oil in 86% yield (1.37 g, 2.33 mmol, α:β mixture).

¹H NMR (600 MHz, CDCl₃, major anomer) δ 7.66 (ddd, J=8.5, 5.4, 3.3 Hz, 1H), 7.39-7.31 (m, 6H), 7.31-7.25 (m, 7H), 7.25-7.21 (m, 1H), 7.19-7.14 (m, 2H), 7.11 (apparent td, J=9.3, 4.2 Hz, 1H), 5.59 (dd, J=3.1, 2.0 Hz, 1H), 5.33 (dd, J=3.9, 2.0 Hz, 1H), 4.86 (d, J=10.8 Hz, 1H), 4.77 (d, J=11.4 Hz, 1H), 4.62 (d, J=12.2 Hz, 1H), 4.59 (d, J=11.4 Hz, 1H), 4.54 (d, J=12.1 Hz, 1H), 4.48 (d, J=10.9 Hz, 1H), 4.14 (dd, J=9.4, 3.0 Hz, 1H), 4.13-4.09 (m, 1H), 3.87 (apparent t, J=9.6 Hz, 1H), 3.74 (dd, J=10.4, 2.2 Hz, 1H), 3.69 (dd, J=10.5, 5.8 Hz. 1H), 3.65 (d, J=3.7 Hz, 1H).

¹³C NMR (150 MHz, CDCl₃, major anomer) δ 162.34, 162.33, 162.32, 162.30, 159.08, 159.07, 158.73, 158.71, 157.37, 157.36, 157.11, 157.09, 138.10, 137.83, 137.82, 128.32, 128.29, 127.99, 127.91, 127.67, 127.66, 127.65, 121.57, 121.51, 121.41, 121.35, 119.43, 119.38, 119.35, 119.30, 118.53, 118.47, 118.44, 118.36, 118.31, 118.27, 92.13, 77.53, 75.13, 74.51, 73.31, 71.69, 71.19, 70.11, 69.28.

IR (ATR-FTIR, thin film) 3406, 3087, 3064, 3032, 2924, 2868, 1738, 1720, 1627, 1596, 1496, 1454, 1428, 1363, 1342, 1309, 1270, 1254, 1242, 1188, 1119, 1075, 1063, 1038, 978, 943, 910, 892, 825 cm⁻¹.

[α]²¹_{D} (c 1.9, CH₂Cl₂) −25.5.

LRMS (ESI+) m/z calc'd for [M+Na]⁺ (C₃₄H₃₂F₂NaO₇) requires 613.2. found 613.3.

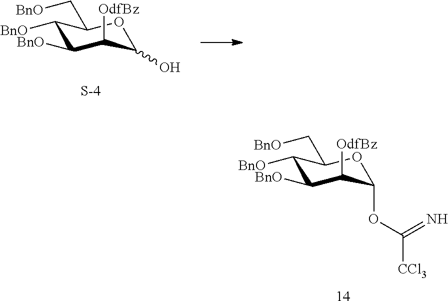

### 3,4,6-Tri-O-benzyl-2-O-(2,5-difluorobenzoyl)-α-D-mannopyranosyl trichloroacetimidate (14)

To a cooled (0° C.) solution of anomeric alcohol S-4 (1.35 g, 2.29 mmol) and trichloroacetonitrile (2.3 mL, 22.9 mmol) in CH₂Cl₂ (9.0 mL) was added and 1,8-diazabicyclo[5.4.0]undec-7-ene (40 μL, 0.267 mmol) dropwise via syringe. The resulting clear, yellow solution was stirred at 0° C. for 4 h. The reaction mixture was loaded directly on a short silica gel column and purified by flash chromatography (20% EtOAc/hexanes) to afford trichloroacetimidate 14 as a clear, yellow oil in 96% yield (1.61 g, 2.19 mmol, ˜95% α-anomer).

¹H NMR (600 MHz, CDCl₃, α-anomer) δ 8.71 (s, 1H), 7.70 (ddd, J=8.5, 5.4, 3.2 Hz, 1H), 7.37-7.23 (m, 14H), 7.22-7.18 (m, 2H), 7.11 (apparent td, J=9.4, 4.2 Hz, 1H), 6.42 (d, J=2.1 Hz, 1H), 5.71 (apparent t, J=2.6 Hz, 1H), 4.87 (d, J=10.7 Hz, 1H), 4.79 (d, J=11.4 Hz, 1H), 4.70 (d, J=12.1 Hz, 1H), 4.64 (d, J=11.4 Hz, 1H), 4.55 (d, J=10.6 Hz, 1H), 4.52 (d, J=12.1 Hz, 1H), 4.18 (apparent t, J=9.6 Hz, 1H), 4.13 (dd, J=9.5, 3.0 Hz, 1H), 4.03 (ddd, J=9.8, 3.9, 1.8 Hz, 1H), 3.86 (dd, J=11.1, 3.8 Hz, 1H), 3.75 (dd, J=11.2, 1.9 Hz, 1H).

¹³C NMR (150 MHz, CDCl₃, α-anomer) δ 162.12, 162.10, 162.09, 162.08, 159.91, 159.18, 159.17, 158.77, 158.75, 157.47, 157.45, 157.15, 157.14, 138.22, 137.98, 137.38, 128.42, 128.37, 128.28, 128.25, 128.18, 127.91, 127.82, 127.61, 127.48, 121.86, 121.80, 121.70, 121.64, 119.06, 119.01, 118.98, 118.93, 118.62, 118.57, 118.55, 118.46, 118.40, 118.38, 95.07 (¹J_{CH}=180.5 Hz), 90.71, 77.14, 75.52, 74.48, 73.66, 73.34, 72.06, 68.43, 68.35.

IR (ATR-FTIR, thin film) 3337, 3087, 3064, 3032, 2904, 2869, 1742, 1726, 1675, 1627, 1596, 1496, 1454, 1428, 1362, 1320, 1307, 1267, 1239, 1187, 1164, 1101, 1076, 1067, 1046, 1028, 972, 946, 929, 828 cm⁻¹.

[α]²²_{D} (c 1.1, CH₂Cl₂) +14.9.

LRMS (ESI+) m/z calc'd for [M+Na]⁺ (C₃₆H₃₂³⁵Cl₃F₂NNaO₇) requires 756.1. found 756.1.

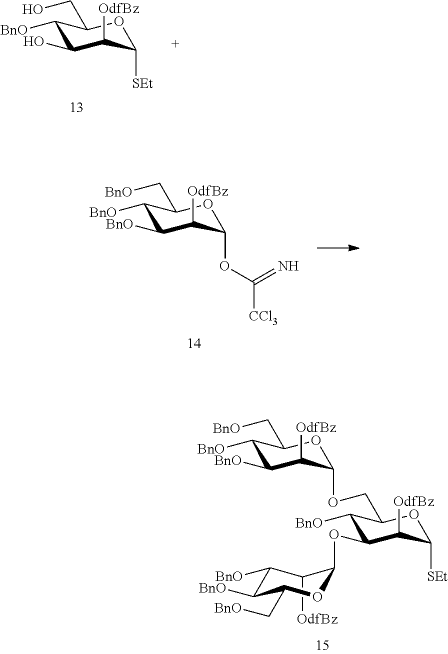

### Ethyl 3,4,6-tri-O-benzyl-2-O-(2,5-difluorobenzoyl)-α-D-mannopyranosyl-(1→3)-[3,4,6-tri-O-benzyl-2-O-(2,5-difluorobenzoyl)-α-D-mannopyranosyl-(1→6)]-4-O-benzyl-2-O-(2,5-difluorobenzoyl)-1-thio-α-D-mannopyranoside (15)

Diol acceptor 14 (294 mg, 0.647 mmol) and trichloroacetimidate donor 13 (1.17 g, 1.60 mmol) were azeotroped three times with benzene then dried for 2 h in vacuo. The residue was dissolved in anhydrous CH₂Cl₂ (6.5 mL), and the clear, yellow solution was stirred in the presence of acid-washed molecular sieves (AW-300, 1.6 mm pellets, 900 mg) for 15 min at room temperature. The mixture was cooled to 0° C., then trimethylsilyl trifluoromethanesulfonate (5% in CH₂Cl₂, 0.24 mL, 66.4 mol) was added dropwise via syringe. After stirring for 2 h at 0° C., the reaction medium was neutralized with a few drops of triethylamine, then filtered and concentrated. Purification by flash chromatography (0-1% EtOAc/CH₂Cl₂) afforded trisaccharide 15 as a white foam in 75% yield (771 mg, 0.482 mmol). Measurement of ¹J_{CH} coupling constants confirmed the α-anomeric configuration at the newly formed glycosidic bonds (data listed below).

¹H NMR (600 MHz, CDCl₃) δ 7.68-7.58 (m, 3H), 7.39-7.19 (m, 28H), 7.19-7.11 (m, 8H), 7.11-7.04 (m, 5H), 5.72 (apparent t, J=2.5 Hz, 1H), 5.59 (apparent t, J=2.3 Hz, 1H), 5.54 (dd, J=2.7, 1.5 Hz, 1H), 5.38 (d, J=1.1 Hz, 1H), 5.30 (d, J=1.9 Hz, 1H), 5.11 (d, J=2.1 Hz, 1H), 4.83 (d, J=10.8 Hz, 1H), 4.81-4.77 (m, 2H), 4.76 (d, J=11.1 Hz, 1H), 4.71 (d, J=12.1 Hz, 1H), 4.66 (d, J=12.2 Hz, 1H), 4.61-4.53 (m, 3H), 4.49-4.38 (m, 5H), 4.27 (dd, J=9.4, 3.1 Hz, 1H), 4.21-4.15 (m, 1H), 4.08 (dd, J=9.3, 3.1 Hz, 1H), 4.06-3.98 (m, 3H), 3.97-3.91 (m, 2H), 3.85-3.79 (m, 2H), 3.77-3.72 (m, 2H), 3.68 (dd, J=10.8, 3.6 Hz, 1H), 3.66-3.61 (m, 2H), 2.67-2.49 (m, 2H), 1.23 (t, J=7.4 Hz, 3H).

¹³C NMR (150 MHz, CDCl₃) δ 162.85, 162.84, 162.82, 162.81, 162.08, 162.06, 162.05, 162.03, 162.00, 161.99, 161.97, 161.96, 159.12, 159.10, 158.90, 158.89, 158.86, 158.85, 158.71, 157.41, 157.39, 157.25, 157.23, 157.20, 157.19, 157.10, 138.37, 138.37, 138.25, 138.20, 137.78, 137.69, 137.55, 128.47, 128.35, 128.23, 128.21, 128.17, 128.13, 128.06, 127.89, 127.85, 127.72, 127.63, 127.60, 127.59, 127.46, 127.36, 121.86, 121.80, 121.70, 121.64, 121.61, 121.55, 121.53, 121.47, 121.46, 121.39, 121.37, 121.31, 119.45, 119.40, 119.37, 119.32, 119.28, 119.23, 119.20, 119.18, 119.15, 119.10, 118.90, 118.85, 118.73, 118.68, 118.50, 118.46, 118.45, 118.33, 118.29, 99.62 (¹J_{CH}=173.4 Hz), 97.64 (¹J_{CH}=174.0 Hz), 81.72, 78.30, 78.13, 77.64, 75.32, 75.21, 75.12, 74.75, 74.68, 74.16, 73.90, 73.32, 73.26, 72.60, 71.82, 71.69, 71.56, 71.44, 69.93, 69.31, 68.59, 68.23, 65.62, 25.63, 14.95.

IR (ATR-FTIR, thin film) 3087, 3066, 3031, 2928, 2869, 1739, 1722, 1627, 1595, 1496, 1454, 1428, 1362, 1308, 1269, 1239, 1187, 1145, 1079, 1028, 981, 943, 910, 892, 826 cm⁻¹.

[α]²²_{D} (c 1.0, CH₂Cl₂) +14.3.

LRMS (ESI+) m/z calc'd for [M+Na]⁺ (C₉₀H₈₄F₆NaO₁₈S) requires 1621.5. found 1621.3.

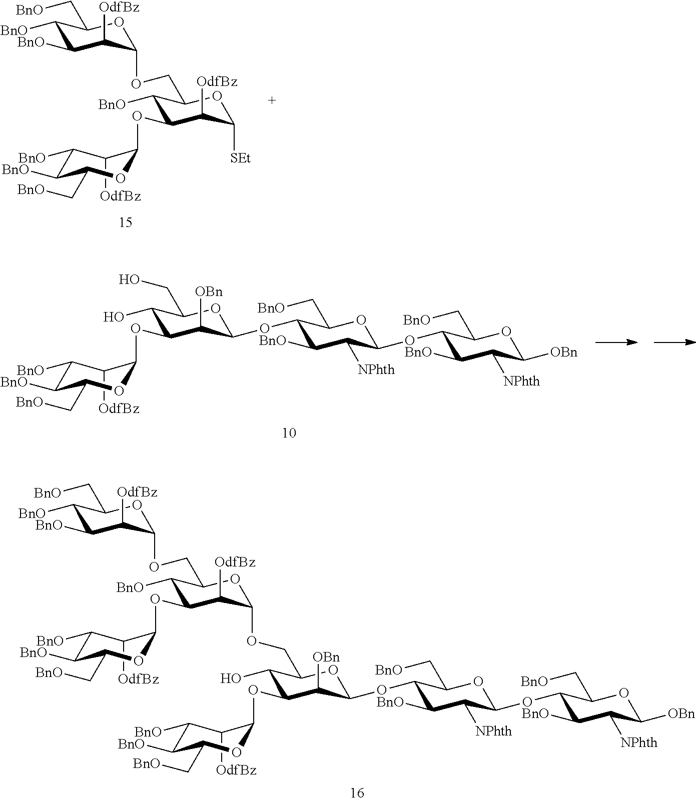

### Benzyl[3,4,6-tri-O-benzyl-2-O-(2,5-difluorobenzoyl)-α-D-mannopyranosyl-(1→3)]-[[3,4,6-tri-O-benzyl-2-O-(2,5-difluorobenzoyl)-α-D-mannopyranosyl-(1→3)]-[3,4,6-tri-O-benzyl-2-O-(2,5-difluorobenzoyl)-α-D-mannopyranosyl-(1→6)]]-4-O-benzyl-2-O-(2,5-difluorobenzoyl)-α-D-mannopyranosyl-(1→6)]-2-O-benzyl-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside (16)

A mixture of tetrasaccharide acceptor 10 (1.0 g, 0.53 mmol) and trimannose thioglycoside donor 15 (0.86 g, 0.54 mmol) was dissolved in anhydrous CH₂Cl₂ (40 mL). Freshly activated AW-300 MS (1.8 g) was added and stirred at r.t. for 1 h. The mixture was cooled to 0° C., NIS (180 mg, 0.8 mmol) and TMSOTf (20 μl, 0.11 mmol) were added sequentially, and the mixture was allowed to warm up to r.t. over 4 h. The mixture was filtered through a pad of Celite and the organic layer was washed with saturated aqueous Na₂S₂O₃, saturated aqueous NaHCO₃, water, brine, dried over MgSO₄ and concentrated. The residue was purified by flash chromatography (hexanes:EtOAc, 2:1) to give the heptasaccharide 16 (1.35 g, 75%) as an amorphous white solid. The regioselectivity of glycosylation was confirmed by analogy to the case of the pentasaccharide, noting a shift in the C-6 carbon of the central, branched β-Man from 62.6 ppm to 65.6 ppm. Measurement of ¹J_{CH} coupling constants confirmed the anomeric configuration at each inter-residue glycosidic bond (data listed below).

¹H NMR (600 MHz, CDCl₃) δ 7.81-6.56 (m, 100H), 5.69 (t, J=2.5 Hz, 1H), 5.67 (t, J=2.5 Hz, 1H), 5.56 (t, J=2.6 Hz, 1H), 5.45-5.41 (m, 1H), 5.39 (d, J=1.9 Hz, 1H), 5.20 (d, J=7.7 Hz, 1H), 5.18 (d, J=1.9 Hz, 1H), 5.09 (d, J=1.9 Hz, 1H), 4.93 (d, J=12.3 Hz, 1H), 4.91 (d, J=8.6 Hz, 1H), 4.87 (d, J=1.8 Hz, 1H), 4.84-4.69 (m, 8H), 4.69-4.61 (m, 3H), 4.60-4.37 (m, 17H), 4.37-4.30 (m, 3H), 4.23 (dd, J=9.5, 3.2 Hz, 1H), 4.20 (d, J=12.1 Hz, 1H), 4.18-4.03 (m, 7H), 4.03-3.91 (m, 6H), 3.91-3.83 (m, 3H), 3.80 (m, 2H), 3.78-3.67 (m, 5H), 3.65 (m, 2H), 3.60 (dd, J=10.7, 1.9 Hz, 1H), 3.58-3.53 (m, 1H), 3.53-3.39 (m, 5H), 3.35 (m, 2H), 3.24 (ddd, J=9.7, 4.0, 1.8 Hz, 1H), 3.14 (dt, J=9.8, 2.5 Hz, 1H), 3.05 (dt, J=9.4, 3.8 Hz, 1H).

¹³C NMR (150 MHz, CDCl₃) δ 168.21, 167.59, 167.40, 162.59, 162.13, 161.91, 159.06, 158.78, 158.71, 157.35, 157.16, 157.09, 138.66, 138.62, 138.50, 138.46, 138.38, 138.07, 137.94, 137.89, 137.83, 137.63, 137.21, 133.77, 133.49, 133.40, 131.82, 131.71, 131.48, 128.52, 128.46, 128.39, 128.35, 128.32, 128.27, 128.22, 128.17, 128.12, 128.08, 128.05, 128.01, 127.98, 127.88, 127.84, 127.81, 127.77, 127.74, 127.68, 127.63, 127.54, 127.53, 127.49, 127.44, 127.41, 127.38, 127.32, 127.29, 127.24, 127.17, 127.12, 126.84, 126.75, 123.56, 123.06, 121.37, 121.20, 119.62, 119.58, 119.55, 119.53, 119.49, 119.48, 119.45, 119.43, 119.40, 119.35, 119.18, 119.13, 119.10, 119.04, 118.79, 118.74, 118.62, 118.54, 118.47, 118.40, 118.37, 118.30, 118.23, 118.19, 101.99 (¹J_{CH}=158.8 Hz, β-Man), 99.59 (¹J_{CH}=174.5 Hz, α-Man), 98.62 (¹J_{CH}=175.2 Hz, α-Man), 97.83 (¹J_{CH}=175.0 Hz, α-Man), 97.08 (×2) (¹J_{CH}=168.3 Hz, β-GlcN), 97.06 (¹J_{CH}=174.9 Hz, α-Man), 80.75, 79.69, 78.22, 78.19, 77.99, 77.91, 77.80, 76.70, 75.95, 75.37, 75.21, 74.97, 74.74, 74.58, 74.55, 74.51, 74.40, 74.38, 74.35, 74.31, 74.19, 73.90, 73.42, 73.29, 73.13, 73.01, 72.65, 72.61, 72.60, 72.18, 71.84, 71.50, 70.98, 70.44, 69.90, 69.83, 69.36, 69.25, 68.65, 68.32, 68.14, 67.68, 67.62, 67.49, 65.54.

IR (ATR-FTIR, thin film) 3031, 2929, 2869, 1715, 1495, 1387, 1269, 1078, 738, 698 cm⁻¹.

[α]²⁴_{D} (c 1.0, CH₂Cl₂) −11.5.

LRMS (ESI+) m/z calc'd for [M+Na]⁺ (C₁₉₈H₁₈₂F₈N₂NaO₄₂) requires 3434.2. found 3434.0.

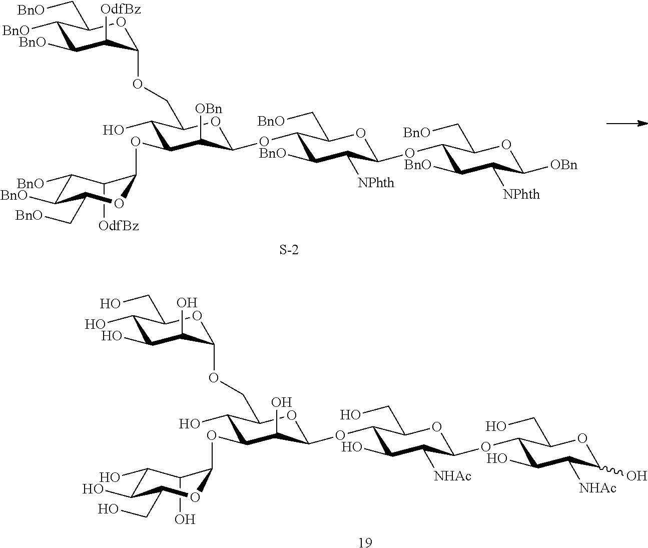

### [α-D-mannopyranosyl-(1→3)]-[α-D-mannopyranosyl-(1→6)]-β-D-mannopyranosyl-(1→4)-2-deoxy-2-N-acetyl-β-D-glucopyranosyl-(1→4)-2-deoxy-2-N-acetyl-D-glucopyranoside (19)

To a solution of oligosaccharide S-2 (846 mg, 0.35 mmol) in CH₂Cl₂/MeOH (1:9, 20 mL), was added Na-metal (16 mg, 0.69 mmol). The mixture was stirred at r.t. for 4 h, quenched with Dowex 50 W X8 resin, filtered, and evaporated to dryness. The residue was dissolved in toluene (16 mL), n-butanol (32 mL), ethylenediamine (9.6 mL), and heated at 90° C. for 24 h. The mixture was co-evaporated with toluene.

The residue was dissolved in MeOH (40 mL). Acetic anhydride (2.6 mL) and triethylamine (4.0 mL) were sequentially added to the mixture and stirred at Lt. for 12 h. The reaction was monitored by LCMS at each stage. The residue was purified by flash chromatography (hexanes:CH₂Cl₂:acetone, 1:1:1) to give the partially deprotected oligosaccharide (620 mg) as an amorphous white solid.

To a solution of partially deprotected pentasaccharide (620 mg) in MeOH (60 mL) was added H₂O (6.0 mL) dropwise, at r.t. under an atmosphere of argon. Pd(OH)₂/C (20% by wt., 620 mg) was added to the mixture under argon atmosphere. Argon was replaced by hydrogen and the mixture was stirred at r.t. for 12 h under 1 atm pressure. The mixture was filtered by PTFE GL 0.45 μm cartridge, evaporated, and purified using C18 SepPak column. The product eluted in neat H₂O. The pure fractions were combined and lyophilized to give compound 19 (240 mg, 0.26 mmol) as a mixture of anomers in 74% overall yield over 4 steps.

¹H NMR data were consistent with previously published values (Paulsen, H.; Lebuhn, R. Carbohydr. Res. 1984, 130, 85-101.).

LRMS (ESI+) m/z calc'd for [M+H]⁺ (C₃₄H₅₉N₂O₂₆) requires 911.3. found 911.5.

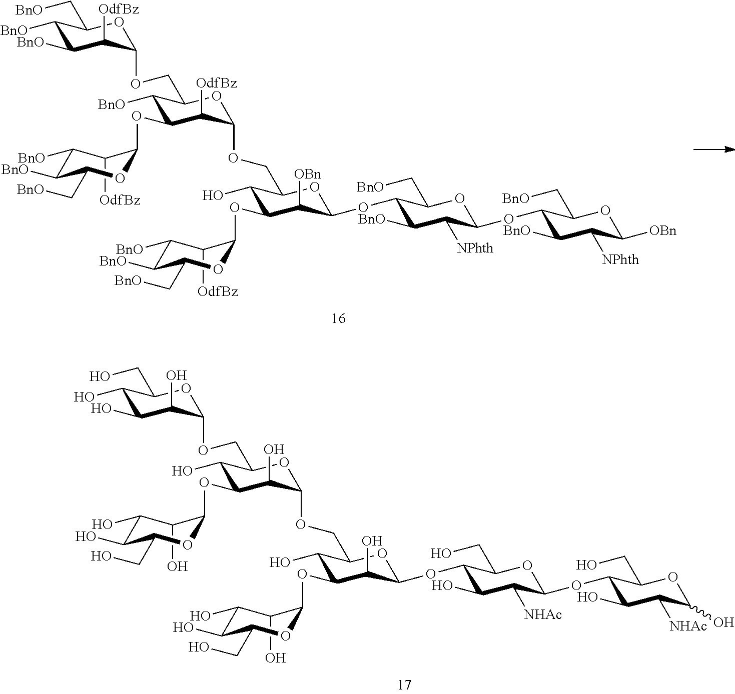

### [α-D-mannopyranosyl-(1→3)]-[[α-D-mannopyranosyl-(1→3)]-[α-D-mannopyranosyl-(1→6)]]-α-D-mannopyranosyl-(1→6)]-β-D-mannopyranosyl-(1→4)-2-deoxy-2-N-acetyl-β-D-glucopyranosyl-(1→4)-2-deoxy-2-N-acetyl-D-glucopyranoside (17)

To a solution of oligosaccharide 16 (1.2 g, 0.35 mmol) in CH₂Cl₂/MeOH (1:10, 22 mL), was added Na-metal (33 mg, 1.4 mmol). The mixture was stirred at r.t. for 8 h, quenched with Dowex 50 W X8 resin, filtered, and evaporated to dryness. The residue was dissolved in toluene (16 mL), n-butanol (32 mL), ethylenediamine (9.6 mL), and heated at 90° C. for 24 h. The mixture was co-evaporated with toluene.

The residue was dissolved in MeOH (40 mL). Acetic anhydride (2.6 mL) and triethylamine (4.0 mL) were sequentially added to the mixture and stirred at r.t. for 12 h. The reaction was monitored by LCMS at each stage. The residue was purified by flash chromatography (hexanes:CH₂Cl₂:acetone, 1:1:1) to give the partially deprotected oligosaccharide (940 mg) as an amorphous white solid.

To a solution of partially deprotected heptasaccharide (800 mg) in MeOH (60 mL) was added H₂O (6.0 mL) dropwise, at r.t. under an atmosphere of argon. Pd(OH)₂/C (20% by wt., 800 mg) was added to the mixture under argon atmosphere. Argon was replaced by hydrogen and the mixture was stirred at r.t. for 12 h under 1 atm pressure. The mixture was filtered by PTFE GL 0.45 μm cartridge, evaporated, and purified using C18 SepPak column. The product eluted in neat H₂O. The pure fractions were combined and lyophilized to give compound 17 (288 mg, 0.23 mmol) as a mixture of anomers in 77% overall yield over 4 steps.

¹H NMR (600 MHz, D₂O, α-anomer) δ 5.19 (d, J=2.6 Hz, 1H), 5.12-5.07 (m, 2H), 4.91 (d, J=1.9 Hz, 1H), 4.87 (br d. 1H), 4.63-4.57 (m, 1H), 4.26 (d, J=2.6 Hz, 1H), 4.15 (dd, J=3.4, 1.7 Hz, 1H), 4.08 (dd, J=3.4, 1.7 Hz, 1H), 4.07 (dd, J=3.6, 1.7 Hz, 1H), 4.03-3.58 (m, 38H), 2.07 (s, 3H), 2.04 (s, 3H).

LRMS (ESI+) m/z calc'd for [M+H]⁺ (C₄₆H₇₉N₂O₃₆) requires 1235.4. found 1235.6.

### General Procedures for Peptide and Glycopeptide Synthesis.

Solid-Phase Peptide Synthesis by Fmoc-Strategy.

Automated peptide synthesis was performed on an Applied Biosystems Pioneer continuous S3 flow peptide synthesizer. Peptides were synthesized under standard automated Fmoc protocols on Fmoc-Arg(Pbf)-TGT resin or TG Sieber resin. The deblock mixture was a mixture of 100:2:2 of DMF/piperidine/DBU. The following Fmoc amino acids from Novabiochem were employed: Fmoc-Ala-OH, Fmoc-Arg(Pbf)-OH, Fmoc-Asn(Dmcp)-OH, Fmoc-Asp(OAll)-OH, Fmoc-Asp(OtBu)-OH, Fmoc-Asp(OMpe)-OH, Boc-Cys(Trt)-OH, Fmoc-Gln(Dmcp)-OH, Fmoc-Glu(OtBu)-OH, Fmoc-His(Trt)-OH, Fmoc-Ile-OH, Fmoc-Leu-OH, Fmoc-Lys(Boc)-OH, Fmoc-Phe-OH, Fmoc-Pro-OH, Fmoc-Ser(tBu)-OH, Fmoc-Thr(tBu)-OH, Fmoc-Tyr(tBu)-OH, Fmoc-Val-OH. The following pseudoproline dipeptides were used: Fmoc-Ile-Thr(Ψ^{Me,Me}pro)-OH (Novabiochem) and Fmoc-Met-Thr(Ψ^{Me,Me}pro)-OH (S-8, synthesized in the laboratory).

### Acid-Labile Protecting Group Removal.

Cocktail B.

Peptides were subjected to Cocktail B (1 mL/10 mg of peptide) consisting of trifluoroacetic acid (88% by volume), water (5% by volume), phenol (5% by weight), and i-Pr₃SiH (2% by volume). The resulting solution was triturated in ice-cold diethyl ether (3×15 mL) to give a white precipitate, which was centrifuged. The supernatant was discarded and the precipitate was solubilized in water/acetonitrile (1:1, 0.05% trifluoroacetic acid), lyophilized and the resulting solid was purified by HPLC.

Cocktail R.

Peptides were subjected to Cocktail R (3 mL/100 mg of peptide) consisting of trifluoroacetic acid (90% by volume), thioanisole (5% by volume), 1,2-ethanedithiol (3% by weight), and anisole (2% by volume). The resulting solution was triturated in ice-cold diethyl ether (3×15 mL) to give a white precipitate, which was centrifuged. The supernatant was discarded and the precipitate was solubilized in water/acetonitrile (1:1, 0.05% trifluoroacetic acid), lyophilized and the resulting solid was purified by HPLC.

HPLC.

All separations involved a mobile phase of 0.05% TFA (v/v) in water (solvent A)/0.04% TFA in acetonitrile (solvent B).

HPLC LC-MS analytical separations were performed using a Waters 2695 Separations Module and a Waters 2996 Photodiode Array Detector equipped with Varian Microsorb C18 column (150×2 mm) or Waters C8 X-Bridge column (150×2.1 mm) or Varian 300-5 C4 column (250×2 mm) at a flow rate of 0.2 mL/min.

UPLC LC-MS analytical separations were performed using a Waters Acquity system equipped with an Acquity UPLC BEH C4 column (100×2.1 mm).

Preparatory HPLC separations were performed using a WATERS 2545 Binary Gradient Module equipped with a WATERS 2996 Photodiode Array Detector using either Microsorb 100-5 C18 column (250×21.4 mm), Microsorb 100-5 C8 column (250×21.4 mm) or Waters C8 X-Bridge column (150×19 mm) at a flow rate of 16 mL/min.

Native Chemical Ligation (NCL) Buffer.

The buffer required for native chemical ligation (NCL) was freshly prepared prior to the reaction. Na₂HPO₄ (56.6 mg, 0.4 mmol) was solubilized in water (1 mL), Guanidine.HCl (1.146 g, 12 mmol), and TCEP.HCl (10.8 mg, 0.04 mmol) were then added and solubilized. The pH was brought to 7 with a solution of NaOH (5 M, 20 μL). After 15 min degassing with argon, 4-mercaptophenylacetic acid (MPAA) (67 mg, 0.4 mmol) was added and the pH was brought to 7.2 with a solution of NaOH (5 M, 120 μL). After 15 min degassing the solution was ready for use.

Glycan Anomeric Amine Installation (Kochetkov Reaction).

Oligosaccharide was dissolved in water (5 mL) and added to (NH₄)HCO₃ (6 g, BioUltra, 99.5% (T), Cat. No. 09830 Fluka). The resultant slurry was warmed to 40° C. and stirred very slowly at this temperature for three days. After three days, the clear supernatant was filtered through a plug of cotton. The remaining material was rinsed with the same amount of cold water (2×5 mL), filtered, pooled with the clear supernatant, immediately frozen and lyophilized. The lyophilization was deemed complete when the mass of the product remained constant. This provided the glycosyl amine as a white solid (quantitative). Oligosaccharides were stored at room temperature on the lyophilizer.

### Experimental Procedures: Peptides and Glycopeptides.

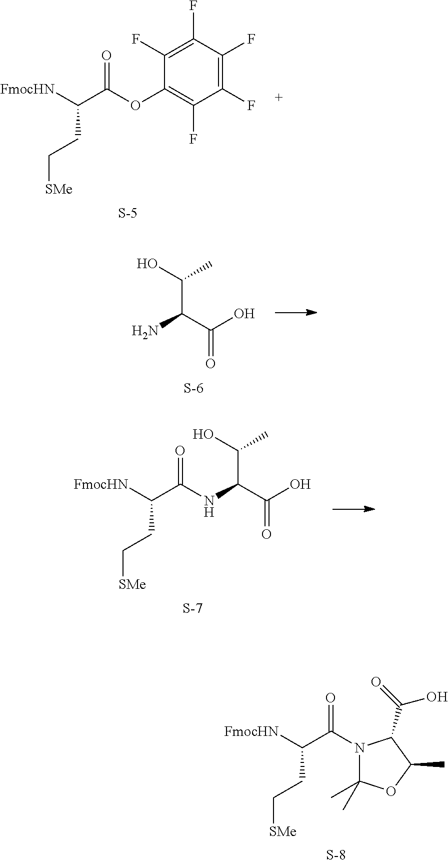

Fmoc-Met-Thr(Ψ^{Me,Me}pro)-OH (S-8)

(General procedure for pseudoproline synthesis: Wohr, T.; Wahl, F.; Nefzi, A.; Rohwedder, B.; Sato, T.; Sun, X.; Mutter, M. J. Am. Chem. Soc. 1996, 118, 9218-9227.). L-Threonine (S-6) (1.03 g, 8.7 mmol) was dissolved in a minimal volume of aqueous sodium carbonate (10% w/v) at pH 9 (9 mL), and the solution was added to a suspension of Fmoc-Met-OPfp (S-5) (1.55 g, 2.9 mmol) in acetone (23 mL). After vigorous stirring for 3 h, the reaction mixture was cooled to 0° C. and acidified with 1 N HCl to pH ˜1. The solution was then concentrated in vacuo to less than half of the initial volume and ethyl acetate (100 mL) and water (60 mL) were added. The layers were separated and the aqueous layer was extracted with ethyl acetate (2×60 mL). The combined organic extracts were washed with water (30 mL) and brine (2×30 mL), dried over MgSO₄, filtered and evaporated to dryness. The residue was crystallized from ethyl acetate/hexane to give Fmoc-Met-Thr-OH (S-7) as a white solid.

Dipeptide S-7 (2.88 mmol) was then suspended in dry THF (55 mL), and pyridyl toluene-4-sulfonate (145 mg, 0.58 mmol) and 2,2-dimethoxypropane (1.8 mL, 14.4 mmol) were added. The suspension was then heated to reflux overnight under Ar, the condensate being bypassed over molecular sieves (4 Å). After cooling, triethylamine was added (120 μL, 0.86 mmol) and the mixture was evaporated to dryness. The residue was taken up in ethyl acetate (100 mL), and washed with water (2×50 mL). The aqueous layer was extracted with ethyl acetate (2×60 mL) and the combined organics were dried over MgSO₄, filtered and concentrated. The residue was purified by flash chromatography (20:1 to 10:1 CH₂Cl₂/MeOH) to give the desired pseudoproline dipeptide Fmoc-Met-Thr(Ψ^{Me,Me}pro)-OH (S-8) (1.3 g, 88% yield) as a white solid.

¹H NMR (600 MHz, CDCl₃) δ 7.79-7.69 (m, 3H), 7.62-7.46 (m, 3H), 7.44-7.33 (m, 3H), 7.33-7.23 (m, 4H), 5.86 (d, J=8.8 Hz, 1H), 4.48-4.33 (m, 3H), 4.33-4.23 (m, 3H), 4.21-4.10 (m, 2H), 2.60-2.41 (m, 3H), 2.09 (s, 3H), 2.01-1.84 (m, 3H), 1.67 (s, 3H), 1.59 (s, 3H), 1.49 (d, J=6.0 Hz, 3H), 1.42 (d, J=5.9 Hz, 1H).

¹³C NMR (150 MHz, CDCl₃) δ 172.2, 170.0, 156.2, 143.7, 143.2, 141.1, 141.0, 127.6, 127.0, 125.1, 125.0, 119.81, 119.79, 97.2, 74.9, 72.7, 67.5, 65.5, 52.8, 46.8, 33.6, 29.8, 26.2, 23.4, 19.9, 15.4.

LRMS (ESI+) m/z calc'd for [M+Na]⁺ (C₂₇H₃₂N₂O₆SNa) requires 535.6. found 535.3; m/z calc'd for [M+K]⁺ (C₂₇H₃₂N₂O₆SK) requires 551.7. found 551.2.

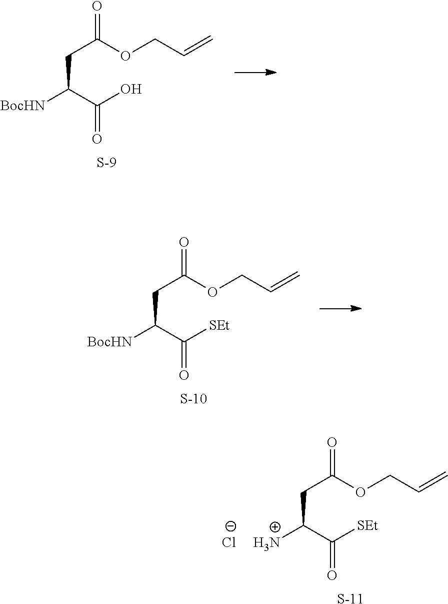

H-Asp(OAll)-SEt.HCl (S-11).

Boc-Asp(OAll)-OH (S-9) (2.73 g, 10 mmol) was solubilized in dichloromethane (50 mL). To this solution EDC (1.77 mL, 10 mmol), HOBt (4.05 g, 30 mmol) and ethanethiol (3.6 mL, 50 mmol) were added. The mixture was stirred for 3 h 30 min, concentrated in vacuo and purified by flash chromatography (10-15% EtOAc/hexanes) to afford after concentration and lyophilization Boc-Asp(OAll)-SEt (S-10) (1.11 g, 3.5 mmol, 35% yield) as a white solid.

Boc-Asp(OAll)-SEt (454 mg, 1.4 mmol) was directly solubilized in a solution of HCl in dioxane (4 M, 24 mL). After 1 h 30 min at room temperature, the solution was concentrated in vacuo, resuspended in water and lyophilized twice to afford H-Asp(OAll)-SEt.HCl (S-11) as white solid (373 mg, 1.4 mmol, quantitative yield).

¹H NMR (600 MHz, DMSO-d₆) δ 8.83 (br s, 3H), 5.91 (ddt, J=17.3, 10.7, 5.5 Hz, 1H), 5.33 (apparent dq, J=17.3, 1.6 Hz, 1H), 5.24 (apparent dq, J=10.5, 1.4 Hz, 1H), 4.60 (apparent dq, J=5.5, 1.3 Hz, 2H), 4.45 (t, J=5.7 Hz, 1H), 3.13 (dd, J=17.5, 5.4 Hz, 2H), 3.08 (dd, J=17.5, 6.1 Hz, 1H), 3.00-2.90 (m, 2H), 1.19 (t, J=7.3 Hz, 3H).

¹³C NMR (150 MHz, DMSO-d₆) δ 195.2, 168.4, 132.1, 118.3, 65.4, 54.9, 35.1, 23.3, 14.4.

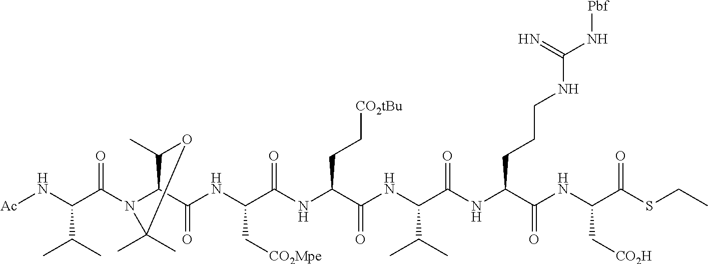

Protected N-Terminal Fragment (S-12).

Upon completion of automated synthesis on 0.2 mmol of Fmoc-Arg(Pbf)-NovaSynTGT resin, the peptide-resin was subjected to acetylation. The peptide-resin was washed with DMF into a peptide synthesis vessel and treated with acetic anhydride (366 μL, 4 mmol), DIEA (768 μL, 4.4 mmol) in DMF (4 mL) for 25 min. The peptide-resin was then washed with DMF, dichloromethane and methanol. After drying, the resin was subjected to a cleavage cocktail (1:1:8 of acetic acid/trifluoroethanol/methylene chloride) 3 times for 30 min. The resulting portions of cleavage solution were pooled and concentrated at room temperature. The oily residue was resuspended in a minimum amount of trifluoroethanol and precipitated with water. The resulting mixture was immediately lyophilized to afford the peptide as white solid (175 mg, 73% yield).

To a solution of this peptide (157 mg, 130 μmol) in chloroform (10 mL) was added EDC (57.6 μL, 325.4 μmol), HOOBt (51.5 mg, 315.7 μmol) and finally H-Asp(OAll)-SEt.HCl (S-11) (96 mg, 378.3 μmol). The mixture was stirred for 1 h 30 min at room temperature. After concentration, the oily residue was resuspended in a minimum amount of trifluoroethanol and precipitated with water containing 0.05% trifluoroacetic acid. The resulting mixture was immediately lyophilized. The peptide was solubilized in chloroform (10 mL), then Pd(PPh₃)₄ (93.5 mg, 80.9 μmol) was added, followed by phenylsilane (75.7 μL, 614.2 μmol). The reaction was stirred in the dark for 20 min. After concentration, the oily residue was resuspended in a minimum amount of trifluoroethanol and diluted in water/acetonitrile (1:1, 0.05% trifluoroacetic acid). The resulting mixture was immediately lyophilized. The lyophilized mixture was resuspended in water/acetonitrile (1:1, 0.05% trifluoroacetic acid) and pre-purified on Sephadex LH-20 equilibrated with water/acetonitrile (1:1, 0.05% trifluoroacetic acid). The peptide-containing fractions were pooled and immediately lyophilized. The pre-purified peptide was solubilized in water/acetonitrile (1:1, 0.05% trifluoroacetic acid) and purified to homogeneity by RP-HPLC (C4 semiprep, 40% to 85% acetonitrile/water over 30 min, 16 mL/min). Product eluted at 18 min. Lyophilization of the collected fractions provided peptide S-12 (77 mg, 43% yield) as a white solid.

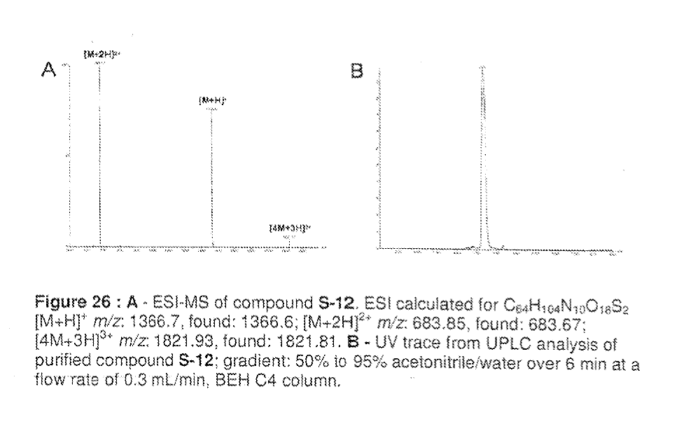

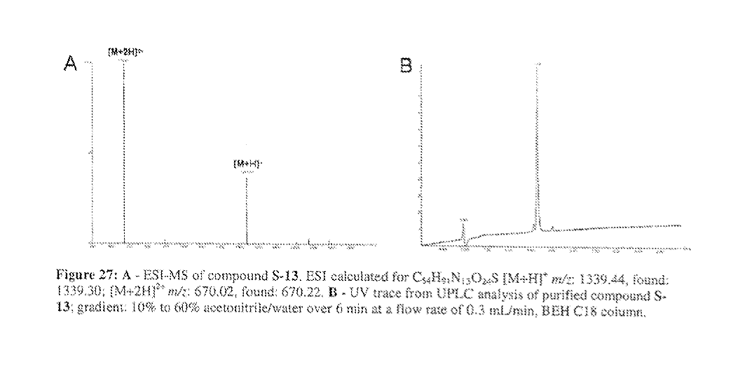

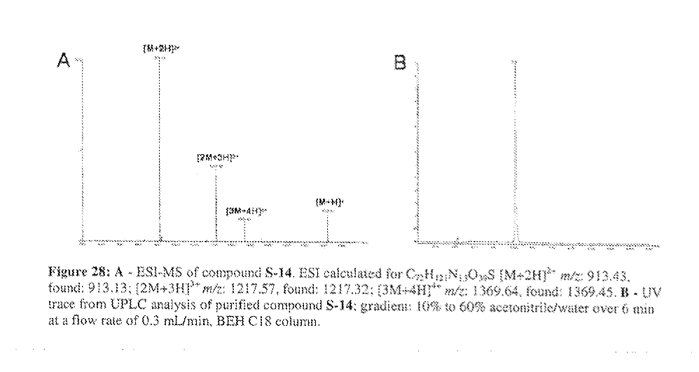

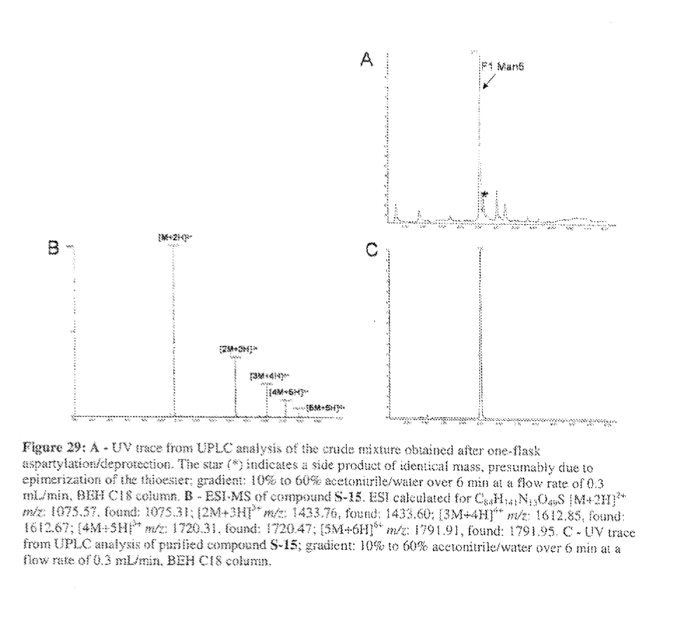

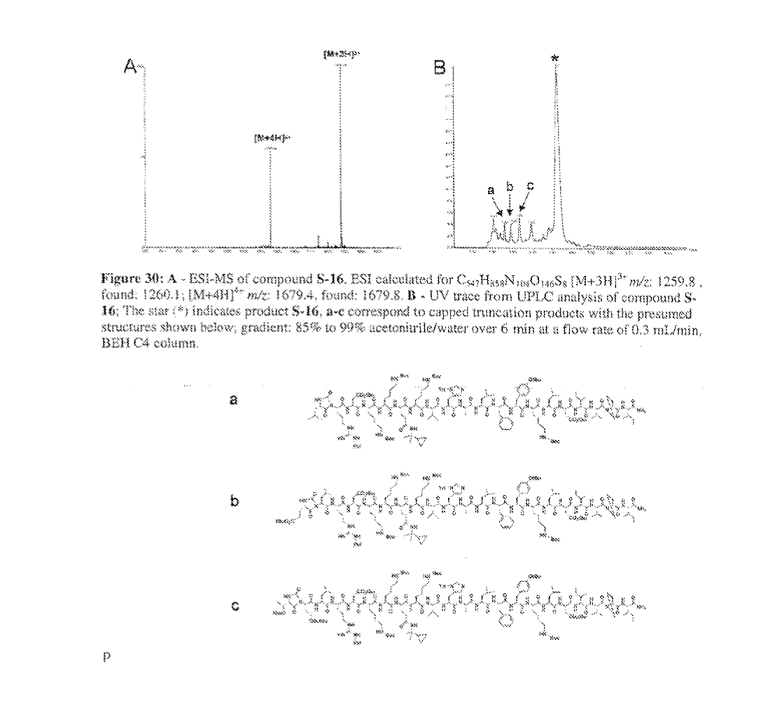

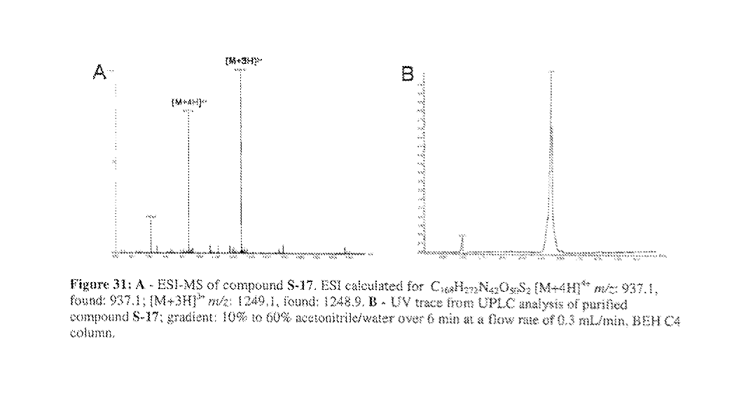

See FIG. 26.

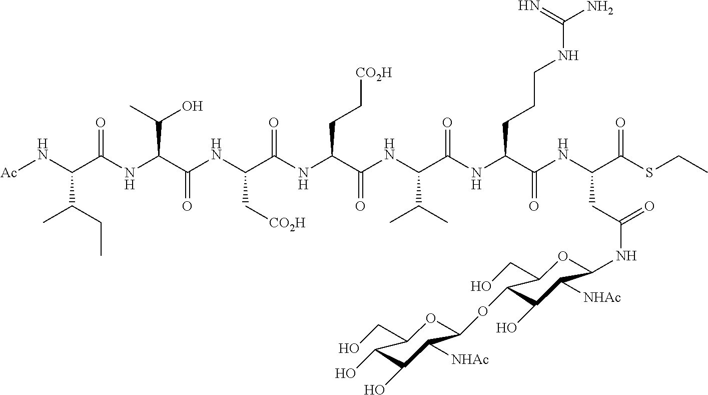

GlcNAc₂ N-Terminal Fragment (S-13).

Peptide S-12 (15 mg. 11 mop and chitobiose anomeric amine (13 mg, 30.7 mop were combined and solubilized in anhydrous DMSO (343 μL). To this mixture, a freshly prepared solution of PyAOP in anhydrous DMSO (0.5 mg/μL, 15.6 μL, 15 μmol) was added, followed by DIEA (4 μL, 23 mol). The solution turned a deep, golden-yellow color and this was stirred for 30 min. The reaction mixture was then frozen and lyophilized.

The protected glycopeptide was then subjected to Cocktail B for 1 h 15 min, precipitated, centrifuged, resuspended and lyophilized as described in the general procedure. The crude peptide was purified to homogeneity by RP-HPLC (C18 semiprep, 10% to 35% acetonitrile/water over 30 min, 16 mL/min). Product eluted at 18.4 min. Lyophilization of the collected fractions provided peptide S-13 (8 mg, 54% yield) as a white solid.

See FIG. 27.

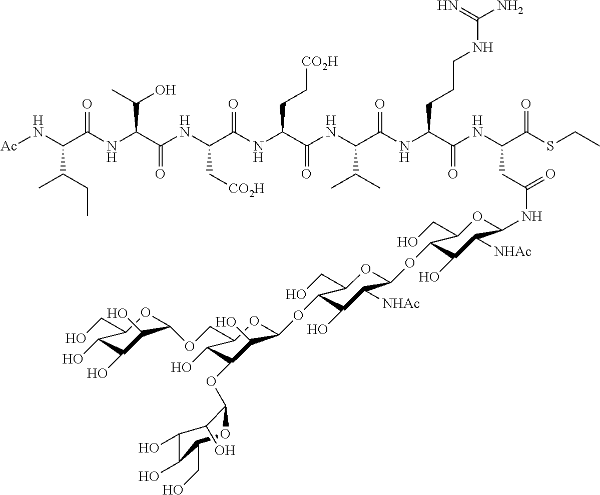

Man₃GlcNAc₂N-Terminal Fragment (S-14).

Peptide S-12 (50.4 mg, 36.9 μmol, 1.2 equiv) and glycosyl amine 20 (28 mg, 30.8 μmol, 1 equiv) were combined and solubilized in anhydrous DMSO (288 μL). To this mixture, a freshly prepared solution of PyAOP in anhydrous DMSO (288 μL, 0.25 mg/μL, 138.6 μmol, 4.5 equiv) was added, followed by DIEA (22.2 μL, 127.7 μmol, 4.1 equiv). The solution turned a deep, golden-yellow color and this was stirred for 30 min. The reaction mixture was then frozen and lyophilized.

The glycopeptide was then subjected to Cocktail B (1.5 mL) for 1 h 15 min. The peptide was precipitated, centrifuged, resuspended and lyophilized as described in the general procedure. The resulting solid was purified to homogeneity by RP-HPLC (C18 semiprep, 10% to 35% acetonitrile/water over 30 min, 16 mL/min). Product eluted at 16.27 min. Lyophilization of the collected fractions provided peptide S-14 (20.6 mg, 37% yield) as a white solid.

See FIG. 28.

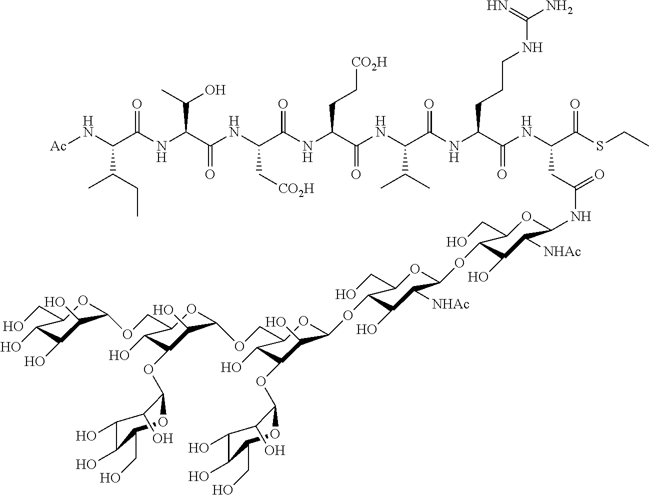

Man₅GlcNAc₂N-Terminal Fragment (S-15).

Peptide S-12 (37.8 mg, 27.7 μmol, 1.2 equiv) and glycosyl amine 18 (28.4 mg, 22.2 μmol, 1 equiv) were combined and solubilized in anhydrous DMSO (216 μL). To this mixture, a freshly prepared solution of PyAOP in anhydrous DMSO (216 μL, 0.25 mg/μL, 104 μmol, 4.5 equiv) was added, followed by DIEA (16.6 μL, 95.5 μmol, 4.1 equiv). The solution turned a deep, golden-yellow color and this was stirred for 30 min. The reaction mixture was then frozen and lyophilized.

The glycopeptide was then subjected to Cocktail B (1.5 mL) for 1 h 15 min. The peptide was precipitated, centrifuged, resuspended and lyophilized as described in the general procedure. The resulting solid was purified to homogeneity by RP-HPLC (C18 semiprep, 10% to 35% acetonitrile/water over 30 min, 16 mL/min). Product eluted at 15.95 min. Lyophilization of the collected fractions provided peptide S-15 (21.1 mg, 44% yield) as a white solid.

See FIG. 29.

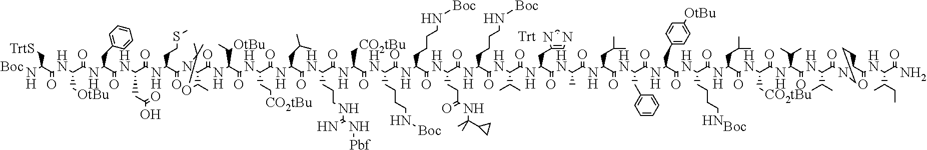

Protected C-Terminal Fragment (S-16).

Upon completion of automated synthesis on 0.05 mmol of TG Sieber resin, the peptide-resin was subjected to deallylation. The peptide-resin was washed with a mixture of dichloromethane/DMF (1:1) into a peptide synthesis vessel and treated with Pd(PPh₃)₄ (5 mg, 4.3 μmol, 0.086 equiv) and phenylsilane (50 μL, 0.4 mmol, 8.6 equiv) in dichloromethane/DMF (1:1, 2.5 mL). After 20 min, the Pd(PPh₃)₄/phenylsilane treatment was repeated once. The peptide-resin was then washed with DMF, dichloromethane and methanol. After drying, the peptide-resin was subjected to a cleavage cocktail (1:99 of trifluoracetic acid/methylene chloride, 2 mL) 5 times for 5 min, (2:98 of trifluoracetic acid/methylene chloride, 2 mL) 5 times for 5 min, and (3:97 of trifluoracetic acid/methylene chloride, 2 mL) 5 times for 5 min. The resulting portions of cleavage solution were systematically pooled in cold diethyl ether and concentrated. The oily residue was resuspended in a minimum amount of trifluoroethanol and precipitated with water. The resulting mixture was immediately lyophilized to give peptide S-16 as a white solid (150 mg). The peptide was used without further purification.

See FIG. 30.

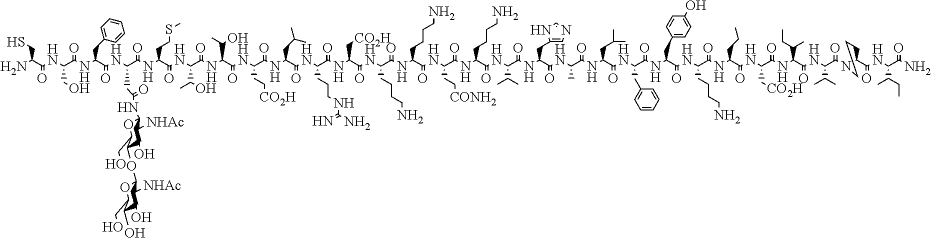

GlcNAc₂ C-Terminal Fragment (S-17).

Peptide S-16 (40 mg, 7.95 μmol, 1 equiv) and chitobiose anomeric amine (10.4 mg, 24.6 μmol, 3 equiv) were combined and solubilized in anhydrous DMSO (643 μL). To this mixture, a freshly prepared solution of PyAOP in anhydrous DMSO (0.5 mg/μL) was added (23.2 μL, 22.2 μmol, 2.8 equiv), followed by DIEA (3.2 μL, 18.5 μmol, 2.3 equiv). The solution turned a deep, golden-yellow color and this was stirred for 30 min. The reaction was then quenched by the addition of 1.5 mL of ice-cold water+0.05% trifluoracetic acid. The precipitate formed was isolated by centrifugation, resuspended in water/acetonitrile (1:1, 0.05% trifluoroacetic acid) and immediately lyophilized.

The dry solid was then subjected to Cocktail R for 1 h 30 min. The peptide was precipitated, centrifuged, and lyophilized. The crude peptide was purified to homogeneity by RP-HPLC (C8 semiprep, 25% to 55% acetonitrile/water over 30 min, 16 mL/min). Product eluted at 12.2 min. Lyophilization of the collected fractions provided peptide S-17 (7.1 mg, 25% yield) as a white solid.

See FIG. 31.

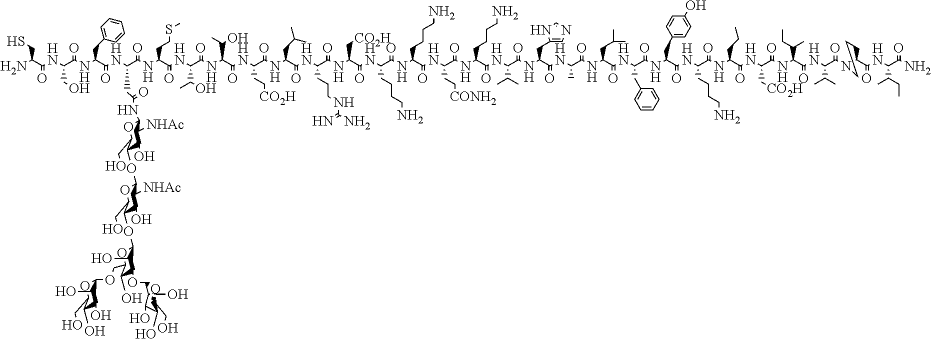

Man₃GlcNAc₂ C-Terminal Fragment (S-18).

Peptide S-16 (45.4 mg, 9 μmol, 1 equiv) and glycosyl amine 20 (10.8 mg, 11.86 mol, 1.3 equiv) were combined and solubilized in anhydrous DMSO (300 μL). To this mixture, a freshly prepared solution of PyAOP in anhydrous DMSO (2.7 mg/μL) was added (50 μL, 25.6 μmol, 2.8 equiv), followed by DIEA (3.9 μL, 22.6 μmol, 2.5 equiv). The solution turned a deep, golden-yellow color and this was stirred for 30 min. The reaction was quenched by addition of 1.5 mL of ice-cold water+0.05% trifluoracetic acid. The precipitate formed was isolated by centrifugation, resuspended in water/acetonitrile (1:1, 0.05% trifluoroacetic acid) and immediately lyophilized.

The glycopeptide was then subjected to Cocktail R (3 mL) for 1 h 30 min. The peptide was precipitated, centrifuged, resuspended and desalted by size exclusion chromatography (Bio-Gel P-6, fine, acetonitrile/water (2:8, 0.05% trifluoroacetic acid)). The crude peptide was purified to homogeneity by RP-HPLC (C8 X-bridge semiprep, 20% to 40% acetonitrile/water over 30 min, 16 mL/min). Product eluted at 12.9 min Lyophilization of the collected fractions provided peptide S-18 (11.9 mg, 31% yield) as a white solid.

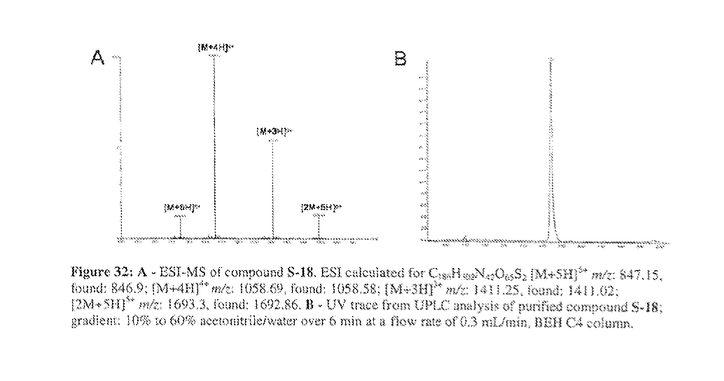

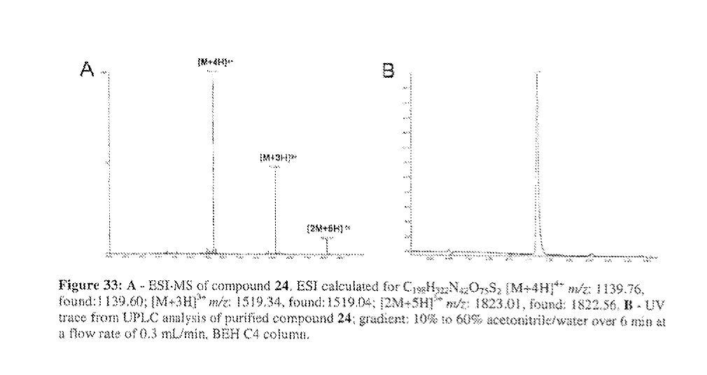

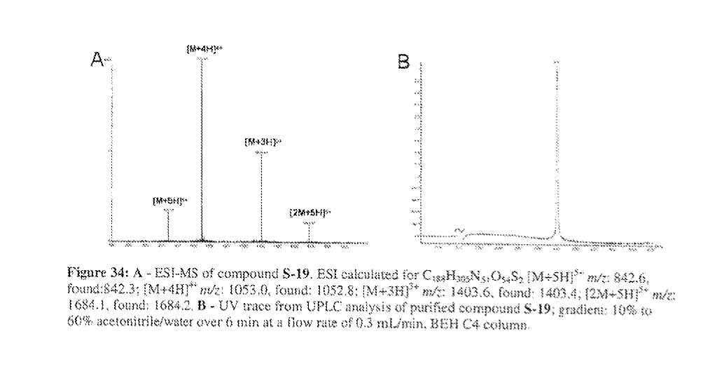

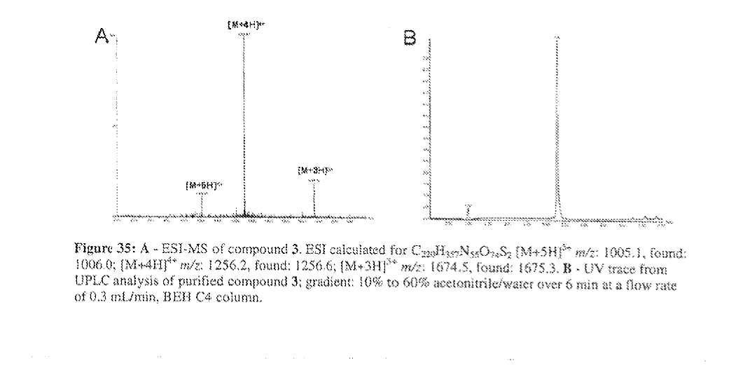

See FIG. 32.

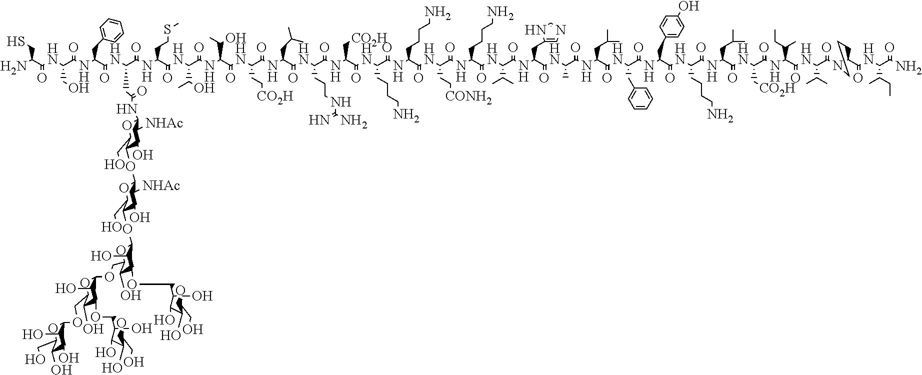

Man₅GlcNAc₂ C-Terminal Fragment (24).

Peptide S-16 (45.4 mg, 9 μmol, 1 equiv) and glycosyl amine 18 (14.6 mg, 11.9 μmol, 1.3 equiv) were combined and solubilized in anhydrous DMSO (300 μL). To this mixture, a freshly prepared solution of PyAOP in anhydrous DMSO (2.7 mg/4) was added (50 μL, 25.6 μmol, 2.8 equiv), followed by DIEA (3.9 μL, 22.6 μmol, 2.5 equiv). The solution turned a deep, golden-yellow color and this was stirred for 30 min. The reaction was quenched by addition of 1.5 mL of ice-cold water+0.05% trifluoracetic acid. The precipitate formed was isolated by centrifugation, resuspended in water/acetonitrile (1:1, 0.05% trifluoroacetic acid) and immediately lyophilized.

The glycopeptide was then subjected to Cocktail R (3 mL) for 1 h 30 min. The peptide was precipitated, centrifuged, resuspended and desalted by size exclusion chromatography (Bio-Gel P-6, fine, acetonitrile/water (2:8, 0.05% trifluoroacetic acid)). The crude peptide was purified to homogeneity by RP-HPLC (C8 X-bridge semiprep, 20% to 40% acetonitrile/water over 30 min, 16 mL/min). Product eluted at 13.25 min. Lyophilization of the collected fractions provided peptide 24 (9 mg, 22% yield) as a white solid.

See FIG. 33.

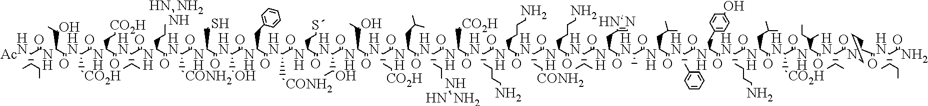

Aglycone V1V2 (S-19).

See FIG. 34

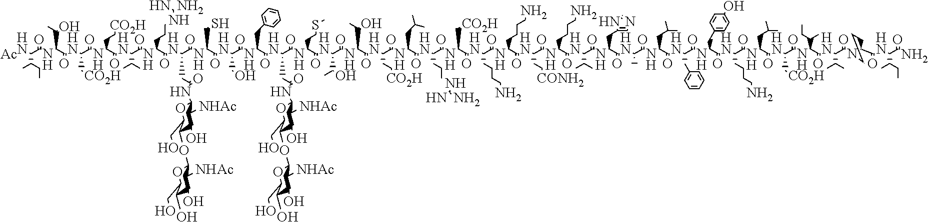

GlcNAc₂ V1V2. (3) Freshly purified N-terminal fragment S.13 (8 mg, 5.98 μmol) and C-terminal fragment S.17 (10 mg, 2.67 μmol) were combined and solubilized it NCL buffer (3.24 μL, 7 mM, prepared as described in general procedure). To this mixture was added neutral TCEP solution (0.5 M, 36 μL). After 2 h another portion of neutral TCEP solution (0.5 M, 36 μL) was added and the reaction was stirred for 3 h 30 min. After completion of the ligation, the mixture was diluted dropwise with water/acetonitrile (1:1, 0.05% trifluoroacetic acid) and desalted by size exclusion chromatography (Bio-Gel P-6, medium, acetonitrile/water (2:8, 0.05% trifluoroacetic acid)). The crude peptide was purified homogeneity by RP-HPLC (C8 semiprep, 2036 to 45% acetonitrile/water over 30 min, 16 mL/min). Product eluted at 20.25 min. Lyophilization of the collected fractions provided 3 (4.2 mg, 34% yield) as a white solid.

see FIG. 35.

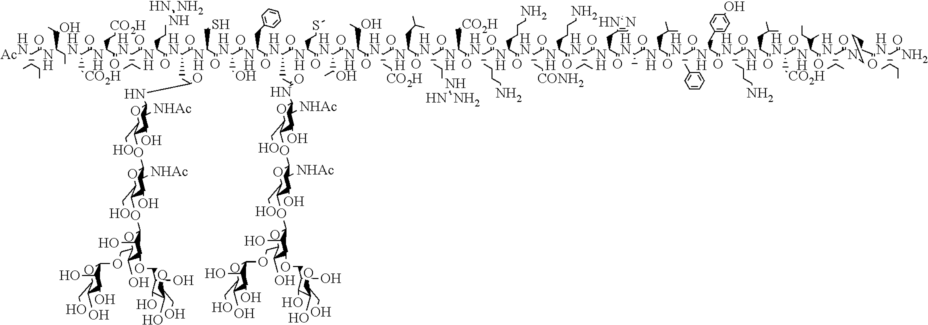

Man₃GlcNAc₂ V1V2 (2).

Freshly purified N-terminal fragment S-14 (9.7 mg, 5.3 μmol) and C-terminal fragment S-18 (7.5 mg, 1.77 mol) were combined and solubilized in NCL buffer (224 μL, 7 mM, prepared as described in general procedure). To this mixture was added neutral TCEP solution (0.5 M. 24 μL). After 2 h another portion of neutral TCEP solution (0.5 M, 24 μL) was added and the reaction was stirred for 6 h. After completion of the ligation, the mixture was diluted dropwise with water/acetonitrile (1:1, 0.05% trifluoroacetic acid) and desalted by size exclusion chromatography (Bio-Gel P-6, fine, acetonitrile/water (2:8, 0.05% trifluoroacetic acid)). The crude peptide was purified to homogeneity by RP-HPLC (C8 X-bridge semiprep, 20% to 40% acetonitrile/water over 30 min, 16 mL/min). Product eluted at 14.78 min. Lyophilization of the collected fractions provided 2 (5 mg, 47% yield) as a white solid.

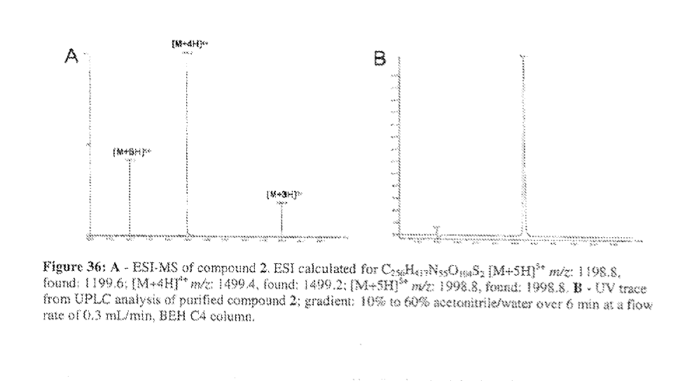

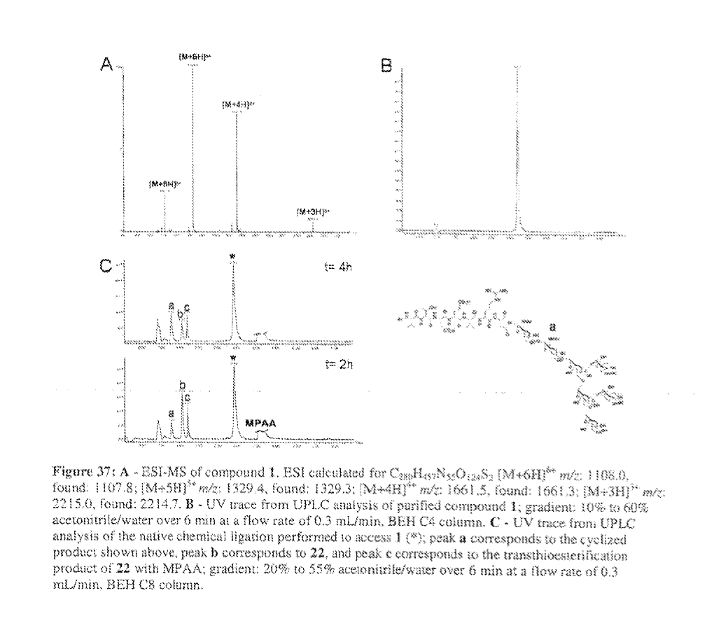

See FIG. 36.

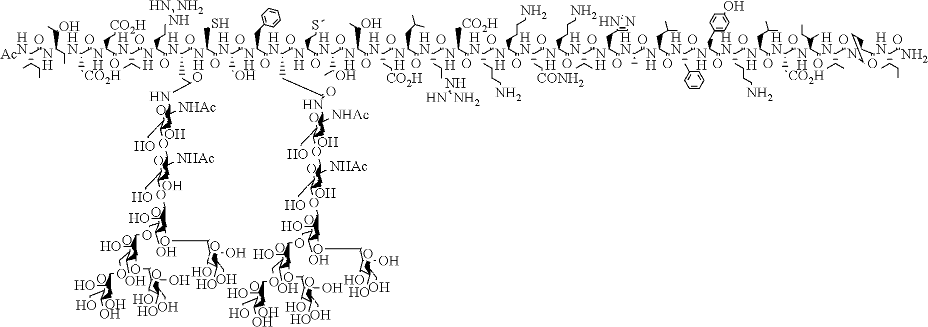

Man₅GlcNAc₂ V1V2 (1).

Freshly purified N-terminal fragment 22 (11.4 mg, 5.3 μmol) and C-terminal fragment 24 (8.1 mg, 1.77 μmol) were combined and solubilized in NCL buffer (224 μL, 7 mM, prepared as described in general procedure). To this mixture was added neutral TCEP solution (0.5 M. 24 μL). After 2 h another portion of neutral TCEP solution (0.5 M, 24 μL) was added and the reaction was stirred for 6 h. After completion of the ligation, the mixture was diluted dropwise with water/acetonitrile (1:1, 0.05% trifluoroacetic acid) and desalted by size exclusion chromatography (Bio-Gel P-6, fine, acetonitrile/water (2:8, 0.05% trifluoroacetic acid)). The crude peptide was purified to homogeneity by RP-HPLC (C8 X-bridge semiprep, 20% to 40% acetonitrile/water over 30 min, 16 mL/min). Product eluted at 14.25 min. Lyophilization of the collected fractions provided 1 (6.5 mg, 55% yield) as a white solid.

See FIG. 37.

Surface Plasmon Resonance.

V1V2 glycopeptide binding K_{d} and rate constant measurements were carried out on a BIAcore 3000 instrument using an anti-human Ig Fc capture assay as described earlier (Alam, S. M.; McAdams, M.; Boren, D.; Rak, M.; Scearce, R. M.; Gao, F.; Camacho, Z. T.; Gewirth, D.; Kelsoe, G.; Chen, P.; Haynes, B. F. J. Immunol. 2007, 178, 4424-4435.). Anti-human IgG Fc antibody (Sigma Chemicals) was immobilized on a CM5 sensor chip to about 10000 response units (RU), and each antibody was captured to about 300 RU. Anti-RSV Synagis mAb was captured on the same sensor chip as a control surface. Non-specific binding and drift in signal was double referenced by subtracting binding to the control surface and blank buffer flow for each of the peptide binding interactions. V1V2 glycopeptides were injected at concentrations ranging from 1 to 40 μg/mL as indicated in FIG. 39. All curve-fitting analyses were performed using global fit of multiple titrations to the 1:1 Langmuir model. All data analysis was performed using the BIAevaluation 4.1 analysis software (GE Healthcare).

Results

Design and Strategy.

The structure of the gp120 V1V2 domain in the context of a bound PG9 mAb Fab consisted of four anti-parallel β-strands (A-D) that folded into what is known as a Greek key motif (FIG. 38B).^{vii} Based on these x-ray crystallographic data, PG9 makes contacts with the C β-strand, and with the Man₅GlcNAc₂ glycans^{xii} (FIG. 38C) at Asn¹⁶⁰ and Asn¹⁵⁶, which reside on strand B. Since most of the structural features recognized by PG9 appear to be localized on the B and C strands,^{xiii} we reasoned that an epitope mimic should, at the very least, encompass this region. Our initial prototype is shown in FIG. 38D. The 35-amino acid peptide corresponds to positions 148-184 of gp120 (HXB2 numbering) derived from the A244 sequence,^{xiv} an Env variant that is known to bind PG9 in monomeric form (i.e., without requiring trimerization).^{xv} With regard to the glycan structure, Man₅GlcNAc₂ was thought to be the best candidate on the basis of prior studies involving perturbations of glycan processing.^{xvi,xvii} The primary target that emerged from this analysis was glycopeptide 1 with Man₅GlcNAc₂ units installed at the two glycosylation sites, Asn¹⁶⁰ and Asn¹⁵⁶; we also planned to gain access to simpler glycoforms 2 and 3 bearing Man3GlcNAc₂ and chitobiose (GlcNAc₂), respectively.^{xvii} These could be used to probe the importance of the outer mannose residues for recognition.

We term the general synthetic approach that our laboratory has applied to complex glycoprotein targets as convergent assembly.^{xi} In our usual modus operandi, N-linked sugars are installed via aspartylation of unprotected glycosyl amines, drawing from the precedent of Lansbury,^{xix} which we^{xx} and others^{xxi} have extended in substantive ways. As we examined goal structures 1-3 in particular, we noted that the close spacing of the two glycans, especially with larger oligosaccharides, could present a difficult challenge for their incorporation. We anticipated that application of our methods in this demanding context would afford valuable teachings regarding the synthesis of the required clustered N-glycan motifs. As for the sugars themselves, Man₃GlcNAc₂ constitutes the common pentasaccharide core of all N-glycans; it has been synthesized previously by our laboratory and others.^{xxii} By contrast, Man₅GlcNAc₂ seems to have received less attention as a synthetic target.^{xxiii,xxiv} We start by describing our route to the desired glycans.

Synthesis of Man₅GlcNAc₂ and Man₃GlcNAc₂ Glycans.

Our studies commenced with experiments directed to the syn synthesis of the Man₅GlcNAc₂ heptasaccharide. The β-mannosyl linkage of the core trisaccharide 6 was constructed as we have in prior contexts^{xxv} by uniting Crich donor 4^{xxvi,xxvii} with chitobiose acceptor 5^{xxvb} (Scheme 1). Fortunately, the minor quantities of undesired α-isomer formed (<10%) could be separated by careful chromatography to afford trisaccharide 6 as a single diastereomer in 86% yield. The PMB group was removed in 83% yield. Coupling of the resulting acceptor 7 with thioglycoside donor 8 was accomplished under NIS/TMSOTf activation conditions, yielding tetrasaccharide 9. Cleavage of the benzylidene acetal with aqueous acetic acid afforded diol 10 in 63% overall yield from 7.

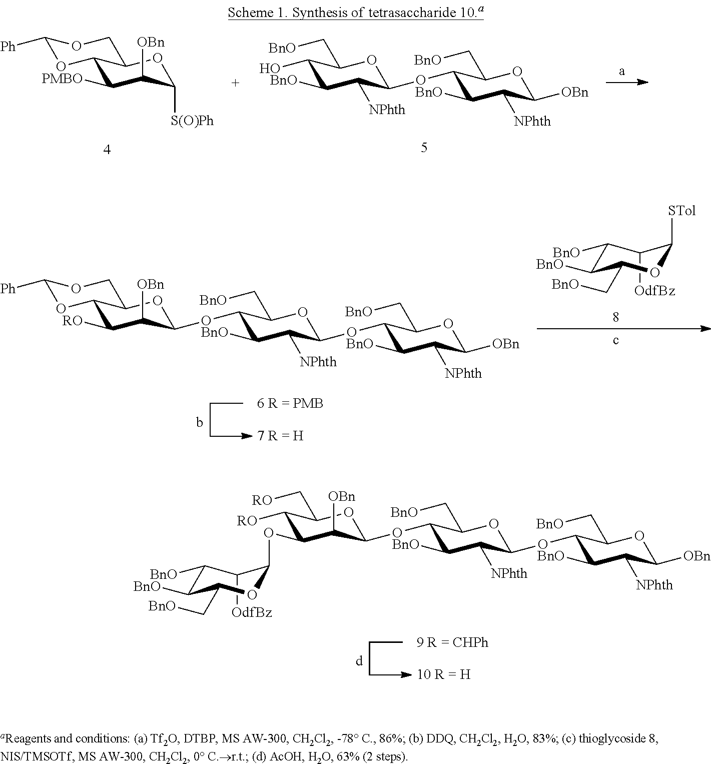

Assembly of the heptasaccharide at first in protected form was accomplished convergently by selective mannosylation at C-6 of 10 with branched donor 15. Synthesis of the requisite trisaccharide 15 was achieved by elaboration of mannosyl building block 11 (Scheme 2). Reductive ring opening was accomplished selectively with borane-THF complex in the presence of copper triflate in 96% yield.^{xxviii} Cleavage of the PMB group afforded the 3,6-diol, 13, which underwent double mannosylation with imidate donor 14 to furnish the bis-α-mannosylated trisaccharide 15 in 75% yield.

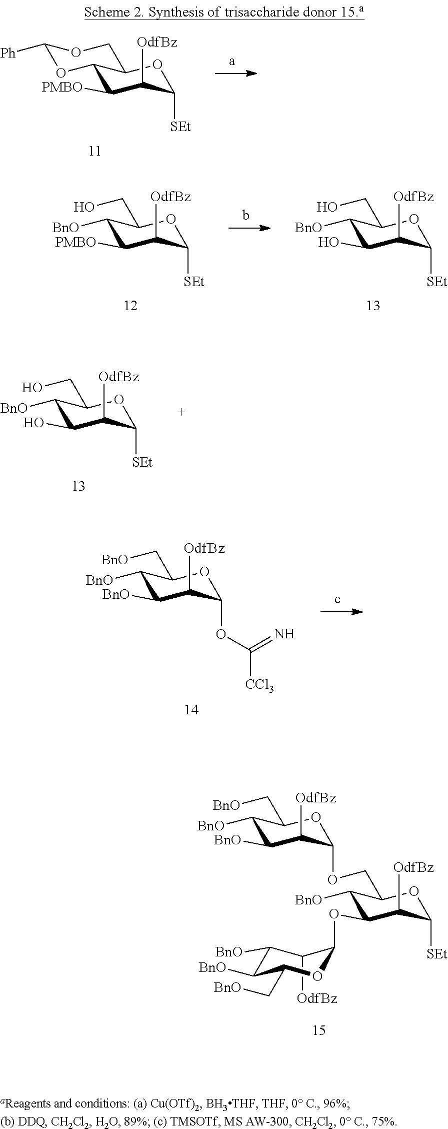

With the stage set for the key coupling, 15 was activated (NIS/TMSOTf) and joined with 10, thus providing the fully elaborated protected heptasaccharide 16 in 64% yield (Scheme 3). A four-step sequence involving ester saponification, phthalimide cleavage, N-acetylation, and hydrogenolysis proceeded smoothly to give fully deprotected heptasaccharide 17 as a mixture of anomeric alcohols in 77% yield. This compound underwent apparently quantitative conversion to the β-anomeric amine 18 under Kochetkov amination conditions.^{xxix}

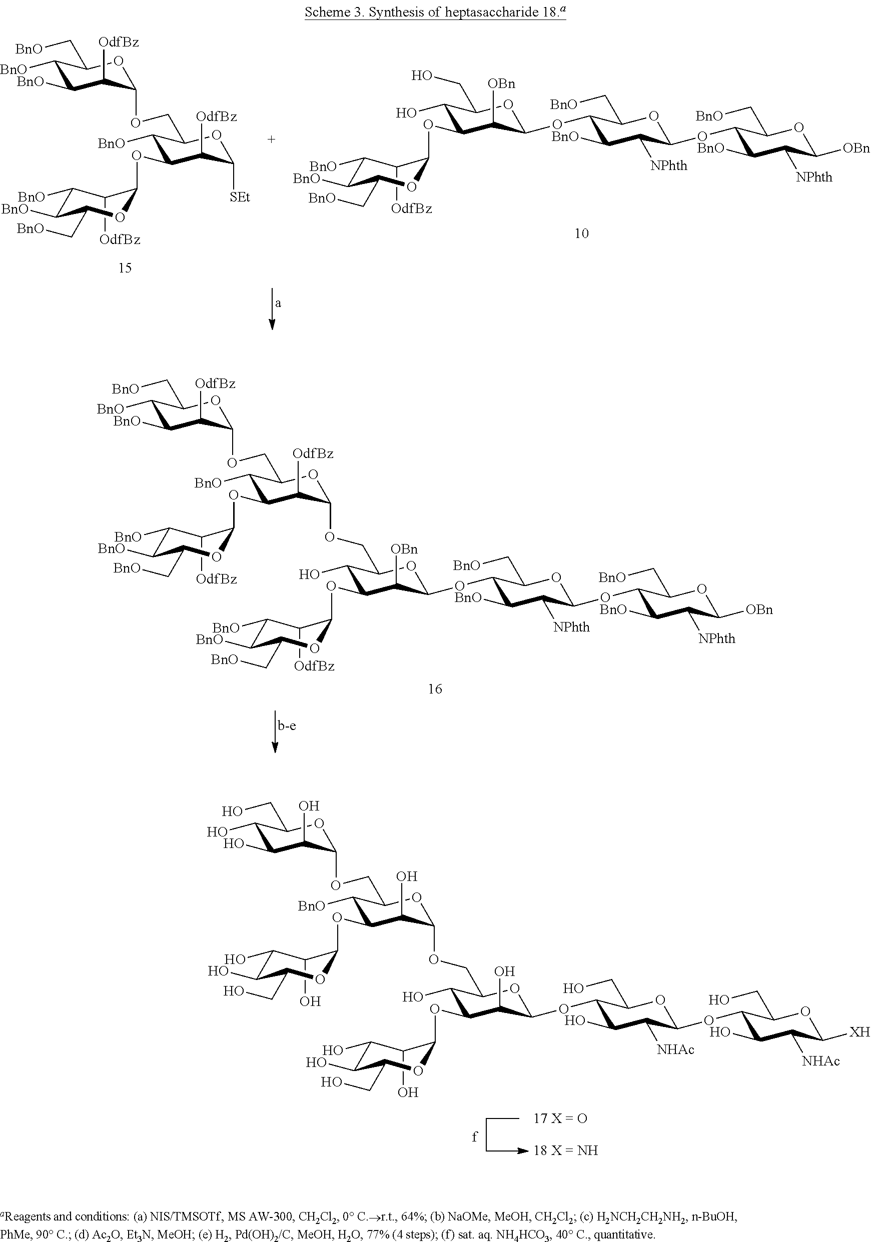

The pentasaccharide, Man₃GlcNAc₂, was obtained from tetrasaccharide intermediate 10 by selectively coupling donor 8 to the C-6 hydroxyl group (Scheme 4). Although this reaction was complicated by a small amount of bis-glycosylation, the protected Man₃GlcNAc₂ unit was isolated in 94% yield. Subjection of this material to the 4-step global deprotection protocol described above resulted in a 74% overall yield of fully deprotected pentasaccharide 19 as a mixture of anomers. The β-anomeric amine 20 was subsequently generated by application of the Kochetkov conditions.

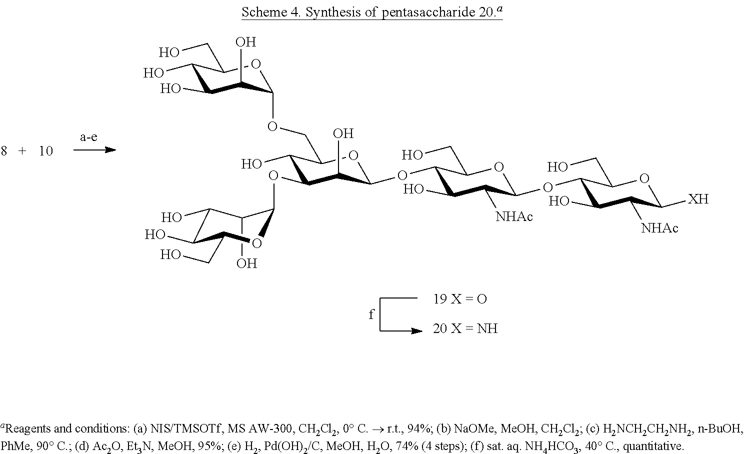

Convergent Assembly of V1V2 Glycopeptides.

The most risky phase of the effort involved the assembly of the peptide domain of the targeted glycopeptide constructs, and their coupling to different oligosaccharides. Two basic strategies were considered. Our first thoughts envisioned installing both glycans simultaneously on the full-length peptide, bearing in mind our prior successes with two- and three-fold aspartylations on cyclic scaffolds.^{ivd} Pilot experiments using chitobiose as a model glycan, however, yielded only unmanageable mixtures of mono- and bis-glycosylated forms, presumably due to the steric demands imposed by the close proximity of aspartylation sites. Anticipating that driving the reaction to completion with larger oligosaccharides might require a substantial excess of precious glycosyl amine, we decided to pursue an alternative approach involving the ligation of two pre-built glycopeptide fragments. Here, the presence of Cys¹⁵⁷ served to raise the possibility of native chemical ligation (NCL).^{xxx}

We anticipated that this approach would not be without its own complications, given the close positioning of the glycans. Indeed, one of the coupling partners (Ile¹⁴⁸-Asn¹⁵⁶) must carry the sterically demanding oligosaccharide on its C-terminal thioester-bearing amino acid. Nevertheless, this scheme was successfully reduced to practice, as described below.

In the event, N-terminal fragment, peptide thioester 21, was obtained by Fmoc solid phase peptide synthesis (SPPS) and post-resin C-terminal functionalization procedures^{xxxi} used by our laboratory in the context of other glycopeptide endeavors (Scheme 5).^{xxxii} Using our recently reported one-flask aspartylation/deprotection protocol, the free carboxylic acid side chain at position 156 was joined to the Man₅GlcNAc₂ glycosyl amine 18, followed by TFA treatment to provide glycopeptide thioester 22 in 44% yield after purification by reversed-phase HPLC. The formation of a side product of identical mass was observed in small quantities (5-10%), presumably due to base-induced epimerization of the thioester during the aspartylation. Fortunately, it could be easily separated during the purification.

For the C-terminal fragment, a similar one-flask sequence was used to convert protected peptide 23 to deprotected glycopeptide 24 in 22% yield. As has been previously observed, emplacement of a pseudoproline motif at Thr¹⁶² (n+2 relative to Asp¹⁶⁰) was helpful in suppressing undesired aspartimide formation during the aspartylation.^{xx,xxi} The isolated yield for this fragment was eroded by factors that complicated the final purification of glycopeptide 24, including near overlap of the unglycosylated peptide, and the persistence of capped truncation products that had formed during the course of the SPPS (by an as yet undefined mechanism). Despite these obstacles, sufficient quantities of fragments of 22 and 24 could be synthesized and joined by NCL to afford the fully elaborated glycopeptide 1 bearing Man₅GlcNAc₂ units at Asn¹⁶⁰ and Asn¹⁵⁶ in 55% yield. The simpler glycoforms 2 and 3, possessing two Man₃GlcNAc₂ and two chitobiose glycans, respectively, were prepared by an analogous route (see Supporting Information for details).

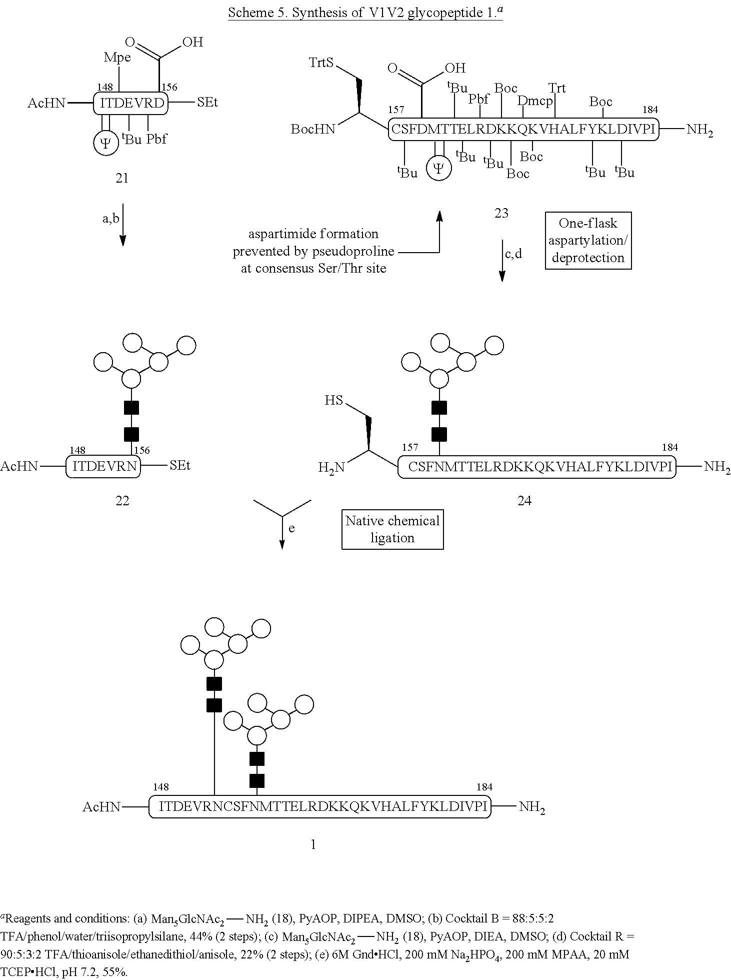

In all cases, the final ligation proved to be difficult. Indeed, three equivalents of thioester were required for the reaction to progress to completion.^{xxxiii} Careful control of the reaction pH was needed to avoid apparent epimerization or excessive formation of succinimide (via cyclization of the asparagine side chain nitrogen onto the thioester). While certainly less than optimal, these ligations represent, to the best of our knowledge, the first examples of NCL with peptide thioesters carrying an N-glycan directly at the C-terminus. Furthermore, no other syntheses have been reported of linear glycopeptides bearing such closely spaced N-glycans. i.e., separated by three amino acids or less.^{xxxiv} While the yields for the overall sequence are likely to benefit from optimization,^{xxxv} our concerns at first were focused more on the purity of the synthetic constructs rather than on maximizing material throughput. Fortunately, the synthesis, even in its present form, has produced sufficient quantities to initiate the biological studies now underway both in vitro and in vivo to chart a path forward to a clinically evaluable HIV-1 vaccine (vide infra).

Antigenicity Studies.

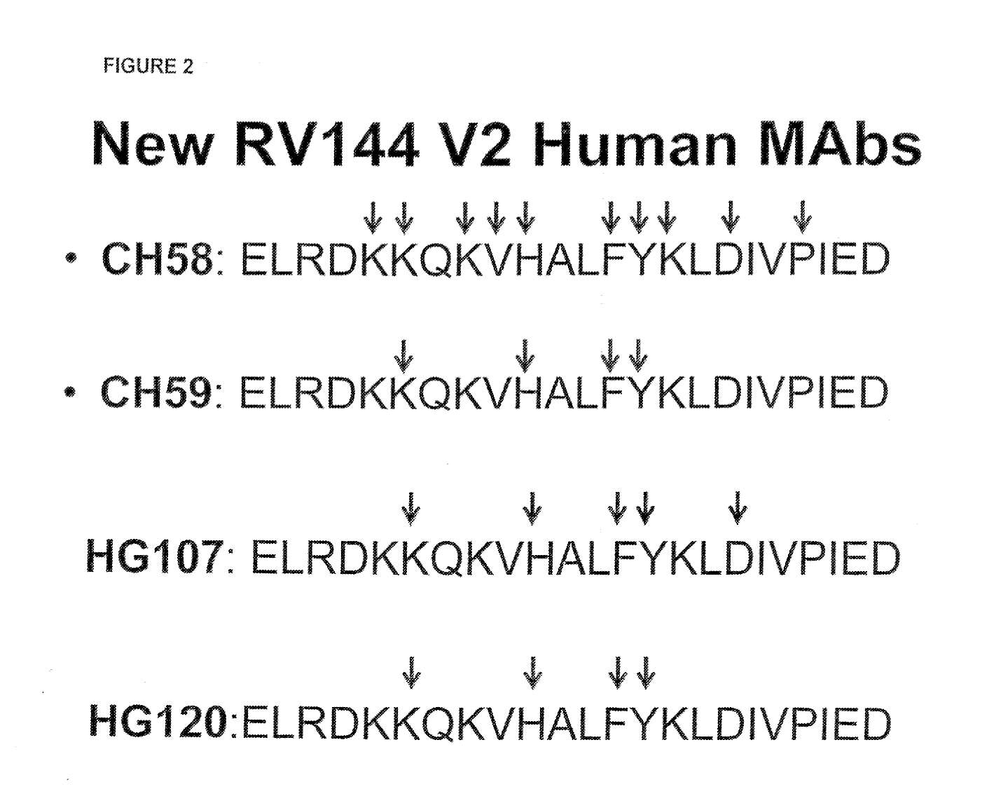

To assess the extent to which our synthetic V1V2 glycopeptides are able to recapitulate the mAb PG9 V1V2 BnAb epitope, we studied the binding of constructs 1-3 to PG9 by surface plasmon resonance (SPR) analysis (FIG. 39). PG9 was captured by surface-immobilized anti-human Ig Fc, and the V1V2 glycopeptide constructs were injected as analytes on BIAcore 3000 instruments as described previously.^{xxxvi} We found that the Man₅GlcNAc₂ V1V2 (1) and Man₃GlcNAc₂ V1V2 (2) glycopeptides both exhibited significant affinity for mAb PG9 (FIGS. 2A and 2B), with K_{d}'s of 311 and 119 nM, respectively (obtained by using a global fit of multiple titrations to a 1:1 Langmuir model). By contrast, the chitobiose-bearing construct 3 did not bind mAb PG9 (FIG. 39C), suggesting that the presence of α-linked mannose residues on the glycans is important for recognition. Furthermore, binding by the unglycosylated V1V2 peptide (i.e., “aglycone”) (FIG. 39D) or the solitary protein-free Man₅GlcNAc₂ and Man₃GlcNAc₂ oligosaccharides was not detected (FIGS. 39E and F). Mixtures of “aglycone” and glycan similarly failed to show measurable binding (not shown).

Taken together, these data demonstrate that PG9 recognition of our V1V2 constructs is critically dependent on both the peptide and carbohydrate domains. Covalent linkage between them is essential, since the apparent affinities for each individual component in isolation are very low. Indeed, NMR studies have shown that the K_{d} for binding of PG9 to Man₅GlcNAc₂-Asn alone is ˜1-2 mM,^{vi} consistent with the general trend that individual protein-carbohydrate interactions tend to be weak. The overall high “avidity” observed may be attributed to the synergies afforded by multivalency, wherein the binding to PG9 is enhanced by multiple simultaneous interactions with the C β-strand, and the Asn¹⁶⁰ and Asn¹⁵⁶ glycans.^{vii} Conformational effects may also play a role, as glycosylation of the peptide backbone could have a favorable orienting influence on the involved peptide and/or sugar residues.^{xxxvii} Evidence of such “cross-talk” between peptido and glyco domains has been observed by our laboratory in other settings.^{xxxviii}

In light of these findings, it seems likely that proper evaluation of the optimal glycans and peptide sequences for mimicking the V1V2 BnAb epitope—and other similar glycopeptide antigens—will require their presentation in their native N-linked context (or as some close isostere). Adopting this approach enabled us to make the unexpected discovery that the Man₃GlcNAc₂-based construct 2 binds PG9 just as well, and perhaps even a little better, than construct 1 (bearing Man₅GlcNAc₂).^{xxxix} Studies are underway to better understand the robust recognition of this non-canonical^{xl} glycan by PG9. In the meantime, we note that such fine structure preferences were not detected by previous approaches, such as glycan array analysis, that interrogated PG9 binding to isolated carbohydrates in the absence of a peptide backbone.^{xli}

More profound, perhaps, is the overall question of how the modestly sized glycopeptides 1 and 2 are able to simulate the antigenicity of native envelope glycoproteins so well. PG9 and other BnAbs that target the same V1V2 epitope (e.g., PG16 and CH01 to CH04^{xv}) are thought to be sensitive to quarternary structure, binding Env trimers better than monomeric Env.^{vi,xiii} Indeed, relatively few Env sequences are known to bind PG9 in monomeric form, so it is noteworthy that comparatively small (6-7 kDa), linear 35-mer Env glycopeptide fragments like 1 and 2 are able to bind PG9 with respectable affinities, with K_{d}'s on the order of 10⁻⁷ Such affinities compare favorably with the published K_{d}'s of ˜5×10⁻⁸ M for the full-length A244 Env monomer (from which 1 and 2 are derived),^{xv} and ˜10⁻⁸ M for a recently reported trimeric Env construct.^{xiii} We are actively investigating the nature of the binding interaction with PG9; preliminary results suggest that it cannot be fully explained by a simple “induced-fit” mode of association.

In summary, we have designed and chemically synthesized homogeneous gp120 V1V2 domain glycoforms that demonstrate robust antigenicity (K_{d}˜10⁻⁷) for the HIV-1 gp120 V1V2 BnAb PG9. Key to these initial successes were significant achievements at the level of chemistry, which include the development of a scalable synthetic route to the Man₅GlcNAc₂ heptasaccharide 17, and the execution of, arguably, some of the most ambitious glycopeptide ligations known to date. As a whole, this work represents a promising first step toward the development of experimental vaccine immunogens to be tested for the capacity to elicit gp120 V1V2 BnAb epitope-targeted antibodies. Further work is underway to characterize the antigenic properties of 1 and 2 in detail, and evaluate their immunogenicity in animal models. Similar and perhaps even more ambitious chemical synthesis strategies may be of use in preparing homogeneous glycopeptides for other HIV-1 gp120 BnAb epitopes.^{xiiii} This is an ongoing program whose results will be disclosed in due course.

### Example 6

### Recognition of Synthetic Glycopeptides by HIV-1 Broadly Neutralizing Antibodies and their Unmutated Ancestors

Current HIV-1 vaccines elicit strain-specific neutralizing antibodies. Broadly neutralizing antibodies (BnAbs) are not induced by current vaccines, but are found in plasma in ˜20% of HIV-1-infected individuals, after several years of infection. One strategy for induction of unfavored antibody responses is to produce homogeneous immunogens that selectively express BnAb epitopes but minimally express dominant strain-specific epitopes. It is reported here that synthetic, homogeneously glycosylated peptides that bind avidly to V1V2 BnAbs PG9 and CH01, bind minimally to strain-specific neutralizing V2 antibodies that are targeted to the same envelope polypeptide site. Both oligomannose derivatization and conformational stabilization by disulfide-linked dimer formation of synthetic V1V2 peptides were required for strong binding of V1V2 BnAbs. An HIV-1 vaccine should target BnAb unmutated common ancestor (UCA) B cell receptors of naïve B cells, but to date, no HIV-1 envelope constructs have been found that bind to the UCA of V1V2 BnAb PG9. It is demonstrated herein that V1V2 glycopeptide dimers bearing Man₅GlcNAc₂ glycan units bind with apparent nanomolar affinities to UCAs of V1V2 BnAbs PG9 and CH01 and with micromolar affinity to the UCA of a V2 strain-specific antibody. The higher affinity binding of these V1V2 glycopeptides to BnAbs and their UCAs renders these glycopeptide constructs particularly attractive immunogens for targeting subdominant HIV-1 envelope V1V2 neutralizing antibody producing B cells.

A current key goal of HIV-1 vaccine development is to learn how to induce antibodies that will neutralize many diverse HIV-1 strains. Current HIV-1 vaccines elicit strain-specific neutralizing antibodies, while BnAbs are not induced and only arise in select HIV-1 chronically-infected individuals. One strategy for induction of favored antibody responses is to design and produce homogeneous immunogens with selective expression of BnAb but not dominant epitopes. In this study, binding properties of chemically synthesized V1V2 glycopeptides that bind both to mature HIV-1 envelope broad neutralizing antibodies and the receptors of their naïve B cells are described. These results demonstrate that such synthetic glycopeptides can be immunogens that selectively target BnAb naïve B cells.

It is widely believed that a key characteristic of an effective HIV-1 vaccine would be its ability to induce broadly neutralizing antibodies (BnAbs). Known BnAbs have been shown to target conserved HIV-1 Envelope (Env) regions including glycans, the gp41 membrane proximal region, the gp120 V1/V2 and the CD4 binding site (CD4bs) (Burton et al, Science 337(6091):183-186 (2012), Kwong and Mascola, Immunity 37(3):412-425 (2012), Wu et al, Science 329(5993):856-861 (2010), Wu et al, Science 333(6049):1593-1602 (2011), Scheid et al, Science 333(6049):1633-1637 (2011), Sattentau and Michael, F1000 biology reports 2:60 (2010), Mascola and Haynes, Immunol. Rev. 254(1):225-244 (2013)). Most mature BnAbs have one or more unusual features such as long heavy chain third complementarity-determining regions [HCDR3s], polyreactivity for non-HIV-1 antigens, and high levels of somatic mutations (Kwong and Mascola, Immunity 37(3):412-425 (2012), Mascola and Haynes, Immunol. Rev. 254(1):225-244 (2013), Haynes et al, Nat. Biotechnol. 30(5):423-433 (2012)). In particular, CD4bs BnAbs have extremely high levels of somatic mutations suggesting complex or prolonged maturation pathways (Kwong and Mascola, Immunity 37(3):412-425 (2012), Wu et al, Science 329(5993):856-861 (2010), Wu et al, Science 333(6049):1593-1602 (2011), Scheid et al, Science 333(6049):1633-1637 (2011)). Adding to the challenge has been the difficulty in achieving binding of proposed antigens to germline or unmutated common ancestors (UCAs). Binding to BnAb UCAs would be a desirable characteristic for putative immunogens intended to induce BnAbs (Scheid et al, Science 333(6049):1633-1637 (2011), Chen et al, Human Retrovirol. 24:11-12 (2008), Doores and Burton, J. Virol. 84(20):10510-10521 (2010), Ma et al, PLoS Pathog. 7(9):e1002200 (2011), Pancera et al, J. Virol. 84(16):8098-8110 (2010), Xiao et al, Biochem. Biophys. Res. Commun. 390(3):404-409 (2009)).

Immunization of humans with Env proteins has not resulted in high plasma titers of BnAbs (Haynes et al, N. Engl. J. Med. 366(14):1275-1286 (2012), Montefiori et al, J. Infect. Dis. 206(3):431-441 (2012)). Rather, dominant strain-specific neutralizing epitopes have selectively been induced. This was most clearly seen in the ALVAC/AIDSVAX® RV144 HIV-1 vaccine efficacy trial in which Env immunogens 92TH023 and A244 CRFAE_{—}01 gp120s expressed both a dominant linear V2 epitope and bound with high nM affinity to the glycan-dependent V1V2 BnAbs PG9 and CH01 (Liao et al, Immunity 38(1):176-186 (2013)). Although both linear and glycan-dependent V2 epitopes were expressed on the A244 immunogen, the dominant V2 plasma antibody responses in this trial were targeted to linear V2 epitopes and not to the glycan-dependent BnAb epitope ((Haynes et al, N. Engl. J. Med. 366(14):1275-1286 (2012), Montefiori et al, J. Infect. Dis. 206(3):431-441 (2012), Liao et al, Immunity 38(1):176-186 (2013)). A series of mAbs, the prototype of which is mAb CH58, have been isolated from RV144 vaccines and were shown to bind to linear V2 epitopes that include lysine 169 (Liao et al, Immunity 38(1):176-186 (2013)). However, they are strain-specific and only neutralize laboratory-adapted but not primary isolate HIV-1 strains (Liao et al, Immunity 38(1):176-186 (2013)). Although PG9 and CH01 V1V2 BnAbs also bind to V2 K169 and surrounding amino acids, they also bind to high mannose glycans at N¹⁵⁶ and N¹⁶⁰ (McLellan et al, Nature 480(7377):336-343 (2011)). Crystal structures of the CH58 antibody bound to V2 peptides demonstrated the V2 structure around K169 to be helical (Liao et al, Immunity 38(1):176-186 (2013)), whereas the crystal structure of the PG9 antibody with a V1V2 scaffold showed the same polypeptide region in a beta strand conformation (McLellan et al. Nature 480(7377):336-343 (2011)).

The rationale that undergirded the studies described below envisioned that an optimal immunogen for the V1V2 BnAb peptide-glycan envelope region would be would be one that presented a chemically homogeneous entity that binds to V1V2 BnAbs with high affinity. In addition, an optimal immunogen for the V1V2 BnAb site would be one that binds with high affinity to the V1V2 BnAb UCAs.

Recently, chemically synthesized glycopeptides of the HIV-1 Env V1V2 148-184 aa region with Man₃GlcNAc₂ or Man₅GlcNAc₂ glycan units at N¹⁵⁶ and N¹⁶⁰ were described (Aussedat et al, JACS [Epub ahead of print] (2013)). It was found that these homogeneous glycopeptide constructs with oligomannose units bound avidly to the V1V2 BnAb PG9.

In this study, it is reported that the disulfide-linked dimeric forms of these glycopeptides bound preferentially to the V1V2 BnAb mature antibodies (PG9 and CH01) over the V2 strain-specific mAb CH58, to which the binding was minimal. Importantly, the V1V2 peptide-glycans also bound to both PG9 and CH01 V1V2 BnAb UCAs, thus providing a strong rationale for their evaluation as experimental immunogens.

### Experimental Details

Synthesis of V1 V2 Peptides.

Design and chemical synthesis of the V1/V2 peptides as single glycoforms were as described previously (Aussedat et al, JACS [Epub ahead of print] (2013)). Man₃GlcNAc₂ C157A mutant glycopeptide was synthesized and subjected to de-sulfurization (see procedure below). The aglycone and the GlcNAc2 V1V2, were solubilized in DMSO at 5-10 mg/mL and then diluted in phosphate buffer, pH 7.0 with vortexing and brief sonication. For complete oxidation of glycopeptides, Man₃GlcNAc₂ V1V2 and Man₅GlcNAc₂ V1V2 glycopeptides were solubilized in DMSO at 5-10 mg/mL and then diluted dropwise to 20% DMSO (in 50 mM phosphate buffer, pH 7.0) as above and left overnight at room temperature. V1V2 glycopeptides were further diluted to the required concentration (1-50 μg/mL) for SPR binding analyses in PBS (pH 7.4). Size exclusion chromatography was performed on a Superdex Peptide 10/300 GL column (GE Healthcare) equilibrated in PBS buffer. Molecular size of the V1V2 peptides was determined using protein standards ranging in MW from 25 to 6.5 kDa.

SDS-PAGE analysis of V1V2 peptides was done by solubilizing the glycopeptides (Man3, Man5) in 20% DMSO in 50 mM phosphate buffer, pH 7.0 and incubating at RT overnight to allow dimer formation as described above. Reduced and nonreduced peptide samples, each at 5-10 μg, were heated in a hot water bath for 5 min before subjecting to gel electrophoresis on the NuPage Novex 4-12% Bis-Tris gel (Life Technologies) in 1X MES running buffer (50 mM MES, 50 mM Tris, 0.1% (w/v) SDS, 1 mM EDTA, pH 7.3) at 200 V for ˜45 min. The gel was stained and destained using a heated Coomassie blue protocol. The Precision Plus All Blue Protein Standards (Biorad) and Color Marker Ultra-low Range (Sigma-Aldrich) were added to the respective lanes 1 and 2 for estimates of the peptides' relative molecular weights.

Cysteine Desulfurization Procedure.

The buffer required for desulfurization was freshly prepared prior to the reaction. Na₂HPO₄ (56.6 mg, 0.4 mmol) was solubilized in water (1 mL), guanidine.HCl (1.146 g, 12 mmol), and TCEP.HCl (46 mg, 0.17 mmol) were then added and the pH was brought to 7 with a solution of NaOH (5 M, 110 μL). After 15 min degassing the solution was ready for use. The glycopeptide (1 mg) was solubilized in 1 mL of buffer, tert-butylthiol was added (30 μL, 0.34 mmol) and radical initiator VA-044 (0.1 M in water). The reaction mixture was stirred at 37° C. for 2 h. Upon completion the glycopeptide was desalted by size exclusion chromatography (Bio-Gel P-6. Medium, acetonitrile/water (2:8, 0.05% trifluoroacetic acid)). The crude peptide was purified to homogeneity by RP-HPLC (C8 semiprep, 20% to 45% acetonitrile/water over 30 min, 16 mL/min). Lyophilization of the collected fractions provided the desulfurized glycopeptide (500 μg) as a white solid.

Antibodies.

The isolation of CH01 mAb from IgG+ memory B cells of a broad neutralizer subject have been previously described (Bonsignori et al, J. Virol. 85(19):9998-10009 (2011)). The inference and production of unmutated ancestors of CH01 and PG9 were as described earlier (Bonsignori et al, J. Virol. 85(19):9998-10009 (2011), Munshaw and Kepler, Bioinformatics 26(7):867-872 (2010)). V1/V2 conformational/quaternary mAbs PG9 was provided by Dennis Burton (IAVI, and Scripps Research Institute, La Jolla, Calif.). Synagis (palivizumab; MedImmune LLC, Gaithersburg, Md.), a human respiratory synctytial (RSV) mAb, was used as a negative control.

Surface Plasmon Resonance (SPR) Measurements.

V1V2 glycopeptide binding Kd and rate constant measurements were carried out on BIAcore 3000 instruments as described earlier (Alam et al, Immunol. 178(7):4424-4435 (2007), Alam et al, J. Virol. 82(1):115-125 (2008)). Anti-human IgG Fc antibody (Sigma Chemicals) was immobilized on either a CM3 or CM5 (CM3 for kinetics and Kd determination) sensor chip (to minimize non-specific binding of peptides to chip matrix) to about 5000 Resonance Unit (RU) and each antibody was captured to about 100-200 RU on individual flow cells, in addition to one flow cell with the control Synagis mAb on the same sensor chip. Non-specific binding of V1V2 glycopeptide was double-referenced by substracting the control surface and blank buffer flow for each mAb-V1V2 glycopeptide binding interaction. For rate constants and Kd measurements, each V1V2 peptide was solubilized in 20% DMSO-phosphate buffer and allowed to oxidize to completion (20 h incubation) and then diluted in phosphate buffer and injected at 50 μL/min, at concentrations ranging from 1-40 μg/mL. SPR curve fitting analysis was performed using global fit of multiple titrations to the 1:1 Langmuir model. All data analysis was performed using the BIAevaluation 4.1 analysis software (GE Healthcare).

Circular Dichroism Analysis of V1 V2 Peptides.

Circular dichroism (CD) spectra of V1/V2 peptides were measured on an Aviv model 202 spectropolarimeter using a 1 mm path length quartz cuvette. The 20% DMSO-treated peptides were dialyzed against 20 mM phosphate buffer, pH 7.0 to remove DMSO using a dialysis cassette of MW cut-off 3500 Da. The CD spectra of peptides (at 100 200 μg/ml concentration) in phosphate buffer (pH 7.4) were recorded at 25° C. Three scans of the CD spectra of each peptide were averaged and the CD signal from phosphate buffer was subtracted out.

Results

Biophysical Characterization of Synthetic V1V2 Peptides.

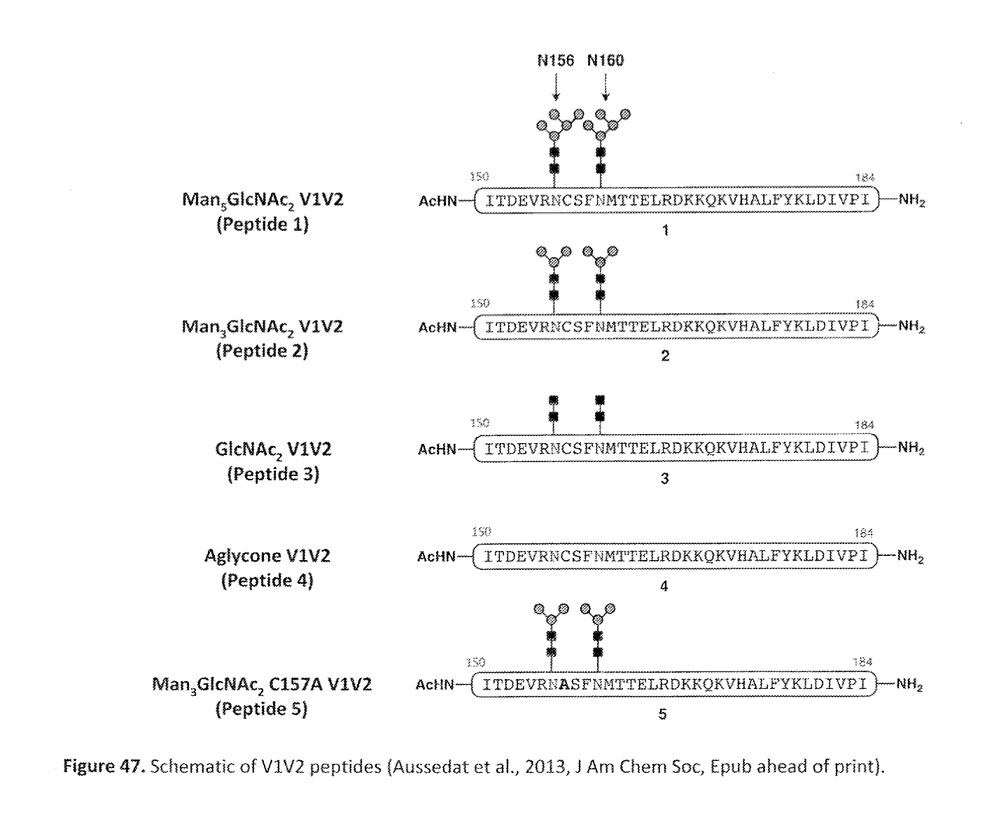

The V1V2 glycopeptides were chemically synthesized as described previously (Aussedat et al, JACS [Epub ahead of print] (2013)) (FIG. 47). These glycopeptides included two glycans with either a terminal mannose₃ GlcNAc₂ (Man₃ V1V2) or a mannose₅GlcNAc₂ (Man₅ V1V2) glycan at the two key N-linked glycosylation sites (Asn¹⁶⁰ and Asn¹⁵⁶) to which PG9 and CH01 V1V2 BnAbs bind (Walker et al, Science 326(5950):285-289 (2009)) (FIG. 47). Two additional V1V2 peptides, one with no glycans (aglycone V1V2) and a second with only the proximal GlcNAc₂ units but with no outer mannose residues (GlcNAc₂ V1V2) were used as controls (Aussedat et al, JACS [Epub ahead of print] (2013)). With these well-defined, biologically promising homogeneous compounds in hand, a question was whether the thiol group at cysteine-157 in these constructs might play a role in their interactions with V1V2 BnAbs. Fortunately, it was not necessary to build a new construct, de novo, to ask this question. Rather, peptide 2 could be readily desulfurized, producing its alanine counterpart peptide 5 (FIG. 47). It was hypothesized that this cysteine to alanine mutation disrupted the active structure responsible for the binding characteristics and that the active structure was not as shown in peptide 1 but rather its oxidized cysteine dimer.

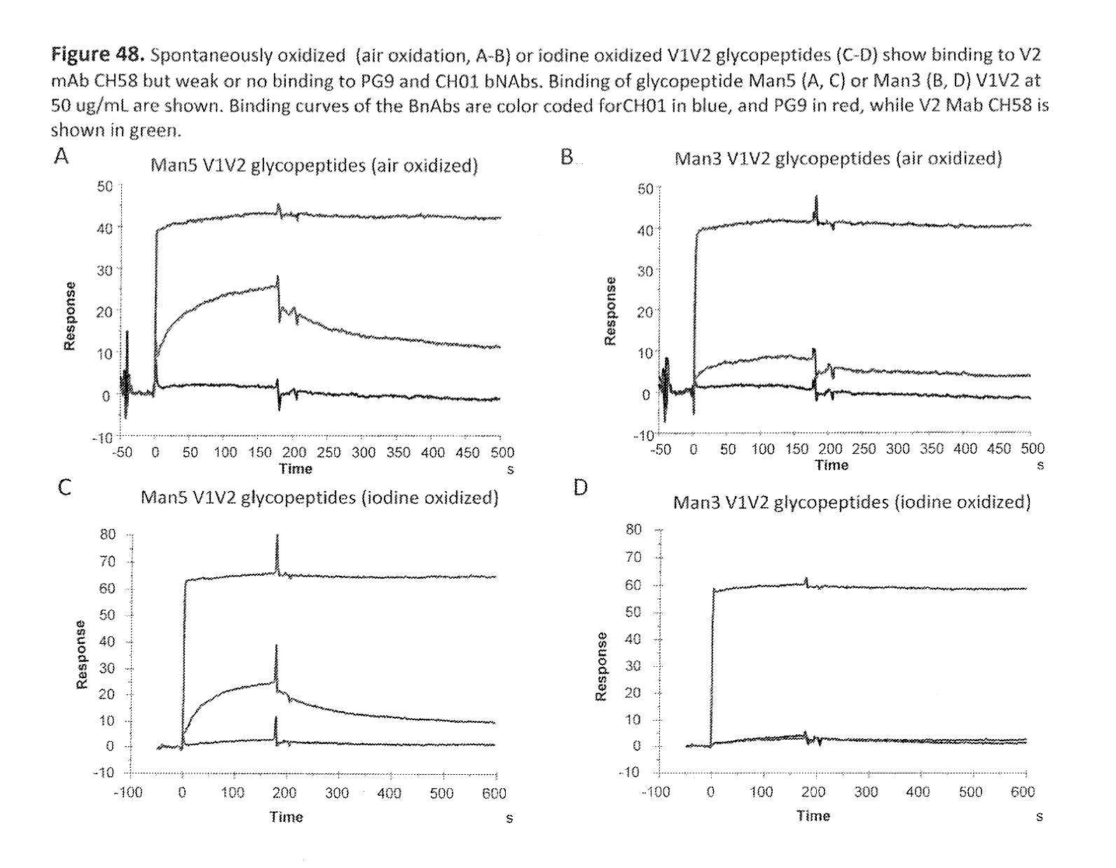

It was initially observed that the synthetic V1V2 peptides could spontaneously undergo air oxidation and formed disulfide-linked dimers. However, when solubilized in phosphate buffer, the V1V2 glycopeptides gave variable, batch-dependent binding results with the BnAbs PG9 and CH01, frequently showing weaker or no binding to the BnAbs and binding more strongly to the V2 mAb CH58 (FIG. 48). To determine whether dimer formation using alternative oxidation protocols might result in more consistent BnAb binding to V1V2 glycopeptides, two different oxidizing agents were tested: iodine and DMSO. The iodine treated V1V2 Man3 glycopeptide bound to the V2 mAb CH58 but not to the BnAbs CH01 or PG9 (FIG. 48). Similarly the iodine-oxidized Man5 glycopeptide showed no binding to CH01 and weak binding to PG9 but bound more strongly to CH58. Size-exclusion chromatography (SEC) analysis showed that iodine treatment resulted in formation of higher order oligomers and aggregates of the glycopeptides, thus suggesting that iodine treatment did not provide stable (i.e., unaggregated) dimer forms of the V1V2 glycopeptides.

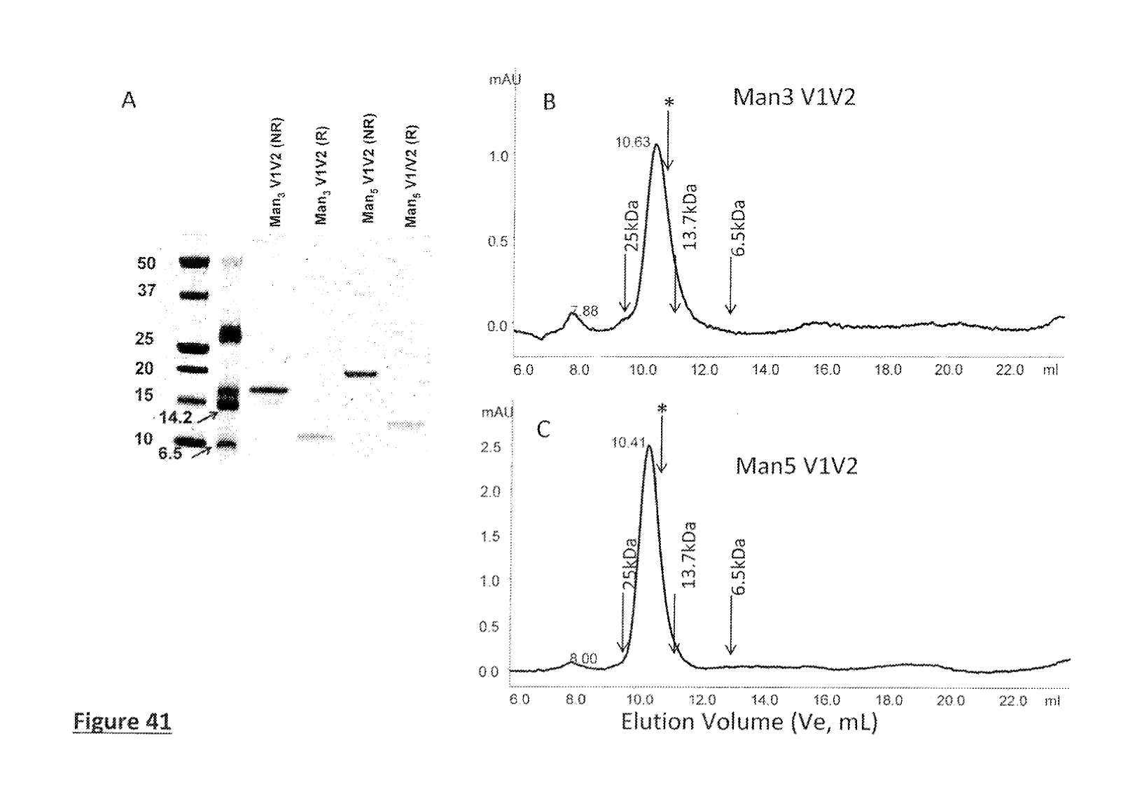

When solubilized in 20% DMSO-phosphate buffer, the glycan-derivatized, Man₃ V1V2 and Man₅ V1V2 glycopeptides were completely oxidized to disulfide-linked dimers (FIG. 41, Experimental Details) (Tam et al, J. Am. Chem. Sco. 113:6657-6662 (1991)). SDS-PAGE analysis of the DMSO-treated Man₃ V1V2 and Man₅ V1V2 peptides confirmed that the constructs had completely dimerized (FIG. 41A). Under reducing conditions, the dimers completely reverted to the monomeric state, consistent with a linkage via disulfide bond formation (FIG. 41A). SEC analysis also showed that both Man3 V1V2 and Man5 V1V2 glycopeptides were dimeric by size, with no detectable monomeric forms or higher order oligomers (FIGS. 41B, 41C), and therefore DMSO-treatment provided stable unaggregated disulfide-linked dimer forms of the V1V2 glycopeptides. Most importantly, the differential binding of DMSO-oxidized glycopeptides to V1V2 BnAbs versus strain-specific V2 mAbs was reversed in favor of the BnAbs (FIG. 42). Following DMSO treatment, both Man5- and Man3 V1V2 glycopeptides bound more strongly to the BnAbs PG9 and CH01, while showing weak binding signal (even at high peptide concentration of 50 μg/mL) to the V2 mAb CH58. By contrast, the binding of the V2 mAb CH58 was retained for the GlcNAc₂- and aglycone V1V2 peptides, while no binding of the V1V2 BnAbs to either of the non-mannosylated V1V2 peptides was observed (FIGS. 42C, 42D). Thus DMSO treatment of the V1V2 glycopeptides provided stable dimer formation and gave selective binding of the V1V2 peptides to the BnAbs PG9 and CH01 over the strain-specific V2 antibodies. The invention contemplates any suitable agent which promotes adoption of an ordered secondary structure of Man5- and Man3 V1V2 glycopeptides as observed after DMSO treatment.

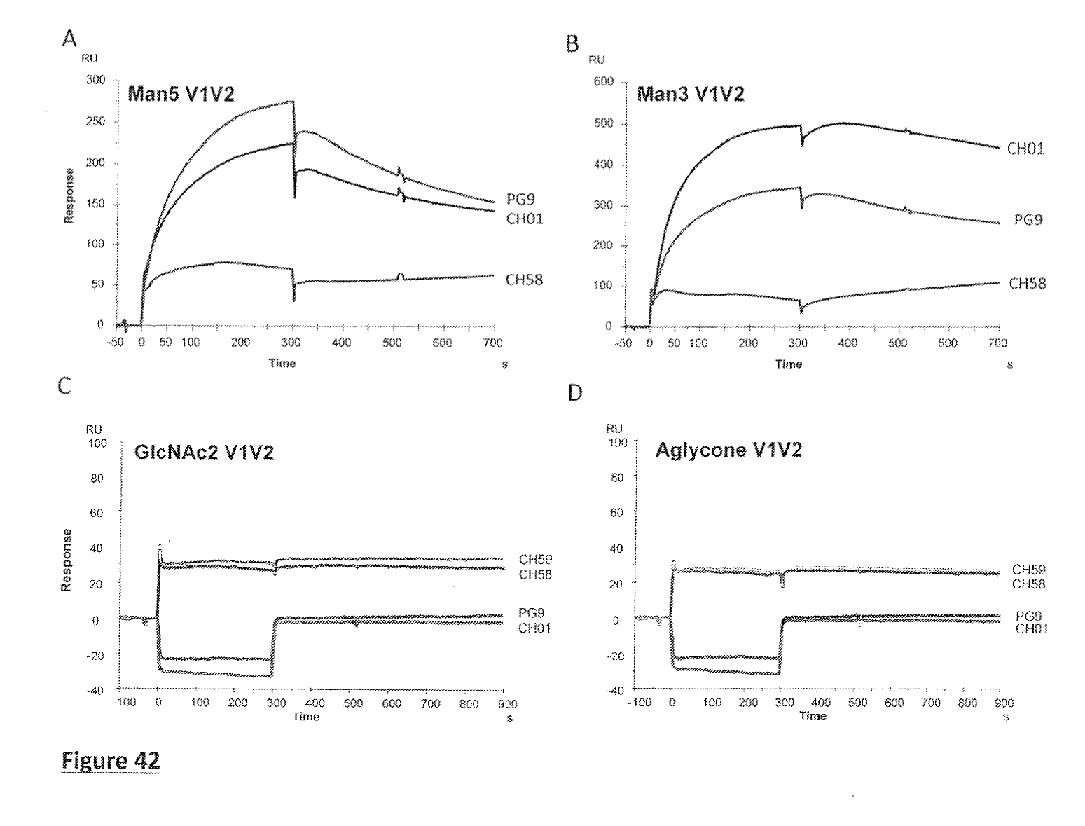

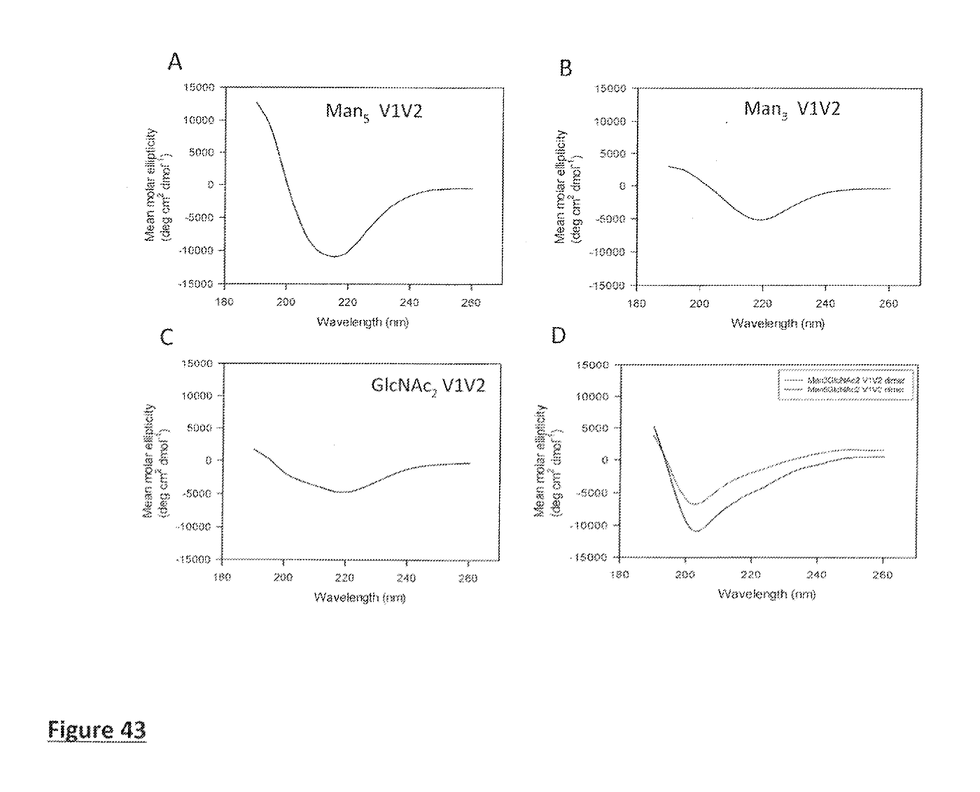

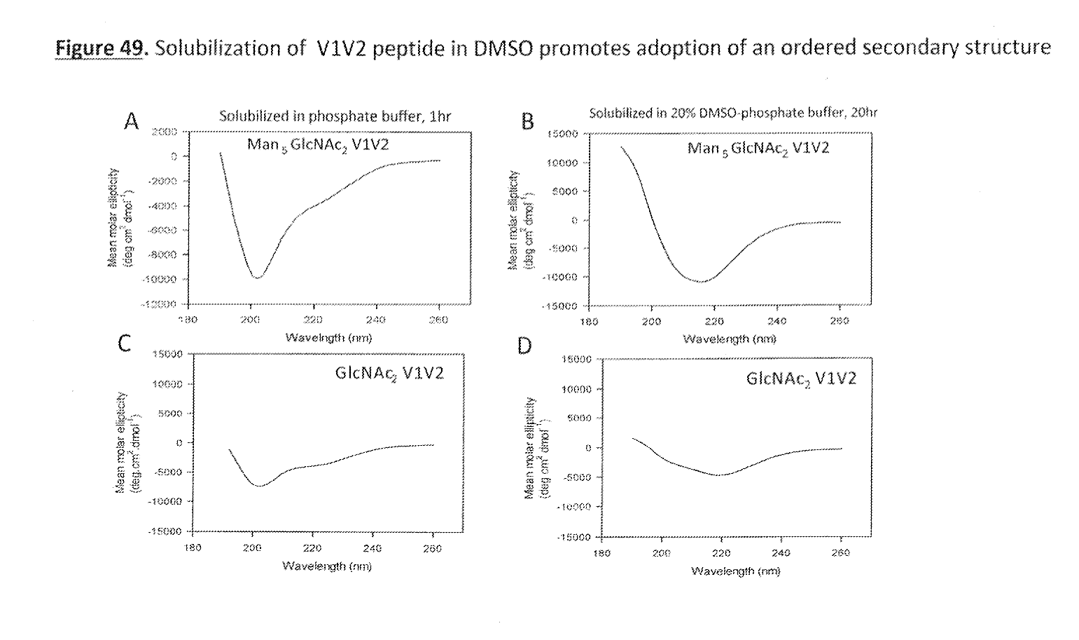

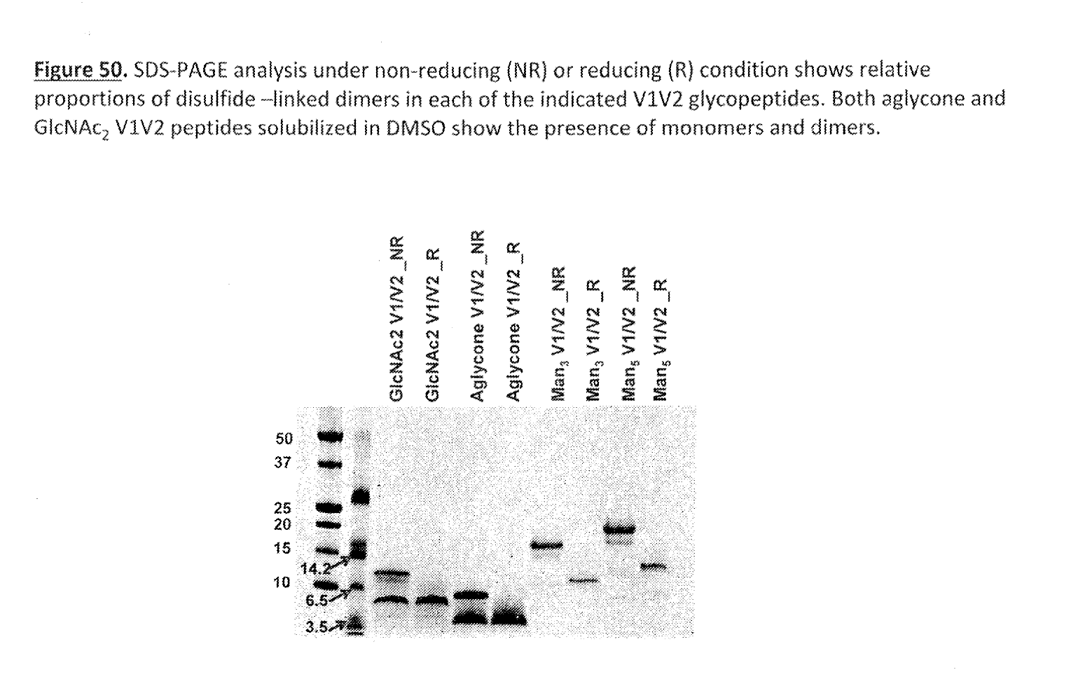

The biophysical properties of the synthetic V1V2 peptides were next analyzed by circular dichroism (CD) analysis to determine whether oxidative dimerization following DMSO treatment resulted in adoption of secondary structure by the V1V2 glycopeptides. CD spectral analysis showed that the V1V2 glycopeptides (with Man₃ or Man₅ glycans) adopted an ordered secondary structure, with spectra exhibiting a strong minimum at 218 nm and a maximum near 195 nm (FIGS. 43A, 43B), characteristics typically observed with peptides with β-sheet conformation (Greenfield, Nat. Protocols 1:2876-2890 (2007)). This β-sheet signature was reliably observed only when the glycopeptides had been treated with DMSO-containing buffer (the DMSO was removed prior to CD measurement, see Experimental Details). When exposed to aqueous buffer alone or when treated with iodine, the CD profile of the glycopeptides was dominated by a strong negative deflection around 200 nm (FIG. 43D, FIG. 49), which is consistent with predominant random coil content (Greenfield, Nat. Protocols 1:2876-2890 (2007)). Similarly the GlcNAc₂-linked peptide displayed a more ordered CD spectrum after DMSO treatment, although the typical β-sheet features were less conspicuous relative to the spectra of the mannosylated glycopeptides (FIG. 43C). Poor aqueous solubility of the aglycone V1V2 peptide precluded CD analysis in phosphate buffer. Thus, the V1V2 peptides presented a more ordered structure in solution when treated with the oxidizing agent DMSO, suggesting the possibility that oxidation of the V1V2 peptides promoted disulfide linkage and contributed to the observed secondary structure with β-strand signature and the resulting selective binding of the V1V2 BnAbs.

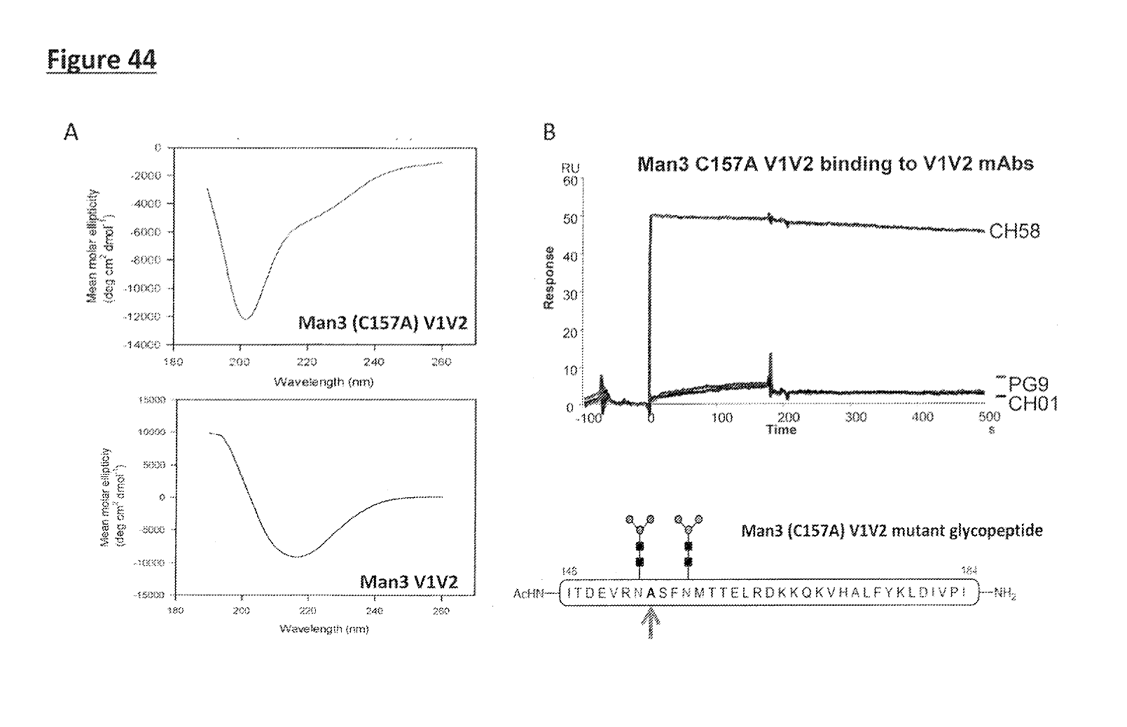

The formation of disulfide-linked dimers was due to the presence a lone Cys residue at position 157 of the V1V2 peptide (FIG. 47). Thus, the requirement of dimer formation of the glycopeptides for binding to BnAbs was further investigated following mutation of Cys¹⁵⁷ to Ala using a chemoselective desulfurization reaction (Wan and Danishefsky, Angew Chem. Int. Ed. Engl. 46(48):9248-9252 (2007)). It was observed that the secondary structure of the resulting mutant Man₃ (C157A) V1V2 glycopeptide reverted to predominantly random coil conformation (FIG. 44A) and resulted in the complete abrogation of the binding of the V1V2 BnAbs and promoted the binding of the V2 mAb CH58 (FIG. 44B). These results are consistent with earlier data showing the glycan dependence of binding of both the V1V2 BnAbs Walker et al, Science 326(5950):285-289 (2009), Bonsignori et al, J. Virol. 85(19):9998-10009 (2011)) and suggested that dimerization and β-sheet secondary structure of V1V2 glycopeptides are important for V1V2 BnAb recognition.

Antigenicity of V1 V2 Peptides for Mature BnAbs.

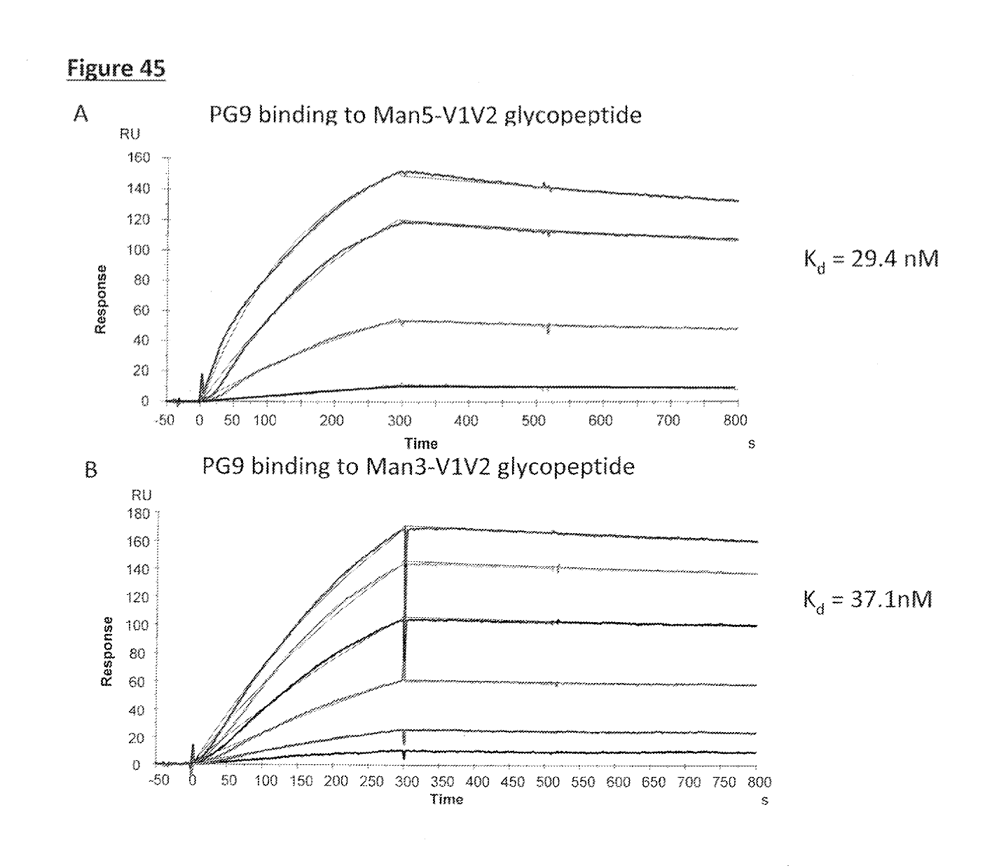

Both V1V2 BnAbs PG9 (Aussedat et al, JACS [Epub ahead of print] (2013)) and CH01 bound only to the synthetic Man3 V1V2 and Man5 V1V2 glycopeptides and not to the aglycone or the GlcNAc₂ V1V2 glycopeptides (FIG. 42). The binding affinities of the V1V2 BnAbs PG9 and CH01 were measured using Man3 or Man5 V1V2 glycopeptides following complete oxidation in DMSO. The V1V2 BnAb PG9 bound to the fully oxidized Man5 and Man3 glycopeptides with K_{d} values of 29 and 37 nM respectively (FIGS. 45A, 45B, Table 1). CH01 BnAb also bound to Man5 and Man3 V1V2 glycopeptides with similar affinities (K_{d}=46 nM and 32 nM respectively) (FIGS. 45C, 45D, Table 1). While both PG9 and CH01 bound to Man3 V1V2 glycopeptide with similar affinities, the binding affinity of PG9 to Man5 V1V2 glycopeptide was about 2-fold higher than that of CH01 mAb binding to the same peptide. Although detectable, the binding of CH58 to either Man3 or Man5 V1V2 glycopeptide did not show a dose dependence, and therefore a Kd could not be reliably measured (FIGS. 45E-45F). Thus the synthetic V1V2 glycopeptides with high mannose glycans (N160/N156) and with secondary structure stabilized by disulfide-linked dimer formation bound selectively and more avidly to both V1V2 BnAbs.

TABLE 1
SPR affinities and kinetics of
V1V2 BnAbs for binding to V1V2 glycopeptides.

| | | kₐ | k_{d} | |
|---|---|---|---|---|
| | | M⁻¹s⁻¹ | s⁻¹ | K_{d} |
| mAb | V1V2 glycopeptide | (×10³) | (×10⁴) | nM |
| PG9 | Man5 GlcNAc2 | 9.2 | 2.71 | 29.4 |
| PG9 UCA | | 7.41 | 7.23 | 97.6 |
| CH01 | | 6.64 | 3.02 | 45.5 |
| CH01 UCA | | 8.49 | 7.21 | 118.0 |
| PG9 | Man3 GlcNAc2 | 3.97 | 1.47 | 37.1 |
| PG9 UCA | | 5.33 | 56.6 | 1060 |
| CH01 | | 9.97 | 3.2 | 32.1 |
| CH01 UCA | | 2.06 | 36.3 | 1776 |

SPR kinetics was measured by injecting V1V2 glycopeptides in solution over mAbs captured onanti-IgG immobilized surfaces as described in Methods. Data shown is representative of three measurements. For PG9 UCA and CH01 UCA, the Kd values were derived using the faster components of the dissociation phase.

Binding of V1V2 Glycopeptides to BnAb Unmutated Common Ancestors.

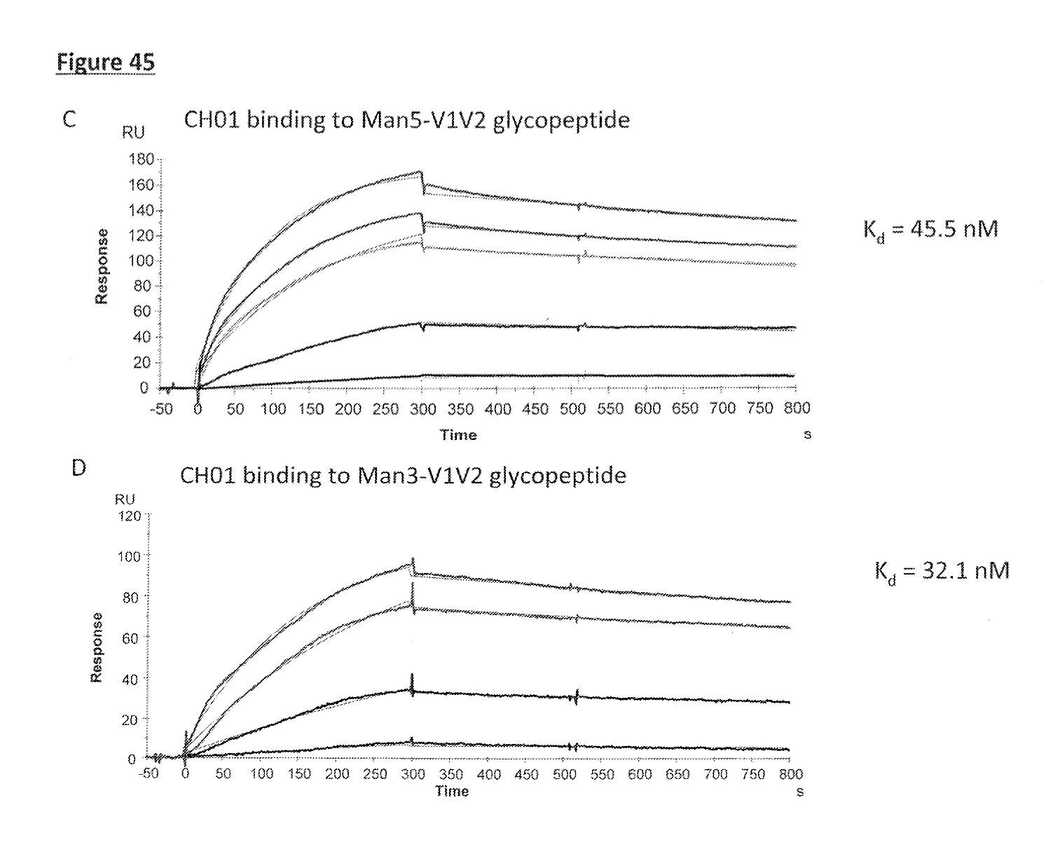

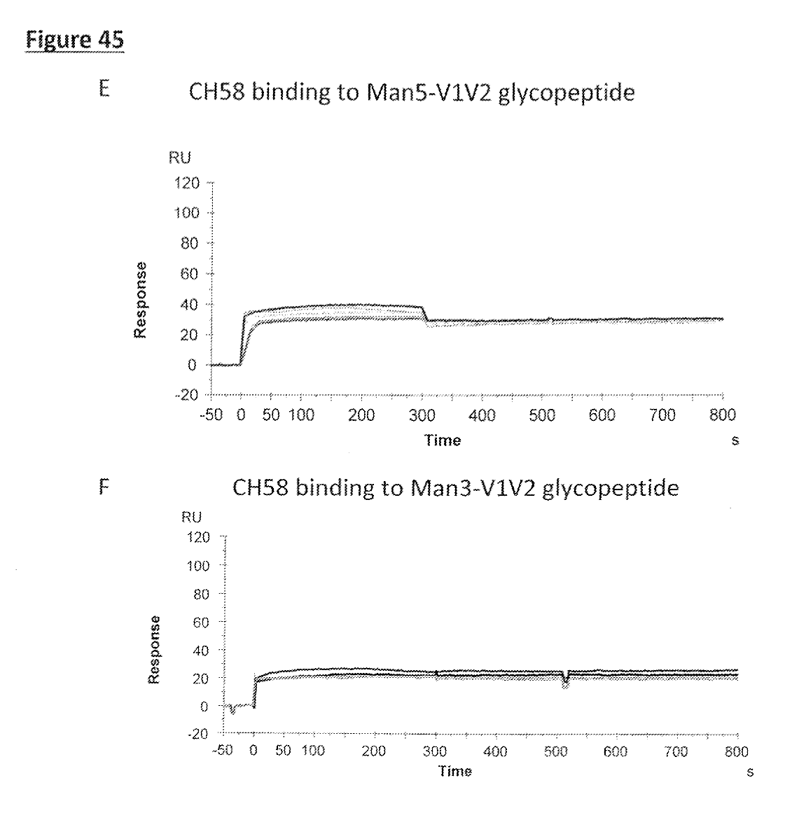

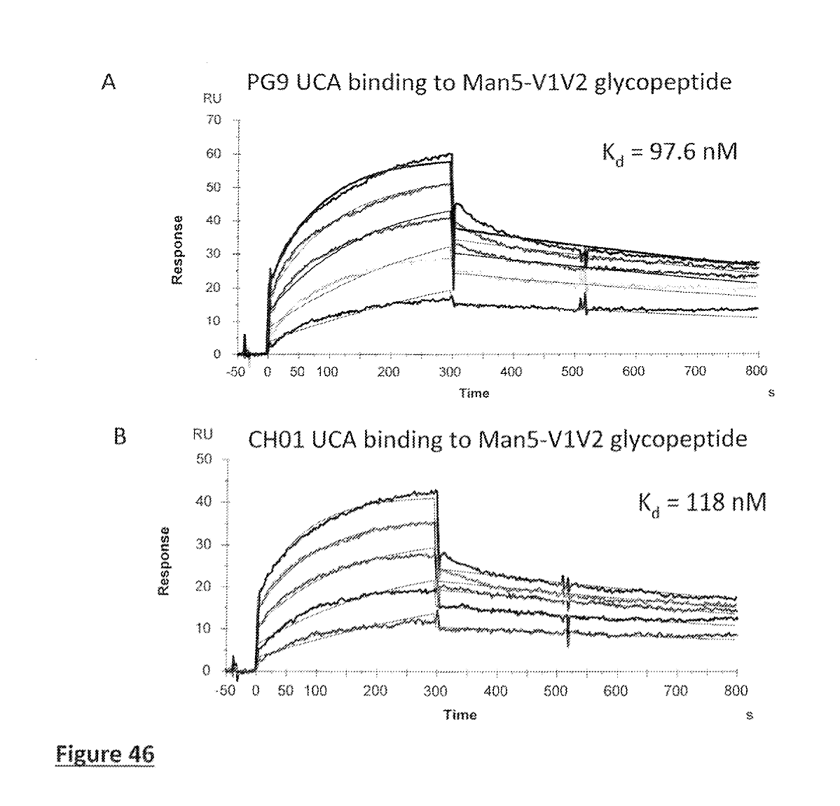

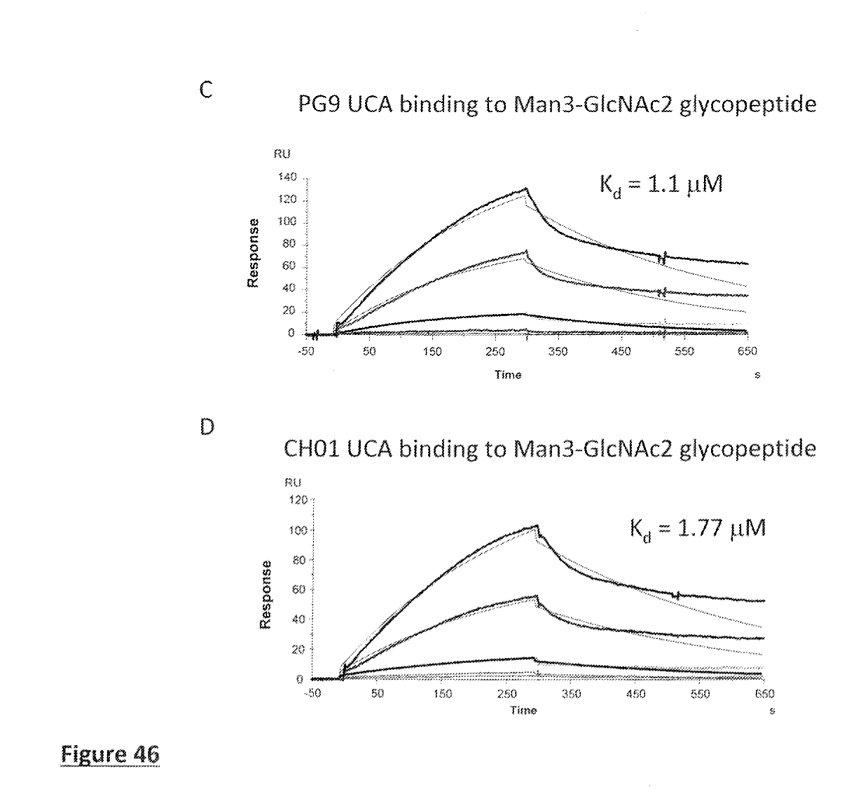

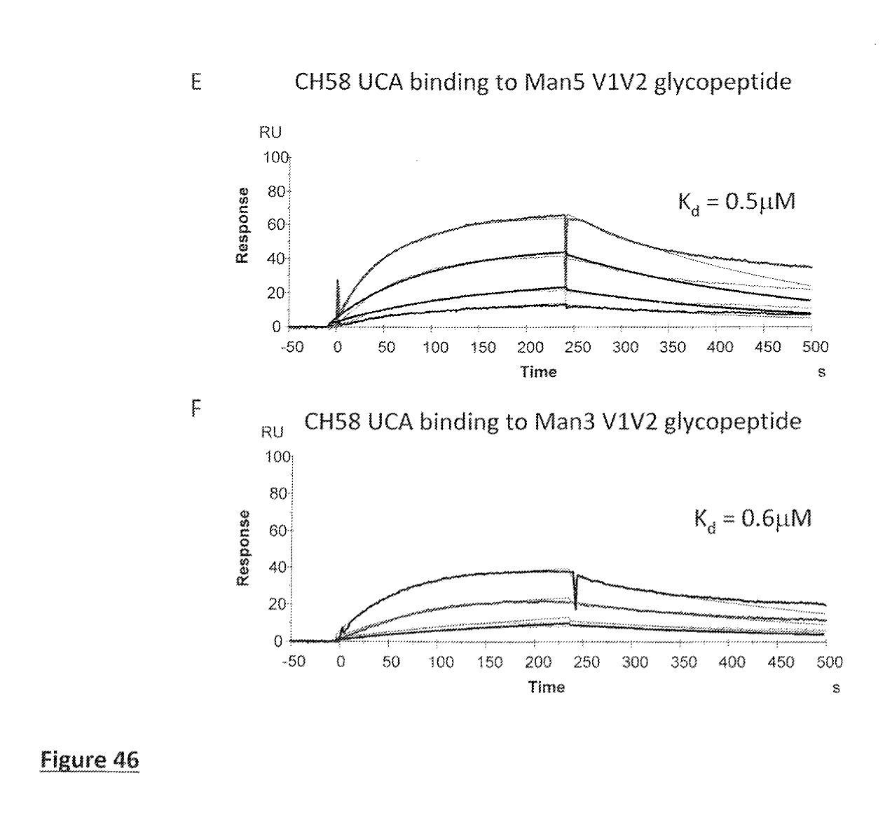

A key characteristic of an immunogen is to not only bind to the mature BnAb but also to bind to the unmutated common ancestors (UCA) of the BnAbs, that are predicted to be the B cell receptors (BCRs) of the BnAb naïve B cell precursors (Haynes et al, Nat. Biotechnol. 30(5):423-433 (2012)). While gp120s have been found that bind the CH01 UCA at K_{d}s of 300 nM to 1 μM (Bonsignori et al, J. Virol. 85(19):9998-10009 (2011)), Alam et al, J. Virol. 87(3):1554-1568 (2013)), no Env construct has been reported that binds to the PG9 UCA. Importantly, both the Man3 and Man5 V1V2 glycopeptides bound the UCAs of PG9 and CH01 (FIG. 46). The binding of both PG9 UCA and CH01 UCA was an order of magnitude stronger to the Man5 V1V2 glycopeptide (K_{d}=98 and 118 nM to PG9 UCA and CH01 UCA, respectively, Table 1) than the Man3-derivatized V1V2 glycopeptide (K_{d}=1.1 and 1.8 μM to PG9 UCA and CH01 UCA, respectively) (FIG. 46, Table 1). The binding of both PG9 and CH01 UCAs gave biphasic dissociation rates, and the rate constant values of the faster component of the dissociation rates showed that both PG9 UCA and CH01 UCA formed complexes with Man5 V1V2 peptide that were more stable than those formed with the Man3 V1V2 glycopeptide (Table 1).

When compared to the mature BnAbs, the binding of both PG9 UCA and CH01 UCA to the Man5 V1V2 glycopeptides was about 3-fold weaker, with the UCAs showing faster dissociation-rates (Table 1). Glycopeptide binding of both PG9 UCA and CH01 UCA required the presence of terminal high-mannose residues since neither UCAs bound to either aglycone V1V2 nor the GlcNAc2 V1V2 peptides. While the PG9 and CH01 UCAs bound to the Man3 V1V2 glycopeptide equally as well as to the mans V1V2 glycopeptide, binding affinities of the PG9 and CH01 UCAs to Man3 was a log less when compared to their binding to Man5 V1V2 glycopeptide (Table 1). Thus, the UCAs specifically required Man5-derivatized V1V2 glycopeptides for optimal binding whereas the mature antibodies did not.

In contrast to the mature strain-specific CH58 mAb, the CH58 UCA showed dose dependent binding to the mannose-derivatized V1V2 glycopeptides (FIGS. 46E, 46F). CH58 UCA bound to both V1V2 glycopeptides with K_{d} values of 0.5 and 0.6 μM for Man5 and Man3 V1V2 respectively. By contrast, the BnAb UCAs and the V2 CH58 UCA bound with similar and weaker affinities to Man3 V1V2, but the UCAs of both PG9 and CH01 bound to Man5 V1V2 with higher affinities (5-fold) than the UCA of CH58 (FIG. 46, Table 1). Thus, Man5-derivatized V1V2 glycopeptides showed higher affinity binding to the UCAs of the sub-dominant BnAbs than the UCA of the strain-specific vaccine-induced V2 mAb.

In summary, reported above is a homogeneous synthetic HIV-1 Env V1V2 Man5 glycopeptide capable of binding with apparent nM affinities to both mutated HIV-1 Envelope V1V2 BnAbs and to their UCAs. A rational strategy for vaccine induction of BnAbs has been proposed to target the UCAs and Intermediate antibodies (IA) of BnAb lineages (Haynes et al, Nat. Biotechnol. 30(5):423-433 (2012), Chen et al, Human Retrovirol. 24:11-12 (2008), Dimitrov, Mabs 2(3):347-356 (2010)). Key to this work is the availability of synthetically-derived homogeneous immunogens that display only sub-dominant BnAb epitopes to maximize the opportunity for BnAb B cells to make a robust germinal center response in the absence of dominant competing strain-specific neutralizing B cell lineages (Haynes et al, Nat. Biotechnol. 30(5):423-433 (2012)). The V1V2 Man5 glycopeptides preferentially bound with nM Kds to the V1V2 BnAbs, including their UCAs. The designed synthetic V1V2 glycopeptides exhibit enhanced expression of V1V2 BnAb epitopes by providing both homogenous expression of the critical glycans and restricting the plasticity of the V1V2 peptide backbone to favor the epitope conformation recognized by V1V2 BnAbs over dominant strain-specific linear peptide epitopes.

It has previously been shown that the gp120 V1V2 region recombinant proteins can present multiple conformations to B cells, and V1V2 BnAbs and strain-specific mAbs may bind to conformationally distinct forms of V1V2 (Liao et al, Immunity 38(1):176-186 (2013), McLellan et al, Nature 480(7377):336-343 (2011)). The plasticity of the V1V2 region and the heterogeneity associated with recombinantly-produced proteins poses a challenge for vaccine design. Recombinantly produced gp120 proteins are prone to aberrant dimer formation that can mask sub-dominant BnAb epitopes (Alam et al, J. Virology 87(3):1554-1568 (2013) Epub 2012 Nov. 21, Finzi et al, J. Virol. Methods 168:155-161 (2010)). Furthermore, differential glycosylation results in structural heterogeneity, including protein misfolding, that can exist in the gp120 protein and specialized V1V2-scaffold constructs. Thus, one factor for the inability of recombinantly-produced Env proteins to induce BnAbs could be due to heterogeneous glycosylation and the sub-optimal representation of the glycosylated molecular form that mirrors that of the native trimer. The precise conformation of the V1V2 BnAb epitopes in the context of the native Env trimer awaits determination. That V1V2 glycopeptides reported here bind with K_{d}s in the nM range suggest their conformational similarity to the epitope on the native Env trimer. Furthermore, the requirement of the adoption of β-strand conformation of the V1V2 glycopeptides for PG9 binding is consistent with the reported structure of the PG9 binding to scaffolded V1V2 (McLellan et al, Nature 480(7377):336-343 (2011)). However, the V1V2 described by McLennan et al (Nature 480(7377):336-343 (2011)) consists of four anti-parallel β-strands that are stabilized by a pair of inter-strand disulfide bonds. The V1V2 glycopeptides described here are shorter in length (excludes the A or D strand sequences) and include a single cys residue allowing the peptides to form disulfide-linked dimers and thereby present a β-strand conformation. It would be of interest to determine whether the cationic 3-conformation of the V2 C strand and the mannose glycans are positioned favorably in the glycopeptide dimer and thus account for the avid PG9 binding to Man5 V1V2 glycopeptide. Although how similar the V1V2 glycopeptide bound complex is to the McLellan scaffolded V1V2 can only be resolved by structural data. Thus, structures of PG9 and/or CH01 with the Man5 V1V2 glycopeptide will be informative.

Binding of either PG9 or CH01 to Man₃GlcNAc₂ or Man₅GlcNAc₂ glycans was not detected in the absence of the V2 peptide backbone (Aussedat et al, JACS [Epub ahead of print] (2013)), suggesting that the binding to oligomannose glycans alone is very weak; this conclusion is consistent with the reported inability of the sugars themselves to inhibit V1V2 BnAb binding (Doores and Burton, J. Virol. 84(20):10510-10521 (2010)) and the 1.6 mM K_{d} of PG9 binding to Man₅GlcNAc₂-Asn when measured using the more sensitive saturation transfer difference nuclear magnetic resonance technique (McLellan et al, Nature 480(7377):336-343 (2011)). However, the peptide-linked oligomannose units are required for PG9 and CH01 BnAb binding when presented in the context of the V1V2 backbone. In addition, it was found that disulfide-linked dimer formation was required for the V1V2 BnAbs, but not for the V2 mAb CH58. The sensitivity of BnAb binding to Cys mutation suggests that the N¹⁶⁰ and N¹⁵⁶ glycans are perhaps spatially positioned more favorably in a dimer, thereby allowing for higher avidity binding or recognition of glycans on two V1V2 units. Asymmetric binding to adjacent V1V2 elements has been proposed in a recent model (Julien et al, Proc. Natl. Acad. Sci. USA 110:4351-6 (2013)) to explain the preferential binding of PG9 to Env trimers (Walker et al, Nature 477(7365):466-470 (2011)). It was also found that the introduction of the Cys to Ala mutation resulted in the loss of the secondary structure of the Man3 glycopeptide. The data demonstrate a clear role for the thiol of the Cys side chain in promoting/stabilizing the conformation of the glycopeptides via disulfide-linked dimer formation. Indeed, a known strategy for stabilization of designed n-sheet forming peptides in aqueous solutions involves dimerization (face-to-face or edge-to-edge) and intermolecular disulfide linkage (Khakshoor and Nowick, Org. Lett. 11:3000-3003 (2009), Quinn et al, Proc. Natl. Acad. Sci. USA 91(19):8747-8751 (1994), Venkatraman et al, Am. Chem. Soc. 124(18):4987-4994 (2002), Yan and Erickson, Protein Sci. 3(7):1069-1073 (1994), May et al, Protein Sci. 5(7):1301-1315 (1996)), so the observed secondary structure preferences could very well be due to a similar phenomenon. In this case, the best results were obtained when the oxidative dimerization was performed in aqueous DMSO. The quantitatively dimerized constructs exhibited BnAb affinities that were significantly improved over earlier results with glycopeptides that had not been deliberately treated with oxidizing agent (Aussedat et al, JACS [Epub ahead of print] (2013)). Interestingly, the DMSO appears to play an additional role, since other oxidation protocols, such as treatment with iodine, resulted in material that was largely unstructured in solution and bound minimally to V1V2 BnAbs. It is possible that the DMSO co-solvent facilitates proper “folding” of the V1V2 constructs, and mitigates against the known propensity of β-sheet polypeptides to aggregate in solution (Nesloney and Kelly, Bioorg. Med. Chem. 4:739-766 (1996)).

Short peptides generally exist in aqueous solution as an ensemble of conformations, although some sequences are known to display distinct secondary structure preferences (Dyson and Wright, Annu. Rev. Biophys. Biophys. Chem. 20:519-538 (1991)). From the standpoint of immunogen design, some means of rigidifying the V1V2 backbone to induce an intrinsic β-preference would be desirable for targeting the sub-dominant BnAb response. A seemingly straightforward strategy would involve cyclization using an intramolecular disulfide linkage Santiveri et al, Chemistry 14(2):488-499 (2008)). In this regard, Amin and colleagues recently reported the synthesis of monomeric cyclic V2 peptides with glycans at N¹⁶⁰, N¹⁵⁶/N¹⁷³ (Amin et al, Nature Chemical Biology 9(8):521-526 (2013)). Binding affinity of the cyclized peptide constructs for the BnAbs (PG9 and PG16 Fabs) was low in the μM range, binding to BnAb UCAs was not reported, and it is also unclear to what extent the peptides were structured since the solution conformations were not probed spectroscopically. The results presented here suggest a potentially more effective means to promote the desired conformation in V1V2 glycopeptides, i.e., via quarternary structure level interactions involving homodimerization via intermolecular disulfide bond formation.

Finally, for any peptide to be immunogenic it will need the presence of T helper cell determinate epitopes to be present in the peptide design or have a T helper determinant carrier protein conjugated to the V1V2 peptide. In this regard it is important to note that at least two T helper epitopes have been reported in the sequence of our V1V2 glycopeptides, one from amino acids 167-176 (Steers et al, PLoS One 7(8):e42579 (2012)) and another at amino acids 172 through 184 (de Souza et al, J. Immunol. 188(10):5166-5176 (2012)).

Thus, use of chemically-synthesized glycopeptides as described in this study can be a useful strategy for producing V1V2 constructs that preferentially bind to V1V2 BnAbs. Such constructs should serve as rationally-designed immunogens for targeting B cells capable of producing broad neutralizing antibody lineages.

### Example 7

The Man₃GlcNAc₂ V1V2 (“Man₅ V1V2”) glycopeptide will be used in various non-limiting examples of immunogenicity regimens. In one embodiment, Man₅ V1V2 glycopeptide is used in repetitive immunizations intramuscularly (IM) alone with an adjuvant for example but not limited to as a squalene based adjuvant, for example MF59, or a Toll-like receptor 4 agonist, for example GLA/SE (see Baldwin et al. J Immunol; Prepublished online 30 Jan. 2012). In another embodiment, the Man₅ V1V2 glycopeptide will be used as a prime IM prior to IM boost with an V1V2 broad neutralizing epitope such as AE.A244 gp120 (Alam, S M et al. J. Virology 87: 1554-68, 2012). In another embodiment, the Man₅ V1V2 glycopeptide will be used as an IM boost for a prime by AE.A244 gp120. In certain embodiments, the Man₅ V1V2 glycopeptide is administered as a dimer. In other embodiments, the Man5 V1V2 glycopeptide is administered as a monomer.

Skilled artisans can readily determine any suitable adjuvant. In a non-limiting embodiment, the adjuvant is STS+R848+oCpGs (STR8S-C).

### REFERENCES

(ⁱ) Myers, G.; Lenroot, R. AIDS Res. Hum. Retroviruses 1992, 8, 1459-1460.
(ⁱⁱ) (a) Trkola, A.; Purtscher, M.; Muster, T.; Ballaun, C.; Buchacher, A.; Sullivan, N.; Srinivasan, K.; Sodroski, J.; Moore, J. P.; Katinger, H. J. Virology 1996, 70, 1100-1108. (b) Sanders, R. W.; Venturi, M.; Schiffner, L.; Kalyanaraman, R.; Katinger, H.; Lloyd, K. O.; Kwong, P. D.; Moore, J. P. J. Virol. 2002, 76, 7293-7305. (c) Scanlan, C. N.; Pantophlet, R.; Wormald, M. R.; Ollmann Saphire, E.; Stanfield, R.; Wilson, I. A.; Katinger, H.; Dwek, R. A.; Rudd, P. M.; Burton, D. R. J. Virology 2002, 76, 7306-7321. (d) Calarese, D. A.; Scanlan, C. N.; Zwick, M. B.; Deechongkit, S.; Mimura, Y.; Kunert, R.; Zhu, P.; Wormald, M. R.; Stanfield, R. L.; Roux, K. H.; Kelly, J. W.; Rudd, P. M.; Dwek, R. A.; Katinger, H.; Burton. D. R.; Wilson, I. A. Science 2003, 300, 2065-2071.
(ⁱⁱⁱ) For recent reviews, see: (a) Morelli, L.; Poletti, L.; Lay, L. Eur. J. Org. Chem. 2011, 2011, 5723-5777. (b) Wang, L.-X. Carbohydrate-Based Vaccines Against HIV/AIDS. In Glycobiology and Drug Design; Klyosov, A. A., Ed.; ACS Symposium Series 1102; American Chemical Society: Washington, D.C., 2012; pp 157-186.
(^{iv}) (a) Mandal, M.; Dudkin, V. Y; Geng, X.; Danishefsky, S. J. Angew. Chem. Int. Ed. 2004, 43, 2557-2561. (b) Geng, X.; Dudkin, V. Y.; Mandal, M.; Danishefsky, S. J. Angew. Chem. Int. Ed. 2004, 43, 2562-2565. (c) Dudkin, V. Y.; Orlova, M.; Geng, X.; Mandal, M.; Olson, W. C.; Danishefsky, S. J. J. Am. Chem. Soc. 2004, 126, 9560-9562. (d) Krauss, I. J.; Joyce, J. G.; Finnefrock, A. C.; Song, H. C.; Dudkin, V. Y.; Geng, X.; Warren, J. D.; Chastain, M.; Shiver, J. W.; Danishefsky, S. J. J. Am. Chem. Soc. 2007, 129, 11042-11044. (e) Joyce, J. G; Krauss, I. J.; Song, H. C.; Opalka, D. W.; Grimm, K. M.; Nahas, D. D.; Esser, M. T.; Hrin, R.; Feng, M.; Dudkin, V. Y.; Chastain, M.; Shiver, J. W.; Danishefsky, S. J. Proc. Natl. Acad. Sci. U.S.A. 2008, 105, 15684-15689.
(^{v}) Haynes, B. F.; Kelsoe, G.; Harrison, S. C.; Kepler, T. B. Nat. Biotechnol. 2012, 30, 423-433.
(^{vi}) Walker, L. M.; Phogat, S. K.; Chan-Hui, P.-Y.; Wagner, D.; Phung, P.; Goss, J. L.; Wrin, T.; Simek, M. D.; Fling, S.; Mitcham, J. L.; Lehrman, J. K.; Priddy, F. H.; Olsen, O. A.; Frey, S. M.; Hammond, P. W.; Protocol G Principal Investigators; Kaminsky, S.; Zamb, T.; Moyle, M.; Koff, W. C.; Poignard, P.; Burton, D. R. Science 2009, 326, 285-289.
(^{vii}) McLellan, J. S.; Pancera, M.; Carrico, C.; Gorman, J.; Julien, J.-P.; Khayat, R.; Louder, R.; Pejchal, R.; Sastry, M.; Dai, K.; O/'Dell, S.; Patel, N.; Shahzad-ul-Hussan, S.; Yang, Y; Zhang, B.; Zhou, T.; Zhu, J.; Boyington, J. C.; Chuang, G.-Y.; Diwanji, D.; Georgiev, I.; Do Kwon, Y.; Lee, D.; Louder, M. K.; Moquin, S.; Schmidt, S. D.; Yang, Z.-Y; Bonsignori, M.; Crump, J. A.; Kapiga, S. H.; Sam, N. E.; Haynes, B. F.; Burton, D. R.; Koff, W. C.; Walker, L. M.; Phogat, S.; Wyatt, R.; Orwenyo, J.; Wang, L.-X.; Arthos, J.; Bewley, C. A.; Mascola, J. R.; Nabel, G. J.; Schief, W. R.; Ward, A. B.; Wilson, I. A.; Kwong, P. D. Nature 2011, 480, 336-343.
(^{viii}) For recent reviews, see: (a) Yuan, Y.; Chen, J.; Wan, Q.; Wilson, R. M.; Danishefsky, S. J. Biopolymers 2010, 94, 373-384. (b) Wilson, R. M.; Dong, S.; Wang, P.; Danishefsky, S. J. Angew. Chem. Int. Ed. 2013, 52, 7646-7665.
(^{ix}) A recent analysis of the bivalent gp120 subunit vaccine AIDSVAX B/E found 16 isoforms of the clade B immunogen and 24 isoforms of the clade E immunogen: Yu, B.; Morales, J. F.; O'Rourke, S. M.; Tatsuno, G. P.; Berman, P. W. PLoS ONE 2012, 7, e43903.
(^{x}) An abstract was published by Wang referring to work on homogeneous gp120 V1V2 glycopeptides: (a) Wang, L.-X. Abstracts of Papers, 245^{th} National Meeting of the American Chemical Society, New Orleans, Apr. 7-11, 2013; American Chemical Society: Washington, D.C., 2013; CARB-13. The Wang constructs were prepared by chemoenzymatic assembly using semisynthesis-derived glycans, as detailed in a subsequent publication: (b) Amin, M. N.; McLellan, J. S.; Huang, W.; Orwenyo, J.; Burton, D. R.; Koff, W. C.; Kwong, P. D.; Wang, L.-X. Nat. Chem. Biol. 2013, 9, 521-526.
(^{xi}) (a) Miller, J. S.; Dudkin, V. Y.; Lyon, G. J.; Muir, T. W.; Danishefsky, S. J. Angew. Chem. Int. Ed. 2003, 42, 431-434. (b) Warren, J. D.; Miller, J. S.; Keding, S. J.; Danishefsky, S. J. J. Am. Chem. Soc. 2004, 126, 6576-6578.
(^{xii}) The scaffolded V1V2 construct was expressed in HEK 293S GnTI^{-/-} cells, which are unable to process the Man₅GlcNAc₂ intermediate into hybrid and complex-type glycans. The entire Man₅GlcNAc₂ oligosaccharide is evident in the electron density map for the Asn¹⁶⁰ glycan, but only four mannose residues are visible for the Asn¹⁵⁶ glycan.
(^{xiii}) In the context of the Env trimer, PG9 may engage a third glycan on Asn¹⁶⁰ of a neighboring protomer: Julien, J.-P.; Lee, J. H.; Cupo, A.; Murin, C. D.; Derking, R.; Hoffenberg, S.; Caulfield, M. J.; King, C. R.; Marozsan, A. J.; Klasse, P. J.; Sanders, R. W.; Moore, J. P.; Wilson, I. A.; Ward, A. B. Proc. Natl. Acad. Sci. U.S.A. 2013, 110, 4351-4356.
(^{xiv}) An A244 strain recombinant gp120 glycoprotein (as a mixture of glycoforms) was used as part of the vaccine regimen in the recent, modestly efficacious RV144 HW-1 vaccination clinical trial: Rerks-Ngarm, S.; Pitisuttithum, P.; Nitayaphan, S.; Kaewkungwal, J.; Chiu, J.; Paris, R.; Premsri, N.; Namwat, C.; De Souza, M.; Adams, E.; Benenson, M.; Gurunathan, S.; Tartaglia, J.; McNeil, J. G.; Francis, D. P.; Stablein, D.; Birx, D. L.; Chunsuttiwat, S.; Khamboonruang, C.; Thongcharoen, P.; Robb, M. L.; Michael, N. L.; Kunasol, P.; Kim, J. H. N. Engl. J. Med. 2009, 361, 2209-2220.
(^{xv}) Bonsignori, M.; Hwang, K.-K.; Chen, X.; Tsao, C.-Y.; Morris, L.; Gray, E.; Marshall, D. J.; Crump, J. A.; Kapiga, S. H.; Sam, N. E.; Sinangil, F.; Pancera, M.; Yongping, Y.; Zhang, B.; Zhu, J.; Kwong, P. D.; O'Dell, S.; Mascola, J. R.; Wu, L.; Nabel, G. J.; Phogat, S.; Seaman, M. S.; Whitesides, J. F.; Moody, M. A.; Kelsoe, G.; Yang, X.; Sodroski, J.; Shaw, G. M.; Montefiori, D. C.; Kepler, T. B.; Tomaras, G. D.; Alam, S. M.; Liao, H.-X.; Haynes, B. F. J. Virology 2011, 85, 9998-10009.
(^{xvi}) Doores, K. J.; Burton, D. R. J. Virology 2010, 84, 10510-10521.
(^{xvii}) A recent report suggests that there may be greater promiscuity with regard to recognition of the glycan at Asn¹⁵⁶ by PG16 and PG9: Pancera, M.; Shahzad-ul-Hussan, S.; Doria-Rose, N. A.; McLellan, J. S.; Bailer, R. T.; Dai, K.; Loesgen, S.; Louder, M. K.; Staupe, R. P.; Yang, Y.; Zhang, B.; Parks, R.; Eudailey, J.; Lloyd, K. E.; Blinn, J.; Alam, S. M.; Haynes, B. F.; Amin, M. N.; Wang, L.-X.; Burton, D. R.; Koff, W. C.; Nabel, G. J.; Mascola, J. R.; Bewley, C. A.; Kwong, P. D. Nat. Struct. Mol. Biol. 2013, 20, 804-813.
(^{xviii}) All of our V1V2 glycopeptide constructs are chemically different from the glycopeptides prepared by Amin et al. (ref. xb). Although one of their constructs carried Man₅GlcNAc₂ at Asn¹⁶⁰ and Asn¹⁵⁶, we used an Env sequence that was derived from a different HIV-1 strain (A244) than the two variants used in their study (CAP45 and ZM109). Moreover, in our constructs, the peptide domains are longer (35 amino acids versus 24), and we retained the native A244 sequence without mutating any residues or adding any affinity tags. Amin et al. mutated Lys¹⁵⁵ and Phe¹⁷⁶ to Cys, allowing for cyclization of their constructs via disulfide bond formation, and they incorporated a biotin tag at the N-terminus.
(^{xix}) (a) Anisfeld, S. T.; Lansbury, P. T. J. Org. Chem. 1990, 55, 5560-5562. (b) Cohen-Anisfeld, S. T.; Lansbury, P. T. J. Am. Chem. Soc. 1993, 115, 10531-10537.
(^{xx}) Wang, P.; Aussedat, B.; Vohra, Y.; Danishefsky, S. J. Angew. Chem. Int. Ed. 2012, 51, 11571-11575.
(^{xxi}) Ullmann, V.; Rädisch, M.; Boos, I.; Freund, J.; Pohner, C.; Schwarzinger, S.; Unverzagt, C. Angew. Chem. Int. Ed. 2012, 51, 11566-11570.
(^{xxii}) For previous reports detailing the synthesis of the reducing pentasaccharide, see: (a) Paulsen, H.; Lebuhn, R. Carbohydr. Res. 1984, 130, 85-101. (b) Nakahara, Y.; Shibayama, S.; Nakahara, Y.; Ogawa, T. Carbohydr. Res. 1996, 280, 67-84. (c) Wang, Z.-G.; Zhang, X.; Live, D.; Danishefsky, S. J. Angew. Chem. Int. Ed. 2000, 39, 3652-3656. (d) Totani, K.; Matsuo, I.; Ito, Y. Bioorg. Med. Chem. Lett. 2004, 14, 2285-2289. (e) Hagihara, S.; Totani, K.; Matsuo, I.; Ito, Y. J. Med. Chem. 2005, 48, 3126-3129. (f) Wang, Z.-G.; Warren, J. D.; Dudkin, V. Y.; Zhang, X.; Iserloh, U.; Visser, M.; Eckhardt, M.; Seeberger, P. H.; Danishefsky, S. J. Tetrahedron 2006, 62, 4954-4978.
(^{xxiii}) Indeed, we were only able to locate a single report dealing with its preparation, not as the reducing sugar, but suitably functionalized for the construction of carbohydrate microarrays: Sema, S.; Etxebarria, J.; Ruiz, N.; Martin-Lomas, M.; Reichardt, N.-C. Chem. Eur. J. 2010, 16, 13163-13175.)
(^{xxiv}) Interestingly, Cohen-Anisfeld and Lansbury did demonstrate their method using Man₅GlcNAc₂, but it was obtained from the urine of sheep with swainsonine-induced α-mannosidosis (see ref. xixb).
(^{xxv}) (a) Wang, P.; Zhu, J.; Yuan, Y; Danishefsky, S. J. J. Am. Chem. Soc. 2009, 131, 16669-16671. (b) Walczak, M. A.; Danishefsky, S. J. J. Am. Chem. Soc. 2012, 134, 16430-16433. (c) Walczak, M. A.; Hayashida, J.; Danishefsky, S. J. J. Am. Chem. Soc. 2013, 135, 4700-4703.
(^{xxvi}) (a) Crich, D.; Sun, S. J. Org. Chem. 1996, 61, 4506-4507. (b) Crich, D.; Sun, S. J. Org. Chem. 1997, 62, 1198-1199. (c) Crich, D.; Sun, S. J. Am. Chem. Soc. 1998, 120, 435-436. (d) Crich, D.; Sun, S. Tetrahedron 1998, 54, 8321-8348.
(^{xxvii}) Crich, D.; Li. H.; Yao, Q.; Wink, D. J.; Sommer, R. D.; Rheingold, A. L. J. Am. Chem. Soc. 2001, 123, 5826-5828.
(^{xxviii}) Shie, C.; Tzeng, Z.; Kulkarni, S. S.; Uang, B.; Hsu, C.; Hung, S. Angew. Chem. Int. Ed. 2005, 44, 1665-1668.
(^{xxix}) Likhosherstov, L. M.; Novikova, O. S.; Derevitskaja, V. A.; Kochetkov, N. K. Carbohydr. Res. 1986, 146, C1-C5.
(^{xxx}) Dawson, P. E.; Muir, T. W.; Clark-Lewis, I.; Kent, S. B. Science 1994, 266, 776-779.
(^{xxxi}) Kuroda, H.; Chen, Y; Kimura, T.; Sakakibara, S. Int. J. Pept. Prot. Res. 1992, 40, 294-299.
(^{xxxii}) Nagorny, P.; Fasching, B.; Li, X.; Chen, G.; Aussedat, B.; Danishefsky, S. J. J. Am. Chem. Soc. 2009, 131, 5792-5799.
(^{xxxiii}) One equivalent (combined) of MPAA-exchanged and unreacted ethyl thioester can be recovered after the reaction.
(^{xxxiv}) Closely spaced N-linked glycans on cyclic peptide scaffolds have been synthesized previously. For example, see: Sprengard, U.; Schudok, M.; Schmidt, W.; Kretzschmar, G.; Kunz, H. Angew. Chem. Int. Ed. 1996, 35, 321-324.
(^{xxxv}) Work is also in progress to evaluate alternative synthetic strategies that would yield a more “process-friendly” route.
(^{xxxvi}) Alam, S. M.; McAdams, M.; Boren, D.; Rak, M.; Scearce, R. M.; Gao, F.; Camacho, Z. T.; Gewirth, D.; Kelsoe, G.; Chen, P.; Haynes, B. F. J. Immunol. 2007, 178, 4424-4435.
(^{xxxvii}) The conformational consequences of N-glycosylation have been well studied. For reviews, see: (a) Imperiali, B.; O'Connor, S. E. Curr. Opin. Chem. Biol. 1999, 3, 643-649. (b) Meyer. B.; Möller, H. Conformation of Glycopeptides and Glycoproteins. In Glycopeptides and Glycoproteins: Synthesis, Structure, and Application; Wittmann, V., Ed.; Topics in Current Chemistry, Vol. 267; Springer: Berlin, 2007; pp 187-251.
(^{xxxviii}) (a) Live, D. H.; Kumar, R. A.; Beebe, X.; Danishefsky, S. J. Proc. Natl. Acad. Sci. U.S.A. 1996, 93, 12759-12761. (b) Live, D. H.; Wang, Z.-G.; Iserloh, U.; Danishefsky, S. J. Org. Lett. 2001, 3, 851-854.
(^{xxxix}) Similarly, using V1V2 glycopeptide isoforms bearing natively N-linked sugars, Amin et al. (ref. xb) detected an astonishing preference of PG9 for a sialylated complex-type glycan at Asn¹⁵⁶.
(^{xl}) Man₃GlcNAc₂, also known as “paucimannose,” is more characteristically associated with plant and invertebrate glycoproteins, although some have argued that it can be expressed in certain pathological states in mammals, such as inflammation or malignancy: Zipser, B.; Bello-DeOcampo, D.; Diestel, S.; Tai, M.-H.; Schmitz, B. J. Carbohydr. Chem. 2012, 31, 504-518.
(^{xli}) Others have remarked on the limitations of glycan array analysis in delineating BnAb anti-glycan specificities: “Protein-free glycan binding by anti-HIV antibodies is not always detectable; e.g., although PG9 recognizes a gp120-associated high-mannose glycan, no binding to protein-free glycans was detected in microarrays. Thus, although a positive result in a glycan microarray implies involvement of a particular glycan in an antibody epitope, a negative result does not rule out glycan recognition.” For the full discussion, see: Mouquet, H.; Scharf, L.; Euler, Z.; Liu, Y; Eden, C.; Scheid, J. F.; Halper-Stromberg, A.; Gnanapragasam, P. N. P.; Spencer, D. I. R.; Seaman, M. S.; Schuitemaker, H.; Feizi, T.; Nussenzweig, M. C.; Bjorkman, P. J. Proc. Natl. Acad. Sci. U.S.A. 2012, 109, E3268-E3277.
(^{xlii}) As a point of comparison, we note that Amin et al. (ref. xb) prepared more than 25 V1V2 glycopeptides bearing various combinations of high mannose and complex-type glycans, and yet their highest affinity constructs bound PG9 Fab with K_{d}'s of ˜5×10⁻⁶ M.)
(^{xliii}) Walker, L. M.; Huber, M.; Doores, K. J.; Falkowska, E.; Pejchal, R.; Julien, J.-P.; Wang, S.-K.; Ramos, A.; Chan-Hui, P.-Y.; Moyle, M.; Mitcham, J. L.; Hammond, P. W.; Olsen, O. A.; Phung, P.; Fling, S.; Wong, C.-H.; Phogat, S.; Wrin, T.; Simek, M. D.; Principal Investigators, P. G.; Koff, W. C.; Wilson, I. A.; Burton, D. R.; Poignard, P. Nature 2011, 477, 466-470.

All documents and other information sources cited herein are hereby incorporated in their entirety by reference.

1. A synthetic peptide comprising sequence ITDEVRNCSFNMTTELRDKKQKVHALFYKLDIVPI (SEQ ID NO: 1), wherein the peptide is glycosylated at positions Asn156 and Asn160 (underlined).

2. The peptide of claim 1, wherein the peptide is glycosylated with oligomannose.

3. The peptide of claim 1, wherein the peptide has Man5GlcNAc2 glycans at positions N156 and N160 or has Man3GlcNAc2 glycans at positions N156 and N160.

4. A synthetic glycopeptide of Formula Man3GlcNAc2 V1V2 “Compound 2/Peptide2” or of Formula Man5GlcNAc2 V1V2 “Compound 1/Peptide 1”.

5. A peptide dimer consisting essentially of the synthetic glycopeptide of Man₅GlcNAc₂ V1V2 (Peptide 1), wherein the dimer is disulfide-linked via oxidized Cys157.

6. A composition comprising the synthetic peptide of claim 1, wherein the composition comprises purified homogenously glycosylated peptides.

7. The composition of claim 6, wherein the glycosylation pattern is homogenous on all peptides of SEQ ID NO: 1 in the composition.

8. The composition of claim 6, wherein the peptide comprises an oxidized Cys157.

9. The composition of claim 6, wherein the peptide is a dimer.

10. The composition of claim 9, wherein the dimer consists essentially of Peptide 1.

11. The composition of claim 6, wherein the dimer is disulfide-linked via oxidized Cys157.

12. The composition of claim 6, wherein the composition is immunogenic.

13. A method of inducing antibodies against HIV-1 in a subject, the method comprising administering to the subject the composition of claim 6 in an amount sufficient to induce the anti-HIV-1 antibodies.

14. The method of claim 13, wherein the composition comprises Man5GlcNAc2 V1V2 as a dimer and an adjuvant.

15. The method of claim 14, wherein the dimer is disulfide-linked via oxidized Cys157.

16. The method of claim 13, wherein the composition is administered as a prime, boost, or both.

17. An isolated antibody which binds to the peptide of claim 1, wherein the antibody does not bind to the non-glycosylated peptide of SEQ ID NO: 1 (Aglycone V1V2 peptide of SEQ ID NO: 1).

18. The isolated antibody of claim 17, wherein the antibody binds to a peptide dimer consisting essentially of the synthetic glycopeptide of Man5GlcNAc2 V1V2 (Peptide 1), wherein the dimer is disulfide-linked via oxidized Cys157, and wherein the antibody does not bind to non-glycosylated peptide of SEQ ID NO: 1.

19. A method for synthesizing the peptide of claim 1, comprising ligating glycopeptide N-terminal fragment 22 and glycopeptide C-terminal fragment 24 in NCL buffer and neutral TCEP solution (Scheme 5 step (e)).

20. A method for synthesizing glycopeptide N-terminal fragment 22, comprising joining the carboxylic acid side chain at position 156 of the thioester peptide ITDEVRD (fragment 21 Scheme 5) to Man₅GlcNAc₂ (heptasaccharide 18) in the presence of PyAOP, DIEA, DMSO, optionally lyophilizing the mixture, and precipitating the glycopeptide by a treatment with 85:5:5:2 TFAphenol/water/triisopropylsilane (Scheme 5).

21. A method for synthesizing glycopeptide C-terminal fragment 24, comprising joining the side chain at position 160 of the peptide of fragment 23 (Scheme 5) to Man₅GlcNAc₂ (heptasaccharide 18) in the presence of PyAOP, DIEA, DMSO, quenching the reaction in TFA, optionally lyophilizing the mixture, and precipitating the glycopeptide by a treatment with 90:5:3:2 TFA/thioanisole/ethanedithiol/anisole (Scheme 5 step (c, d)).

22. A composition comprising the synthetic peptide of claim 2, wherein the composition comprises purified homogenously glycosylated peptides.

23. A composition comprising the synthetic peptide of claim 3, wherein the composition comprises purified homogenously glycosylated peptides.

24. A composition comprising the synthetic peptide of any 4, wherein the composition comprises purified homogenously glycosylated peptides.

25. A composition comprising the synthetic peptide of claim 5, wherein the composition comprises purified homogenously glycosylated peptides.

26. An isolated antibody which binds to the dimer of claim 5, wherein the antibody does not bind to the non-glycosylated peptide of SEQ ID NO: 1 (Aglycone V1V2 peptide of SEQ ID NO: 1).
